(19) United States
(12) Patent Application Publication
(10) Pub. No.: US 2023/0100003 A1
(43) Pub. Date: Mar. 30, 2023

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Ji-Song JUN, Gyeonggi-do (KR); Hong-Se OH, Gyeonggi-do (KR); Young-Kwang KIM, Gyeonggi-do (KR); So-Mi PARK, Gyeonggi-do (KR); Dong-Hyung LEE, Gyeonggi-do (KR); Tae-Jun HAN, Gyeonggi-do (KR)

(21) Appl. No.: 17/833,783

(22) Filed: Jun. 6, 2022

(30) Foreign Application Priority Data
Jul. 14, 2021 (KR) 10-2021-0092041
May 23, 2022 (KR) 10-2022-0062771

(51) Int. Cl. H01L 51/00

(52) U.S. Cl. CPC H01L 51/0036; H01L 51/0073; H01L 51/0069; H01L 51/0054; H01L 51/0072

(57) ABSTRACT

The present disclosure relates to an organic electroluminescent compound represented by formula 1 and an organic electroluminescent device comprising the same. By comprising the compounds according to the present disclosure, it is possible to provide an organic electroluminescent device having improved luminous efficiency and/or lifetime properties.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) consists of a multi-layer structure including a hole injection layer, a hole transport layer, a light-emitting layer, an electron transport layer, and an electron injection layer, etc., in order to improve its efficiency and stability. In this case, the selection of a compound included in the hole transport layer or the like is recognized as one of the means for improving device properties such as the hole transport efficiency to a light-emitting layer, the luminous efficiency, and the lifetime.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used as a hole injection and transport material in an OLED. However, an OLED prepared using these materials have problems of reduction in quantum efficiency and lifetime. This is because, when an OLED is driven under high current, thermal stress occurs between an anode and a hole injection layer, thereby such thermal stress significantly reduces the lifetime of the device. Further, since the organic material used in the hole injection layer has very high hole mobility, there have been problems in that the hole-electron charge balance is broken and the quantum efficiency (cd/A) is lowered.

Therefore, the development of a hole transport material for improving the performance of an OLED is still required.

Korean Patent Application Laid-Open No. 10-2016-0078526 discloses organic selenium compounds including dibenzoselenophene, benzo[b]selenophene, or benzo[c]selenophene, but there is no example in which such compounds are used in a hole transport zone.

### DISCLOSURE OF INVENTION

### Technical Problem

The object of the present disclosure is firstly, to provide an organic electroluminescent compound effective for producing an organic electroluminescent device having improved luminous efficiency and/or lifetime properties, and secondly, to provide an organic electroluminescent device comprising the organic electroluminescent compound.

### Solution to Problem

As a result of intensive studies to solve the technical problem above, the present inventors found that the aforementioned objective can be achieved by an organic electroluminescent compound represented by the following formula 1, so that the present disclosure was completed.

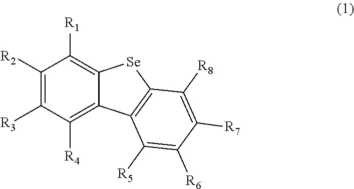

In formula 1,

R₁ to R₄, each independently, represent hydrogen or deuterium;

R₅ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or

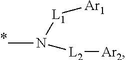

with a proviso that at least one of R₅ to R₈ represents

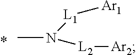

L₁ and L₂, each independently, represent a single bond, a substituted or unsubstituted (C1-C30)alkylene, a substituted or unsubstituted (C6-C30) aryiene, a substituted or unsubstituted (3- to 30-membered)heteroarylene, or a substituted or unsubstituted (C3-C30)cycloalkylene;

Ar₁ and Ar₂, each independently, represent hydrogen, deuterium, a halogen, a cyano, a substituted or unsubstituted (C1-C30)alkyl, a (C6-C30)aryl unsubstituted or substituted with a (C1-C30)alkyl(s), a substituted or unsubstituted (3- to 30-membered)heteroaryl, a substituted or unsubstituted (C3-C30)cycloalkyl, a substituted or unsubstituted mono- or di-(C1-C30)alkylamino, a substituted or unsubstituted mono- or di-(C2-C30)alkenylamino, a mono- or di-(C6-C30)arylamino substituted with a (C1-C30)alkyl(s), a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3-to 30-membered)heteroarylamino, or a substituted or unsubstituted (C6-C30)aryl(3- to 30-membered)heteroarylamino; with a proviso that Ar₁ and Ar₂ do not comprise dibenzoselenophene; and

* represents a bonding position:

with a proviso that the following structures are excluded.

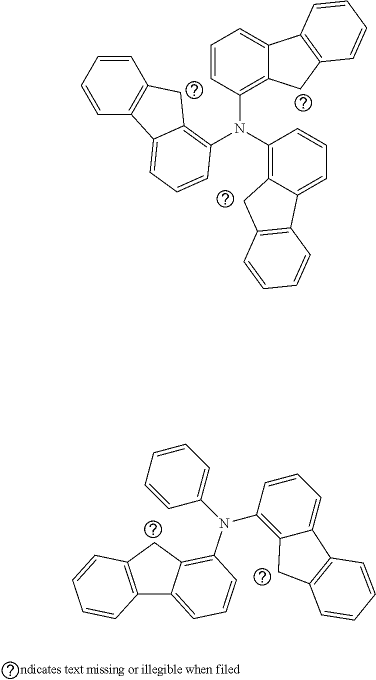 [SMILES: c1ccc2c(c1)Cc1c-2cccc1N(c1cccc2c1Cc1ccccc1-2)c1cccc2c1Cc1ccccc1-2 ; c1ccc(N(c2cccc3c2Cc2ccccc2-3)c2cccc3c2Cc2ccccc2-3)cc1]

### Advantageous Effects of Invention

An organic electroluminescent device having improved luminous efficiency and/or lifetime properties can be provided by using an organic electroluminescent compound according to the present disclosure.

### MODE FOR THE INVENTION

Hereinafter, the present disclosure will be described in detail. However, the following description is intended to explain the disclosure, and is not meant in any way to restrict the scope of the disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including host material and dopant material), an electron buffer material, a hole blocking material, an electron transport material, or an electron injection material, etc. The hole transport zone material may be at least one selected from the group consisting of a hole transport material, a hole injection material, an electron blocking material, a hole auxiliary material, and a light-emitting auxiliary material.

The organic electroluminescent material of the present disclosure may comprise at least one compound represented by formula 1. The compound of formula 1 may be included in at least one layer constituting the organic electroluminescent device, and may be included in at least one layer of the layers constituting a hole transport zone, but is not limited thereto. When the compound of formula 1 is included in a hole transport layer, a hole auxiliary layer, a light-emitting layer or a light-emitting auxiliary layer, it may be included as a hole transport material, a hole auxiliary material, a host material, or a light-emitting auxiliary material.

Herein, the term “(C1-C30)alkyl(ene)” is meant to be a linear or branched alkyl(ene) having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, and tert-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” in the present disclosure is a monocyclic or fused ring radical derived from an aromatic hydrocarbon having 6 to 30 ring backbone carbon atoms, in which the number of the ring backbone carbon atoms is preferably 6 to 25, more preferably 6 to 18, and may be partially saturated. The aryl may comprise a spiro structure. Examples of the aryl include phenyl, biphenyl, terphenyl, naphthyl, binaphthyl, phenylnaphthyl, naphthylphenyl, phenylterphenyl, fluorenyl, phenylfluorenyl, dimethylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-ter-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” in the present disclosure is meant to be an aryl(ene) having 3 to 30 ring backbone atoms and including at least one heteroatom selected from the group consisting of B, N, O, S, Si, and P. The number of heteroatoms is preferably 1 to 4. The above heteroaryl(ene) may be a monocyclic ring, or a fused ring condensed with at least one benzene ring; and may be partially saturated. Also, the above heteroaryl(ene) in the present disclosure may be one formed by linking at least one heteroaryl or aryl group to a heteroaryl(ene) group via a single bond(s), and may comprise a spiro structure. Examples of the heteroaryl may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, benzophenanthrofuranyl, dibenzothiophenyl, benzoimidazolyl, benzothiazolyl, benzoisothiazolyl, benzophenanthrothiophenyl, benzoisoxazolyl, benzoxazolyl, phenanthrooxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may be 1-pyrrolyl, 2-pyrrolyl, 3-pyrrolyl, pyrazinyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl−1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 3-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 3-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 3-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “halogen” in the present disclosure includes F, Cl, Br, and I.

In addition, “ortho (o-),” “eta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted alkylene, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3-to 30-membered)heteroaryl unsubstituted or substituted with a (C6-C30)aryl(s); a (C6-C30)aryl unsubstituted or substituted with at least one of a (C1-C30)alkyl(s), a (C6-C30)aryl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium, a (C1-C6)alkyl, a (5- to 20-membered)heteroaryl, and a (C6-C18)aryl. For example, the substituent(s), each independently, may be at least one selected from the group consisting of deuterium, a methyl, a phenyl, a biphenyl, a naphthyl, a dibenzofuranyl, a dimethylfluorenyl, a phenanthrothiazolyl and a phenanthrooxazolyl.

Hereinafter, the compound represented by formula 1 will be described in more detail.

In formula 1, R₁ to R₄, each independently, represent hydrogen or deuterium; and R₅ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or

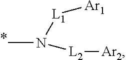

with a proviso that at least one of R₅ to R₈ represents

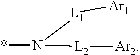

According to one embodiment of the present disclosure, R₁ to R₈ may each independently represent hydrogen or deuterium; with a proviso that at least one of R₅ to R₈ may represent

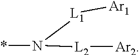

For example, R₁ to R₈ may each independently represent hydrogen or deuterium; with a proviso that any one of R₅ to R₈ may represent

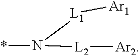

In formula 1, L₁ and L₂, each independently, represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁ and L₂ may each independently represent a single bond, a substituted or unsubstituted (C6-C30)arylene, or a substituted or unsubstituted (5- to 30-membered)heteroarylene. According to another embodiment of the present disclosure, L₁ and L₂ may each independently represent a single bond, a substituted or unsubstituted (C6-C13)arylene, or a substituted or unsubstituted (5- to 25-membered)heteroarylene. For example, L₁ and L₂ may each independently represent a single bond, a phenylene unsubstituted or substituted with a phenyl(s), a biphenylene, a carbazolylene, a phenanthrooxazolylene, a phenanthrothiazolylene, etc.

In formula 1, Ar₁ and Ar₂, each independently, represent hydrogen, deuterium, a halogen, a cyano, a substituted or unsubstituted (C1-C30)alkyl, a (C6-C30)aryl unsubstituted or substituted with a (C1-C30)alkyl(s), a substituted or unsubstituted (3- to 30-membered)heteroaryl, a substituted or unsubstituted (C3-C30)cycloalkyl, a substituted or unsubstituted mono- or di-(C1-C30)alkylamino, a substituted or unsubstituted mono- or di-(C2-C30)alkenylamino, a mono- or di-(C6-C30)arylamino substituted with a (C1-C30)alkyl(s), a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3-to 30-membered)heteroarylamino, or a substituted or unsubstituted (C6-C30)aryl(3- to 30-membered)heteroarylamino; with a proviso that Ar₁ and Ar₂ do not comprise dibenzoselenophene. According to one embodiment of the present disclosure, Ar₁ and Ar₂ may each independently represent hydrogen, deuterium, a substituted or unsubstituted (C1-C6)alkyl, a (C6-C2)aryl unsubstituted or substituted with a (C1-C6)alkyl(s), a substituted or unsubstituted (5- to 28-membered)heteroaryl, a substituted or unsubstituted (C6-C18)aryl(5- to 20-membered)heteroarylamino, or a di(C6-C25)arylamino substituted with a (C1-C10)alkyl(s). According to another embodiment of the present disclosure, Ar₁ and Ar₂ may each independently represent hydrogen, an unsubstituted (C1-C6)alkyl, a (C6-C25)aryl unsubstituted or substituted with a (C1-C6)alkyl(s), a (5- to 25-membered)heteroaryl unsubstituted or substituted with a (C6-C15)aryl(s), a (C6-C12)aryl(5- to 15-membered)heteroarylamino unsubstituted or substituted with a (C6-C12)aryl(s), or a di(C6-C18)arylamino substituted with a (C1-C6)alkyl(s). For example, Ar₁ and Ar₂ may each independently represent hydrogen, a methyl, a phenyl, a biphenyl, a naphthyl, a terphenyl, a dibenzofuranyl, a dimethylfluorenyl, a phenanthrenyl, a triphenylenyl, a dimethylindenophenanthrenyl substituted with a methyl(s), a phenanthrooxazolyl unsubstituted or substituted with a phenyl(s), a phenanthrothiazolyl unsubstituted or substituted with a phenyl(s), a benzophenanthrofuranyl, a benzophenanthrothiophenyl, a (phenylcarbazolyl)phenylamino, a (dimethylfluorenyl)phenylamino, etc.

According to one embodiment of the present disclosure, formula 1 may be represented by any one of the following formulas 1-1 to 1-4.

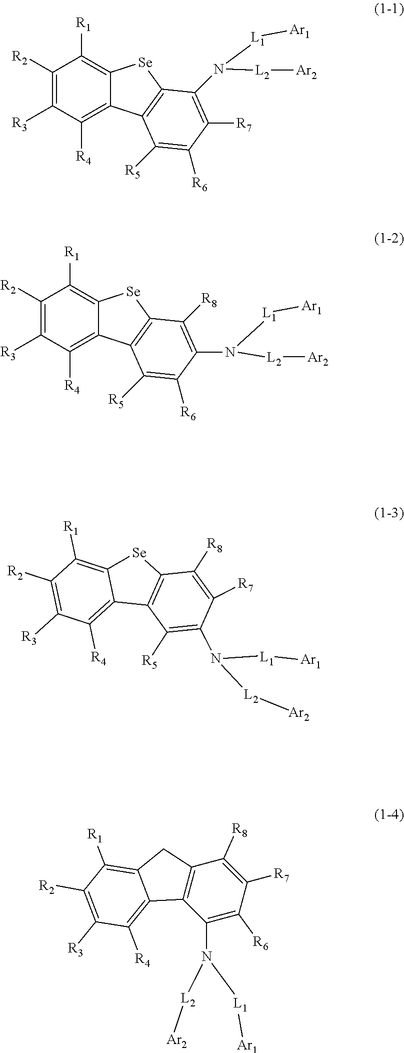

In formulas 1-1 to 1-4,

R₅ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and

R₁ to R₄, L₁, L₂, Ar₁, and Ar₂ are as defined in formula 1.

According to one embodiment of the present disclosure, Ar₁ or Ar₂ may be represented by any one of the following formulas 1-5 to 1-8.

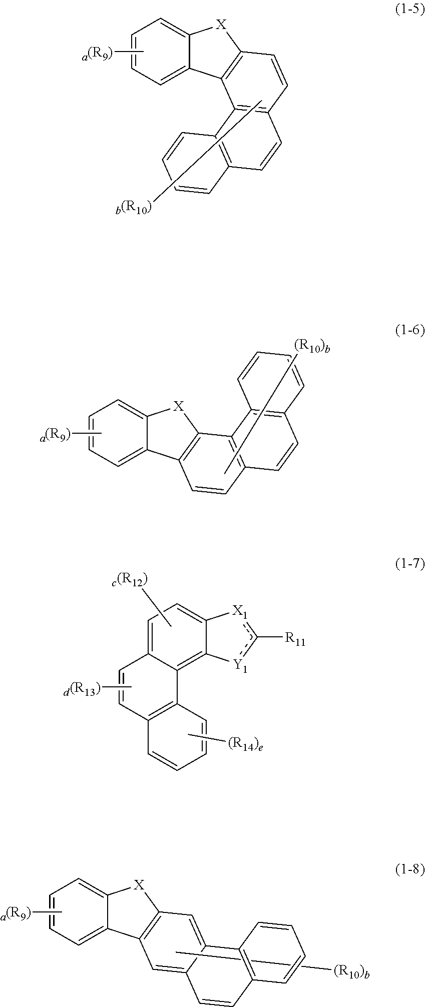

In formulas 1-5, 1-6, and 1-8, X, each independently, represents —O—, —S—, or CR′R″.

R′ and R″, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s);

According to one embodiment of the present disclosure, X, each independently, represents —O—, —S—, or CR′R″, and R′ and R″ may each independently represent a methyl, etc.

In formula 1-7, X₁ and Y₁, each independently, represent —N═, —NR₁₅—, —O— or —S—, with the proviso that any one of X₁ and Y₁ represents —N═, and the other of X₁ and Y₁ represents —NR₁₅—, —O— or —S—. According to one embodiment of the present disclosure, any one of X₁ and Y₁ represents —N═, and the other of X₁ and Y₁ represents —O— or —S—.

In formula 1-7, R₁₁ represents a substituted or unsubstituted (C6-C30)aryl, or a substituted or unsubstituted (3- to 30-membered)heteroaryl, or a bonding position with L₁ or L₂. According to one embodiment of the present disclosure, R₁ may represent an unsubstituted (C6-C12)aryl, or a bonding position with L₁ or L₂. For example, R₁₁ may represent a phenyl, or a bonding position with L₁ or L₂.

In formulas 1-5 to 1-8, R₉, R₁₀, and R₁₂ to R₁₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30) cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s); with a proviso that any one of R₉ and R₁₀ is linked to L₁ or L₂, and any one of R₁₁ to R₁₄ is linked to Li or L₂. For example, Re, R₁₀, and R₁₂ to R₁₅ may each independently represent hydrogen, with a proviso that any one of R₉ and R₁₀ is linked to L₁ or L₂, and any one of R₁₁ to R₁₄ is linked to L₁ or L₂.

In formulas 1-5 to 1-8, a, each independently, represents an integer of 1 to 4, b, each independently, represents an integer of 1 to 8, c and d, each independently, represent an integer of 1 or 2, and e represents an integer of 1 to 4, in which if a to e are an integer of 2 or more, each of R₉, each of R₁₀, and each of R₁₂ to each of R₁₄ may be the same as or different from each other.

According to one embodiment of the present disclosure, in formula 1, R₁ to R₄, each independently, represent hydrogen or deuterium; R₅ to R₈, each independently, represent hydrogen or deuterium, with a proviso that at least one of R₅ to R₈ represents

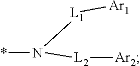

L₁ and L₂, each independently, represent a single bond, a substituted or unsubstituted (C6-C30)arylene, or a substituted or unsubstituted (5- to 30-membered)heteroarylene; and Ar₁ and Ar₂, each independently, represent hydrogen, deuterium, a substituted or unsubstituted (C1-C6)alkyl, a (C6-C18)aryl unsubstituted or substituted with a (C1-C6 )alkyl(s), a substituted or unsubstituted (5- to 20-membered)heteroaryl, a substituted or unsubstituted (C6-C18)aryl(5- to 20-membered)heteroarylamino, or a di(C6-C25)arylamino substituted with a (C1-C10)alkyl(s).

According to another embodiment of the present disclosure, in formula 1, R₁ to R₄, each independently, represent hydrogen or deuterium; R₅ to R₈, each independently, represent hydrogen or deuterium, with a proviso that at least one of R₅ to R₈ represents

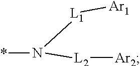

L₁ and L₂, each independently, represent a single bond, a substituted or unsubstituted (C6-C18)arylene, or a substituted or unsubstituted (5- to 20-membered)heteroarylene; and Ar₁ and Ar₂, each independently, represent hydrogen, an unsubstituted (C1-C6)alkyl, a (C6-C18)aryl unsubstituted or substituted with a (C1-C6)alkyl(s), a (5- to 20-membered)heteroaryl unsubstituted or substituted with a (C6-C15)aryl(s), a (C6-C18)aryl(5- to 20-membered)heteroarylamino unsubstituted or substituted with a (C6-C12)aryl(s), or a di(C6-C18)arylamino substituted with a (C1-C6)alkyl(s).

The compound represented by formula 1 may be at least one selected from the group consisting of the following compounds, but is not limited thereto.

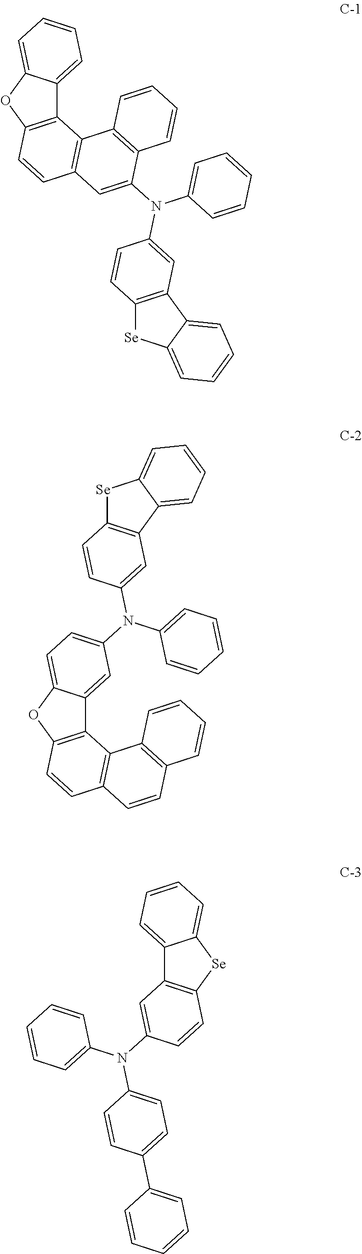 [SMILES: c1ccc(N(c2ccc3[se]c4ccccc4c3c2)c2cc3ccc4oc5ccccc5c4c3c3ccccc23)cc1 ; c1ccc(N(c2ccc3[se]c4ccccc4c3c2)c2ccc3oc4ccc5ccc6ccccc6c5c4c3c2)cc1 ; c1ccc(-c2ccc(N(c3ccccc3)c3ccc4[se]c5ccccc5c4c3)cc2)cc1]

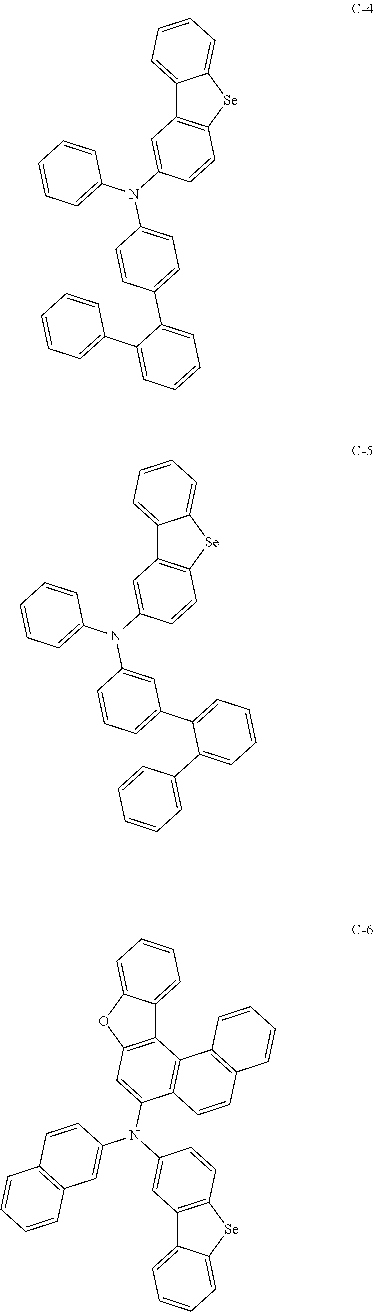 [SMILES: c1ccc(-c2ccccc2-c2ccc(N(c3ccccc3)c3ccc4[se]c5ccccc5c4c3)cc2)cc1 ; c1ccc(-c2ccccc2-c2cccc(N(c3ccccc3)c3ccc4[se]c5ccccc5c4c3)c2)cc1 ; c1ccc2cc(N(c3ccc4[se]c5ccccc5c4c3)c3cc4oc5ccccc5c4c4c3ccc3ccccc34)ccc2c1]

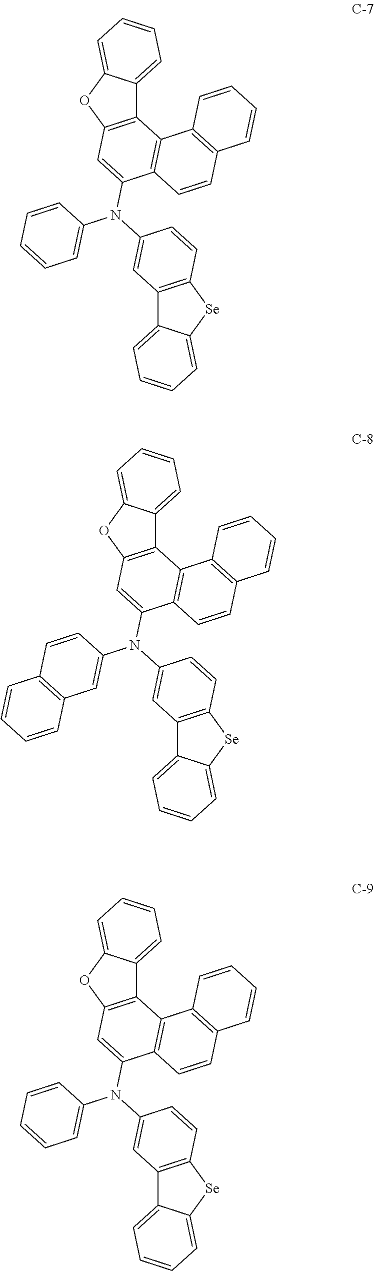 [SMILES: c1ccc(N(c2ccc3[se]c4ccccc4c3c2)c2cc3oc4ccccc4c3c3c2ccc2ccccc23)cc1 ; c1ccc2cc(N(c3ccc4[se]c5ccccc5c4c3)c3cc4oc5ccccc5c4c4c3ccc3ccccc34)ccc2c1 ; c1ccc(N(c2ccc3[se]c4ccccc4c3c2)c2cc3oc4ccccc4c3c3c2ccc2ccccc23)cc1]

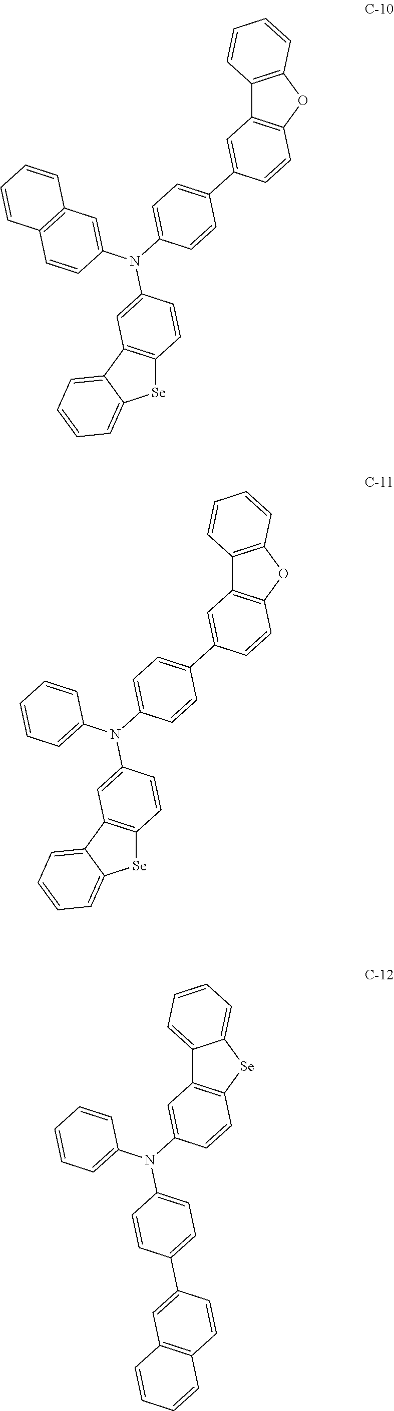 [SMILES: c1ccc2cc(N(c3ccc(-c4ccc5oc6ccccc6c5c4)cc3)c3ccc4[se]c5ccccc5c4c3)ccc2c1 ; c1ccc(N(c2ccc(-c3ccc4oc5ccccc5c4c3)cc2)c2ccc3[se]c4ccccc4c3c2)cc1 ; c1ccc(N(c2ccc(-c3ccc4ccccc4c3)cc2)c2ccc3[se]c4ccccc4c3c2)cc1]

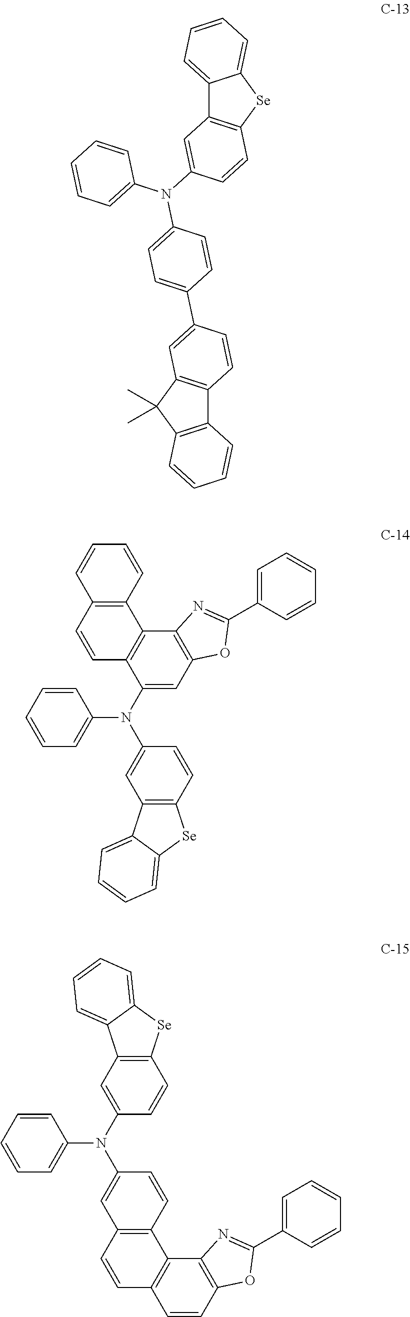 [SMILES: CC1(C)c2ccccc2-c2ccc(-c3ccc(N(c4ccccc4)c4ccc5[se]c6ccccc6c5c4)cc3)cc21 ; c1ccc(-c2nc3c(cc(N(c4ccccc4)c4ccc5[se]c6ccccc6c5c4)c4ccc5ccccc5c43)o2)cc1 ; c1ccc(-c2nc3c(ccc4ccc5cc(N(c6ccccc6)c6ccc7[se]c8ccccc8c7c6)ccc5c43)o2)cc1]

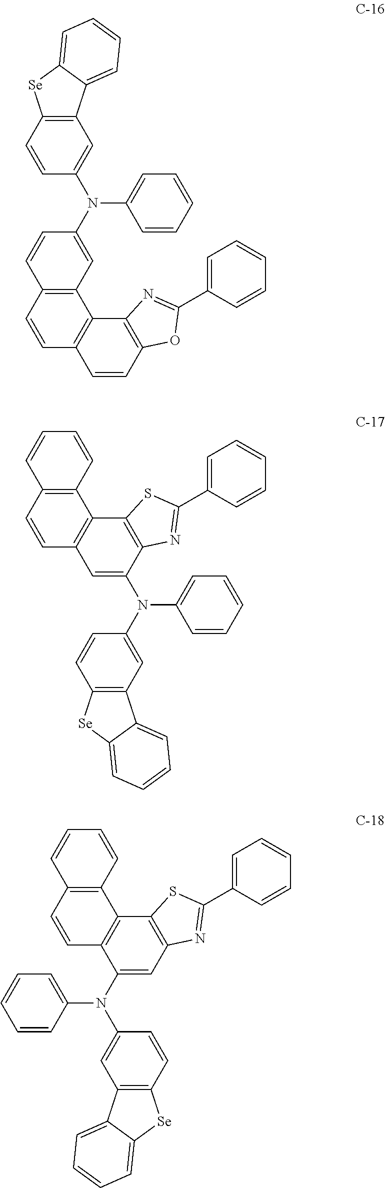 [SMILES: c1ccc(-c2nc3c(ccc4ccc5ccc(N(c6ccccc6)c6ccc7[se]c8ccccc8c7c6)cc5c43)o2)cc1 ; c1ccc(-c2nc3c(N(c4ccccc4)c4ccc5[se]c6ccccc6c5c4)cc4ccc5ccccc5c4c3s2)cc1 ; c1ccc(-c2nc3cc(N(c4ccccc4)c4ccc5[se]c6ccccc6c5c4)c4ccc5ccccc5c4c3s2)cc1]

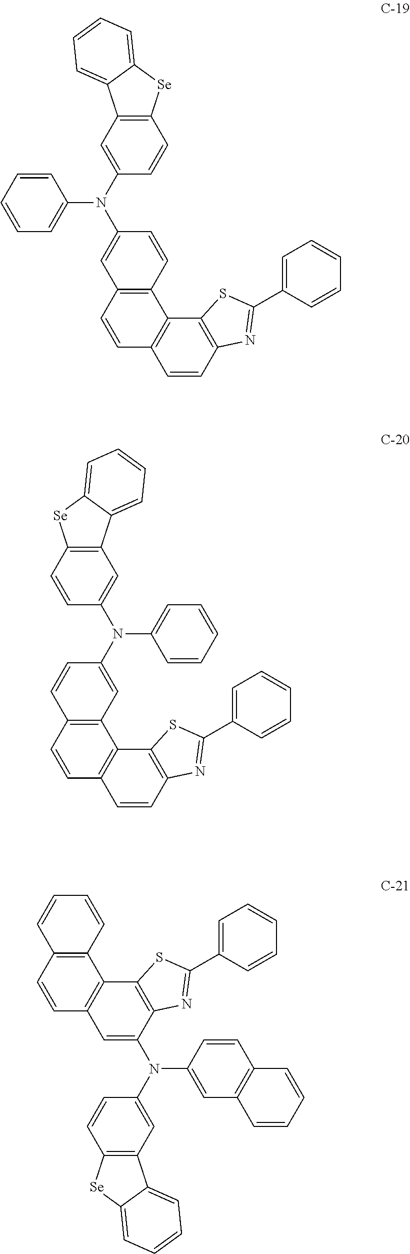 [SMILES: c1ccc(-c2nc3ccc4ccc5cc(N(c6ccccc6)c6ccc7[se]c8ccccc8c7c6)ccc5c4c3s2)cc1 ; c1ccc(-c2nc3ccc4ccc5ccc(N(c6ccccc6)c6ccc7[se]c8ccccc8c7c6)cc5c4c3s2)cc1 ; c1ccc(-c2nc3c(N(c4ccc5ccccc5c4)c4ccc5[se]c6ccccc6c5c4)cc4ccc5ccccc5c4c3s2)cc1]

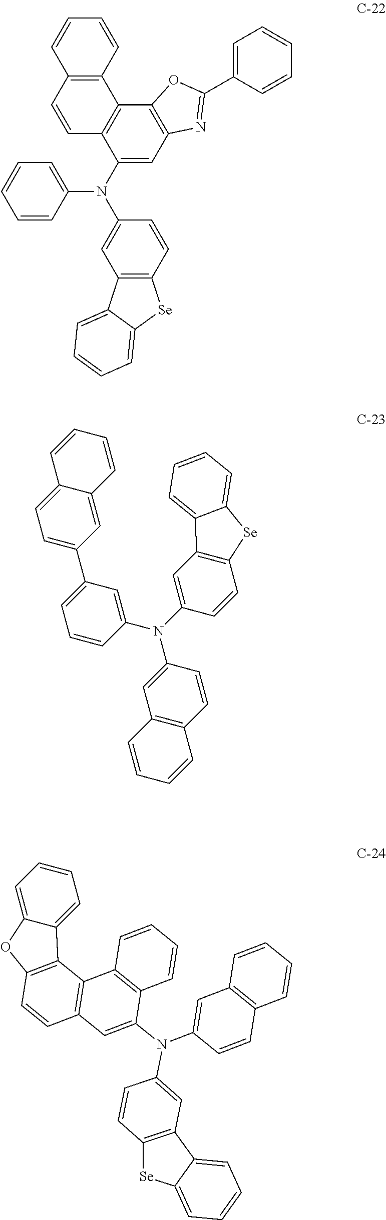 [SMILES: c1ccc(-c2nc3cc(N(c4ccccc4)c4ccc5[se]c6ccccc6c5c4)c4ccc5ccccc5c4c3o2)cc1 ; c1cc(-c2ccc3ccccc3c2)cc(N(c2ccc3ccccc3c2)c2ccc3[se]c4ccccc4c3c2)c1 ; c1ccc2cc(N(c3ccc4[se]c5ccccc5c4c3)c3cc4ccc5oc6ccccc6c5c4c4ccccc34)ccc2c1]

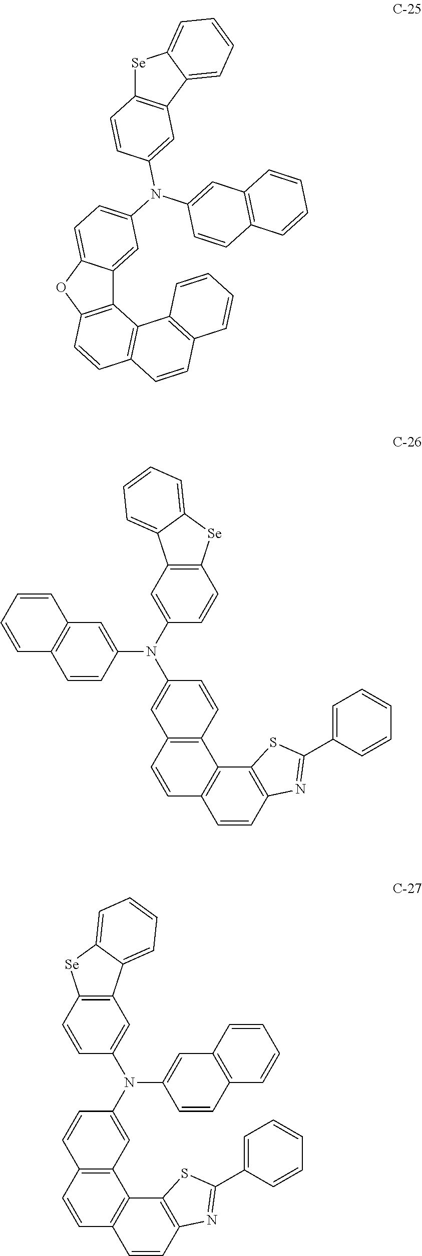 [SMILES: c1ccc2cc(N(c3ccc4[se]c5ccccc5c4c3)c3ccc4oc5ccc6ccc7ccccc7c6c5c4c3)ccc2c1 ; c1ccc(-c2nc3ccc4ccc5cc(N(c6ccc7ccccc7c6)c6ccc7[se]c8ccccc8c7c6)ccc5c4c3s2)cc1 ; c1ccc(-c2nc3ccc4ccc5ccc(N(c6ccc7ccccc7c6)c6ccc7[se]c8ccccc8c7c6)cc5c4c3s2)cc1]

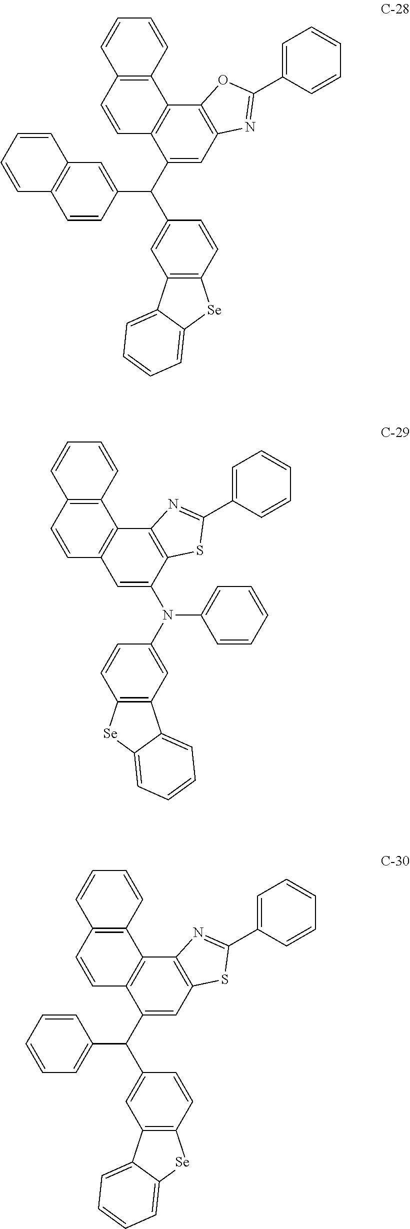 [SMILES: c1ccc(-c2nc3cc(C(c4ccc5ccccc5c4)c4ccc5[se]c6ccccc6c5c4)c4ccc5ccccc5c4c3o2)cc1 ; c1ccc(-c2nc3c(s2)c(N(c2ccccc2)c2ccc4[se]c5ccccc5c4c2)cc2ccc4ccccc4c23)cc1 ; c1ccc(-c2nc3c(cc(C(c4ccccc4)c4ccc5[se]c6ccccc6c5c4)c4ccc5ccccc5c43)s2)cc1]

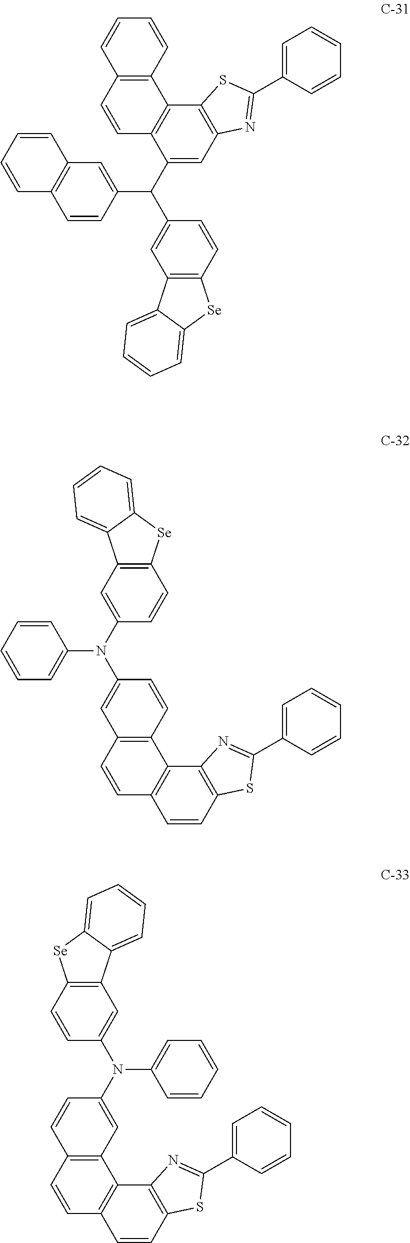 [SMILES: c1ccc(-c2nc3cc(C(c4ccc5ccccc5c4)c4ccc5[se]c6ccccc6c5c4)c4ccc5ccccc5c4c3s2)cc1 ; c1ccc(-c2nc3c(ccc4ccc5cc(N(c6ccccc6)c6ccc7[se]c8ccccc8c7c6)ccc5c43)s2)cc1 ; c1ccc(-c2nc3c(ccc4ccc5ccc(N(c6ccccc6)c6ccc7[se]c8ccccc8c7c6)cc5c43)s2)cc1]

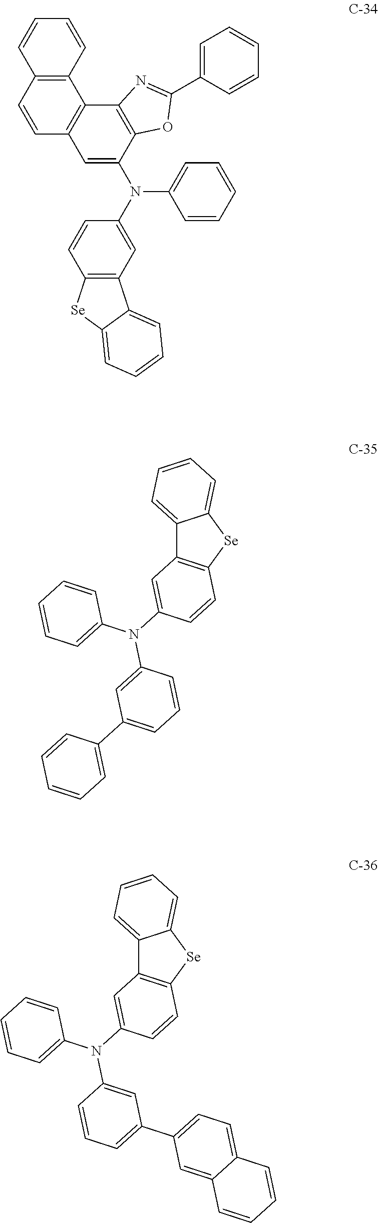 [SMILES: c1ccc(-c2nc3c(o2)c(N(c2ccccc2)c2ccc4[se]c5ccccc5c4c2)cc2ccc4ccccc4c23)cc1 ; c1ccc(-c2cccc(N(c3ccccc3)c3ccc4[se]c5ccccc5c4c3)c2)cc1 ; c1ccc(N(c2cccc(-c3ccc4ccccc4c3)c2)c2ccc3[se]c4ccccc4c3c2)cc1]

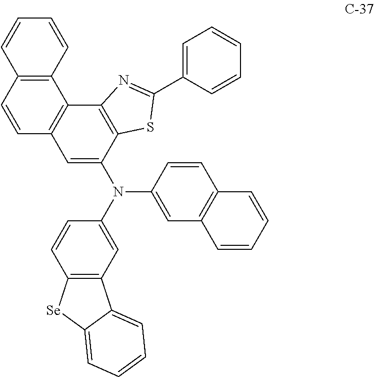 [SMILES: c1ccc(-c2nc3c(s2)c(N(c2ccc4ccccc4c2)c2ccc4[se]c5ccccc5c4c2)cc2ccc4ccccc4c23)cc1]

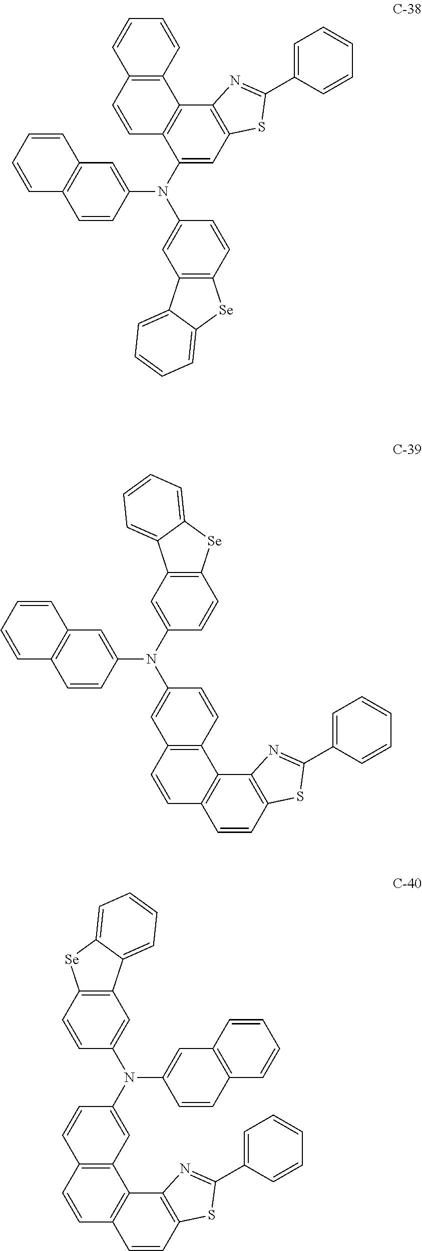 [SMILES: c1ccc(-c2nc3c(cc(N(c4ccc5ccccc5c4)c4ccc5[se]c6ccccc6c5c4)c4ccc5ccccc5c43)s2)cc1 ; c1ccc(-c2nc3c(ccc4ccc5cc(N(c6ccc7ccccc7c6)c6ccc7[se]c8ccccc8c7c6)ccc5c43)s2)cc1 ; c1ccc(-c2nc3c(ccc4ccc5ccc(N(c6ccc7ccccc7c6)c6ccc7[se]c8ccccc8c7c6)cc5c43)s2)cc1]

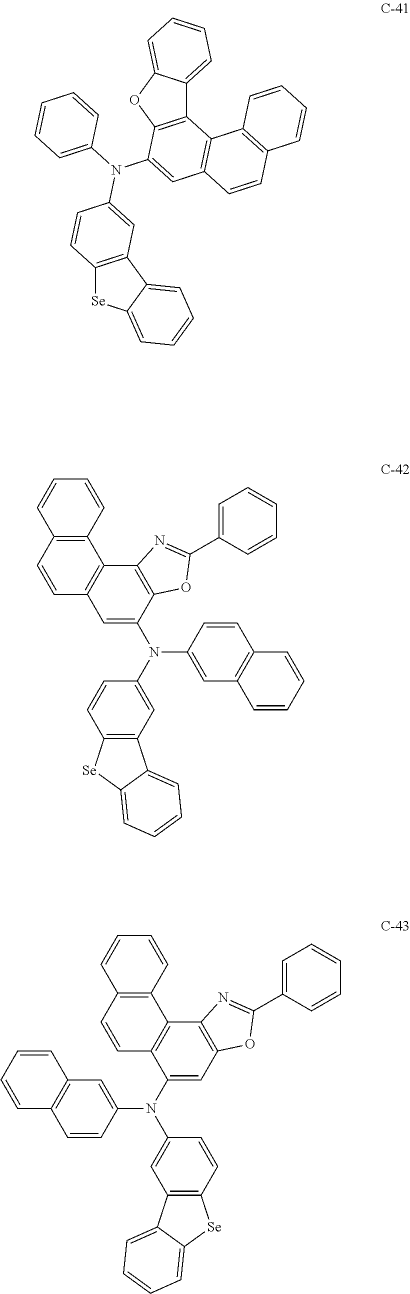 [SMILES: c1ccc(N(c2ccc3[se]c4ccccc4c3c2)c2cc3ccc4ccccc4c3c3c2oc2ccccc23)cc1 ; c1ccc(-c2nc3c(o2)c(N(c2ccc4ccccc4c2)c2ccc4[se]c5ccccc5c4c2)cc2ccc4ccccc4c23)cc1 ; c1ccc(-c2nc3c(cc(N(c4ccc5ccccc5c4)c4ccc5[se]c6ccccc6c5c4)c4ccc5ccccc5c43)o2)cc1]

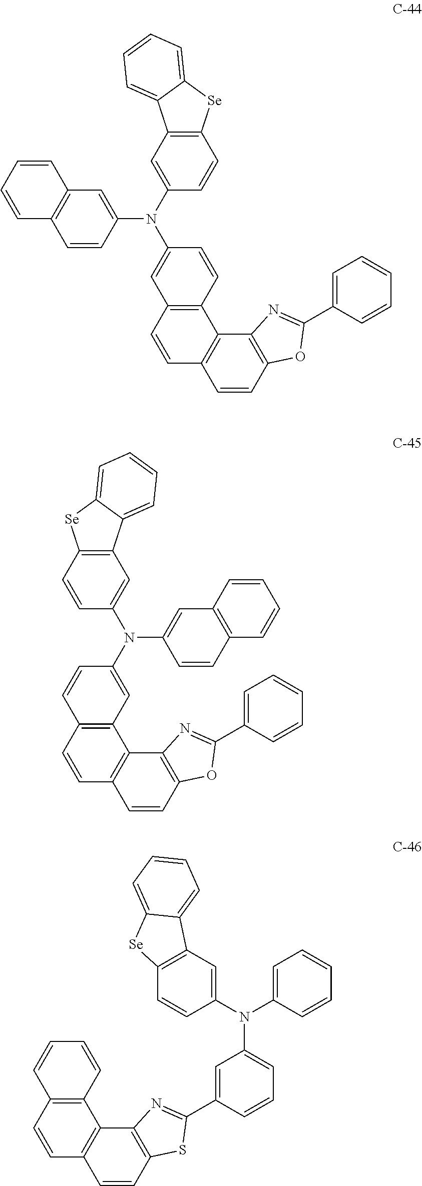 [SMILES: c1ccc(-c2nc3c(ccc4ccc5cc(N(c6ccc7ccccc7c6)c6ccc7[se]c8ccccc8c7c6)ccc5c43)o2)cc1 ; c1ccc(-c2nc3c(ccc4ccc5ccc(N(c6ccc7ccccc7c6)c6ccc7[se]c8ccccc8c7c6)cc5c43)o2)cc1 ; c1ccc(N(c2cccc(-c3nc4c(ccc5ccc6ccccc6c54)s3)c2)c2ccc3[se]c4ccccc4c3c2)cc1]

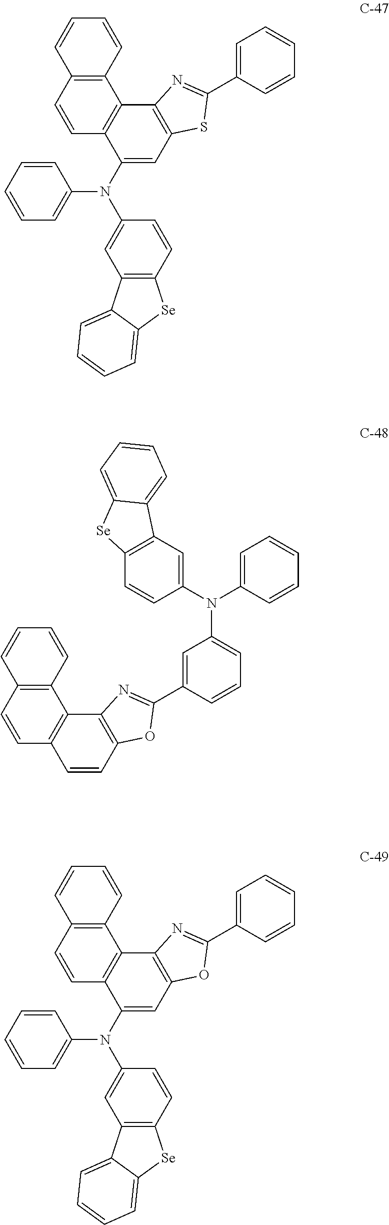 [SMILES: c1ccc(-c2nc3c(cc(N(c4ccccc4)c4ccc5[se]c6ccccc6c5c4)c4ccc5ccccc5c43)s2)cc1 ; c1ccc(N(c2cccc(-c3nc4c(ccc5ccc6ccccc6c54)o3)c2)c2ccc3[se]c4ccccc4c3c2)cc1 ; c1ccc(-c2nc3c(cc(N(c4ccccc4)c4ccc5[se]c6ccccc6c5c4)c4ccc5ccccc5c43)o2)cc1]

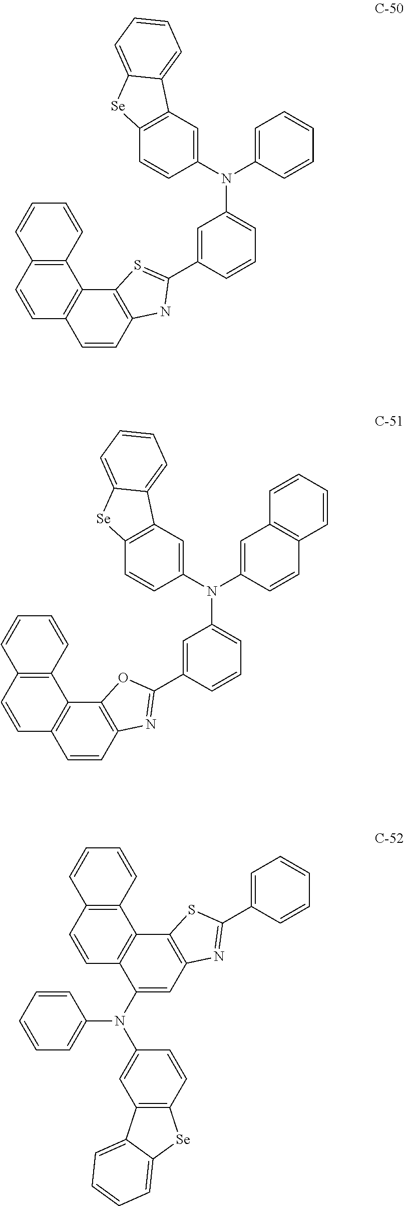

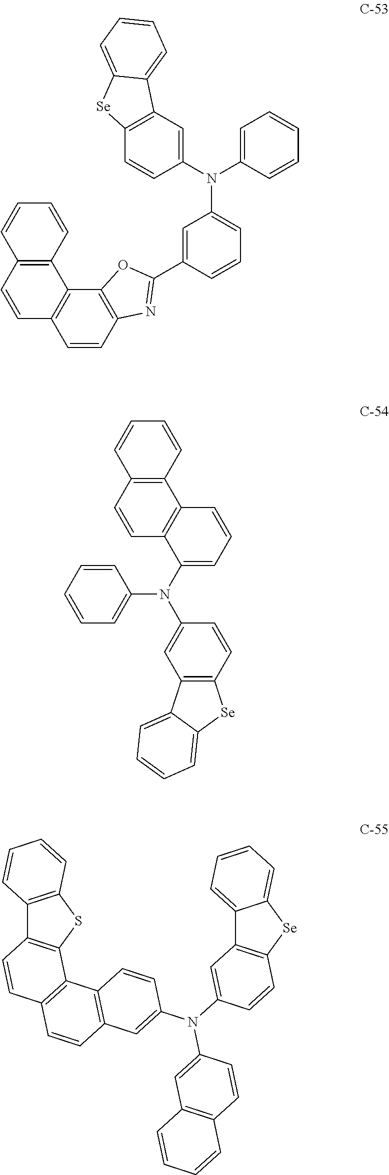 [SMILES: c1ccc(N(c2cccc(-c3nc4ccc5ccc6ccccc6c5c4o3)c2)c2ccc3[se]c4ccccc4c3c2)cc1 ; c1ccc(N(c2ccc3[se]c4ccccc4c3c2)c2cccc3c2ccc2ccccc23)cc1 ; c1ccc2cc(N(c3ccc4c(ccc5ccc6c7ccccc7sc6c54)c3)c3ccc4[se]c5ccccc5c4c3)ccc2c1]

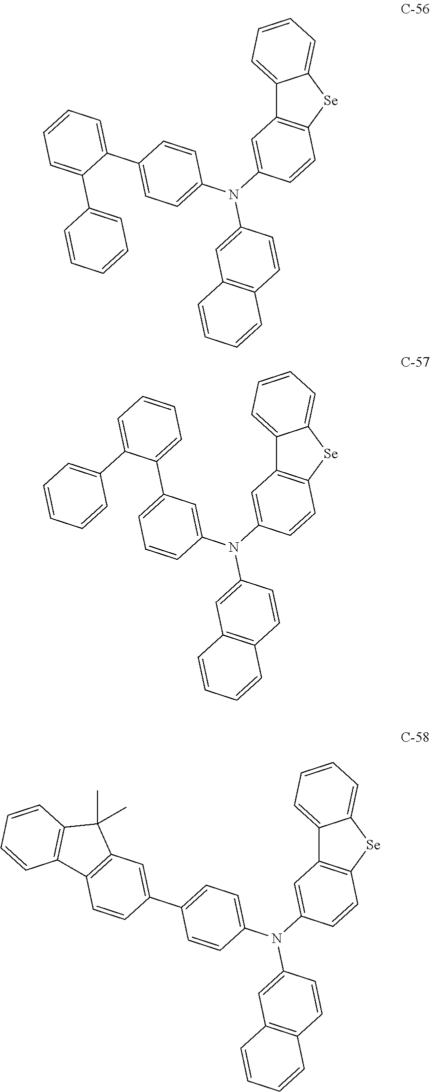

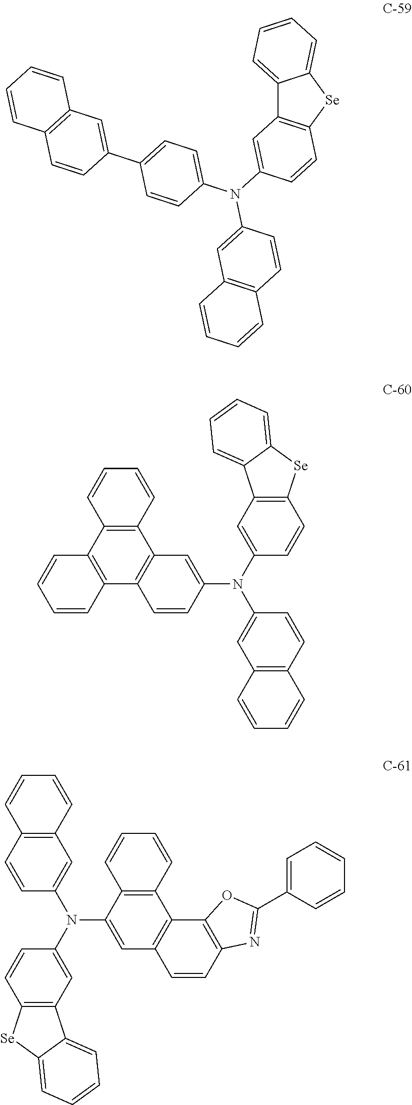 [SMILES: c1ccc2cc(-c3ccc(N(c4ccc5ccccc5c4)c4ccc5[se]c6ccccc6c5c4)cc3)ccc2c1 ; c1ccc2cc(N(c3ccc4[se]c5ccccc5c4c3)c3ccc4c5ccccc5c5ccccc5c4c3)ccc2c1 ; c1ccc(-c2nc3ccc4cc(N(c5ccc6ccccc6c5)c5ccc6[se]c7ccccc7c6c5)c5ccccc5c4c3o2)cc1]

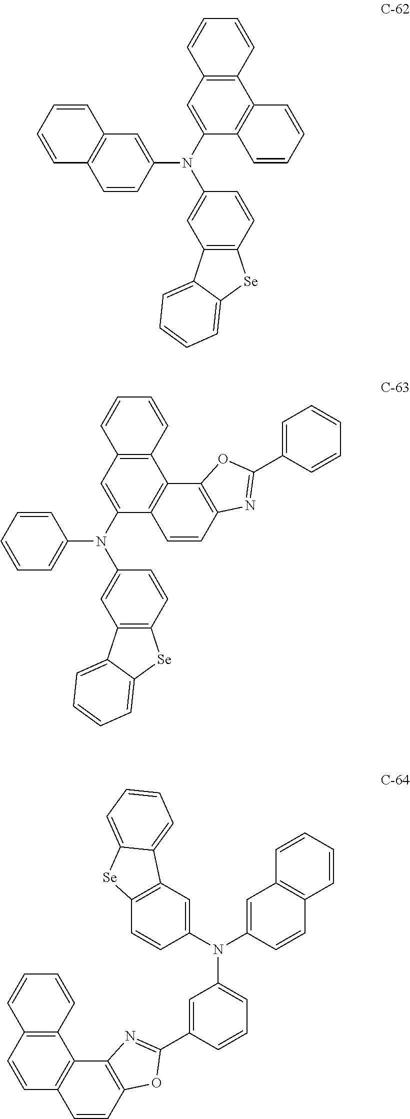 [SMILES: c1ccc2cc(N(c3ccc4[se]c5ccccc5c4c3)c3cc4ccccc4c4ccccc34)ccc2c1 ; c1ccc(-c2nc3ccc4c(N(c5ccccc5)c5ccc6[se]c7ccccc7c6c5)cc5ccccc5c4c3o2)cc1 ; c1cc(-c2nc3c(ccc4ccc5ccccc5c43)o2)cc(N(c2ccc3ccccc3c2)c2ccc3[se]c4ccccc4c3c2)c1]

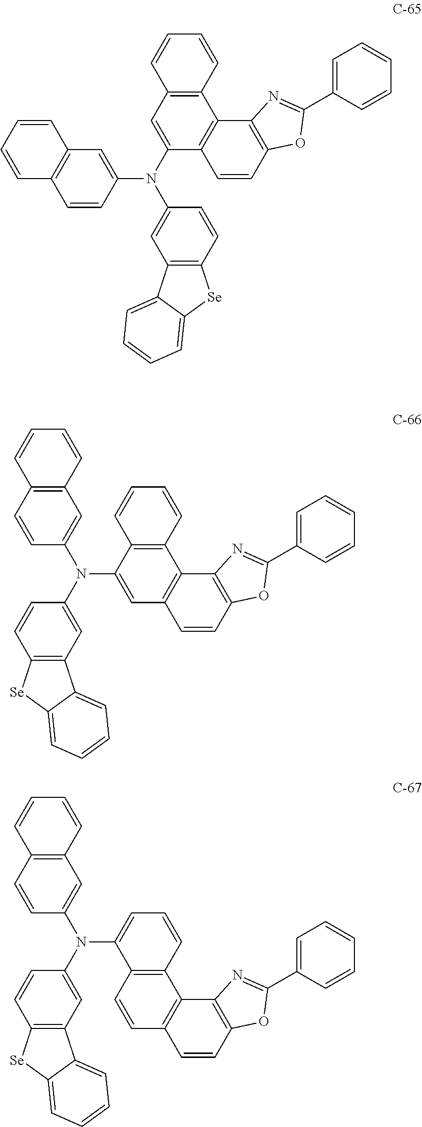

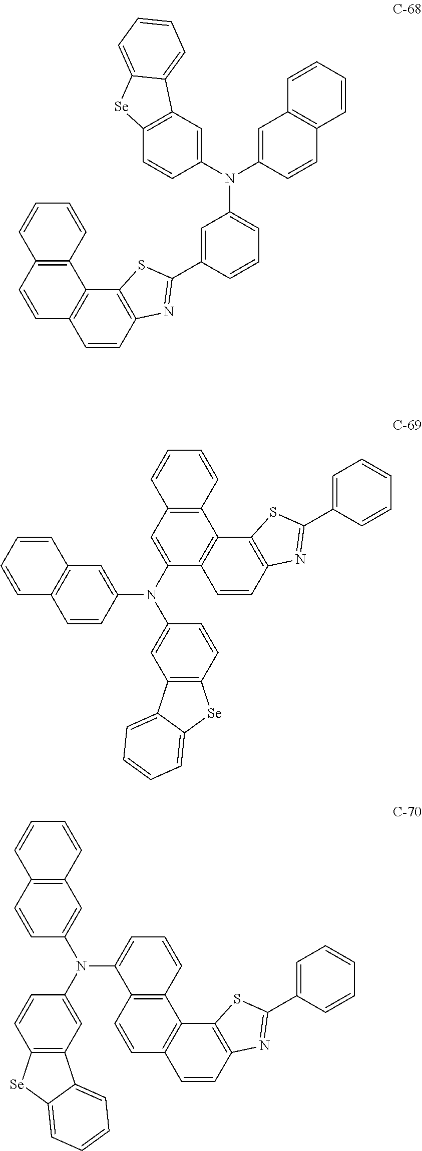 [SMILES: c1cc(-c2nc3ccc4ccc5ccccc5c4c3s2)cc(N(c2ccc3ccccc3c2)c2ccc3[se]c4ccccc4c3c2)c1 ; c1ccc(-c2nc3ccc4c(N(c5ccc6ccccc6c5)c5ccc6[se]c7ccccc7c6c5)cc5ccccc5c4c3s2)cc1 ; c1ccc(-c2nc3ccc4ccc5c(N(c6ccc7ccccc7c6)c6ccc7[se]c8ccccc8c7c6)cccc5c4c3s2)cc1]

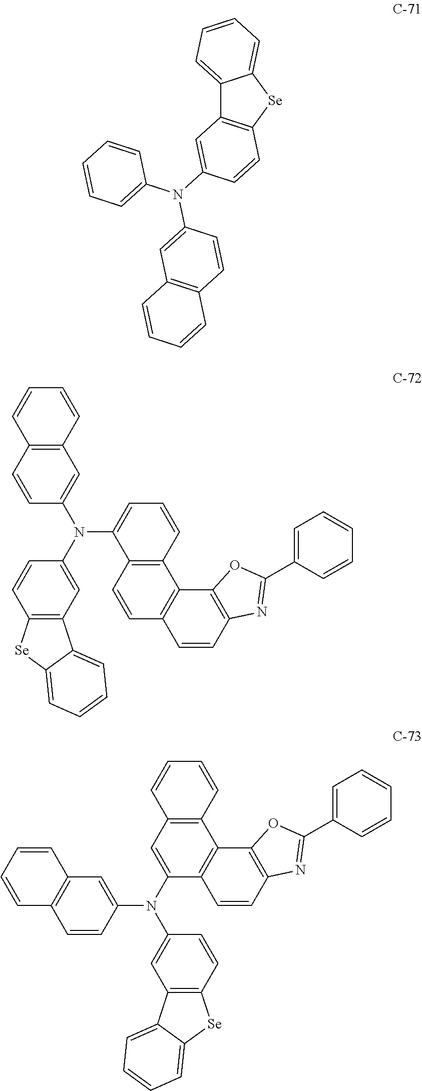 [SMILES: c1ccc(N(c2ccc3ccccc3c2)c2ccc3[se]c4ccccc4c3c2)cc1 ; c1ccc(-c2nc3ccc4ccc5c(N(c6ccc7ccccc7c6)c6ccc7[se]c8ccccc8c7c6)cccc5c4c3o2)cc1 ; c1ccc(-c2nc3ccc4c(N(c5ccc6ccccc6c5)c5ccc6[se]c7ccccc7c6c5)cc5ccccc5c4c3o2)cc1]

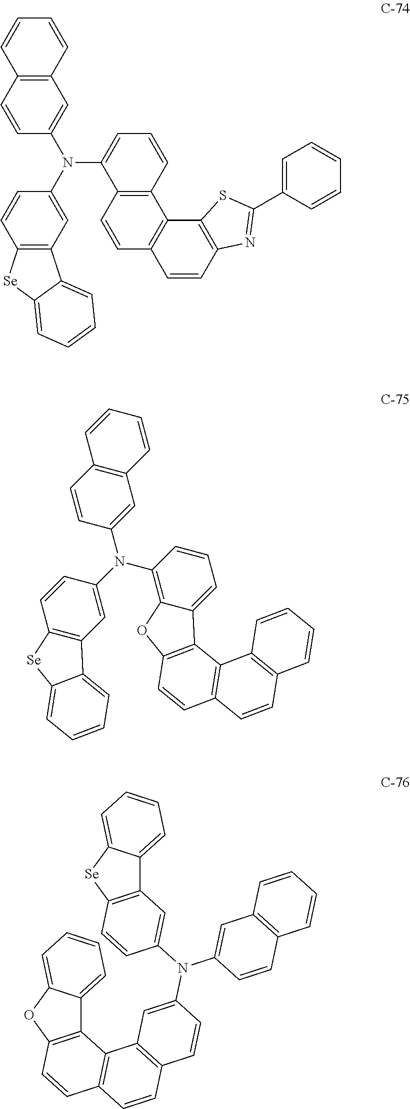 [SMILES: c1ccc(-c2nc3ccc4ccc5c(N(c6ccc7ccccc7c6)c6ccc7[se]c8ccccc8c7c6)cccc5c4c3s2)cc1 ; c1ccc2cc(N(c3ccc4[se]c5ccccc5c4c3)c3cccc4c3oc3ccc5ccc6ccccc6c5c34)ccc2c1 ; c1ccc2cc(N(c3ccc4[se]c5ccccc5c4c3)c3ccc4ccc5ccc6oc7ccccc7c6c5c4c3)ccc2c1]

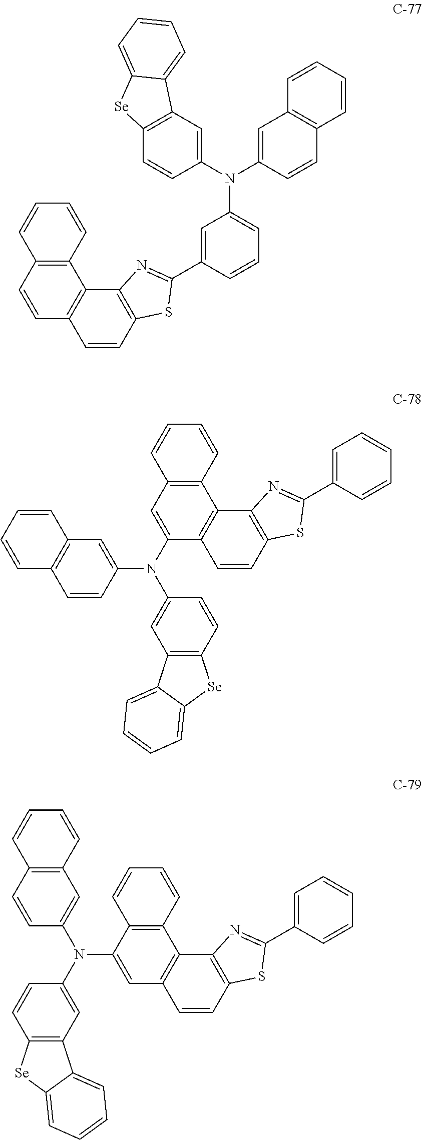 [SMILES: c1cc(-c2nc3c(ccc4ccc5ccccc5c43)s2)cc(N(c2ccc3ccccc3c2)c2ccc3[se]c4ccccc4c3c2)c1 ; c1ccc(-c2nc3c(ccc4c(N(c5ccc6ccccc6c5)c5ccc6[se]c7ccccc7c6c5)cc5ccccc5c43)s2)cc1 ; c1ccc(-c2nc3c(ccc4cc(N(c5ccc6ccccc6c5)c5ccc6[se]c7ccccc7c6c5)c5ccccc5c43)s2)cc1]

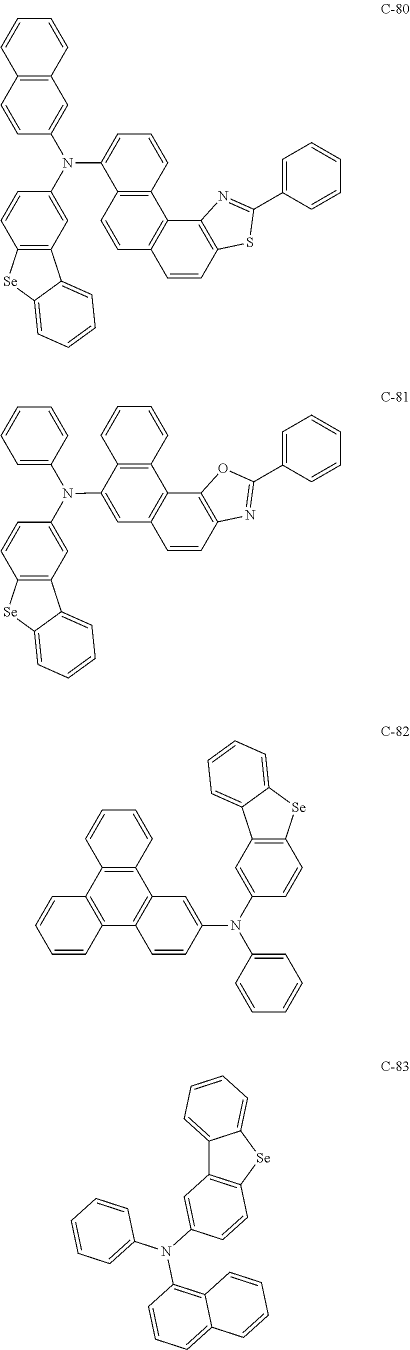 [SMILES: c1ccc(-c2nc3c(ccc4ccc5c(N(c6ccc7ccccc7c6)c6ccc7[se]c8ccccc8c7c6)cccc5c43)s2)cc1 ; c1ccc(-c2nc3ccc4cc(N(c5ccccc5)c5ccc6[se]c7ccccc7c6c5)c5ccccc5c4c3o2)cc1 ; c1ccc(N(c2ccc3[se]c4ccccc4c3c2)c2ccc3c4ccccc4c4ccccc4c3c2)cc1 ; c1ccc(N(c2ccc3[se]c4ccccc4c3c2)c2cccc3ccccc23)cc1]

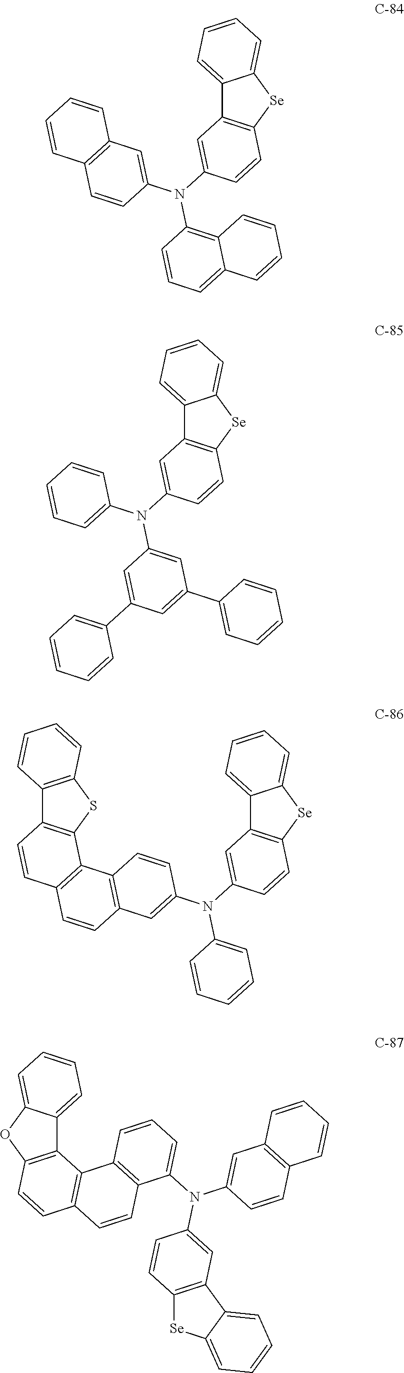 [SMILES: c1ccc2cc(N(c3ccc4[se]c5ccccc5c4c3)c3cccc4ccccc34)ccc2c1 ; c1ccc(-c2cc(-c3ccccc3)cc(N(c3ccccc3)c3ccc4[se]c5ccccc5c4c3)c2)cc1 ; c1ccc(N(c2ccc3c(ccc4ccc5c6ccccc6sc5c43)c2)c2ccc3[se]c4ccccc4c3c2)cc1 ; c1ccc2cc(N(c3ccc4[se]c5ccccc5c4c3)c3cccc4c3ccc3ccc5oc6ccccc6c5c34)ccc2c1]

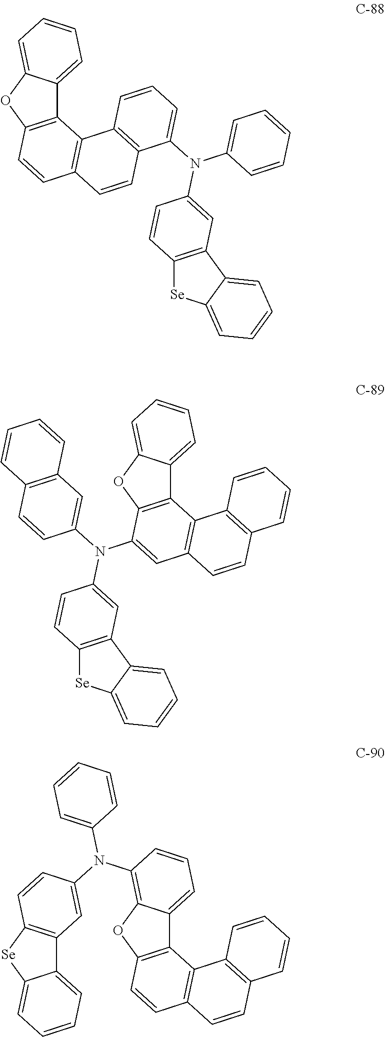 [SMILES: c1ccc(N(c2ccc3[se]c4ccccc4c3c2)c2cccc3c2ccc2ccc4oc5ccccc5c4c23)cc1 ; c1ccc2cc(N(c3ccc4[se]c5ccccc5c4c3)c3cc4ccc5ccccc5c4c4c3oc3ccccc34)ccc2c1 ; c1ccc(N(c2ccc3[se]c4ccccc4c3c2)c2cccc3c2oc2ccc4ccc5ccccc5c4c23)cc1]

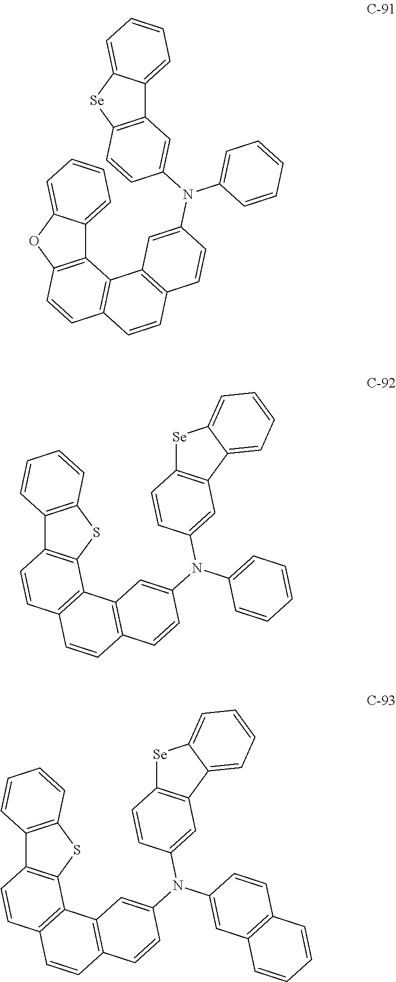 [SMILES: c1ccc(N(c2ccc3[se]c4ccccc4c3c2)c2ccc3ccc4ccc5oc6ccccc6c5c4c3c2)cc1 ; c1ccc(N(c2ccc3[se]c4ccccc4c3c2)c2ccc3ccc4ccc5c6ccccc6sc5c4c3c2)cc1 ; c1ccc2cc(N(c3ccc4[se]c5ccccc5c4c3)c3ccc4ccc5ccc6c7ccccc7sc6c5c4c3)ccc2c1]

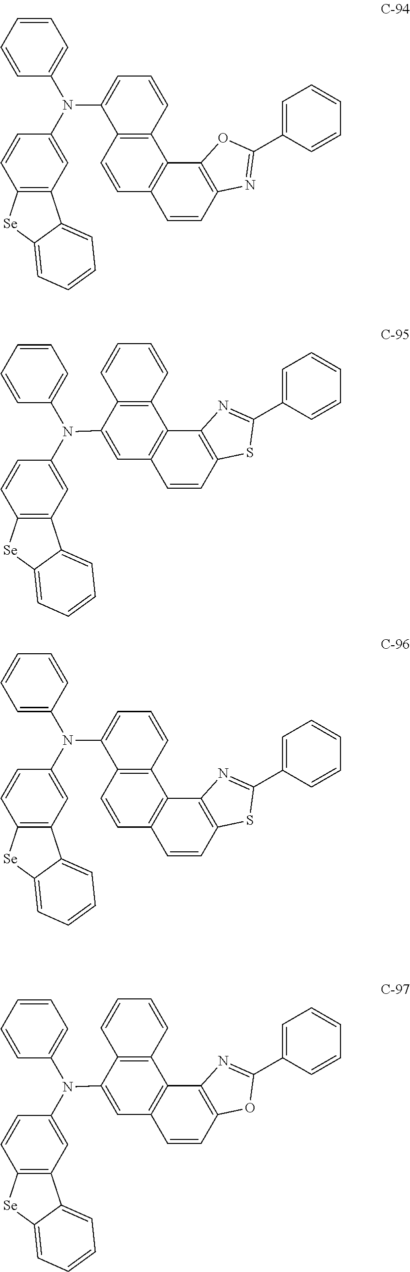

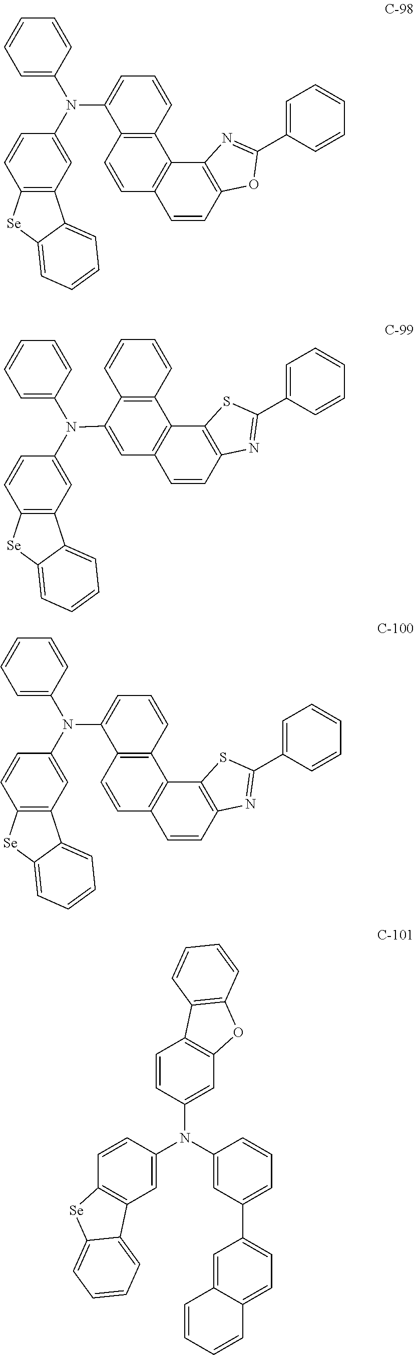

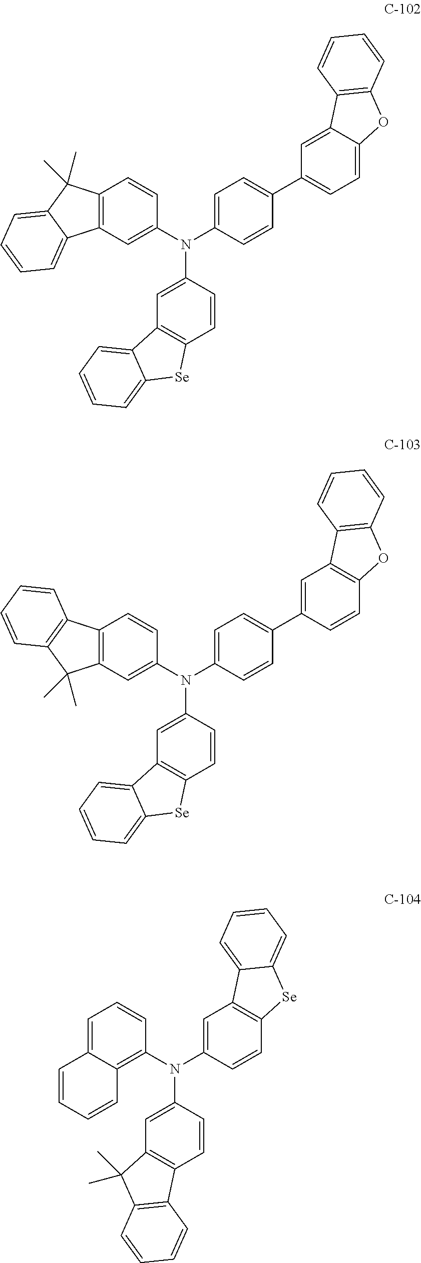 [SMILES: CC1(C)c2ccccc2-c2cc(N(c3ccc(-c4ccc5oc6ccccc6c5c4)cc3)c3ccc4[se]c5ccccc5c4c3)ccc21 ; CC1(C)c2ccccc2-c2ccc(N(c3ccc(-c4ccc5oc6ccccc6c5c4)cc3)c3ccc4[se]c5ccccc5c4c3)cc21 ; CC1(C)c2ccccc2-c2ccc(N(c3ccc4[se]c5ccccc5c4c3)c3cccc4ccccc34)cc21]

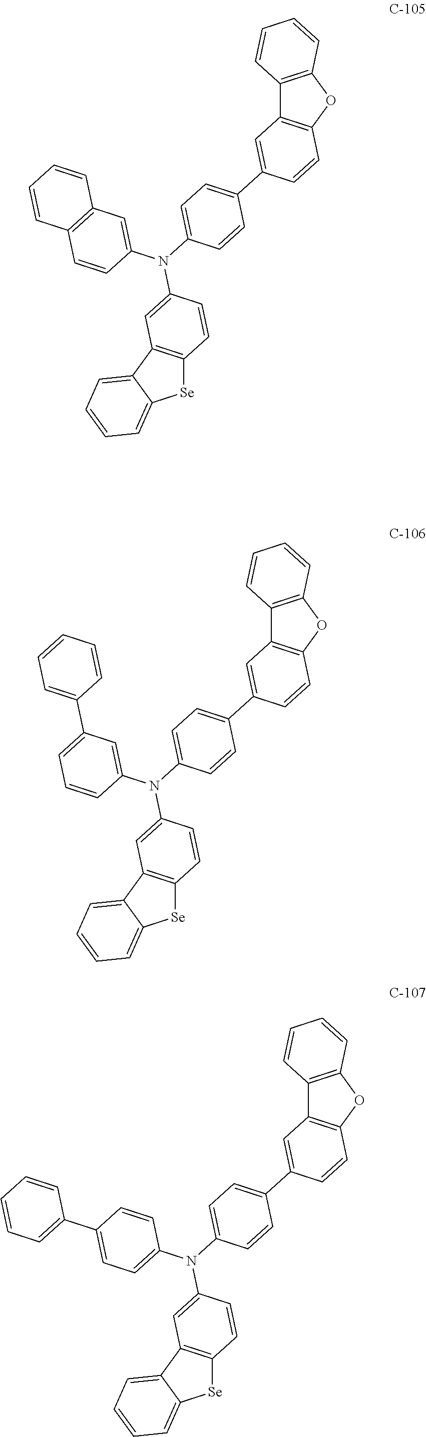 [SMILES: c1ccc2cc(N(c3ccc(-c4ccc5oc6ccccc6c5c4)cc3)c3ccc4[se]c5ccccc5c4c3)ccc2c1 ; c1ccc(-c2cccc(N(c3ccc(-c4ccc5oc6ccccc6c5c4)cc3)c3ccc4[se]c5ccccc5c4c3)c2)cc1 ; c1ccc(-c2ccc(N(c3ccc(-c4ccc5oc6ccccc6c5c4)cc3)c3ccc4[se]c5ccccc5c4c3)cc2)cc1]

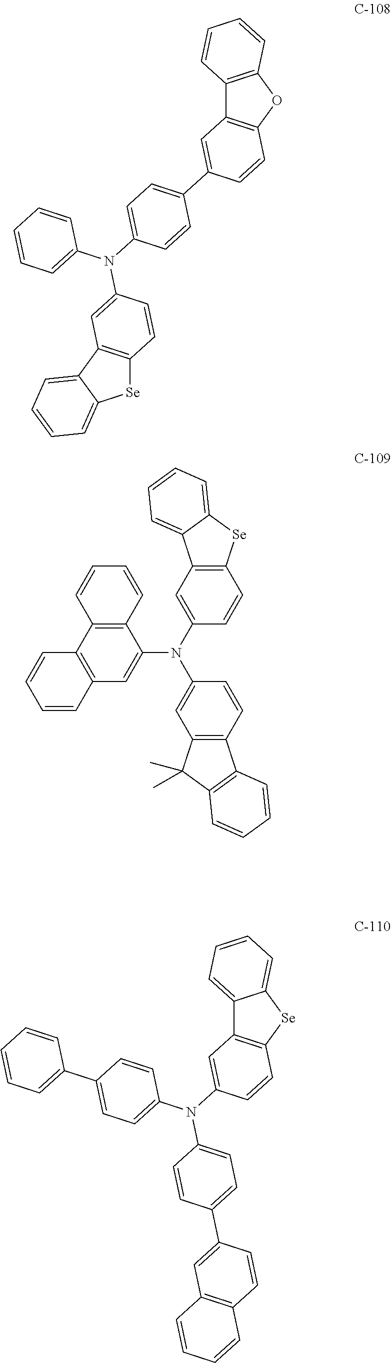 [SMILES: c1ccc(N(c2ccc(-c3ccc4oc5ccccc5c4c3)cc2)c2ccc3[se]c4ccccc4c3c2)cc1 ; CC1(C)c2ccccc2-c2ccc(N(c3ccc4[se]c5ccccc5c4c3)c3cc4ccccc4c4ccccc34)cc21 ; c1ccc(-c2ccc(N(c3ccc(-c4ccc5ccccc5c4)cc3)c3ccc4[se]c5ccccc5c4c3)cc2)cc1]

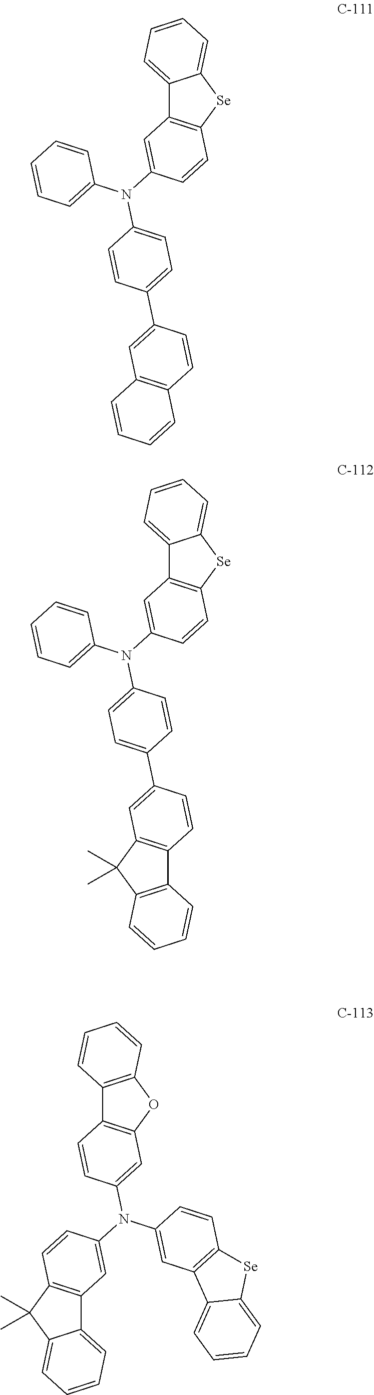

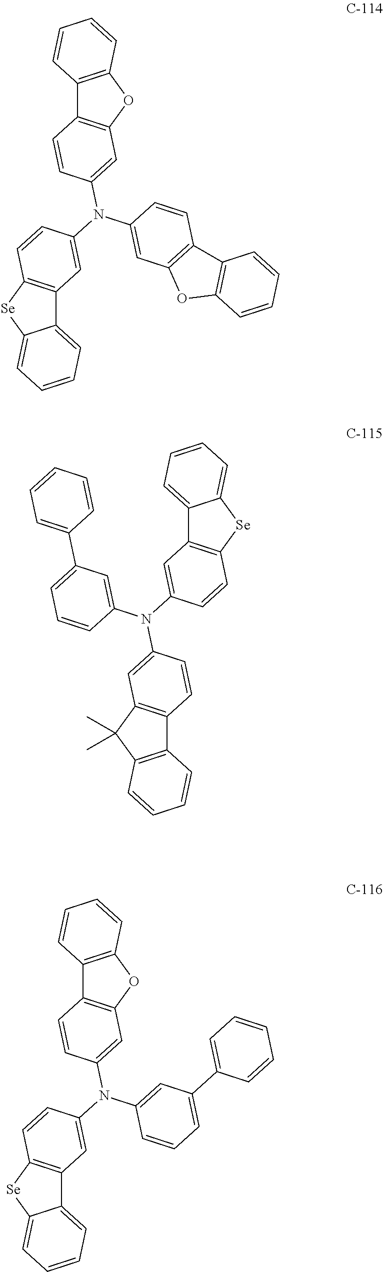 [SMILES: c1ccc2c(c1)oc1cc(N(c3ccc4c(c3)oc3ccccc34)c3ccc4[se]c5ccccc5c4c3)ccc12 ; CC1(C)c2ccccc2-c2ccc(N(c3cccc(-c4ccccc4)c3)c3ccc4[se]c5ccccc5c4c3)cc21 ; c1ccc(-c2cccc(N(c3ccc4c(c3)oc3ccccc34)c3ccc4[se]c5ccccc5c4c3)c2)cc1]

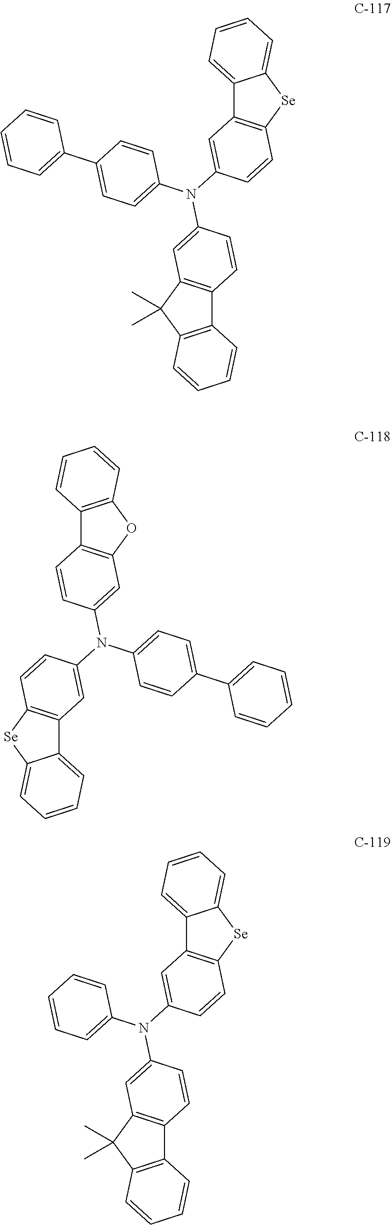 [SMILES: CC1(C)c2ccccc2-c2ccc(N(c3ccc(-c4ccccc4)cc3)c3ccc4[se]c5ccccc5c4c3)cc21 ; c1ccc(-c2ccc(N(c3ccc4c(c3)oc3ccccc34)c3ccc4[se]c5ccccc5c4c3)cc2)cc1 ; CC1(C)c2ccccc2-c2ccc(N(c3ccccc3)c3ccc4[se]c5ccccc5c4c3)cc21]

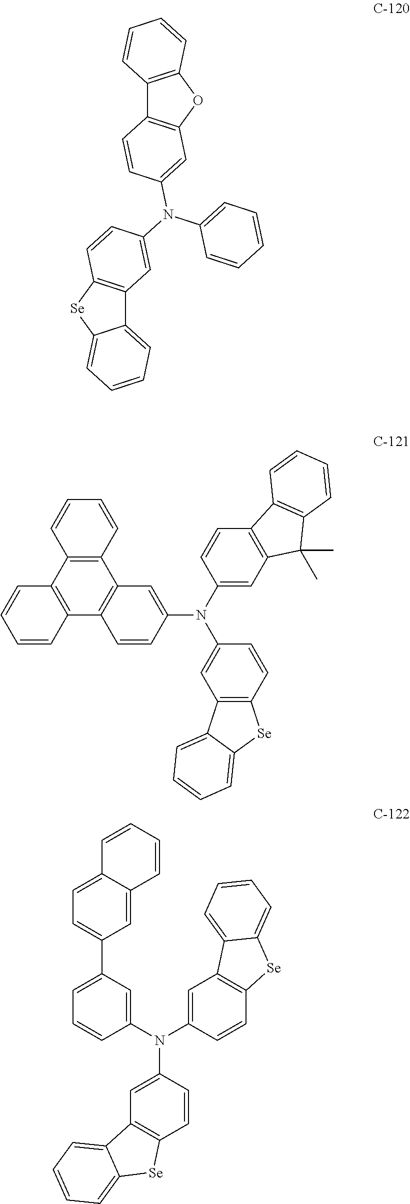 [SMILES: c1ccc(N(c2ccc3c(c2)oc2ccccc23)c2ccc3[se]c4ccccc4c3c2)cc1 ; CC1(C)c2ccccc2-c2ccc(N(c3ccc4[se]c5ccccc5c4c3)c3ccc4c5ccccc5c5ccccc5c4c3)cc21 ; c1cc(-c2ccc3ccccc3c2)cc(N(c2ccc3[se]c4ccccc4c3c2)c2ccc3[se]c4ccccc4c3c2)c1]

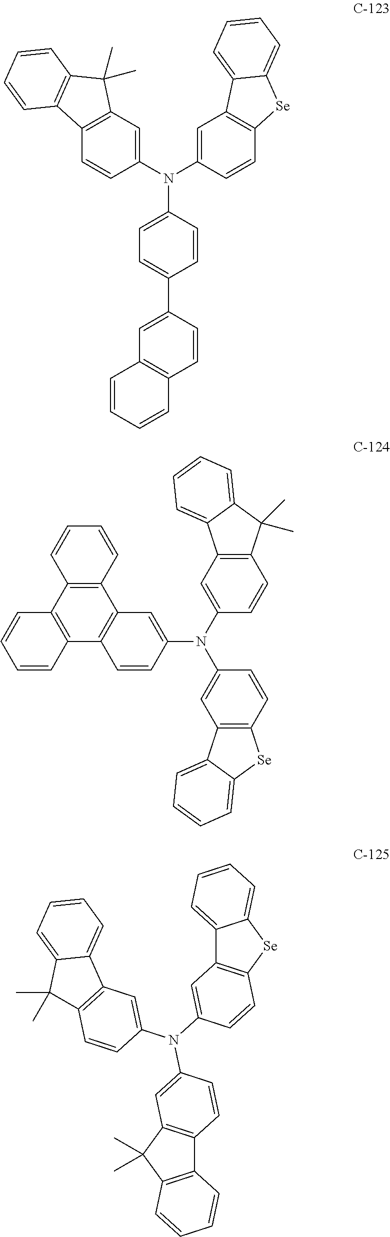

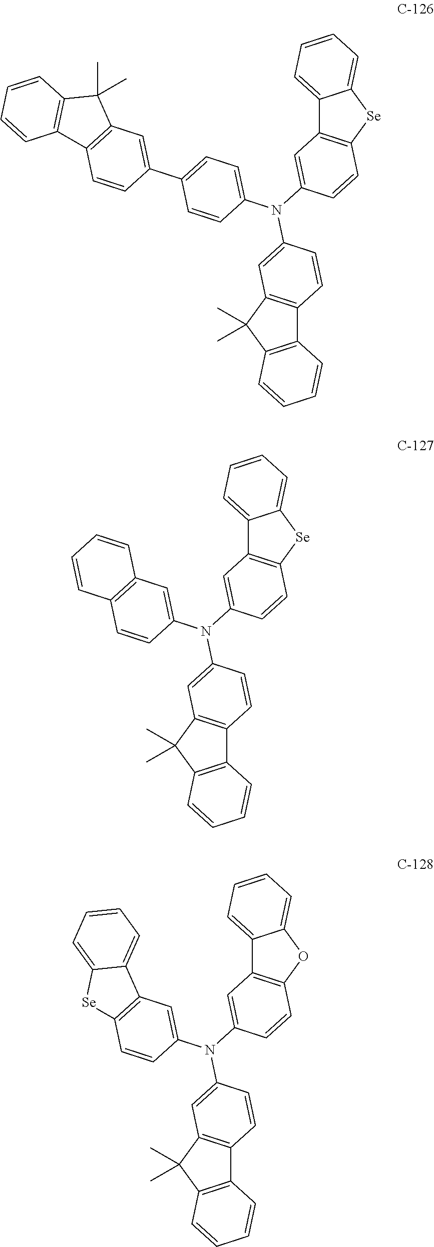 [SMILES: CC1(C)c2ccccc2-c2ccc(-c3ccc(N(c4ccc5c(c4)C(C)(C)c4ccccc4-5)c4ccc5[se]c6ccccc6c5c4)cc3)cc21 ; CC1(C)c2ccccc2-c2ccc(N(c3ccc4ccccc4c3)c3ccc4[se]c5ccccc5c4c3)cc21 ; CC1(C)c2ccccc2-c2ccc(N(c3ccc4oc5ccccc5c4c3)c3ccc4[se]c5ccccc5c4c3)cc21]

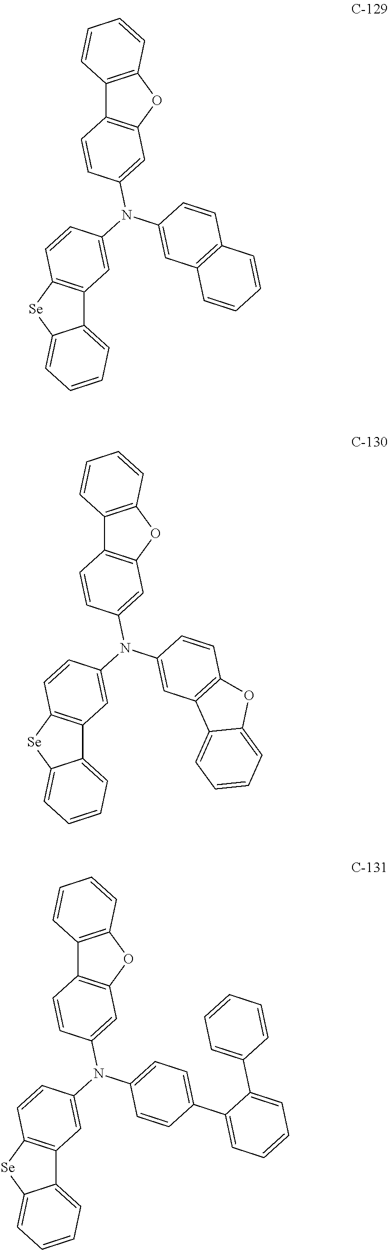 [SMILES: c1ccc2cc(N(c3ccc4c(c3)oc3ccccc34)c3ccc4[se]c5ccccc5c4c3)ccc2c1 ; c1ccc2c(c1)oc1cc(N(c3ccc4oc5ccccc5c4c3)c3ccc4[se]c5ccccc5c4c3)ccc12 ; c1ccc(-c2ccccc2-c2ccc(N(c3ccc4c(c3)oc3ccccc34)c3ccc4[se]c5ccccc5c4c3)cc2)cc1]

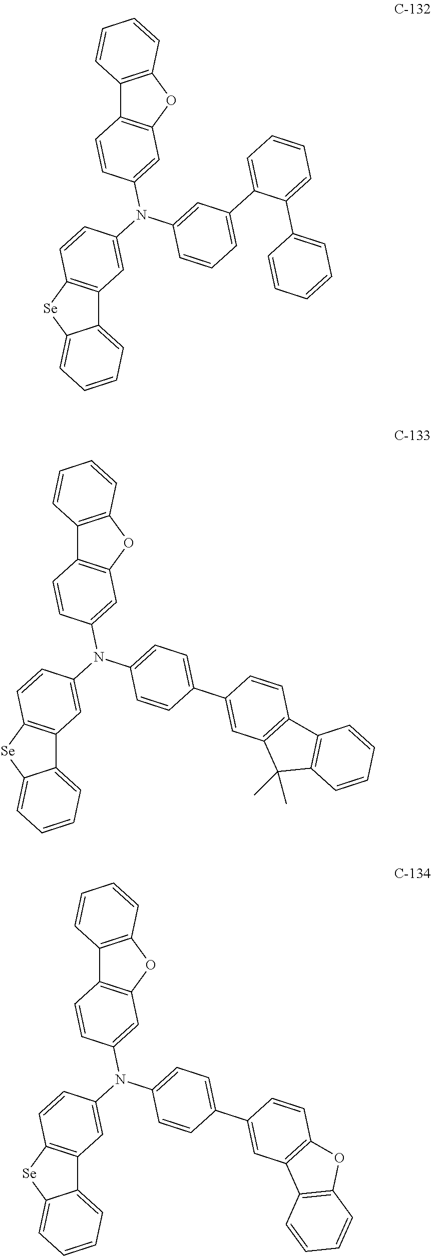 [SMILES: c1ccc(-c2ccccc2-c2cccc(N(c3ccc4c(c3)oc3ccccc34)c3ccc4[se]c5ccccc5c4c3)c2)cc1 ; CC1(C)c2ccccc2-c2ccc(-c3ccc(N(c4ccc5c(c4)oc4ccccc45)c4ccc5[se]c6ccccc6c5c4)cc3)cc21 ; c1ccc2c(c1)oc1cc(N(c3ccc(-c4ccc5oc6ccccc6c5c4)cc3)c3ccc4[se]c5ccccc5c4c3)ccc12]

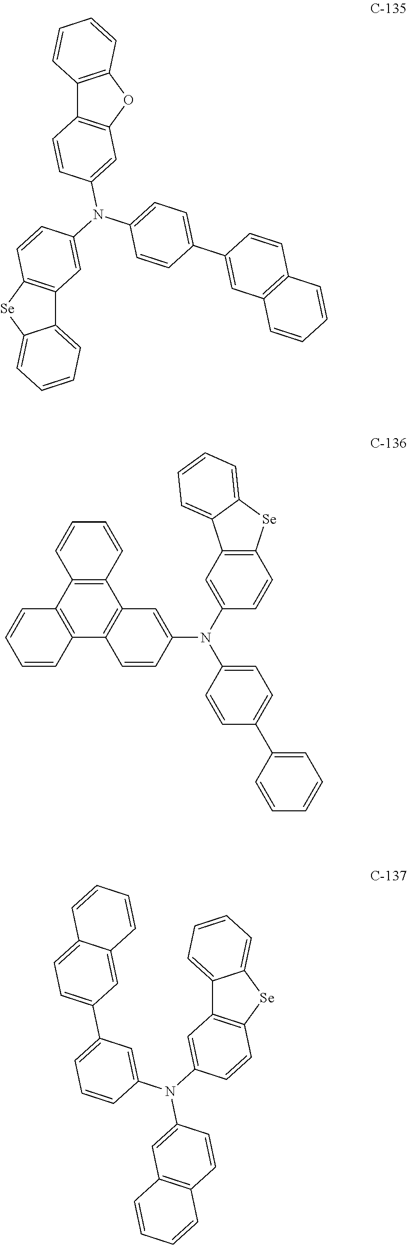 [SMILES: c1ccc2cc(-c3ccc(N(c4ccc5c(c4)oc4ccccc45)c4ccc5[se]c6ccccc6c5c4)cc3)ccc2c1 ; c1ccc(-c2ccc(N(c3ccc4[se]c5ccccc5c4c3)c3ccc4c5ccccc5c5ccccc5c4c3)cc2)cc1 ; c1cc(-c2ccc3ccccc3c2)cc(N(c2ccc3ccccc3c2)c2ccc3[se]c4ccccc4c3c2)c1]

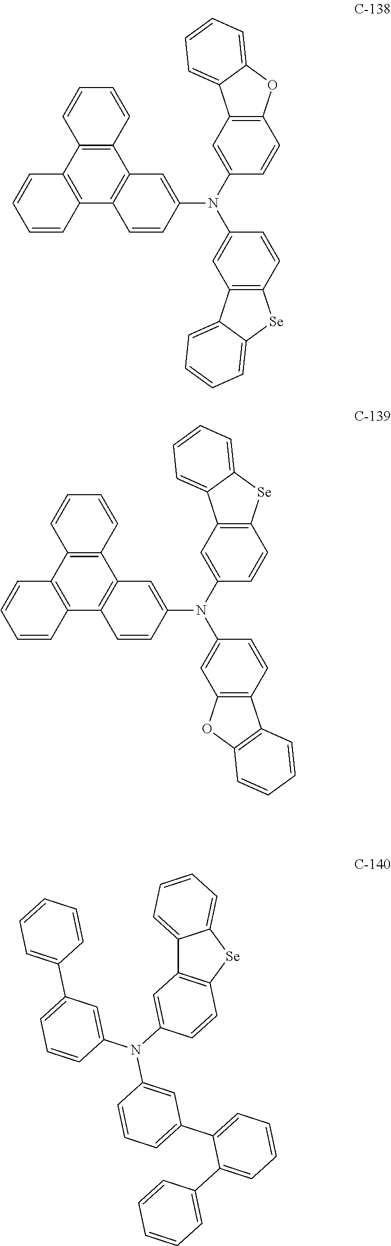

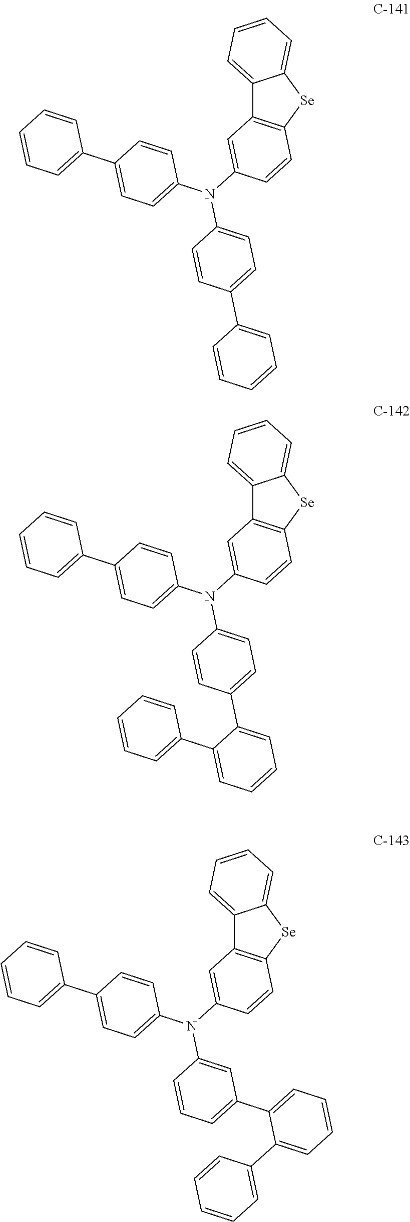

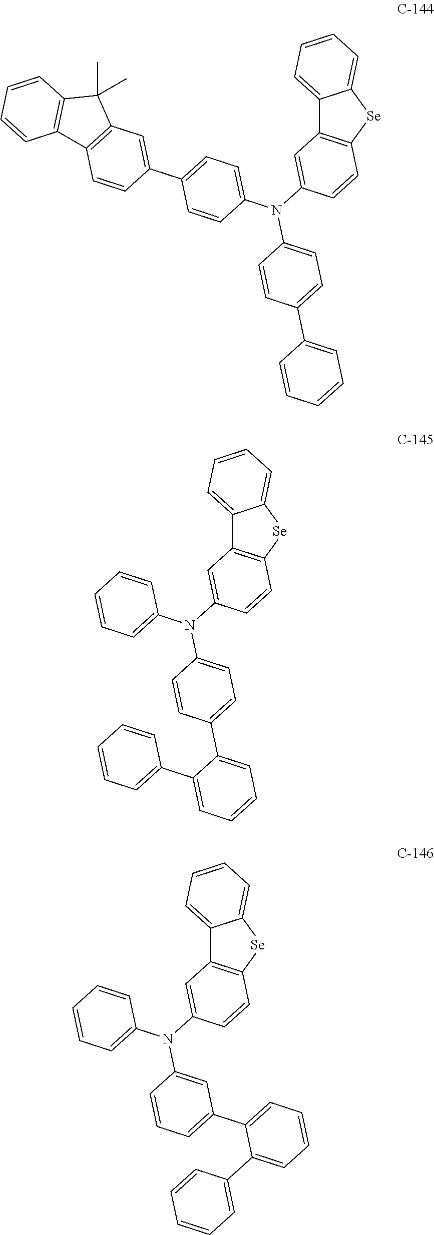

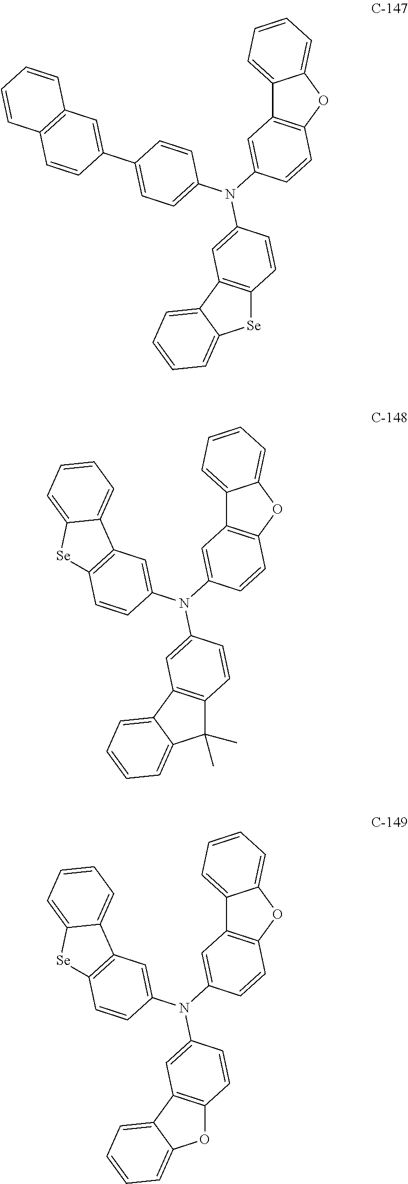 [SMILES: c1ccc2cc(-c3ccc(N(c4ccc5oc6ccccc6c5c4)c4ccc5[se]c6ccccc6c5c4)cc3)ccc2c1 ; CC1(C)c2ccccc2-c2cc(N(c3ccc4oc5ccccc5c4c3)c3ccc4[se]c5ccccc5c4c3)ccc21 ; c1ccc2c(c1)oc1ccc(N(c3ccc4oc5ccccc5c4c3)c3ccc4[se]c5ccccc5c4c3)cc12]

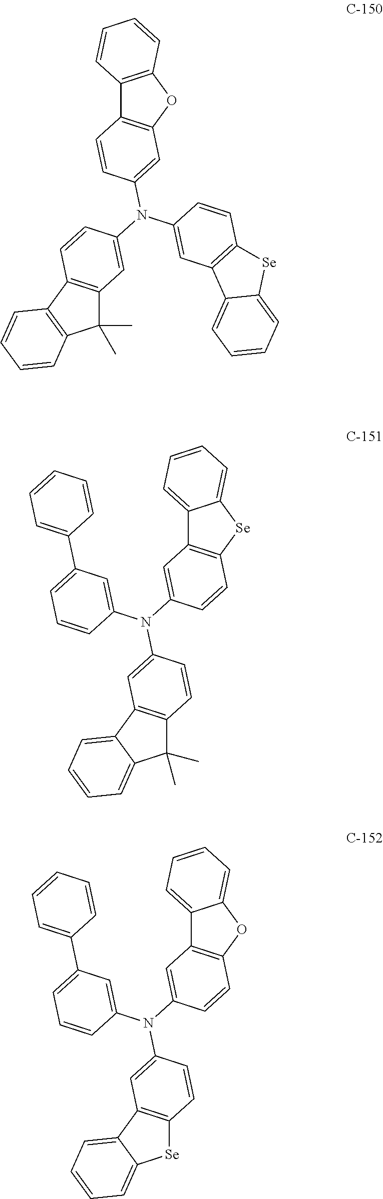 [SMILES: CC1(C)c2ccccc2-c2ccc(N(c3ccc4c(c3)oc3ccccc34)c3ccc4[se]c5ccccc5c4c3)cc21 ; CC1(C)c2ccccc2-c2cc(N(c3cccc(-c4ccccc4)c3)c3ccc4[se]c5ccccc5c4c3)ccc21 ; c1ccc(-c2cccc(N(c3ccc4oc5ccccc5c4c3)c3ccc4[se]c5ccccc5c4c3)c2)cc1]

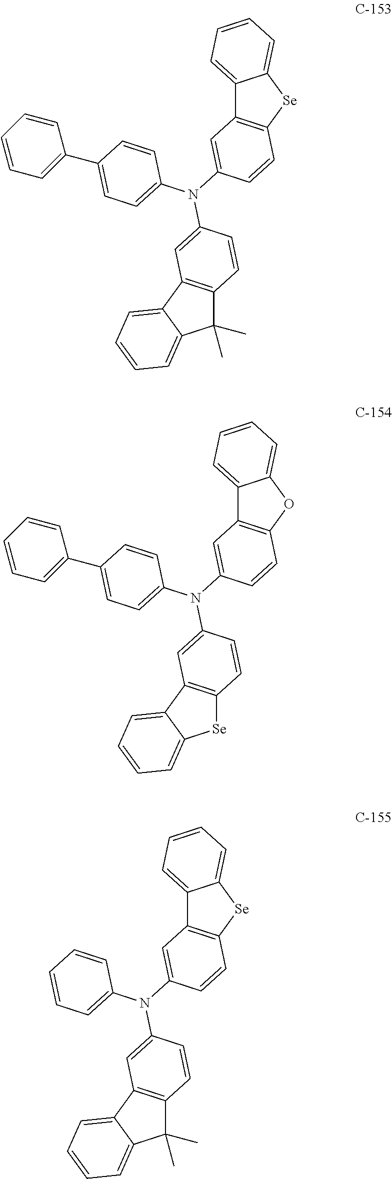 [SMILES: CC1(C)c2ccccc2-c2cc(N(c3ccc(-c4ccccc4)cc3)c3ccc4[se]c5ccccc5c4c3)ccc21 ; c1ccc(-c2ccc(N(c3ccc4oc5ccccc5c4c3)c3ccc4[se]c5ccccc5c4c3)cc2)cc1 ; CC1(C)c2ccccc2-c2cc(N(c3ccccc3)c3ccc4[se]c5ccccc5c4c3)ccc21]

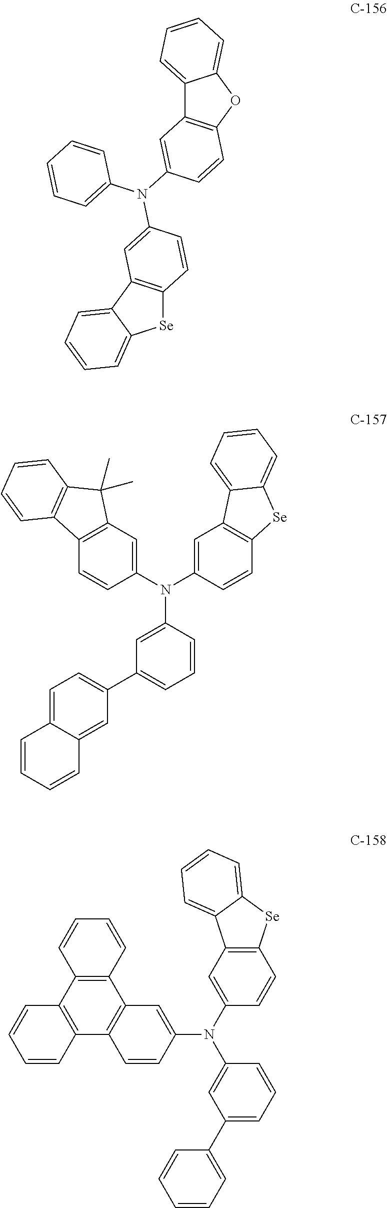 [SMILES: c1ccc(N(c2ccc3oc4ccccc4c3c2)c2ccc3[se]c4ccccc4c3c2)cc1 ; CC1(C)c2ccccc2-c2ccc(N(c3cccc(-c4ccc5ccccc5c4)c3)c3ccc4[se]c5ccccc5c4c3)cc21 ; c1ccc(-c2cccc(N(c3ccc4[se]c5ccccc5c4c3)c3ccc4c5ccccc5c5ccccc5c4c3)c2)cc1]

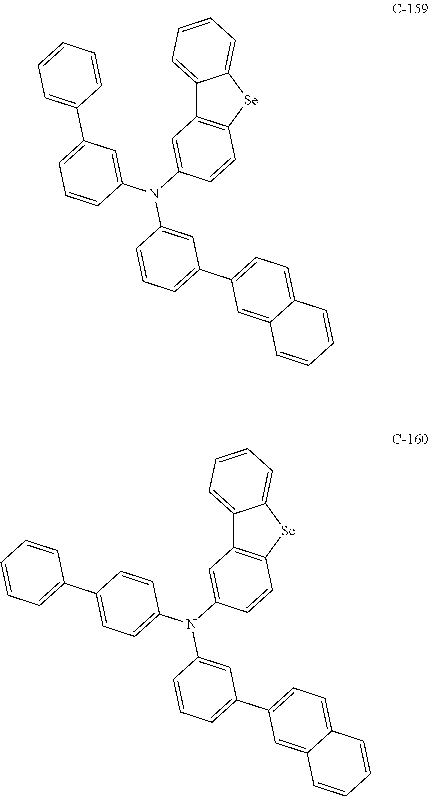 [SMILES: c1ccc(-c2cccc(N(c3cccc(-c4ccc5ccccc5c4)c3)c3ccc4[se]c5ccccc5c4c3)c2)cc1 ; c1ccc(-c2ccc(N(c3cccc(-c4ccc5ccccc5c4)c3)c3ccc4[se]c5ccccc5c4c3)cc2)cc1]

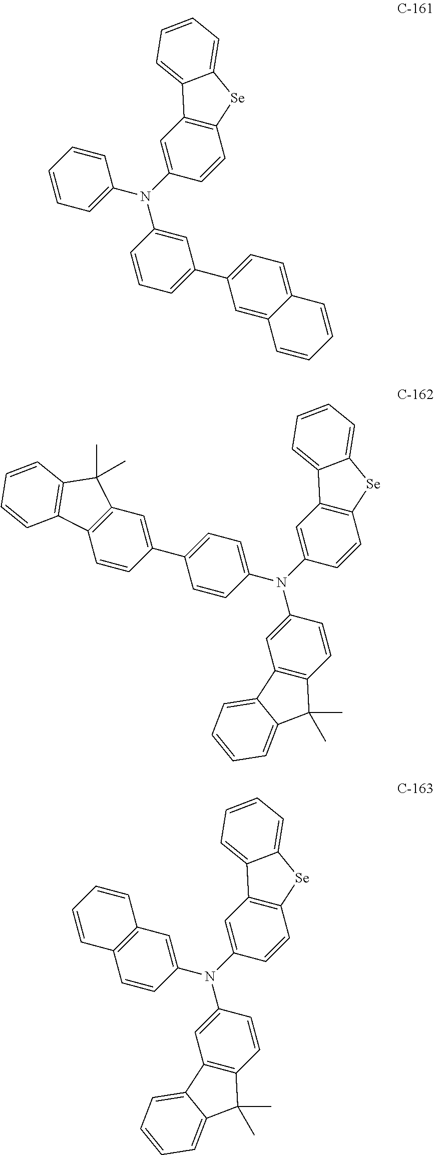

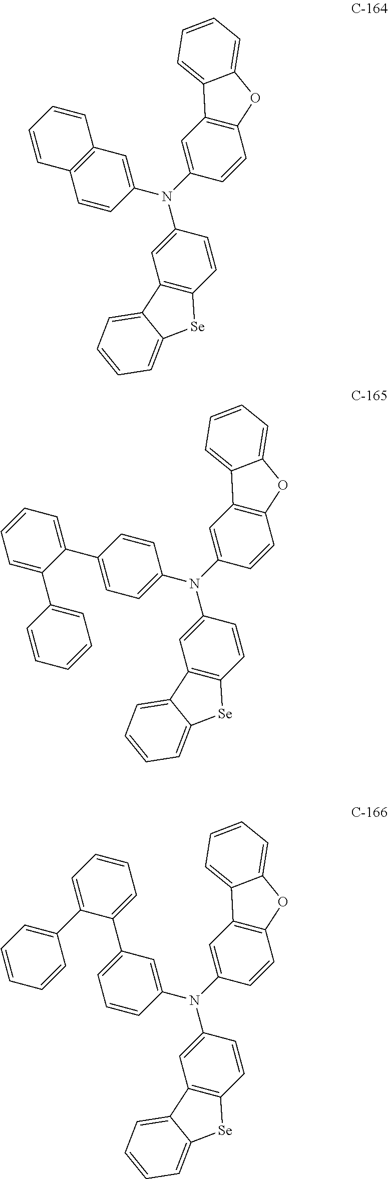 [SMILES: c1ccc2cc(N(c3ccc4oc5ccccc5c4c3)c3ccc4[se]c5ccccc5c4c3)ccc2c1 ; c1ccc(-c2ccccc2-c2ccc(N(c3ccc4oc5ccccc5c4c3)c3ccc4[se]c5ccccc5c4c3)cc2)cc1 ; c1ccc(-c2ccccc2-c2cccc(N(c3ccc4oc5ccccc5c4c3)c3ccc4[se]c5ccccc5c4c3)c2)cc1]

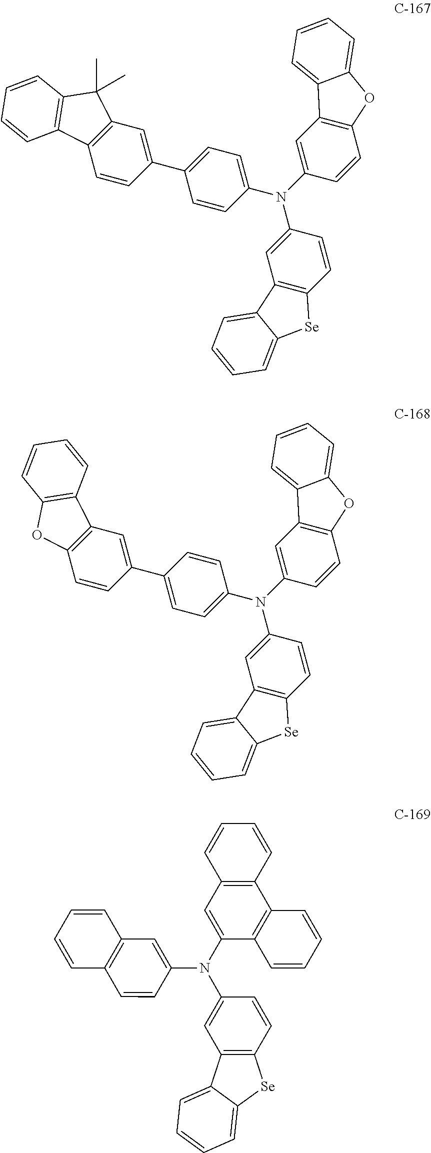

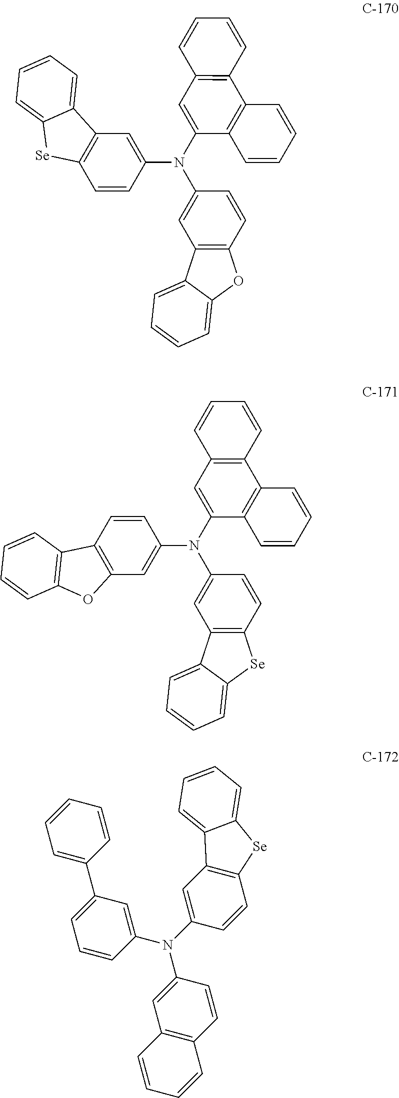

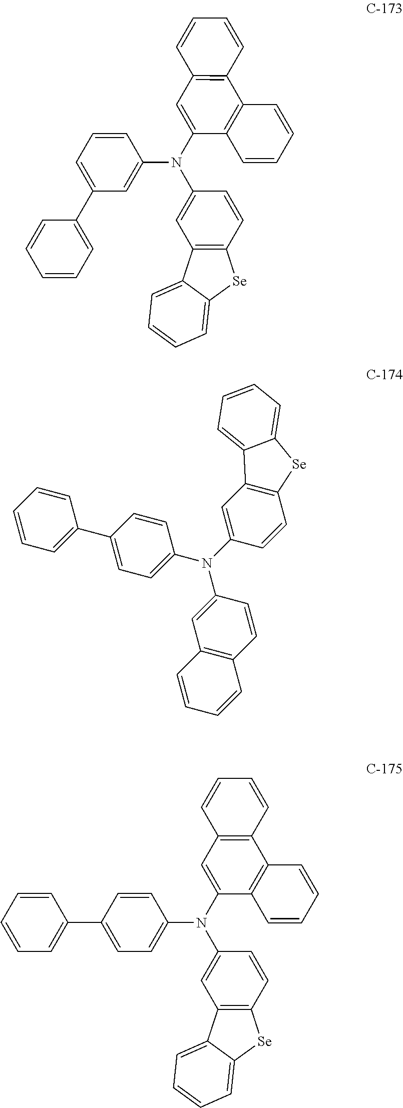 [SMILES: c1ccc(-c2cccc(N(c3ccc4[se]c5ccccc5c4c3)c3cc4ccccc4c4ccccc34)c2)cc1 ; c1ccc(-c2ccc(N(c3ccc4ccccc4c3)c3ccc4[se]c5ccccc5c4c3)cc2)cc1 ; c1ccc(-c2ccc(N(c3ccc4[se]c5ccccc5c4c3)c3cc4ccccc4c4ccccc34)cc2)cc1]

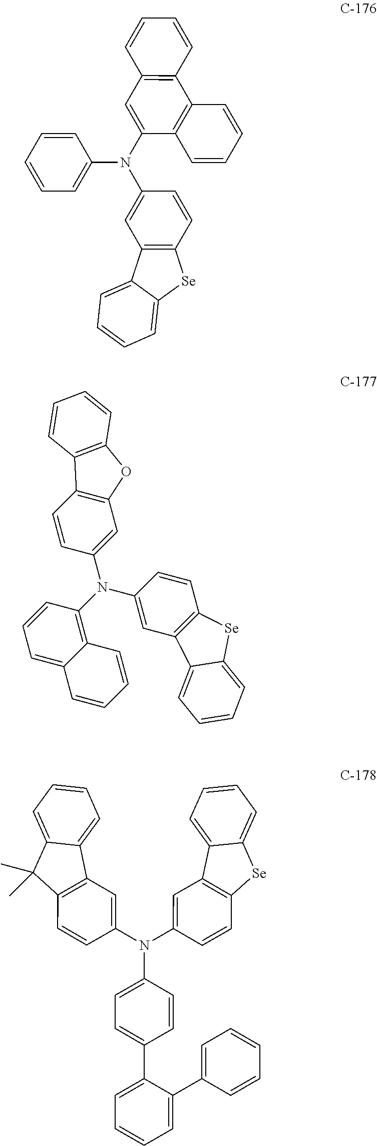 [SMILES: c1ccc(N(c2ccc3[se]c4ccccc4c3c2)c2cc3ccccc3c3ccccc23)cc1 ; c1ccc2c(N(c3ccc4c(c3)oc3ccccc34)c3ccc4[se]c5ccccc5c4c3)cccc2c1 ; CC1(C)c2ccccc2-c2cc(N(c3ccc(-c4ccccc4-c4ccccc4)cc3)c3ccc4[se]c5ccccc5c4c3)ccc21]

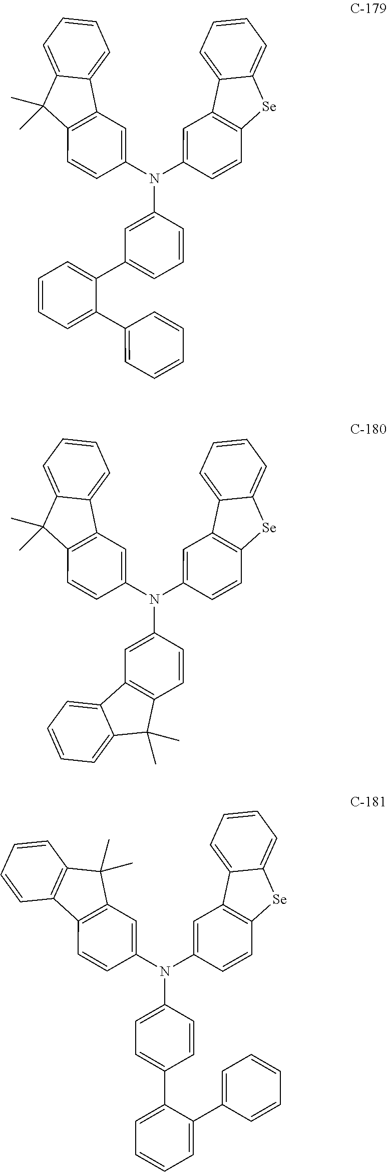 [SMILES: CC1(C)c2ccccc2-c2cc(N(c3cccc(-c4ccccc4-c4ccccc4)c3)c3ccc4[se]c5ccccc5c4c3)ccc21 ; CC1(C)c2ccccc2-c2cc(N(c3ccc4c(c3)-c3ccccc3C4(C)C)c3ccc4[se]c5ccccc5c4c3)ccc21 ; CC1(C)c2ccccc2-c2ccc(N(c3ccc(-c4ccccc4-c4ccccc4)cc3)c3ccc4[se]c5ccccc5c4c3)cc21]

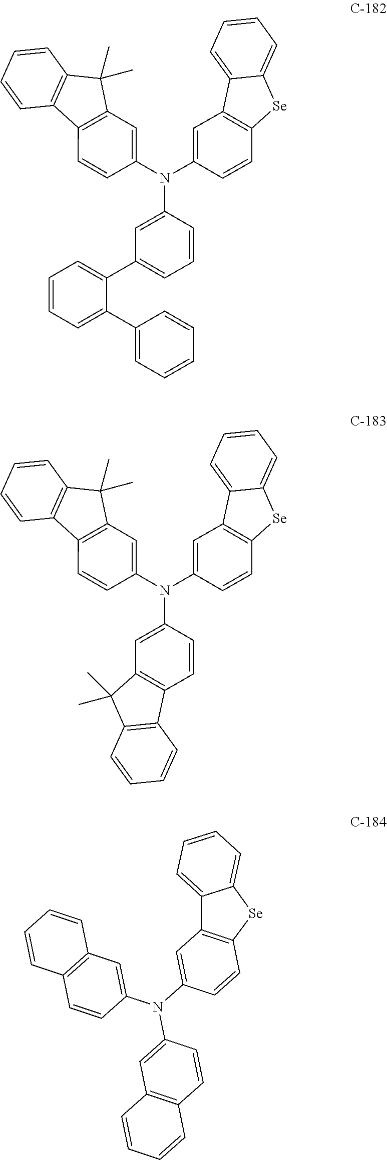 [SMILES: CC1(C)c2ccccc2-c2ccc(N(c3cccc(-c4ccccc4-c4ccccc4)c3)c3ccc4[se]c5ccccc5c4c3)cc21 ; CC1(C)c2ccccc2-c2ccc(N(c3ccc4c(c3)C(C)(C)c3ccccc3-4)c3ccc4[se]c5ccccc5c4c3)cc21 ; c1ccc2cc(N(c3ccc4ccccc4c3)c3ccc4[se]c5ccccc5c4c3)ccc2c1]

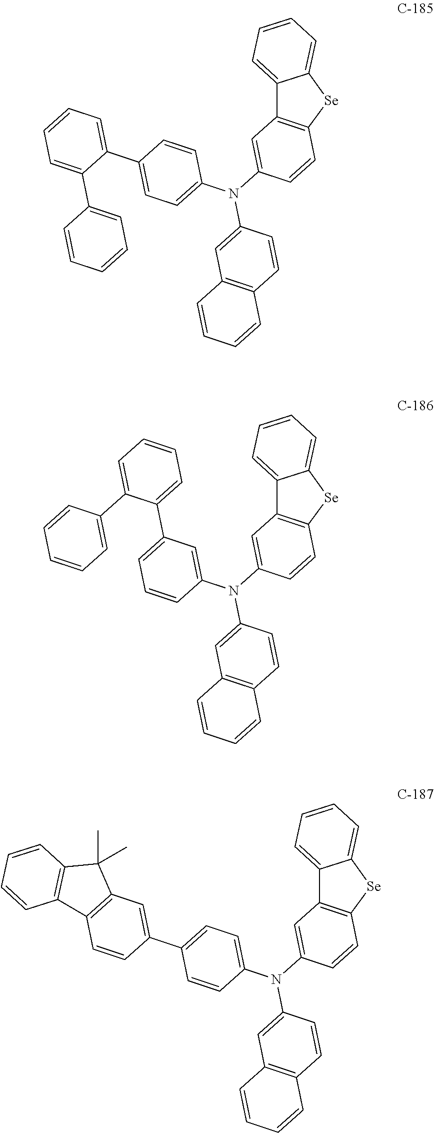 [SMILES: c1ccc(-c2ccccc2-c2ccc(N(c3ccc4ccccc4c3)c3ccc4[se]c5ccccc5c4c3)cc2)cc1 ; c1ccc(-c2ccccc2-c2cccc(N(c3ccc4ccccc4c3)c3ccc4[se]c5ccccc5c4c3)c2)cc1 ; CC1(C)c2ccccc2-c2ccc(-c3ccc(N(c4ccc5ccccc5c4)c4ccc5[se]c6ccccc6c5c4)cc3)cc21]

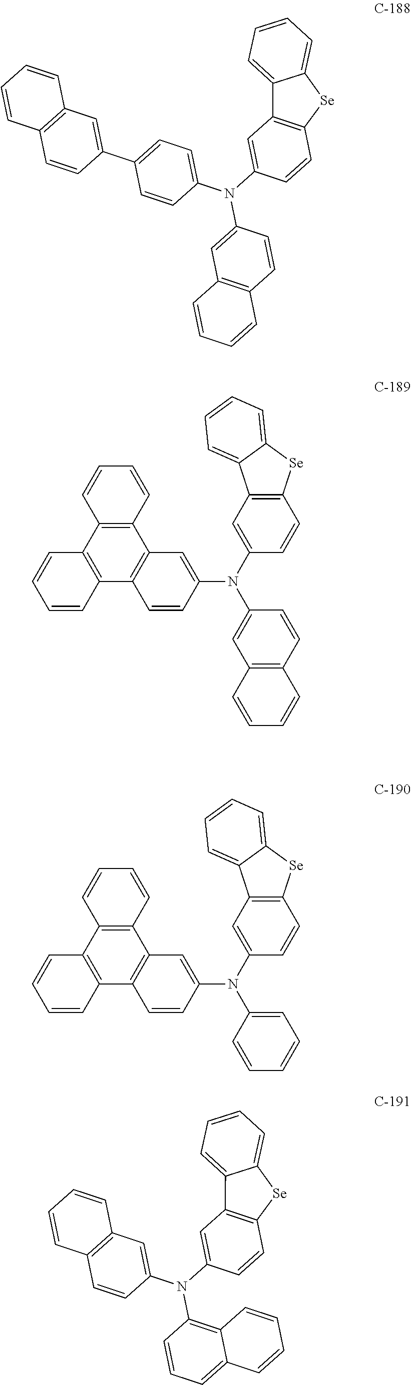

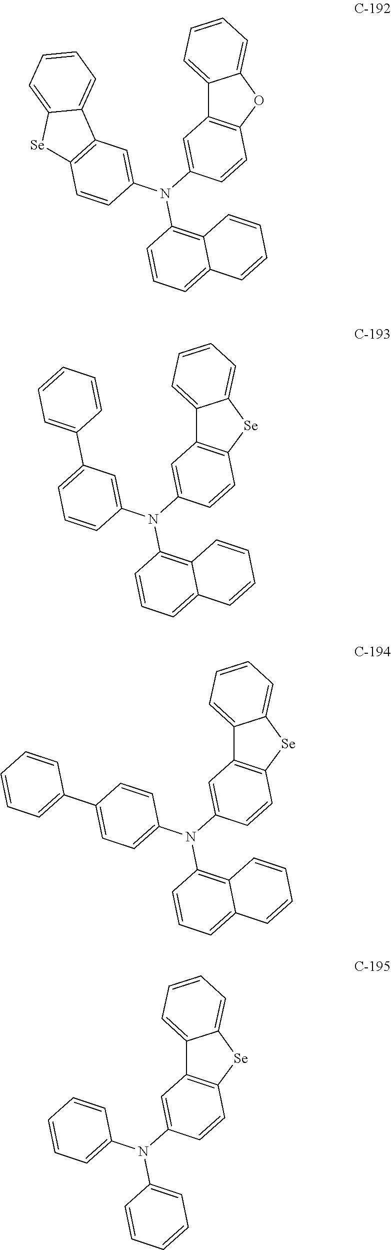

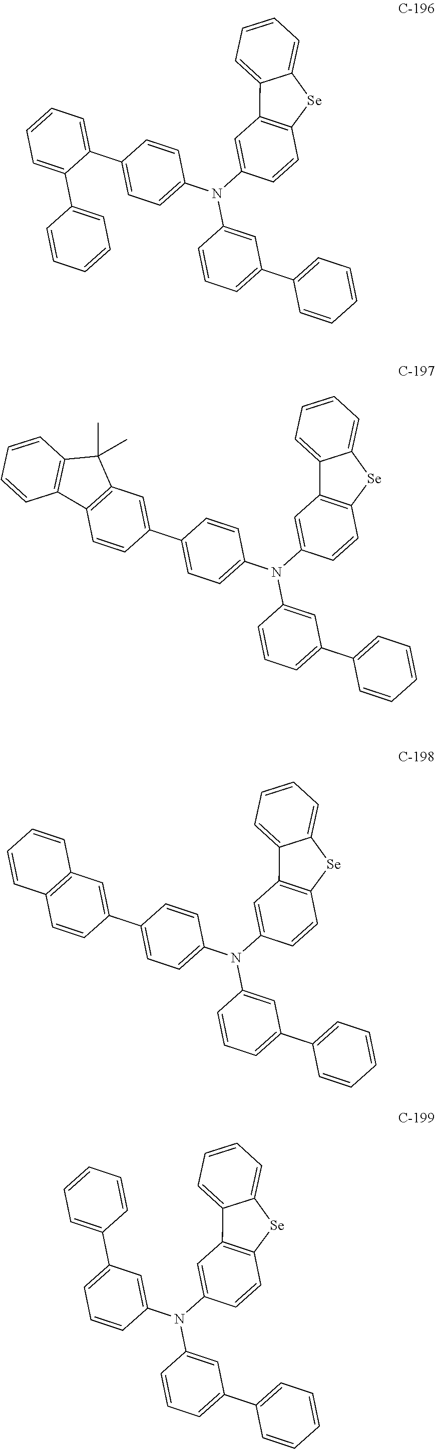 [SMILES: c1ccc(-c2cccc(N(c3ccc(-c4ccccc4-c4ccccc4)cc3)c3ccc4[se]c5ccccc5c4c3)c2)cc1 ; CC1(C)c2ccccc2-c2ccc(-c3ccc(N(c4cccc(-c5ccccc5)c4)c4ccc5[se]c6ccccc6c5c4)cc3)cc21 ; c1ccc(-c2cccc(N(c3ccc(-c4ccc5ccccc5c4)cc3)c3ccc4[se]c5ccccc5c4c3)c2)cc1 ; c1ccc(-c2cccc(N(c3cccc(-c4ccccc4)c3)c3ccc4[se]c5ccccc5c4c3)c2)cc1]

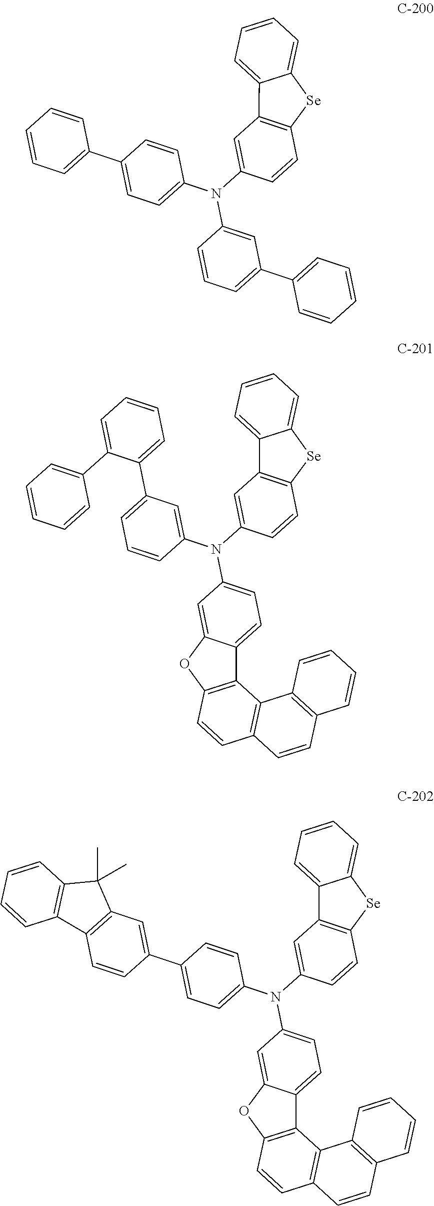 [SMILES: c1ccc(-c2ccc(N(c3cccc(-c4ccccc4)c3)c3ccc4[se]c5ccccc5c4c3)cc2)cc1 ; c1ccc(-c2ccccc2-c2cccc(N(c3ccc4c(c3)oc3ccc5ccc6ccccc6c5c34)c3ccc4[se]c5ccccc5c4c3)c2)cc1 ; CC1(C)c2ccccc2-c2ccc(-c3ccc(N(c4ccc5c(c4)oc4ccc6ccc7ccccc7c6c45)c4ccc5[se]c6ccccc6c5c4)cc3)cc21]

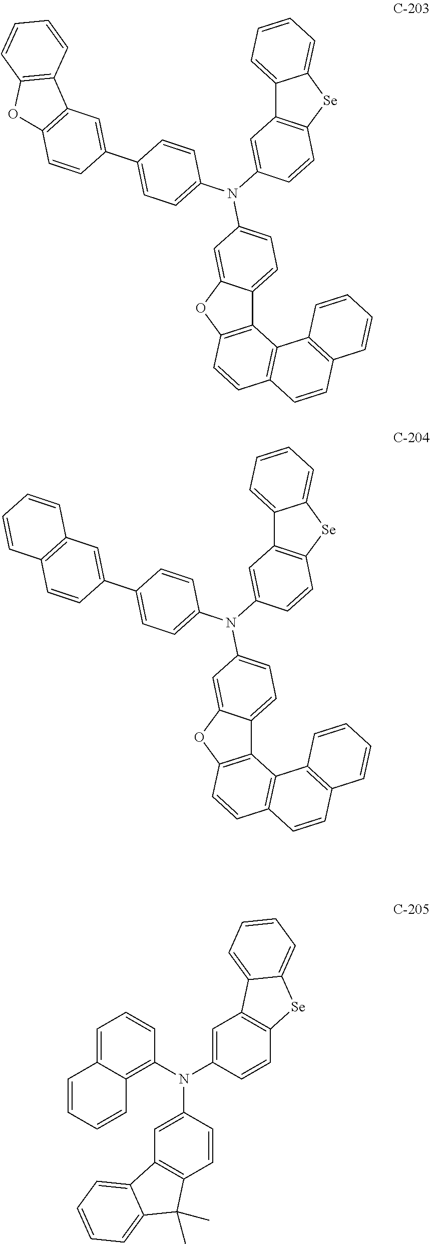

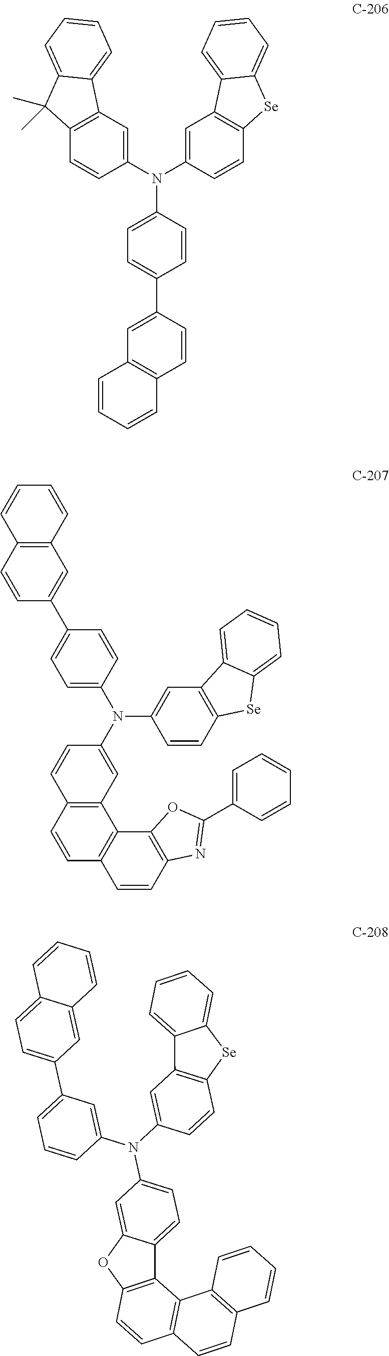 [SMILES: CC1(C)c2ccccc2-c2cc(N(c3ccc(-c4ccc5ccccc5c4)cc3)c3ccc4[se]c5ccccc5c4c3)ccc21 ; c1ccc(-c2nc3ccc4ccc5ccc(N(c6ccc(-c7ccc8ccccc8c7)cc6)c6ccc7[se]c8ccccc8c7c6)cc5c4c3o2)cc1 ; c1cc(-c2ccc3ccccc3c2)cc(N(c2ccc3c(c2)oc2ccc4ccc5ccccc5c4c23)c2ccc3[se]c4ccccc4c3c2)c1]

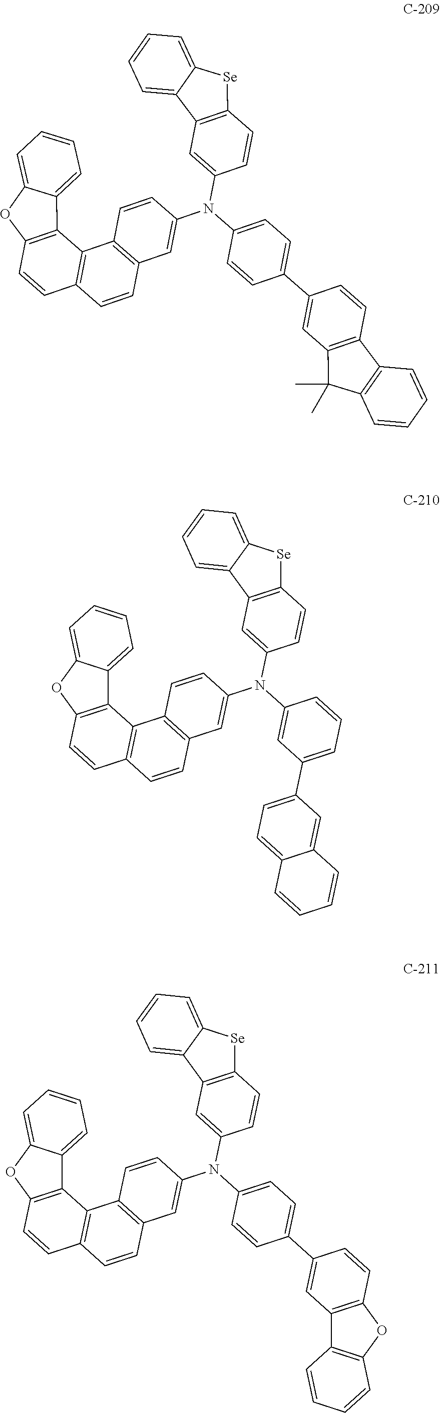 [SMILES: CC1(C)c2ccccc2-c2ccc(-c3ccc(N(c4ccc5c(ccc6ccc7oc8ccccc8c7c65)c4)c4ccc5[se]c6ccccc6c5c4)cc3)cc21 ; c1cc(-c2ccc3ccccc3c2)cc(N(c2ccc3c(ccc4ccc5oc6ccccc6c5c43)c2)c2ccc3[se]c4ccccc4c3c2)c1 ; c1ccc2c(c1)oc1ccc(-c3ccc(N(c4ccc5c(ccc6ccc7oc8ccccc8c7c65)c4)c4ccc5[se]c6ccccc6c5c4)cc3)cc12]

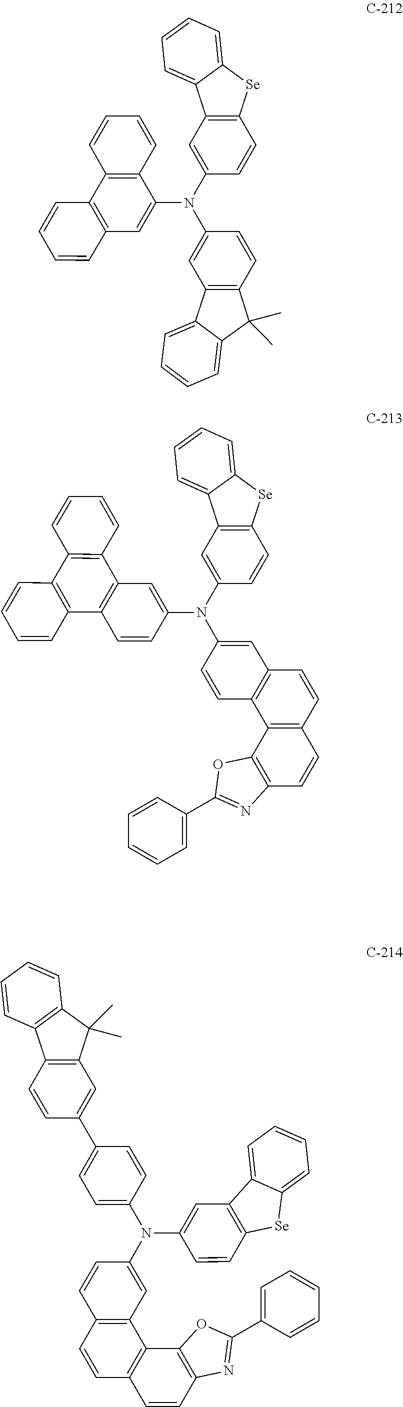

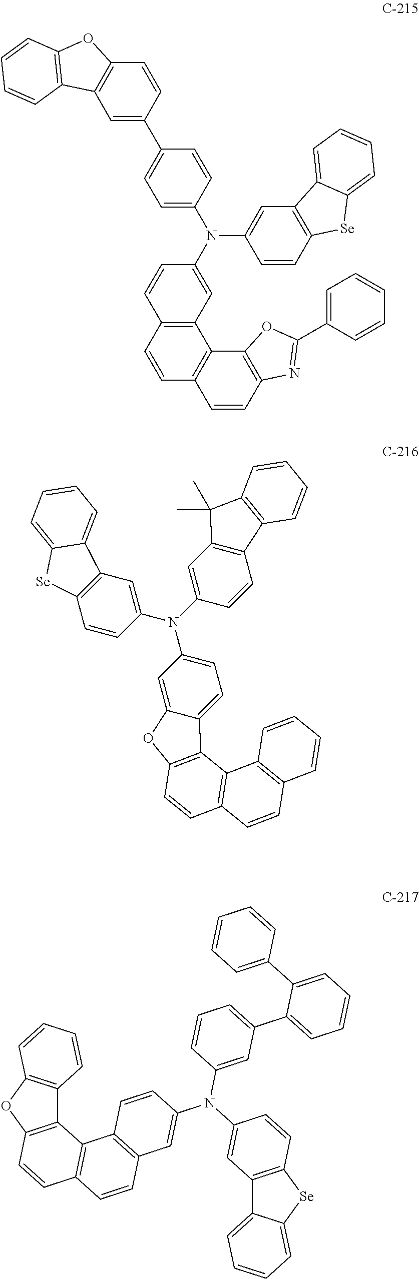 [SMILES: c1ccc(-c2nc3ccc4ccc5ccc(N(c6ccc(-c7ccc8oc9ccccc9c8c7)cc6)c6ccc7[se]c8ccccc8c7c6)cc5c4c3o2)cc1 ; CC1(C)c2ccccc2-c2ccc(N(c3ccc4c(c3)oc3ccc5ccc6ccccc6c5c34)c3ccc4[se]c5ccccc5c4c3)cc21 ; c1ccc(-c2ccccc2-c2cccc(N(c3ccc4c(ccc5ccc6oc7ccccc7c6c54)c3)c3ccc4[se]c5ccccc5c4c3)c2)cc1]

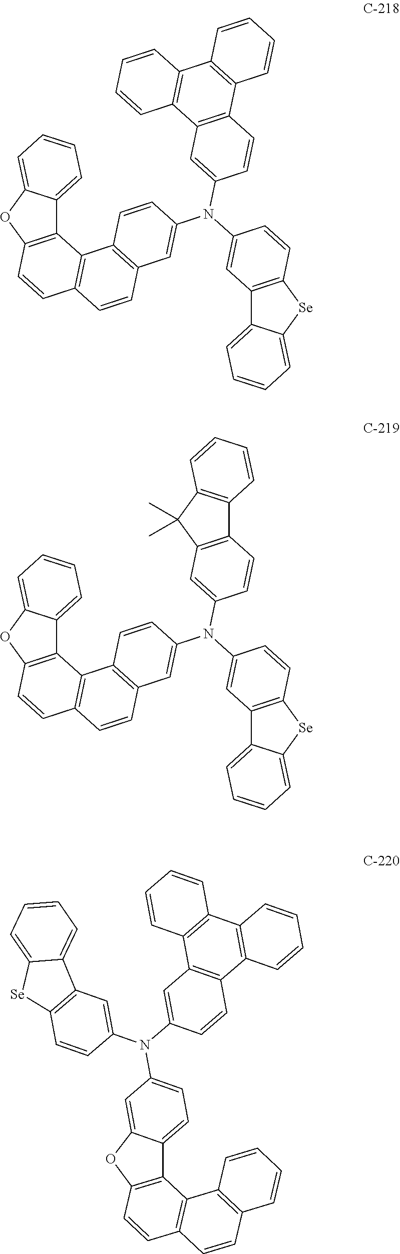 [SMILES: c1ccc2c(c1)oc1ccc3ccc4cc(N(c5ccc6[se]c7ccccc7c6c5)c5ccc6c7ccccc7c7ccccc7c6c5)ccc4c3c12 ; CC1(C)c2ccccc2-c2ccc(N(c3ccc4c(ccc5ccc6oc7ccccc7c6c54)c3)c3ccc4[se]c5ccccc5c4c3)cc21 ; c1ccc2c(c1)ccc1ccc3oc4cc(N(c5ccc6[se]c7ccccc7c6c5)c5ccc6c7ccccc7c7ccccc7c6c5)ccc4c3c12]

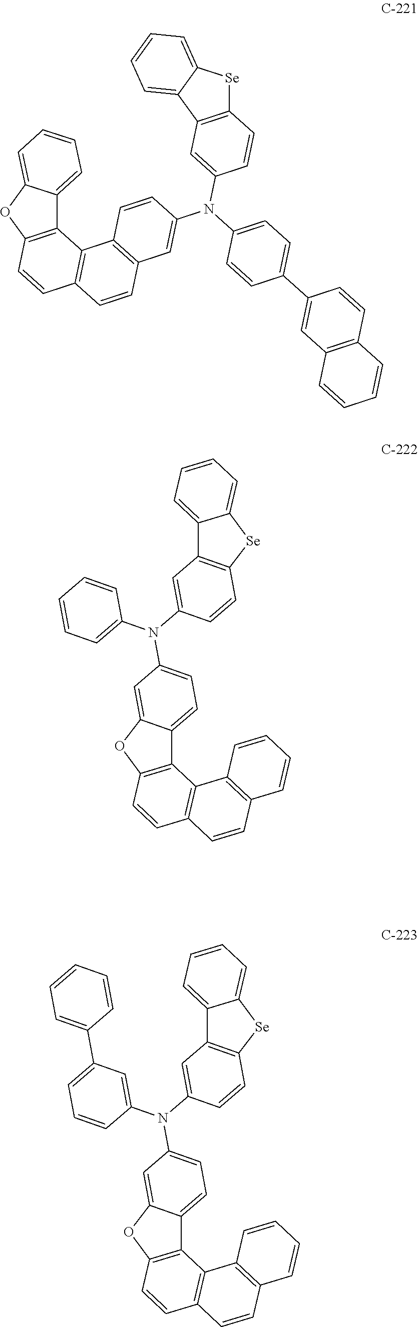 [SMILES: c1ccc2cc(-c3ccc(N(c4ccc5c(ccc6ccc7oc8ccccc8c7c65)c4)c4ccc5[se]c6ccccc6c5c4)cc3)ccc2c1 ; c1ccc(N(c2ccc3c(c2)oc2ccc4ccc5ccccc5c4c23)c2ccc3[se]c4ccccc4c3c2)cc1 ; c1ccc(-c2cccc(N(c3ccc4c(c3)oc3ccc5ccc6ccccc6c5c34)c3ccc4[se]c5ccccc5c4c3)c2)cc1]

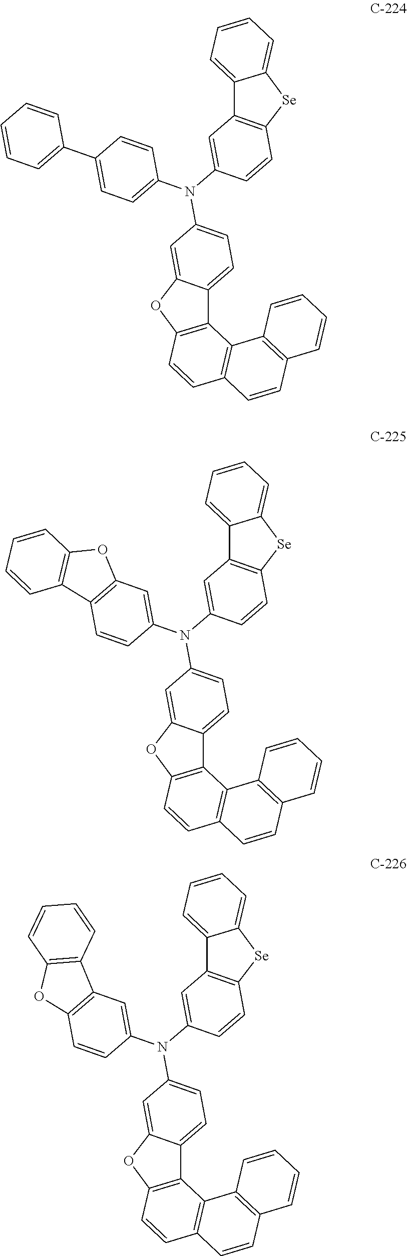

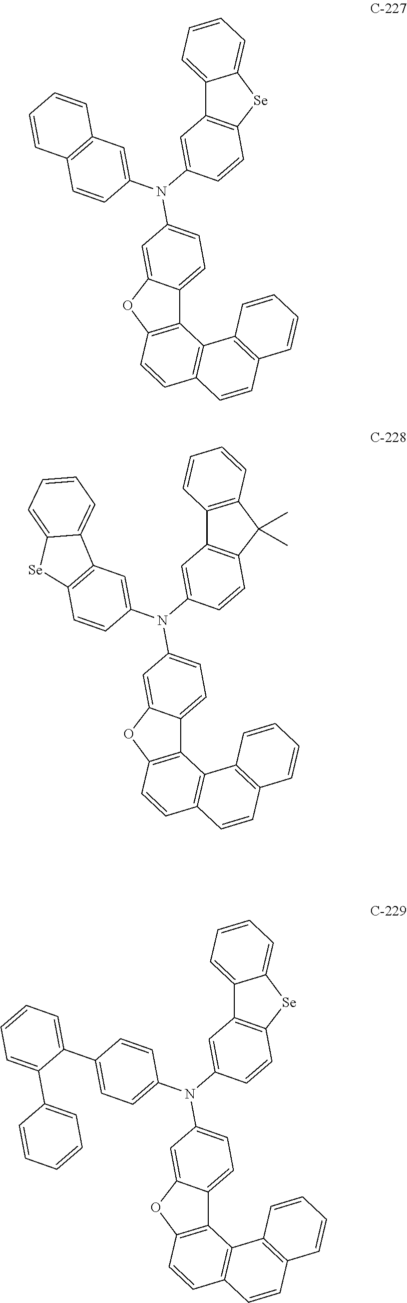 [SMILES: c1ccc2cc(N(c3ccc4c(c3)oc3ccc5ccc6ccccc6c5c34)c3ccc4[se]c5ccccc5c4c3)ccc2c1 ; CC1(C)c2ccccc2-c2cc(N(c3ccc4c(c3)oc3ccc5ccc6ccccc6c5c34)c3ccc4[se]c5ccccc5c4c3)ccc21 ; c1ccc(-c2ccccc2-c2ccc(N(c3ccc4c(c3)oc3ccc5ccc6ccccc6c5c34)c3ccc4[se]c5ccccc5c4c3)cc2)cc1]

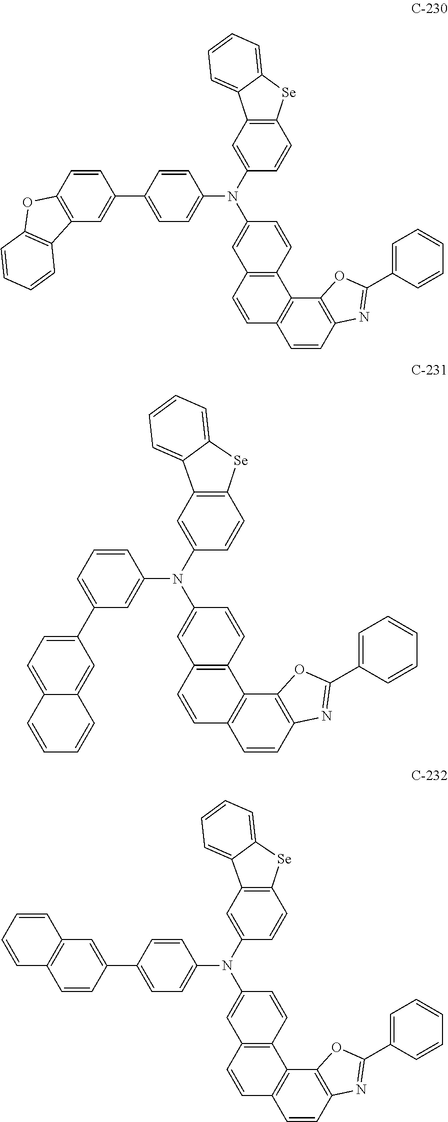 [SMILES: c1ccc(-c2nc3ccc4ccc5cc(N(c6ccc(-c7ccc8oc9ccccc9c8c7)cc6)c6ccc7[se]c8ccccc8c7c6)ccc5c4c3o2)cc1 ; c1ccc(-c2nc3ccc4ccc5cc(N(c6cccc(-c7ccc8ccccc8c7)c6)c6ccc7[se]c8ccccc8c7c6)ccc5c4c3o2)cc1 ; c1ccc(-c2nc3ccc4ccc5cc(N(c6ccc(-c7ccc8ccccc8c7)cc6)c6ccc7[se]c8ccccc8c7c6)ccc5c4c3o2)cc1]

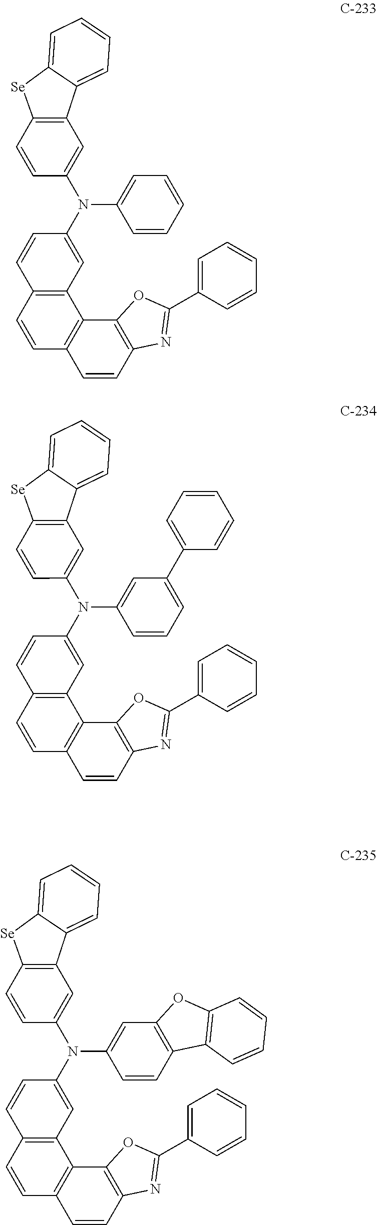

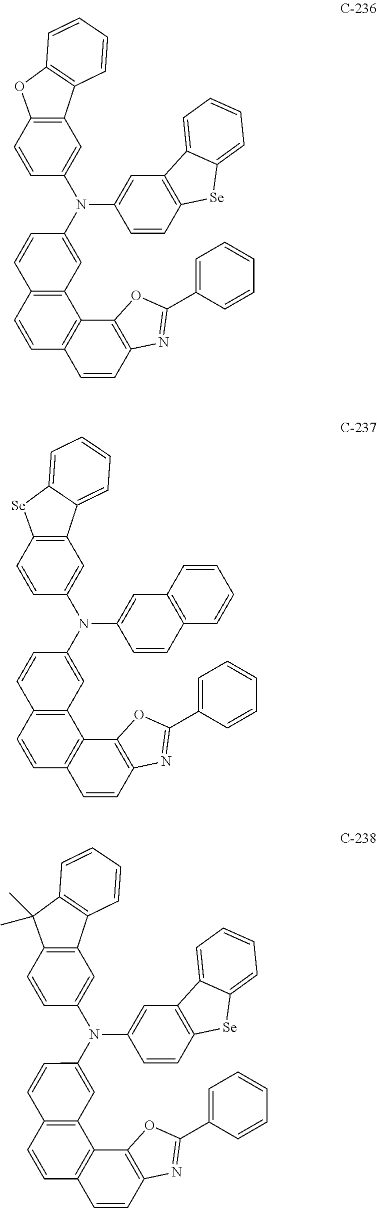 [SMILES: c1ccc(-c2nc3ccc4ccc5ccc(N(c6ccc7oc8ccccc8c7c6)c6ccc7[se]c8ccccc8c7c6)cc5c4c3o2)cc1 ; c1ccc(-c2nc3ccc4ccc5ccc(N(c6ccc7ccccc7c6)c6ccc7[se]c8ccccc8c7c6)cc5c4c3o2)cc1 ; CC1(C)c2ccccc2-c2cc(N(c3ccc4[se]c5ccccc5c4c3)c3ccc4ccc5ccc6nc(-c7ccccc7)oc6c5c4c3)ccc21]

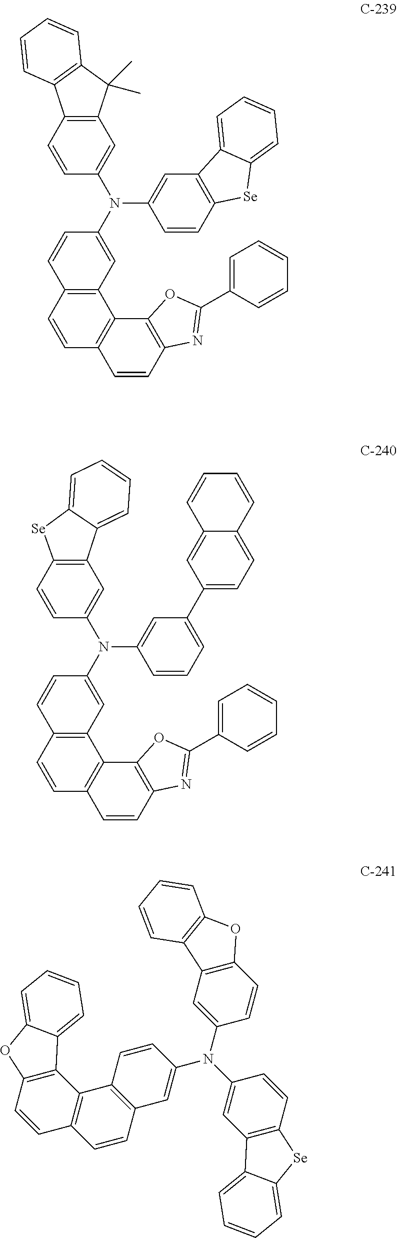 [SMILES: CC1(C)c2ccccc2-c2ccc(N(c3ccc4[se]c5ccccc5c4c3)c3ccc4ccc5ccc6nc(-c7ccccc7)oc6c5c4c3)cc21 ; c1ccc(-c2nc3ccc4ccc5ccc(N(c6cccc(-c7ccc8ccccc8c7)c6)c6ccc7[se]c8ccccc8c7c6)cc5c4c3o2)cc1 ; c1ccc2c(c1)oc1ccc(N(c3ccc4c(ccc5ccc6oc7ccccc7c6c54)c3)c3ccc4[se]c5ccccc5c4c3)cc12]

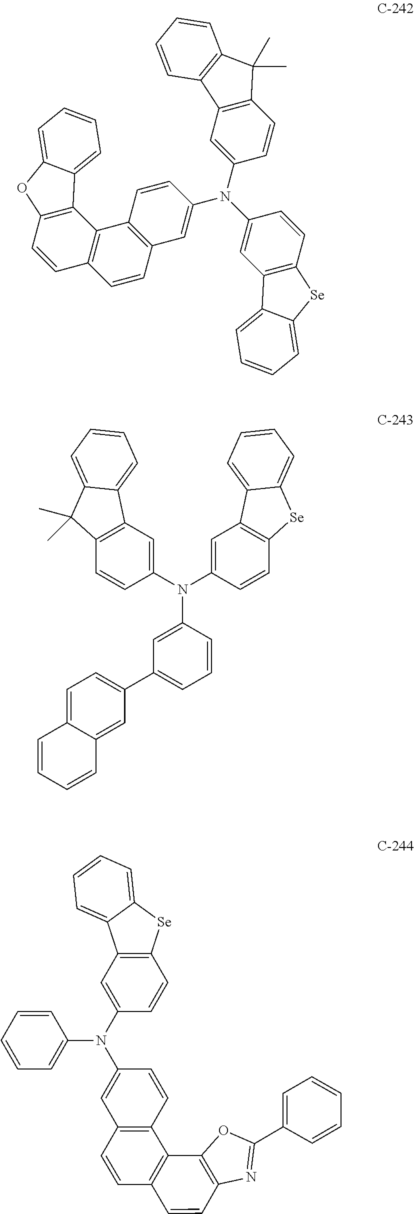 [SMILES: CC1(C)c2ccccc2-c2cc(N(c3ccc4c(ccc5ccc6oc7ccccc7c6c54)c3)c3ccc4[se]c5ccccc5c4c3)ccc21 ; CC1(C)c2ccccc2-c2cc(N(c3cccc(-c4ccc5ccccc5c4)c3)c3ccc4[se]c5ccccc5c4c3)ccc21 ; c1ccc(-c2nc3ccc4ccc5cc(N(c6ccccc6)c6ccc7[se]c8ccccc8c7c6)ccc5c4c3o2)cc1]

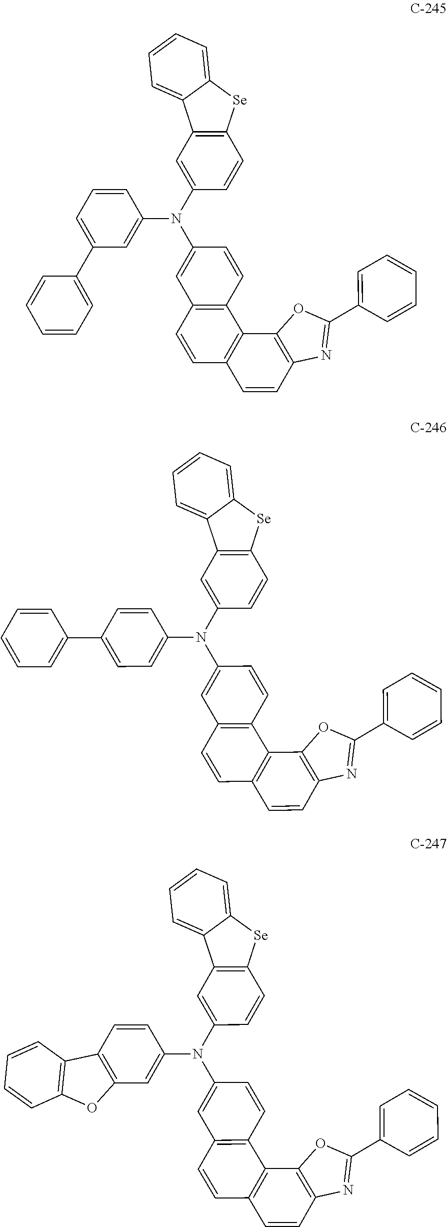 [SMILES: c1ccc(-c2cccc(N(c3ccc4c(ccc5ccc6nc(-c7ccccc7)oc6c54)c3)c3ccc4[se]c5ccccc5c4c3)c2)cc1 ; c1ccc(-c2ccc(N(c3ccc4c(ccc5ccc6nc(-c7ccccc7)oc6c54)c3)c3ccc4[se]c5ccccc5c4c3)cc2)cc1 ; c1ccc(-c2nc3ccc4ccc5cc(N(c6ccc7c(c6)oc6ccccc67)c6ccc7[se]c8ccccc8c7c6)ccc5c4c3o2)cc1]

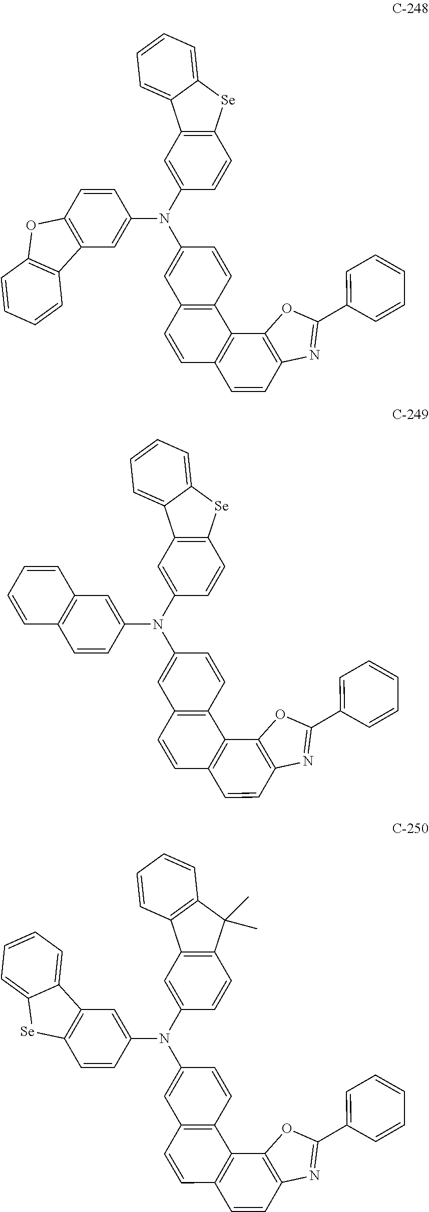 [SMILES: c1ccc(-c2nc3ccc4ccc5cc(N(c6ccc7oc8ccccc8c7c6)c6ccc7[se]c8ccccc8c7c6)ccc5c4c3o2)cc1 ; c1ccc(-c2nc3ccc4ccc5cc(N(c6ccc7ccccc7c6)c6ccc7[se]c8ccccc8c7c6)ccc5c4c3o2)cc1 ; CC1(C)c2ccccc2-c2cc(N(c3ccc4c(ccc5ccc6nc(-c7ccccc7)oc6c54)c3)c3ccc4[se]c5ccccc5c4c3)ccc21]

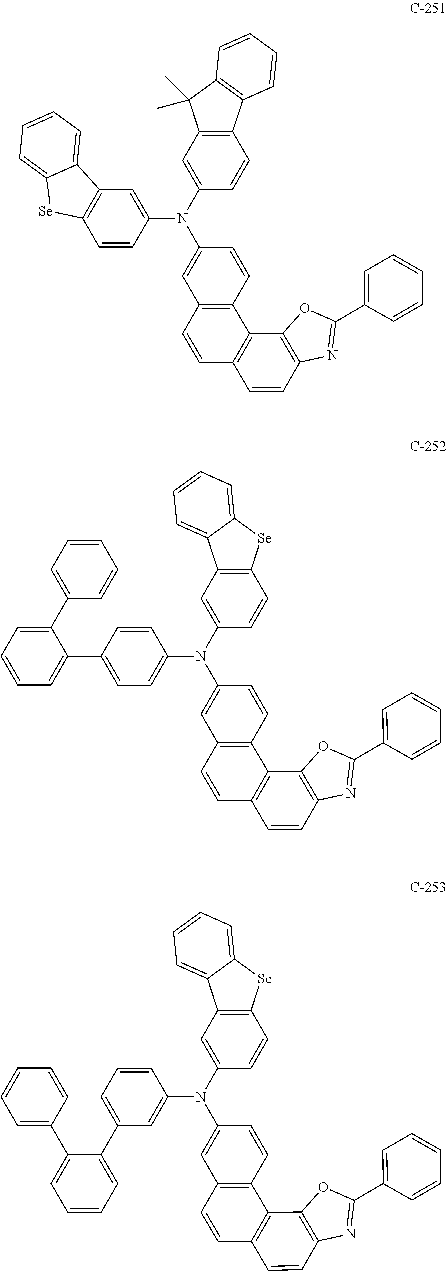 [SMILES: CC1(C)c2ccccc2-c2ccc(N(c3ccc4c(ccc5ccc6nc(-c7ccccc7)oc6c54)c3)c3ccc4[se]c5ccccc5c4c3)cc21 ; c1ccc(-c2nc3ccc4ccc5cc(N(c6ccc(-c7ccccc7-c7ccccc7)cc6)c6ccc7[se]c8ccccc8c7c6)ccc5c4c3o2)cc1 ; c1ccc(-c2nc3ccc4ccc5cc(N(c6cccc(-c7ccccc7-c7ccccc7)c6)c6ccc7[se]c8ccccc8c7c6)ccc5c4c3o2)cc1]

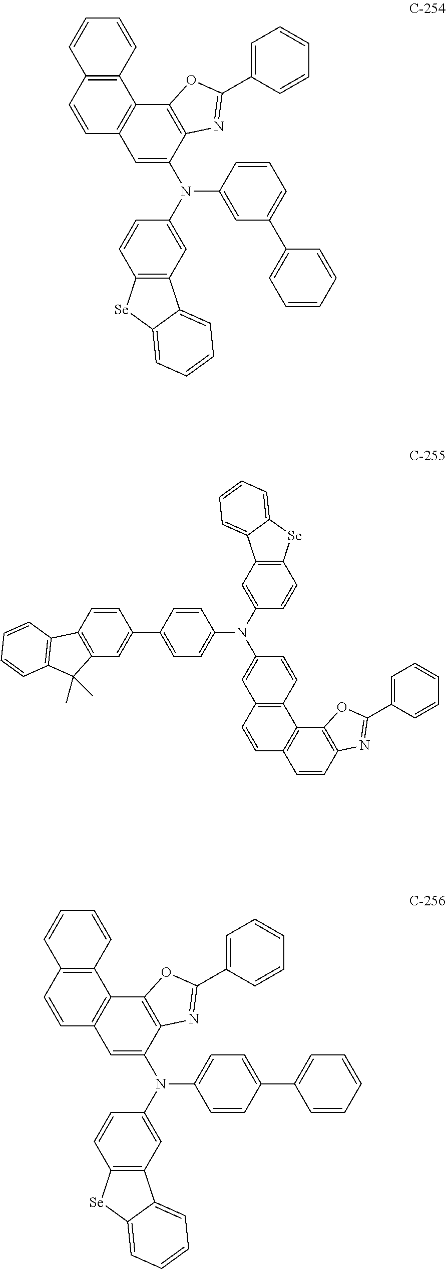 [SMILES: c1ccc(-c2cccc(N(c3ccc4[se]c5ccccc5c4c3)c3cc4ccc5ccccc5c4c4oc(-c5ccccc5)nc34)c2)cc1 ; CC1(C)c2ccccc2-c2ccc(-c3ccc(N(c4ccc5c(ccc6ccc7nc(-c8ccccc8)oc7c65)c4)c4ccc5[se]c6ccccc6c5c4)cc3)cc21 ; c1ccc(-c2ccc(N(c3ccc4[se]c5ccccc5c4c3)c3cc4ccc5ccccc5c4c4oc(-c5ccccc5)nc34)cc2)cc1]

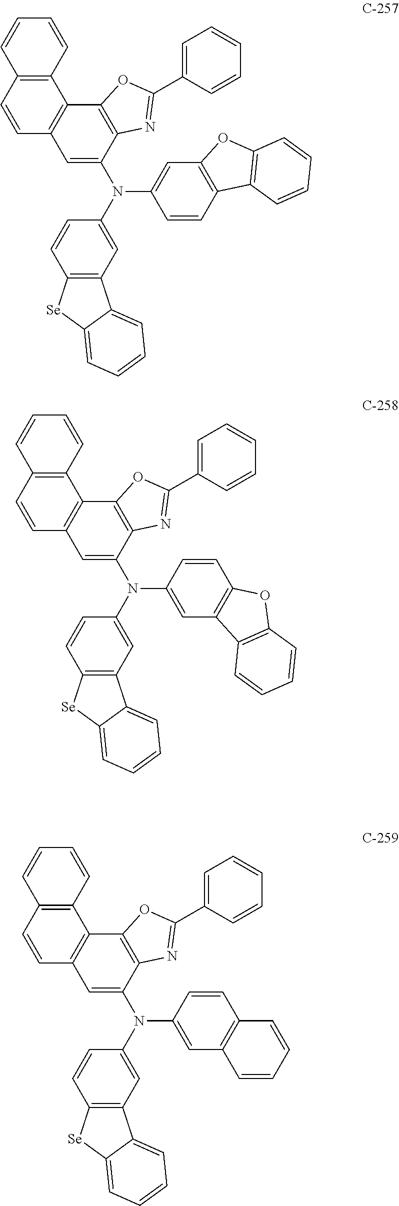 [SMILES: c1ccc(-c2nc3c(N(c4ccc5c(c4)oc4ccccc45)c4ccc5[se]c6ccccc6c5c4)cc4ccc5ccccc5c4c3o2)cc1 ; c1ccc(-c2nc3c(N(c4ccc5oc6ccccc6c5c4)c4ccc5[se]c6ccccc6c5c4)cc4ccc5ccccc5c4c3o2)cc1 ; c1ccc(-c2nc3c(N(c4ccc5ccccc5c4)c4ccc5[se]c6ccccc6c5c4)cc4ccc5ccccc5c4c3o2)cc1]

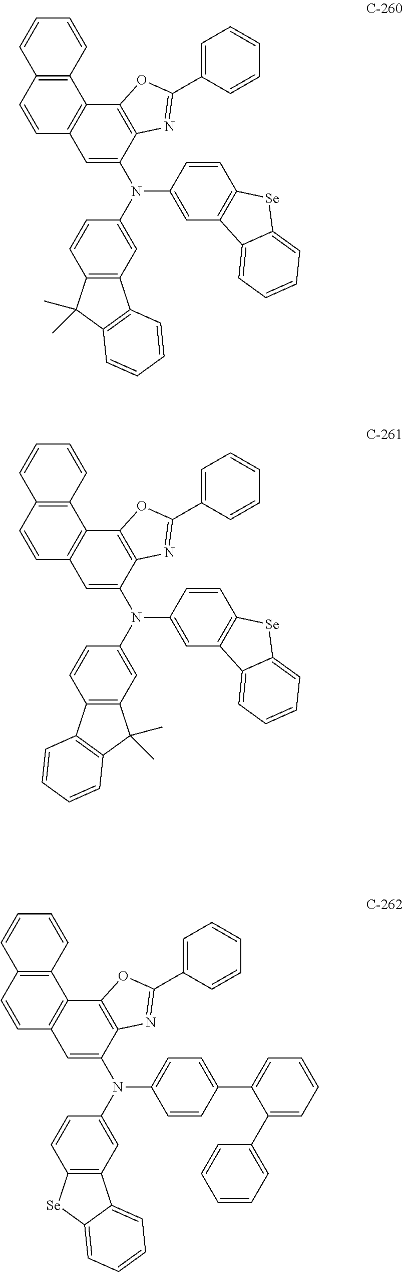 [SMILES: CC1(C)c2ccccc2-c2cc(N(c3ccc4[se]c5ccccc5c4c3)c3cc4ccc5ccccc5c4c4oc(-c5ccccc5)nc34)ccc21 ; CC1(C)c2ccccc2-c2ccc(N(c3ccc4[se]c5ccccc5c4c3)c3cc4ccc5ccccc5c4c4oc(-c5ccccc5)nc34)cc21 ; c1ccc(-c2nc3c(N(c4ccc(-c5ccccc5-c5ccccc5)cc4)c4ccc5[se]c6ccccc6c5c4)cc4ccc5ccccc5c4c3o2)cc1]

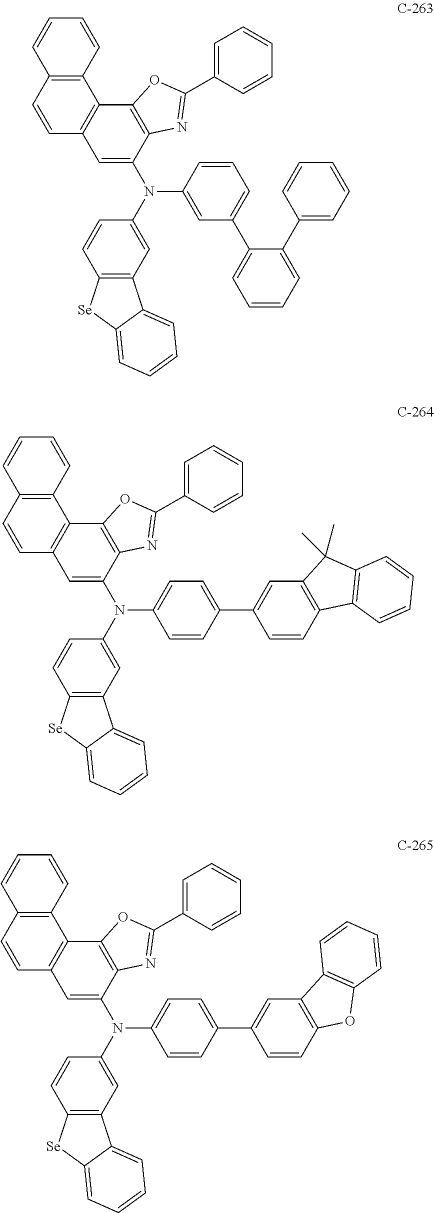 [SMILES: c1ccc(-c2nc3c(N(c4cccc(-c5ccccc5-c5ccccc5)c4)c4ccc5[se]c6ccccc6c5c4)cc4ccc5ccccc5c4c3o2)cc1 ; CC1(C)c2ccccc2-c2ccc(-c3ccc(N(c4ccc5[se]c6ccccc6c5c4)c4cc5ccc6ccccc6c5c5oc(-c6ccccc6)nc45)cc3)cc21 ; c1ccc(-c2nc3c(N(c4ccc(-c5ccc6oc7ccccc7c6c5)cc4)c4ccc5[se]c6ccccc6c5c4)cc4ccc5ccccc5c4c3o2)cc1]

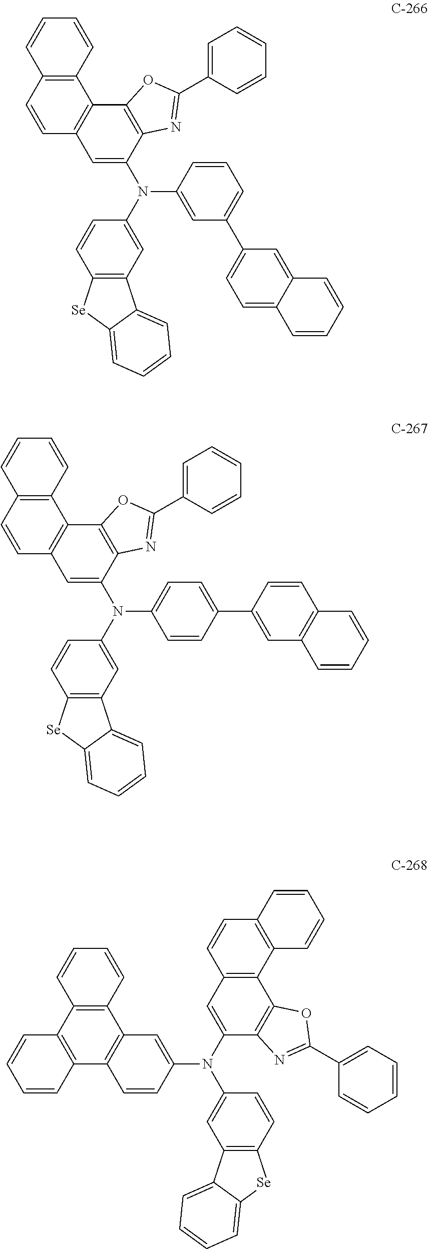 [SMILES: c1ccc(-c2nc3c(N(c4cccc(-c5ccc6ccccc6c5)c4)c4ccc5[se]c6ccccc6c5c4)cc4ccc5ccccc5c4c3o2)cc1 ; c1ccc(-c2nc3c(N(c4ccc(-c5ccc6ccccc6c5)cc4)c4ccc5[se]c6ccccc6c5c4)cc4ccc5ccccc5c4c3o2)cc1 ; c1ccc(-c2nc3c(N(c4ccc5[se]c6ccccc6c5c4)c4ccc5c6ccccc6c6ccccc6c5c4)cc4ccc5ccccc5c4c3o2)cc1]

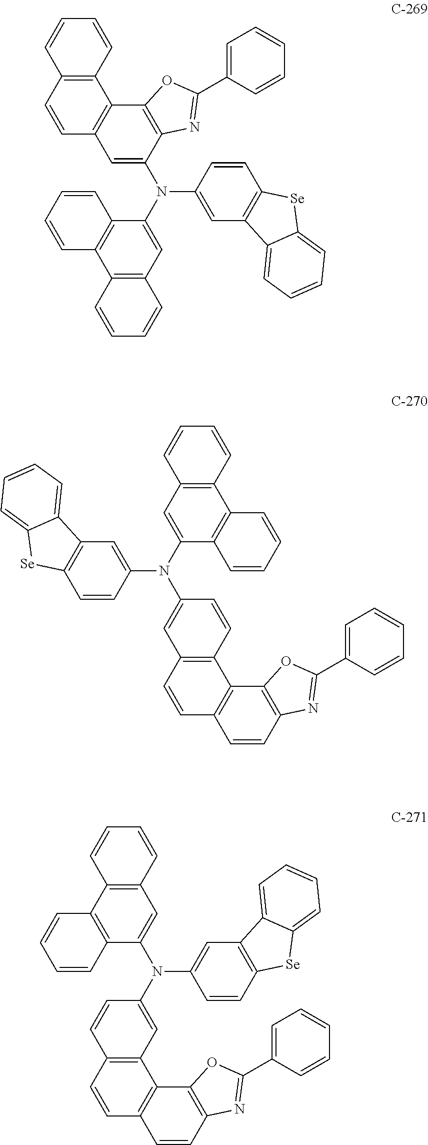 [SMILES: c1ccc(-c2nc3c(N(c4ccc5[se]c6ccccc6c5c4)c4cc5ccccc5c5ccccc45)cc4ccc5ccccc5c4c3o2)cc1 ; c1ccc(-c2nc3ccc4ccc5cc(N(c6ccc7[se]c8ccccc8c7c6)c6cc7ccccc7c7ccccc67)ccc5c4c3o2)cc1 ; c1ccc(-c2nc3ccc4ccc5ccc(N(c6ccc7[se]c8ccccc8c7c6)c6cc7ccccc7c7ccccc67)cc5c4c3o2)cc1]

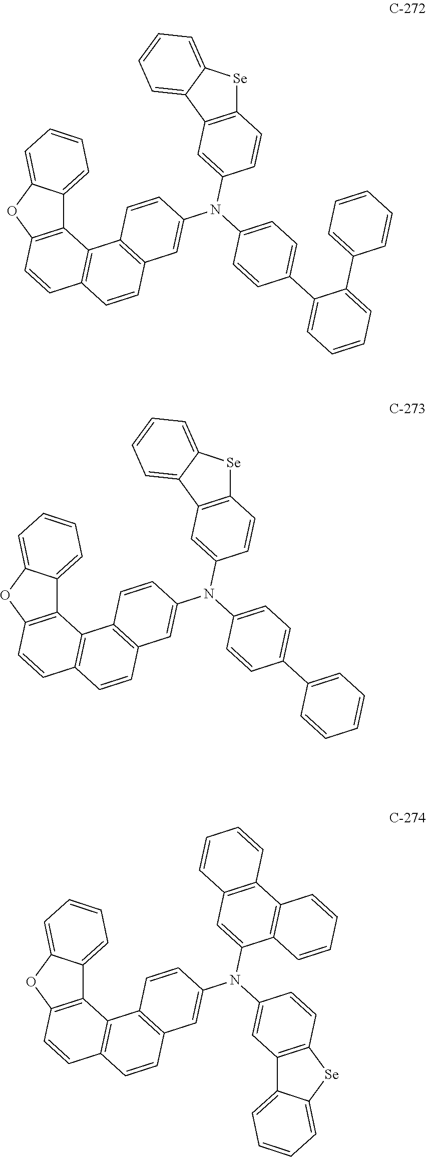 [SMILES: c1ccc(-c2ccccc2-c2ccc(N(c3ccc4c(ccc5ccc6oc7ccccc7c6c54)c3)c3ccc4[se]c5ccccc5c4c3)cc2)cc1 ; c1ccc(-c2ccc(N(c3ccc4c(ccc5ccc6oc7ccccc7c6c54)c3)c3ccc4[se]c5ccccc5c4c3)cc2)cc1 ; c1ccc2c(c1)cc(N(c1ccc3c(ccc4ccc5oc6ccccc6c5c43)c1)c1ccc3[se]c4ccccc4c3c1)c1ccccc12]

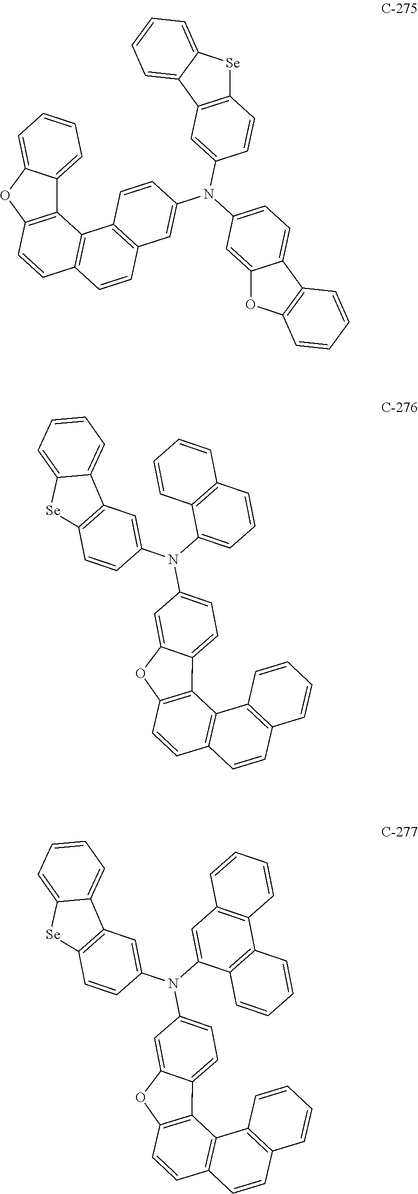 [SMILES: c1ccc2c(c1)oc1cc(N(c3ccc4c(ccc5ccc6oc7ccccc7c6c54)c3)c3ccc4[se]c5ccccc5c4c3)ccc12 ; c1ccc2c(N(c3ccc4c(c3)oc3ccc5ccc6ccccc6c5c34)c3ccc4[se]c5ccccc5c4c3)cccc2c1 ; c1ccc2c(c1)cc(N(c1ccc3c(c1)oc1ccc4ccc5ccccc5c4c13)c1ccc3[se]c4ccccc4c3c1)c1ccccc12]

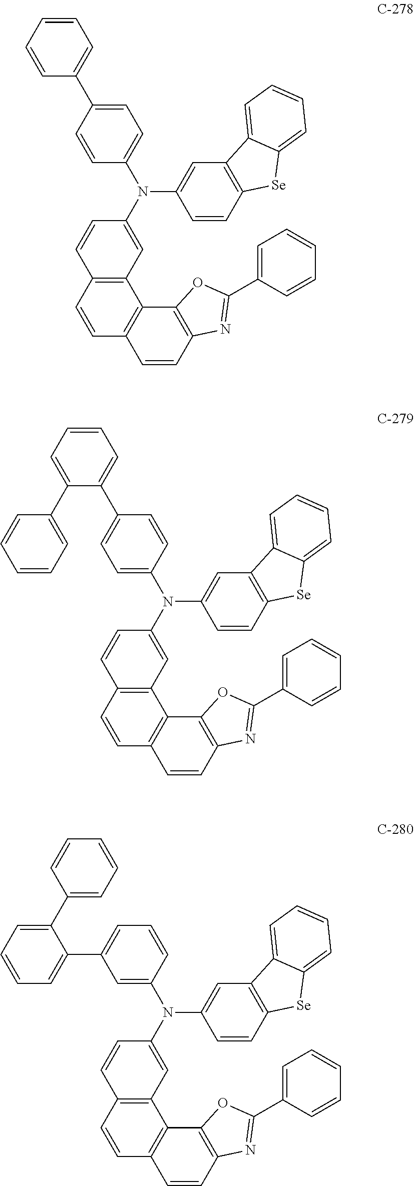 [SMILES: c1ccc(-c2ccc(N(c3ccc4[se]c5ccccc5c4c3)c3ccc4ccc5ccc6nc(-c7ccccc7)oc6c5c4c3)cc2)cc1 ; c1ccc(-c2nc3ccc4ccc5ccc(N(c6ccc(-c7ccccc7-c7ccccc7)cc6)c6ccc7[se]c8ccccc8c7c6)cc5c4c3o2)cc1 ; c1ccc(-c2nc3ccc4ccc5ccc(N(c6cccc(-c7ccccc7-c7ccccc7)c6)c6ccc7[se]c8ccccc8c7c6)cc5c4c3o2)cc1]

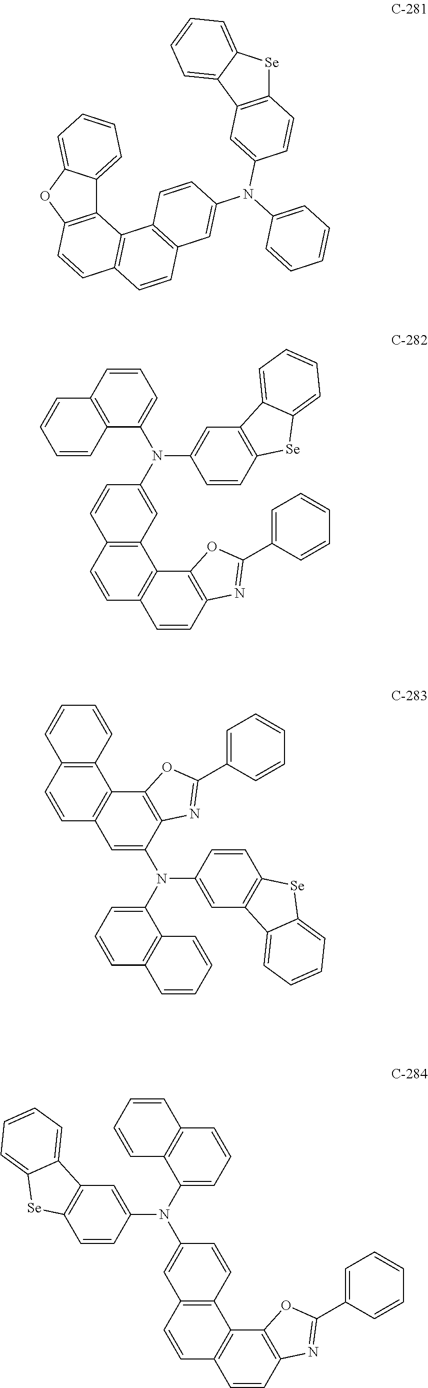 [SMILES: c1ccc(N(c2ccc3c(ccc4ccc5oc6ccccc6c5c43)c2)c2ccc3[se]c4ccccc4c3c2)cc1 ; c1ccc(-c2nc3ccc4ccc5ccc(N(c6ccc7[se]c8ccccc8c7c6)c6cccc7ccccc67)cc5c4c3o2)cc1 ; c1ccc(-c2nc3c(N(c4ccc5[se]c6ccccc6c5c4)c4cccc5ccccc45)cc4ccc5ccccc5c4c3o2)cc1 ; c1ccc(-c2nc3ccc4ccc5cc(N(c6ccc7[se]c8ccccc8c7c6)c6cccc7ccccc67)ccc5c4c3o2)cc1]

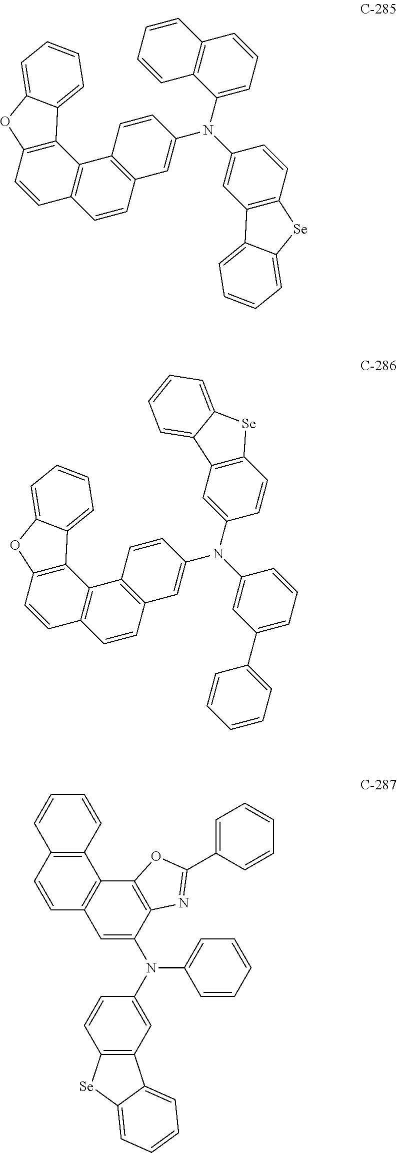 [SMILES: c1ccc2c(N(c3ccc4c(ccc5ccc6oc7ccccc7c6c54)c3)c3ccc4[se]c5ccccc5c4c3)cccc2c1 ; c1ccc(-c2cccc(N(c3ccc4c(ccc5ccc6oc7ccccc7c6c54)c3)c3ccc4[se]c5ccccc5c4c3)c2)cc1 ; c1ccc(-c2nc3c(N(c4ccccc4)c4ccc5[se]c6ccccc6c5c4)cc4ccc5ccccc5c4c3o2)cc1]

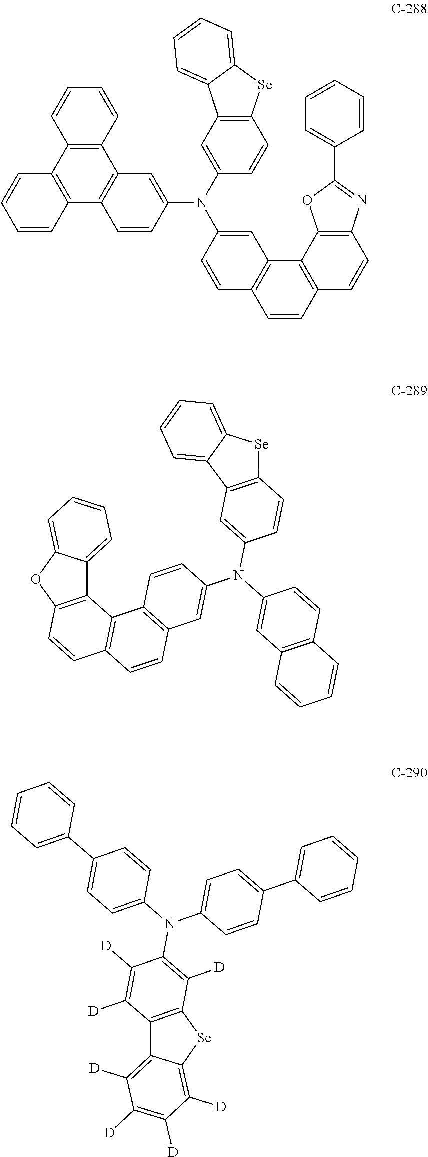 [SMILES: c1ccc(-c2nc3ccc4ccc5ccc(N(c6ccc7[se]c8ccccc8c7c6)c6ccc7c8ccccc8c8ccccc8c7c6)cc5c4c3o2)cc1 ; c1ccc2cc(N(c3ccc4c(ccc5ccc6oc7ccccc7c6c54)c3)c3ccc4[se]c5ccccc5c4c3)ccc2c1 ; [2H]c1c([2H])c([2H])c2c([se]c3c([2H])c(N(c4ccc(-c5ccccc5)cc4)c4ccc(-c5ccccc5)cc4)c([2H])c([2H])c32)c1[2H]]

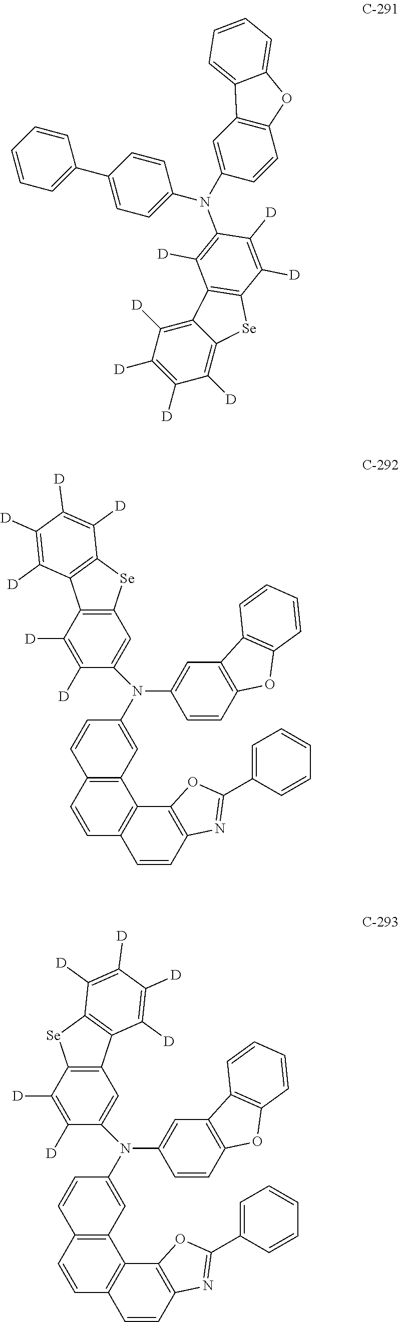 [SMILES: [2H]c1c([2H])c([2H])c2c([se]c3c([2H])c([2H])c(N(c4ccc(-c5ccccc5)cc4)c4ccc5oc6ccccc6c5c4)c([2H])c32)c1[2H] ; [2H]c1c([2H])c([2H])c2c([se]c3cc(N(c4ccc5oc6ccccc6c5c4)c4ccc5ccc6ccc7nc(-c8ccccc8)oc7c6c5c4)c([2H])c([2H])c32)c1[2H] ; [2H]c1c(N(c2ccc3oc4ccccc4c3c2)c2ccc3ccc4ccc5nc(-c6ccccc6)oc5c4c3c2)cc2c([se]c3c([2H])c([2H])c([2H])c([2H])c32)c1[2H]]

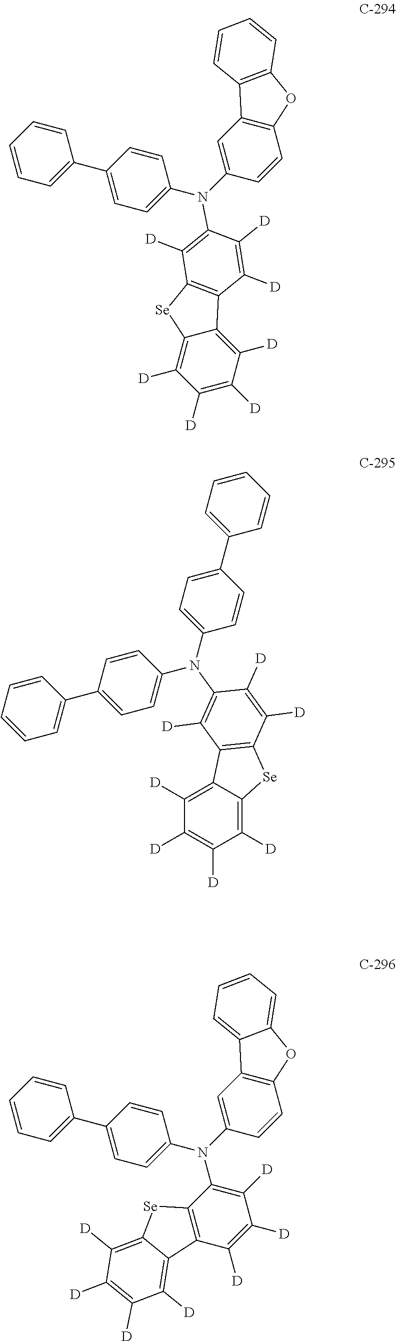 [SMILES: [2H]c1c([2H])c([2H])c2c([se]c3c([2H])c(N(c4ccc(-c5ccccc5)cc4)c4ccc5oc6ccccc6c5c4)c([2H])c([2H])c32)c1[2H] ; [2H]c1c([2H])c([2H])c2c([se]c3c([2H])c([2H])c(N(c4ccc(-c5ccccc5)cc4)c4ccc(-c5ccccc5)cc4)c([2H])c32)c1[2H] ; [2H]c1c([2H])c([2H])c2c([se]c3c(N(c4ccc(-c5ccccc5)cc4)c4ccc5oc6ccccc6c5c4)c([2H])c([2H])c([2H])c32)c1[2H]]

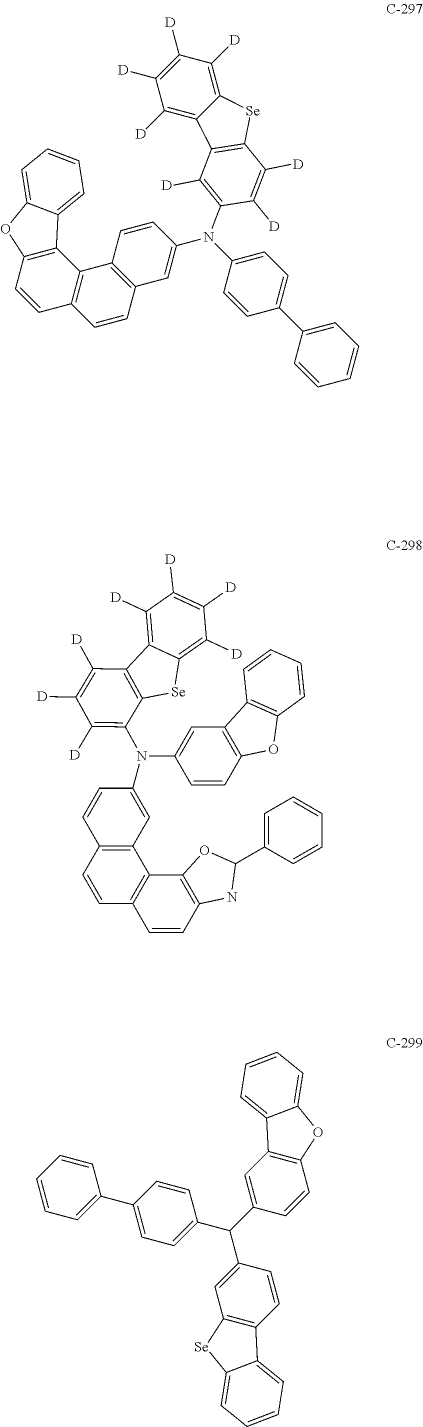 [SMILES: [2H]c1c([2H])c([2H])c2c([se]c3c([2H])c([2H])c(N(c4ccc(-c5ccccc5)cc4)c4ccc5c(ccc6ccc7oc8ccccc8c7c65)c4)c([2H])c32)c1[2H] ; [2H]c1c([2H])c([2H])c2c([se]c3c(N(c4ccc5oc6ccccc6c5c4)c4ccc5ccc6ccc7c(c6c5c4)OC(c4ccccc4)N7)c([2H])c([2H])c([2H])c32)c1[2H] ; c1ccc(-c2ccc(C(c3ccc4c(c3)[se]c3ccccc34)c3ccc4oc5ccccc5c4c3)cc2)cc1]

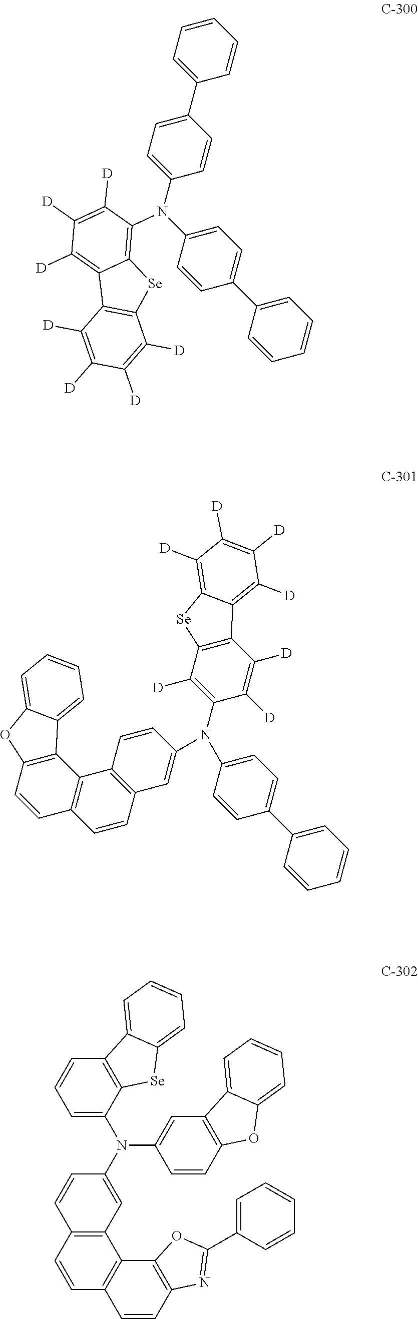 [SMILES: [2H]c1c([2H])c([2H])c2c([se]c3c(N(c4ccc(-c5ccccc5)cc4)c4ccc(-c5ccccc5)cc4)c([2H])c([2H])c([2H])c32)c1[2H] ; [2H]c1c([2H])c([2H])c2c([se]c3c([2H])c(N(c4ccc(-c5ccccc5)cc4)c4ccc5c(ccc6ccc7oc8ccccc8c7c65)c4)c([2H])c([2H])c32)c1[2H] ; c1ccc(-c2nc3ccc4ccc5ccc(N(c6ccc7oc8ccccc8c7c6)c6cccc7c6[se]c6ccccc67)cc5c4c3o2)cc1]

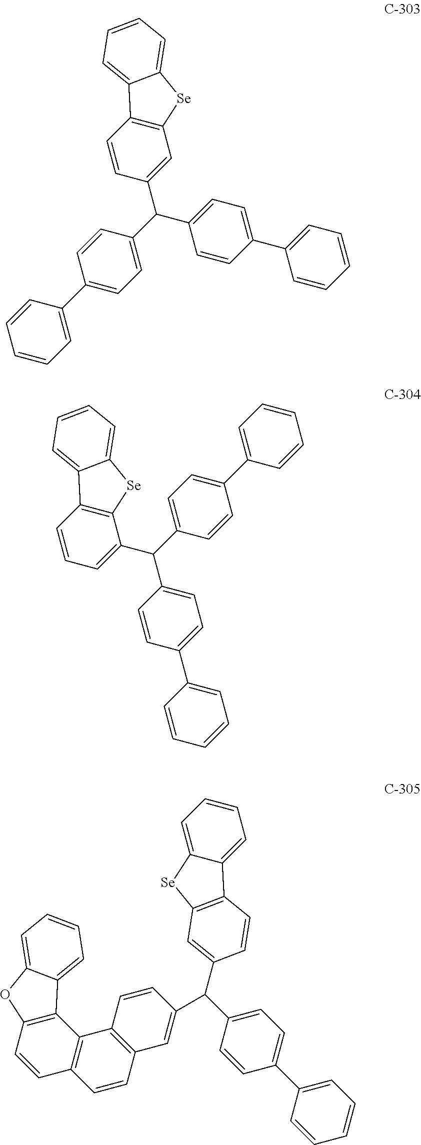 [SMILES: c1ccc(-c2ccc(C(c3ccc(-c4ccccc4)cc3)c3ccc4c(c3)[se]c3ccccc34)cc2)cc1 ; c1ccc(-c2ccc(C(c3ccc(-c4ccccc4)cc3)c3cccc4c3[se]c3ccccc34)cc2)cc1 ; c1ccc(-c2ccc(C(c3ccc4c(ccc5ccc6oc7ccccc7c6c54)c3)c3ccc4c(c3)[se]c3ccccc34)cc2)cc1]

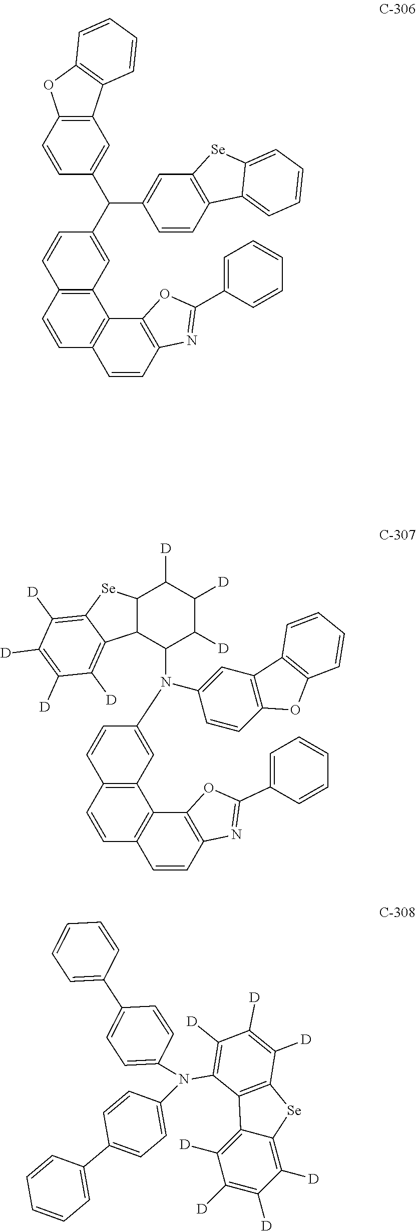 [SMILES: c1ccc(-c2nc3ccc4ccc5ccc(C(c6ccc7c(c6)[se]c6ccccc67)c6ccc7oc8ccccc8c7c6)cc5c4c3o2)cc1 ; [2H]c1c([2H])c([2H])c2c(c1[2H])[Se]C1C([2H])C([2H])C([2H])C(N(c3ccc4oc5ccccc5c4c3)c3ccc4ccc5ccc6nc(-c7ccccc7)oc6c5c4c3)C21 ; [2H]c1c([2H])c([2H])c2c([se]c3c([2H])c([2H])c([2H])c(N(c4ccc(-c5ccccc5)cc4)c4ccc(-c5ccccc5)cc4)c32)c1[2H]]

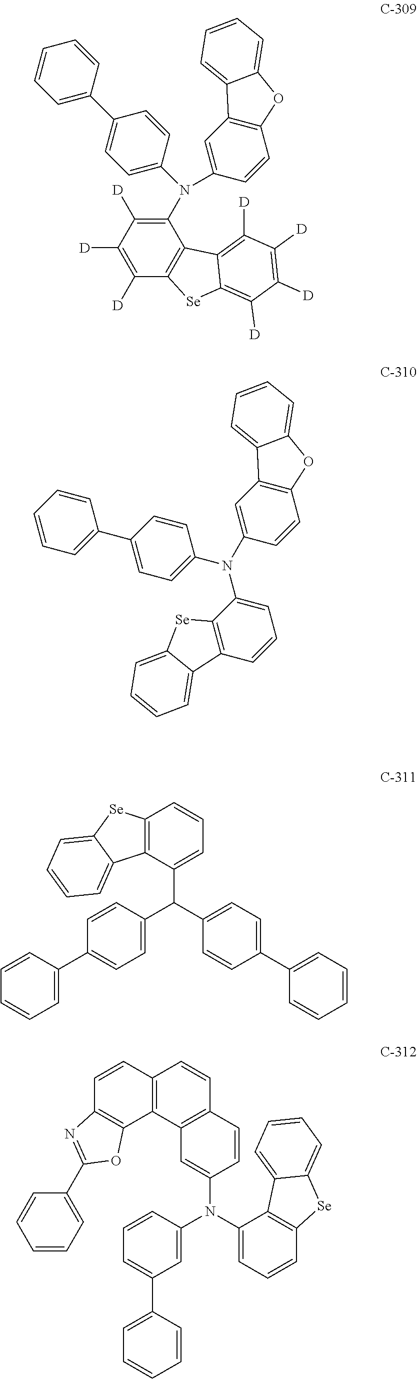

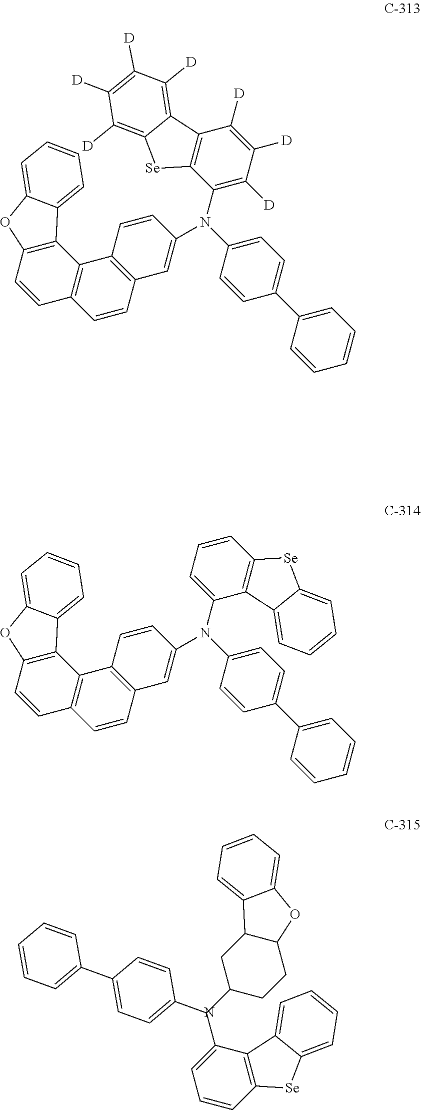

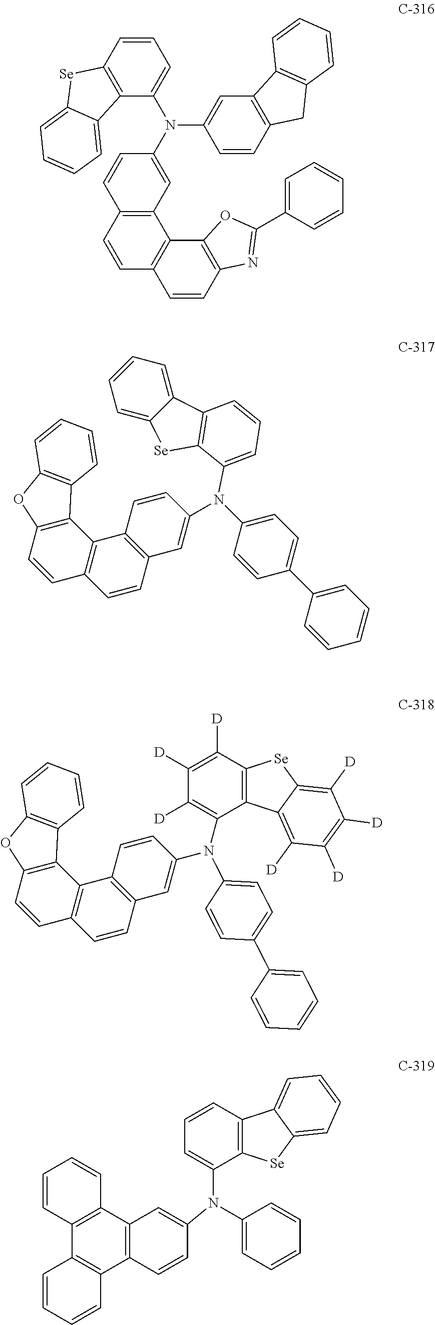 [SMILES: c1ccc(-c2nc3ccc4ccc5ccc(N(c6ccc7c(c6)-c6ccccc6C7)c6cccc7[se]c8ccccc8c67)cc5c4c3o2)cc1 ; c1ccc(-c2ccc(N(c3ccc4c(ccc5ccc6oc7ccccc7c6c54)c3)c3cccc4c3[se]c3ccccc34)cc2)cc1 ; [2H]c1c([2H])c([2H])c2c([se]c3c([2H])c([2H])c([2H])c(N(c4ccc(-c5ccccc5)cc4)c4ccc5c(ccc6ccc7oc8ccccc8c7c65)c4)c32)c1[2H] ; c1ccc(N(c2ccc3c4ccccc4c4ccccc4c3c2)c2cccc3c2[se]c2ccccc23)cc1]

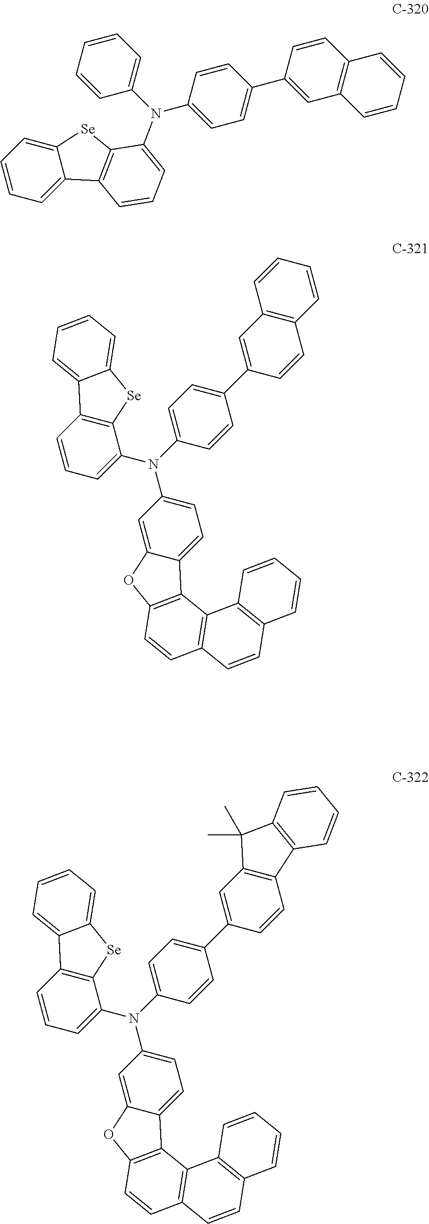 [SMILES: c1ccc(N(c2ccc(-c3ccc4ccccc4c3)cc2)c2cccc3c2[se]c2ccccc23)cc1 ; c1ccc2cc(-c3ccc(N(c4ccc5c(c4)oc4ccc6ccc7ccccc7c6c45)c4cccc5c4[se]c4ccccc45)cc3)ccc2c1 ; CC1(C)c2ccccc2-c2ccc(-c3ccc(N(c4ccc5c(c4)oc4ccc6ccc7ccccc7c6c45)c4cccc5c4[se]c4ccccc45)cc3)cc21]

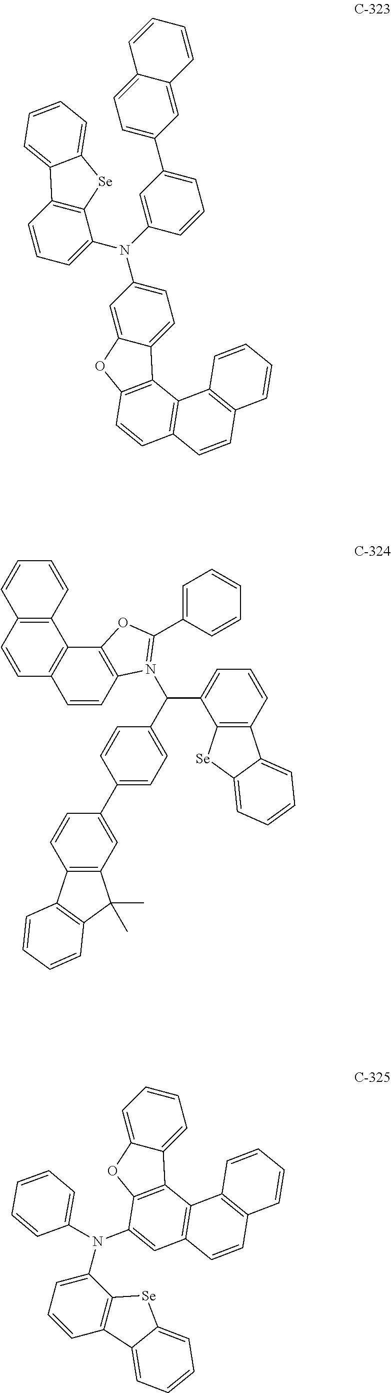

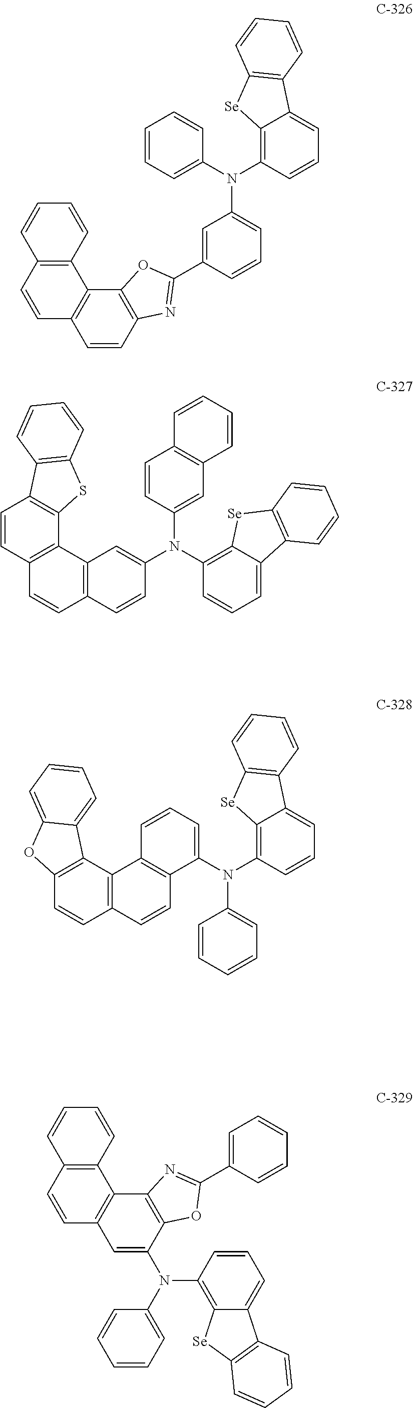 [SMILES: c1ccc(N(c2cccc(-c3nc4ccc5ccc6ccccc6c5c4o3)c2)c2cccc3c2[se]c2ccccc23)cc1 ; c1ccc2cc(N(c3ccc4ccc5ccc6c7ccccc7sc6c5c4c3)c3cccc4c3[se]c3ccccc34)ccc2c1 ; c1ccc(N(c2cccc3c2ccc2ccc4oc5ccccc5c4c23)c2cccc3c2[se]c2ccccc23)cc1 ; c1ccc(-c2nc3c(o2)c(N(c2ccccc2)c2cccc4c2[se]c2ccccc24)cc2ccc4ccccc4c23)cc1]

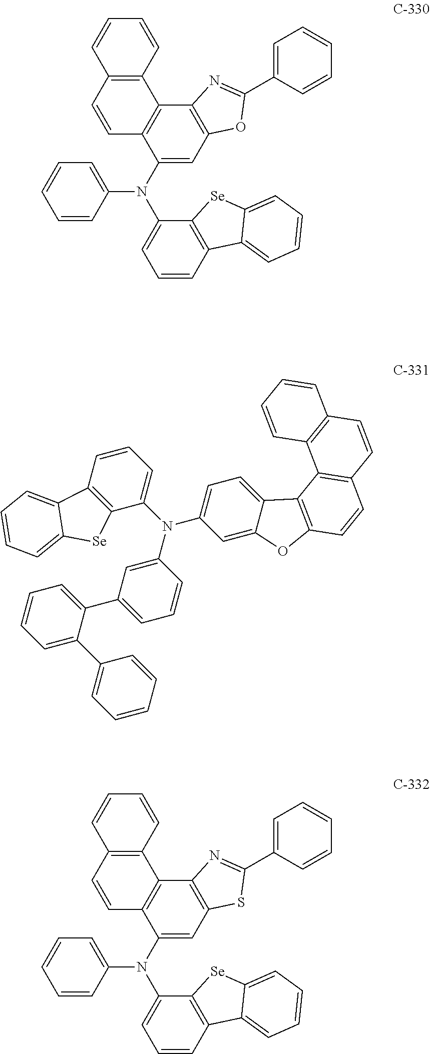 [SMILES: c1ccc(-c2nc3c(cc(N(c4ccccc4)c4cccc5c4[se]c4ccccc45)c4ccc5ccccc5c43)o2)cc1 ; c1ccc(-c2ccccc2-c2cccc(N(c3ccc4c(c3)oc3ccc5ccc6ccccc6c5c34)c3cccc4c3[se]c3ccccc34)c2)cc1 ; c1ccc(-c2nc3c(cc(N(c4ccccc4)c4cccc5c4[se]c4ccccc45)c4ccc5ccccc5c43)s2)cc1]

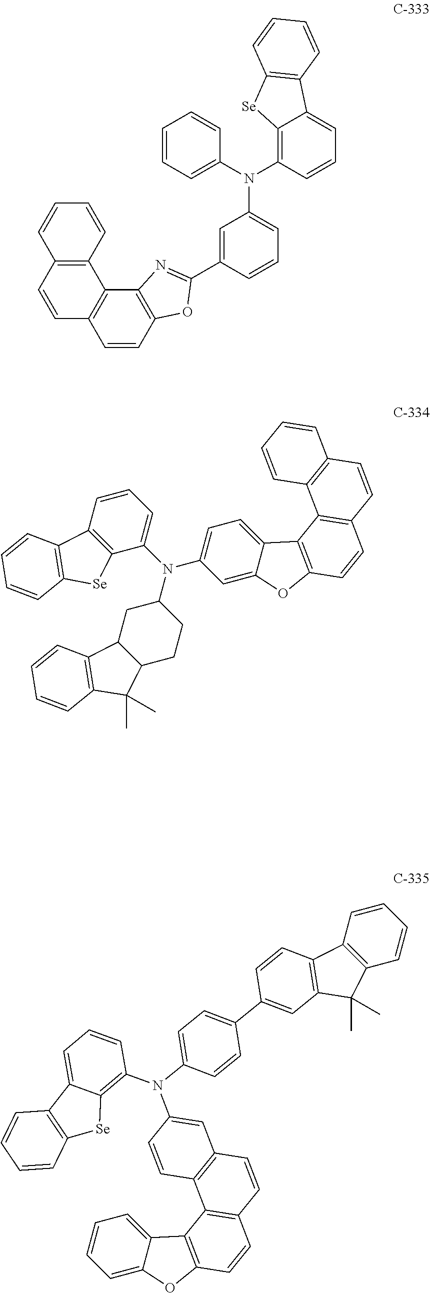 [SMILES: c1ccc(N(c2cccc(-c3nc4c(ccc5ccc6ccccc6c54)o3)c2)c2cccc3c2[se]c2ccccc23)cc1 ; CC1(C)c2ccccc2C2CC(N(c3ccc4c(c3)oc3ccc5ccc6ccccc6c5c34)c3cccc4c3[se]c3ccccc34)CCC21 ; CC1(C)c2ccccc2-c2ccc(-c3ccc(N(c4ccc5c(ccc6ccc7oc8ccccc8c7c65)c4)c4cccc5c4[se]c4ccccc45)cc3)cc21]

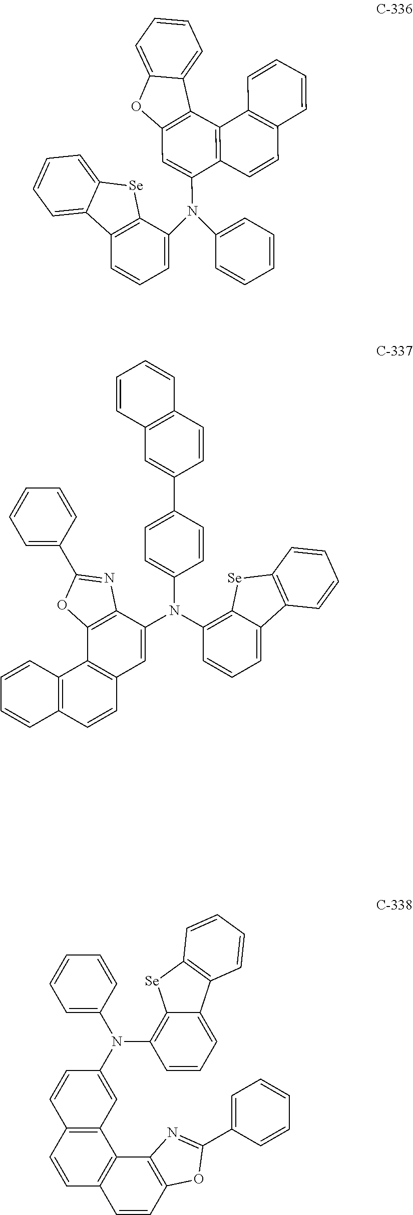 [SMILES: c1ccc(N(c2cc3oc4ccccc4c3c3c2ccc2ccccc23)c2cccc3c2[se]c2ccccc23)cc1 ; c1ccc(-c2nc3c(N(c4ccc(-c5ccc6ccccc6c5)cc4)c4cccc5c4[se]c4ccccc45)cc4ccc5ccccc5c4c3o2)cc1 ; c1ccc(-c2nc3c(ccc4ccc5ccc(N(c6ccccc6)c6cccc7c6[se]c6ccccc67)cc5c43)o2)cc1]

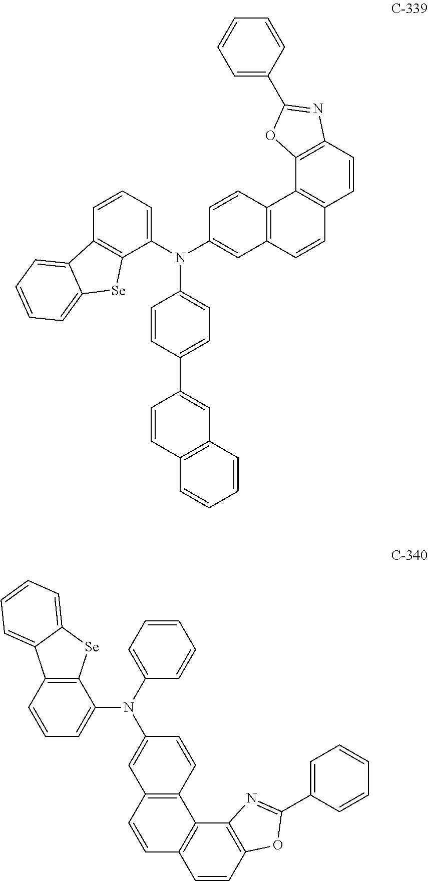 [SMILES: c1ccc(-c2nc3ccc4ccc5cc(N(c6ccc(-c7ccc8ccccc8c7)cc6)c6cccc7c6[se]c6ccccc67)ccc5c4c3o2)cc1 ; c1ccc(-c2nc3c(ccc4ccc5cc(N(c6ccccc6)c6cccc7c6[se]c6ccccc67)ccc5c43)o2)cc1]

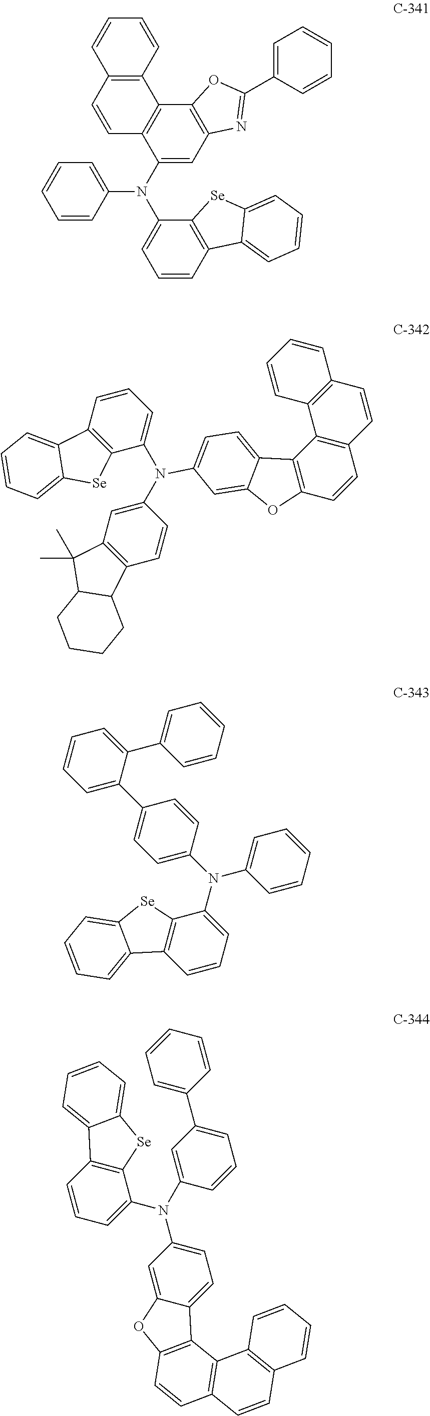 [SMILES: c1ccc(-c2nc3cc(N(c4ccccc4)c4cccc5c4[se]c4ccccc45)c4ccc5ccccc5c4c3o2)cc1 ; CC1(C)c2cc(N(c3ccc4c(c3)oc3ccc5ccc6ccccc6c5c34)c3cccc4c3[se]c3ccccc34)ccc2C2CCCCC21 ; c1ccc(-c2ccccc2-c2ccc(N(c3ccccc3)c3cccc4c3[se]c3ccccc34)cc2)cc1 ; c1ccc(-c2cccc(N(c3ccc4c(c3)oc3ccc5ccc6ccccc6c5c34)c3cccc4c3[se]c3ccccc34)c2)cc1]

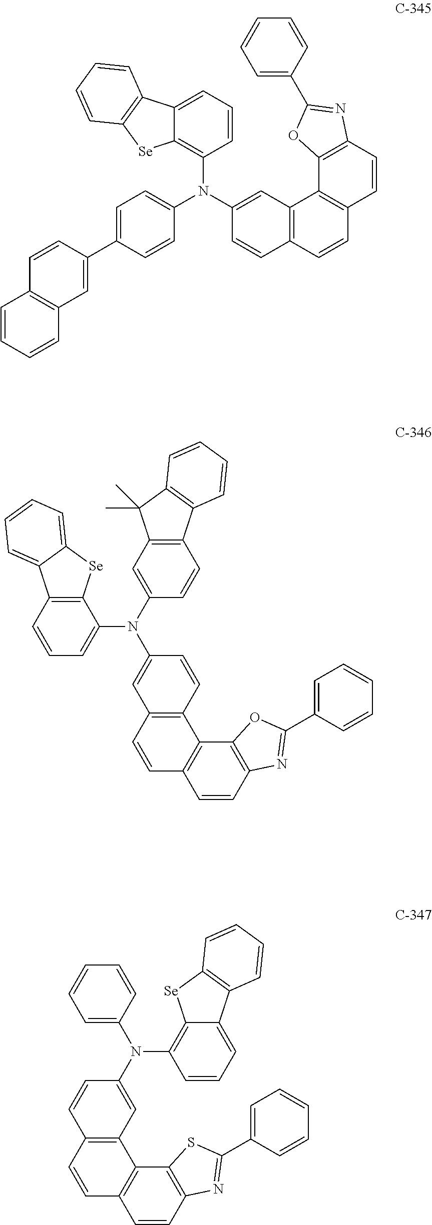 [SMILES: c1ccc(-c2nc3ccc4ccc5ccc(N(c6ccc(-c7ccc8ccccc8c7)cc6)c6cccc7c6[se]c6ccccc67)cc5c4c3o2)cc1 ; CC1(C)c2ccccc2-c2ccc(N(c3ccc4c(ccc5ccc6nc(-c7ccccc7)oc6c54)c3)c3cccc4c3[se]c3ccccc34)cc21 ; c1ccc(-c2nc3ccc4ccc5ccc(N(c6ccccc6)c6cccc7c6[se]c6ccccc67)cc5c4c3s2)cc1]

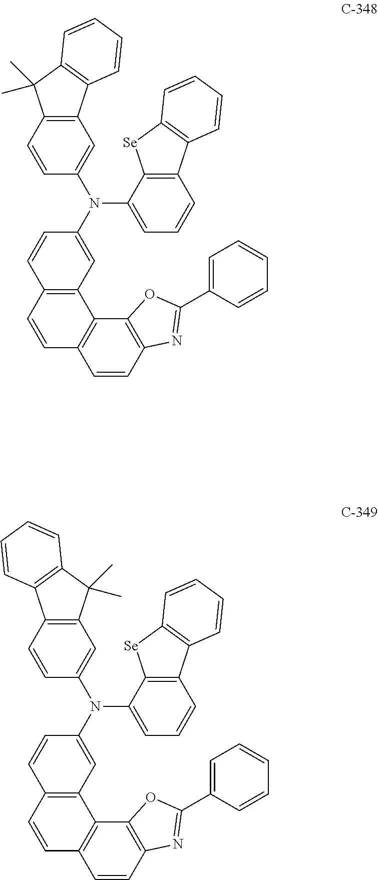 [SMILES: CC1(C)c2ccccc2-c2cc(N(c3ccc4ccc5ccc6nc(-c7ccccc7)oc6c5c4c3)c3cccc4c3[se]c3ccccc34)ccc21 ; CC1(C)c2ccccc2-c2ccc(N(c3ccc4ccc5ccc6nc(-c7ccccc7)oc6c5c4c3)c3cccc4c3[se]c3ccccc34)cc21]

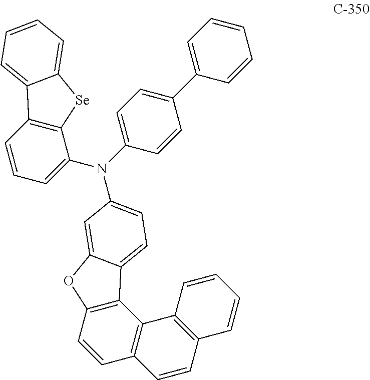 [SMILES: c1ccc(-c2ccc(N(c3ccc4c(c3)oc3ccc5ccc6ccccc6c5c34)c3cccc4c3[se]c3ccccc34)cc2)cc1]

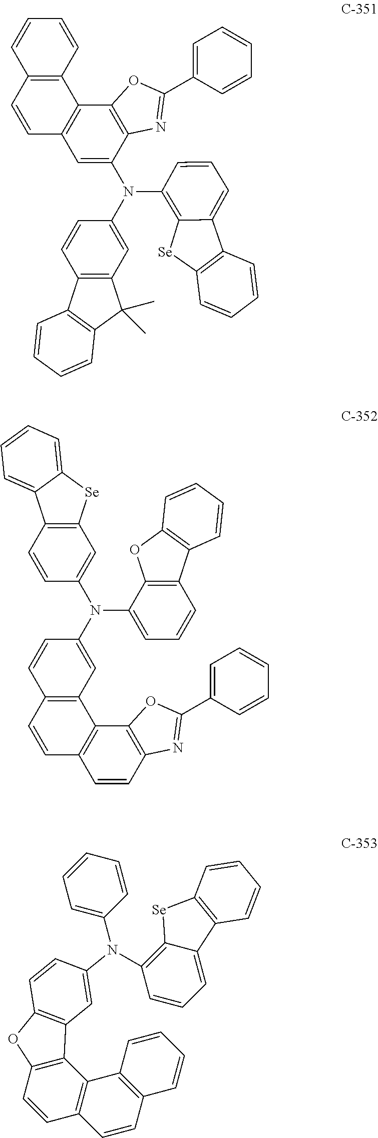 [SMILES: CC1(C)c2ccccc2-c2ccc(N(c3cc4ccc5ccccc5c4c4oc(-c5ccccc5)nc34)c3cccc4c3[se]c3ccccc34)cc21 ; c1ccc(-c2nc3ccc4ccc5ccc(N(c6ccc7c(c6)[se]c6ccccc67)c6cccc7c6oc6ccccc67)cc5c4c3o2)cc1 ; c1ccc(N(c2ccc3oc4ccc5ccc6ccccc6c5c4c3c2)c2cccc3c2[se]c2ccccc23)cc1]

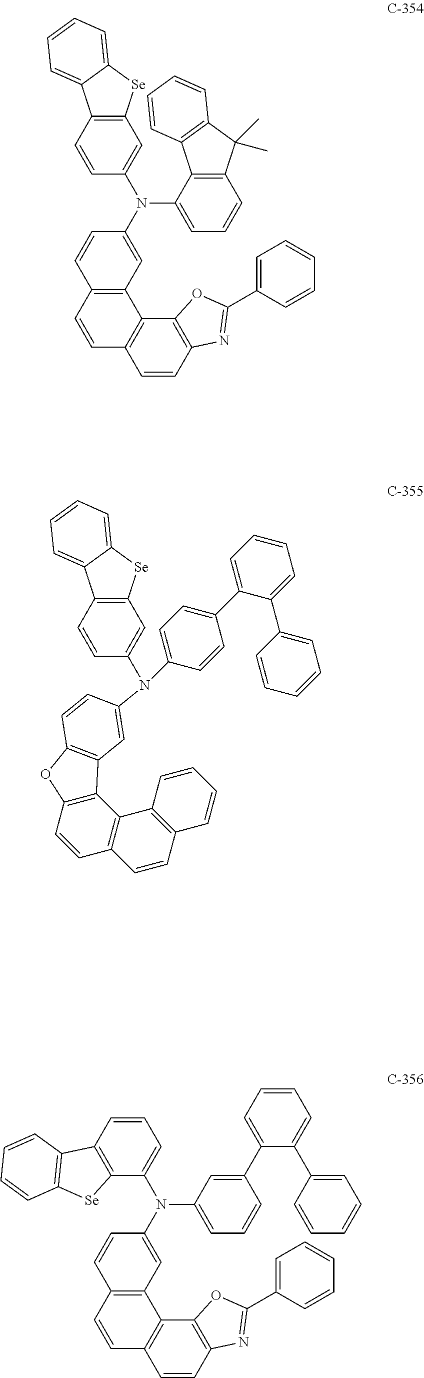 [SMILES: CC1(C)c2ccccc2-c2c(N(c3ccc4c(c3)[se]c3ccccc34)c3ccc4ccc5ccc6nc(-c7ccccc7)oc6c5c4c3)cccc21 ; c1ccc(-c2ccccc2-c2ccc(N(c3ccc4c(c3)[se]c3ccccc34)c3ccc4oc5ccc6ccc7ccccc7c6c5c4c3)cc2)cc1 ; c1ccc(-c2nc3ccc4ccc5ccc(N(c6cccc(-c7ccccc7-c7ccccc7)c6)c6cccc7c6[se]c6ccccc67)cc5c4c3o2)cc1]

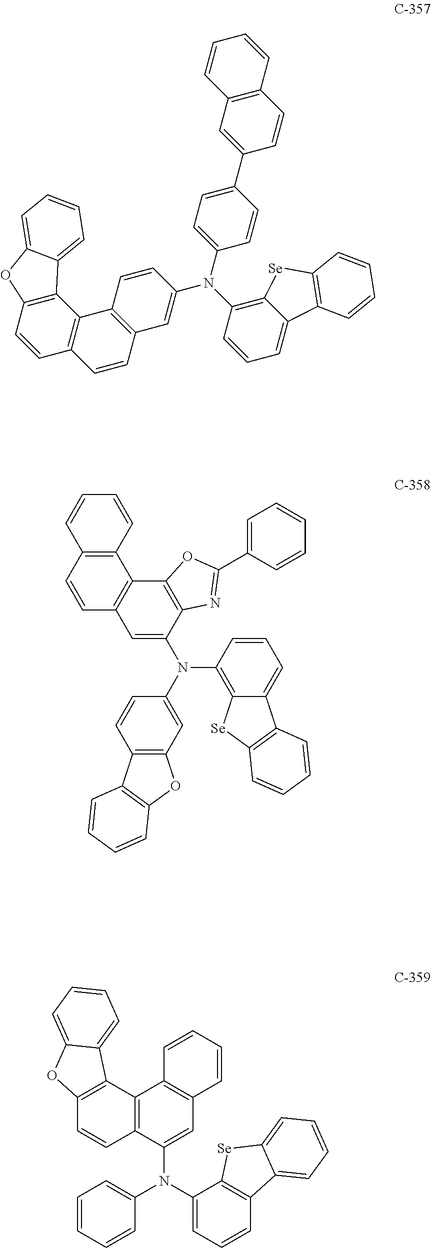 [SMILES: c1ccc2cc(-c3ccc(N(c4ccc5c(ccc6ccc7oc8ccccc8c7c65)c4)c4cccc5c4[se]c4ccccc45)cc3)ccc2c1 ; c1ccc(-c2nc3c(N(c4ccc5c(c4)oc4ccccc45)c4cccc5c4[se]c4ccccc45)cc4ccc5ccccc5c4c3o2)cc1 ; c1ccc(N(c2cc3ccccc3c3c2ccc2oc4ccccc4c23)c2cccc3c2[se]c2ccccc23)cc1]

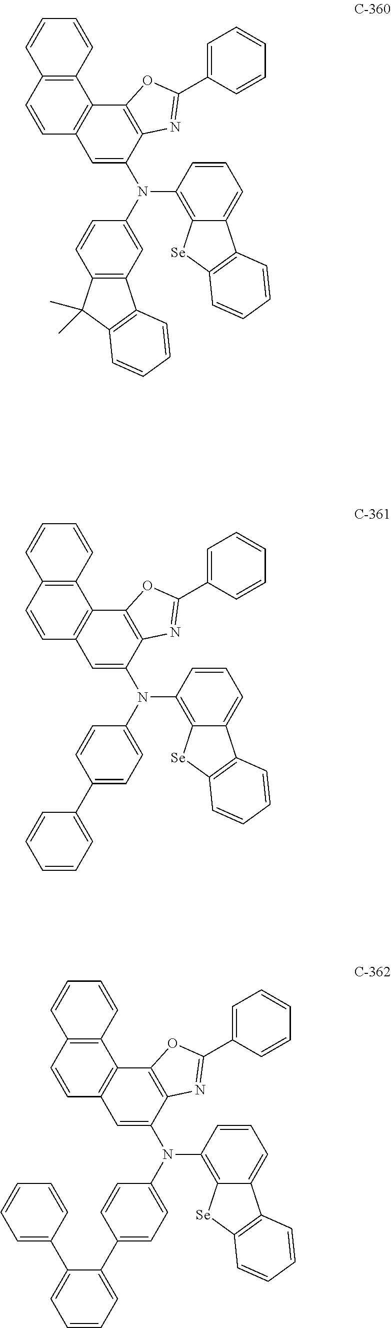 [SMILES: CC1(C)c2ccccc2-c2cc(N(c3cc4ccc5ccccc5c4c4oc(-c5ccccc5)nc34)c3cccc4c3[se]c3ccccc34)ccc21 ; c1ccc(-c2ccc(N(c3cc4ccc5ccccc5c4c4oc(-c5ccccc5)nc34)c3cccc4c3[se]c3ccccc34)cc2)cc1 ; c1ccc(-c2nc3c(N(c4ccc(-c5ccccc5-c5ccccc5)cc4)c4cccc5c4[se]c4ccccc45)cc4ccc5ccccc5c4c3o2)cc1]

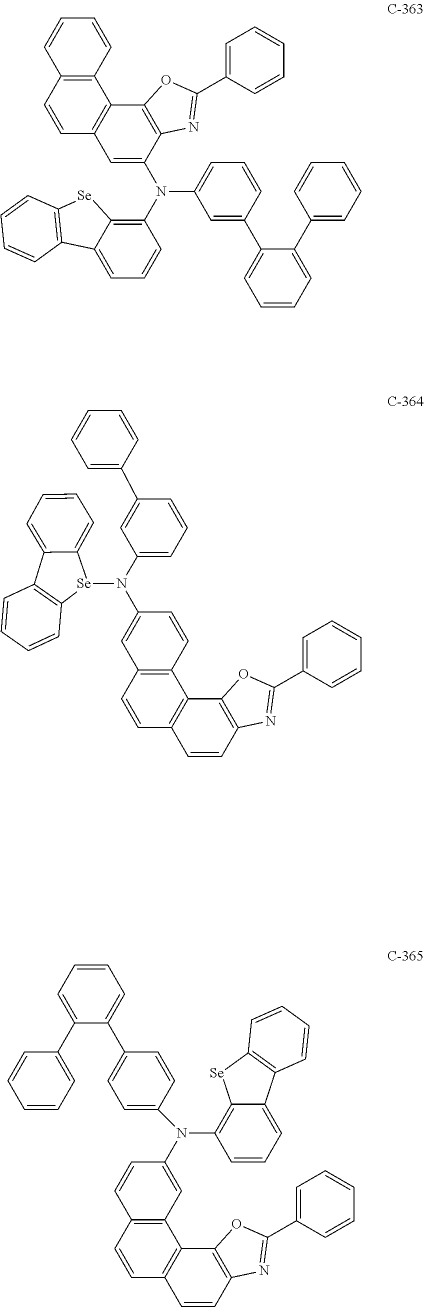

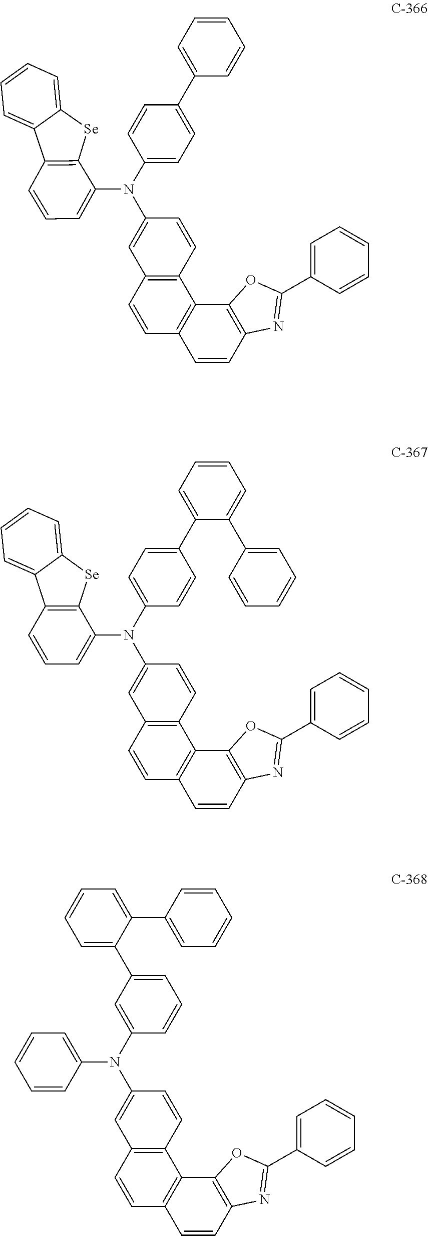 [SMILES: c1ccc(-c2ccc(N(c3ccc4c(ccc5ccc6nc(-c7ccccc7)oc6c54)c3)c3cccc4c3[se]c3ccccc34)cc2)cc1 ; c1ccc(-c2nc3ccc4ccc5cc(N(c6ccc(-c7ccccc7-c7ccccc7)cc6)c6cccc7c6[se]c6ccccc67)ccc5c4c3o2)cc1 ; c1ccc(-c2nc3ccc4ccc5cc(N(c6ccccc6)c6cccc(-c7ccccc7-c7ccccc7)c6)ccc5c4c3o2)cc1]

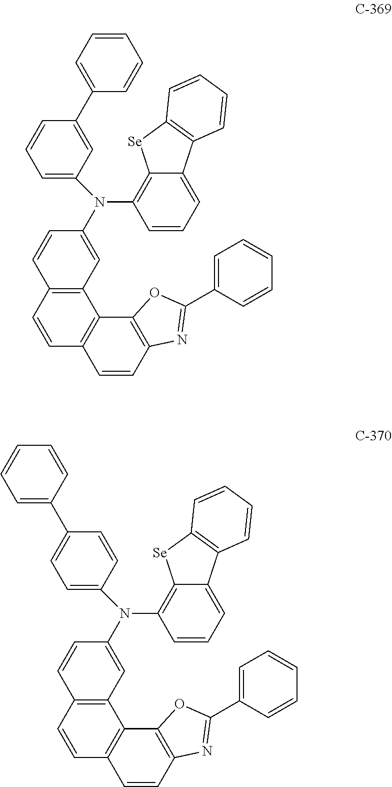 [SMILES: c1ccc(-c2cccc(N(c3ccc4ccc5ccc6nc(-c7ccccc7)oc6c5c4c3)c3cccc4c3[se]c3ccccc34)c2)cc1 ; c1ccc(-c2ccc(N(c3ccc4ccc5ccc6nc(-c7ccccc7)oc6c5c4c3)c3cccc4c3[se]c3ccccc34)cc2)cc1]

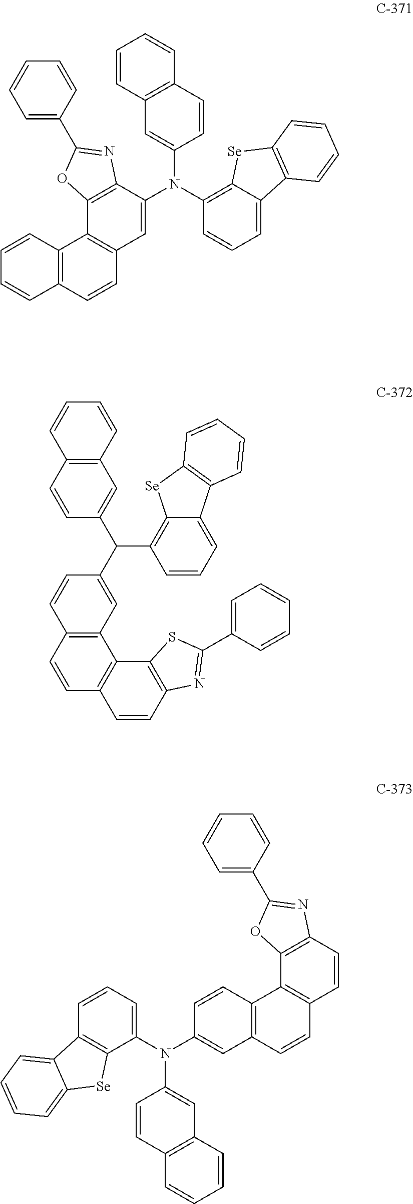 [SMILES: c1ccc(-c2nc3c(N(c4ccc5ccccc5c4)c4cccc5c4[se]c4ccccc45)cc4ccc5ccccc5c4c3o2)cc1 ; c1ccc(-c2nc3ccc4ccc5ccc(C(c6ccc7ccccc7c6)c6cccc7c6[se]c6ccccc67)cc5c4c3s2)cc1 ; c1ccc(-c2nc3ccc4ccc5cc(N(c6ccc7ccccc7c6)c6cccc7c6[se]c6ccccc67)ccc5c4c3o2)cc1]

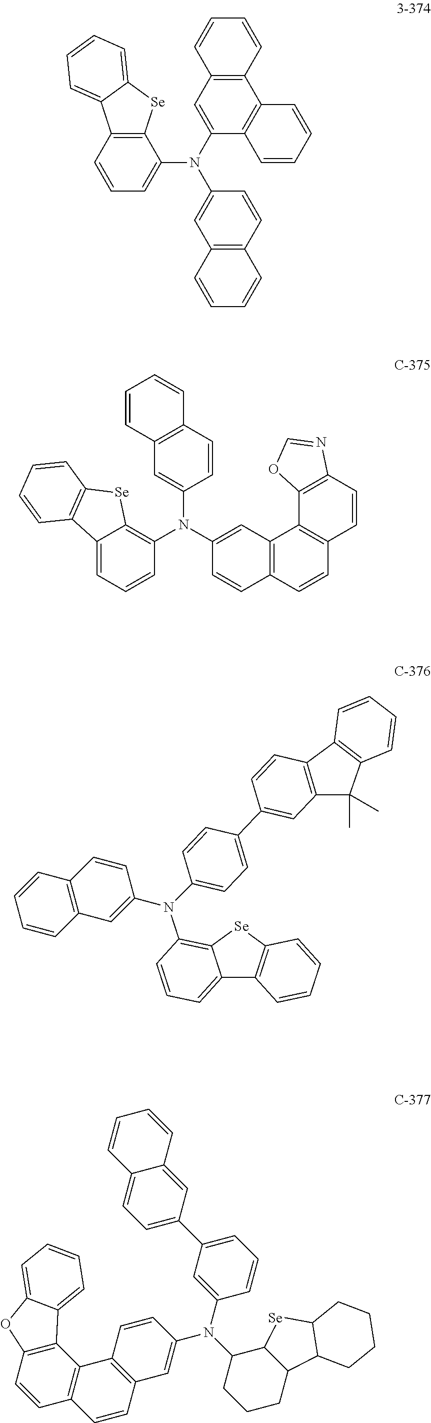 [SMILES: c1ccc2cc(N(c3cc4ccccc4c4ccccc34)c3cccc4c3[se]c3ccccc34)ccc2c1 ; c1ccc2cc(N(c3ccc4ccc5ccc6ncoc6c5c4c3)c3cccc4c3[se]c3ccccc34)ccc2c1 ; CC1(C)c2ccccc2-c2ccc(-c3ccc(N(c4ccc5ccccc5c4)c4cccc5c4[se]c4ccccc45)cc3)cc21 ; c1cc(-c2ccc3ccccc3c2)cc(N(c2ccc3c(ccc4ccc5oc6ccccc6c5c43)c2)C2CCCC3C4CCCCC4[Se]C32)c1]

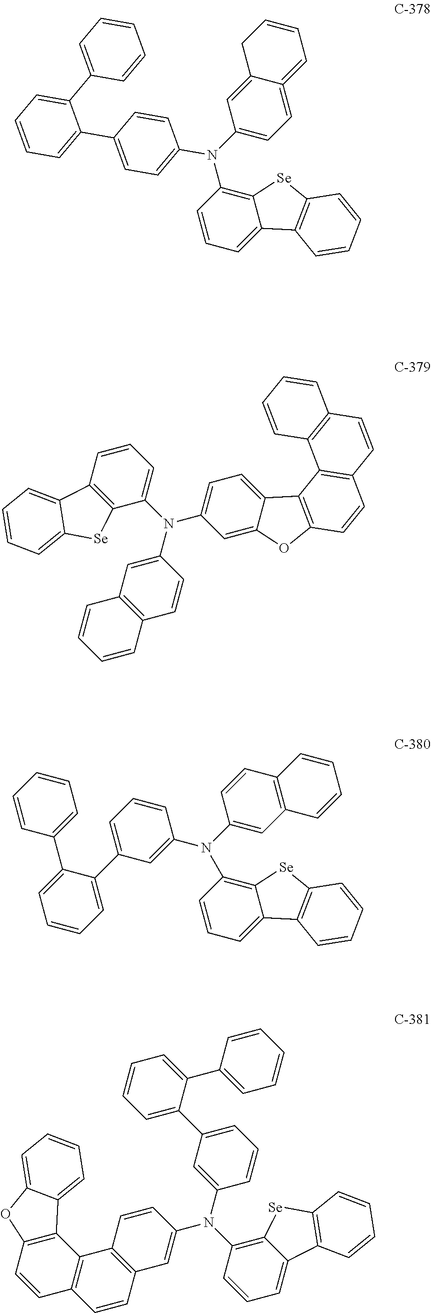

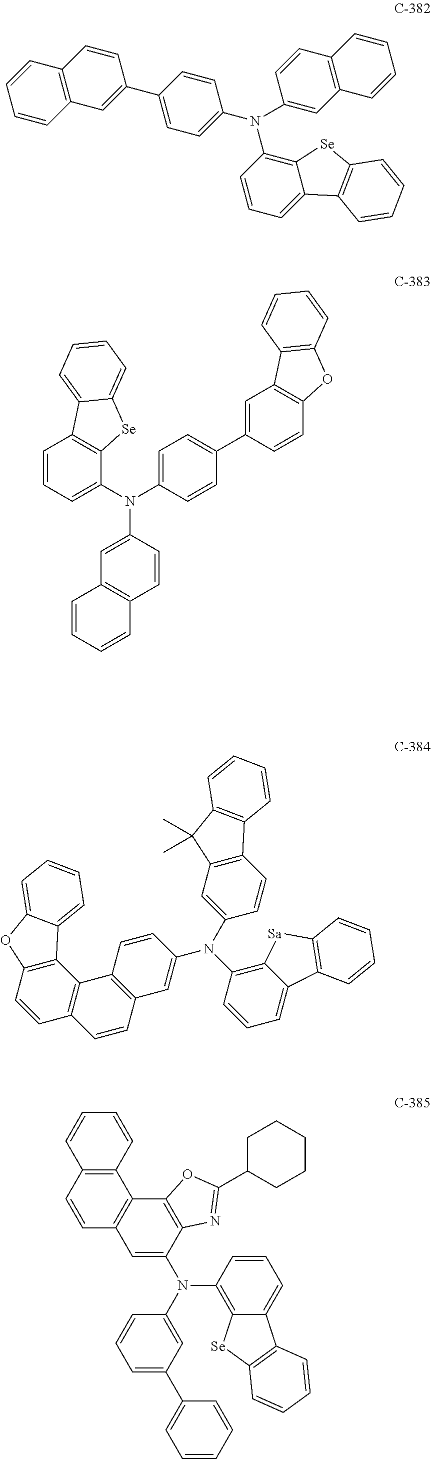

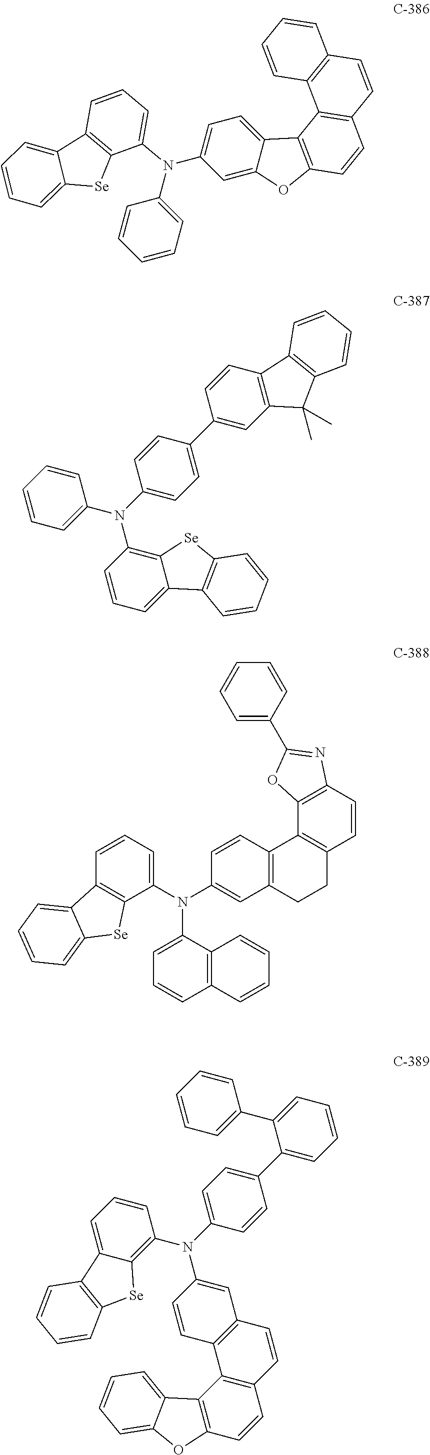 [SMILES: c1ccc(N(c2ccc3c(c2)oc2ccc4ccc5ccccc5c4c23)c2cccc3c2[se]c2ccccc23)cc1 ; CC1(C)c2ccccc2-c2ccc(-c3ccc(N(c4ccccc4)c4cccc5c4[se]c4ccccc45)cc3)cc21 ; c1ccc(-c2nc3ccc4c(c3o2)-c2ccc(N(c3cccc5ccccc35)c3cccc5c3[se]c3ccccc35)cc2CC4)cc1 ; c1ccc(-c2ccccc2-c2ccc(N(c3ccc4c(ccc5ccc6oc7ccccc7c6c54)c3)c3cccc4c3[se]c3ccccc34)cc2)cc1]

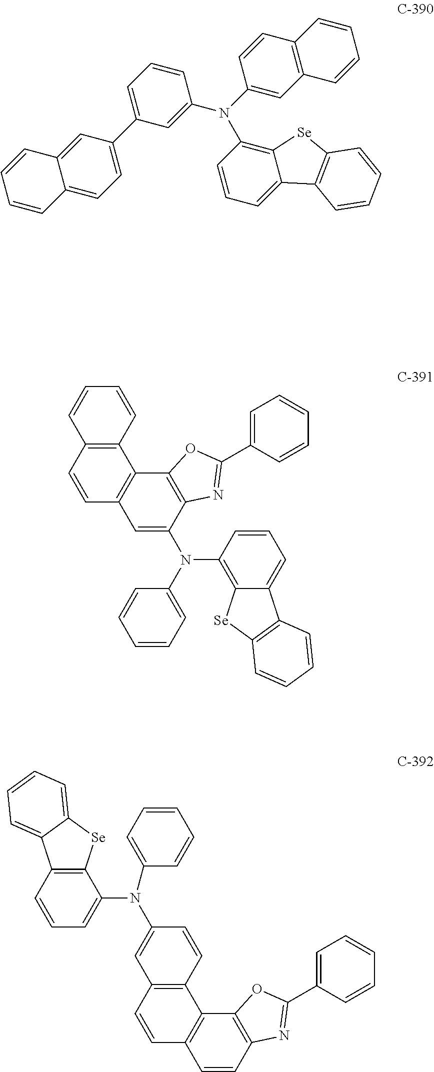 [SMILES: c1cc(-c2ccc3ccccc3c2)cc(N(c2ccc3ccccc3c2)c2cccc3c2[se]c2ccccc23)c1 ; c1ccc(-c2nc3c(N(c4ccccc4)c4cccc5c4[se]c4ccccc45)cc4ccc5ccccc5c4c3o2)cc1 ; c1ccc(-c2nc3ccc4ccc5cc(N(c6ccccc6)c6cccc7c6[se]c6ccccc67)ccc5c4c3o2)cc1]

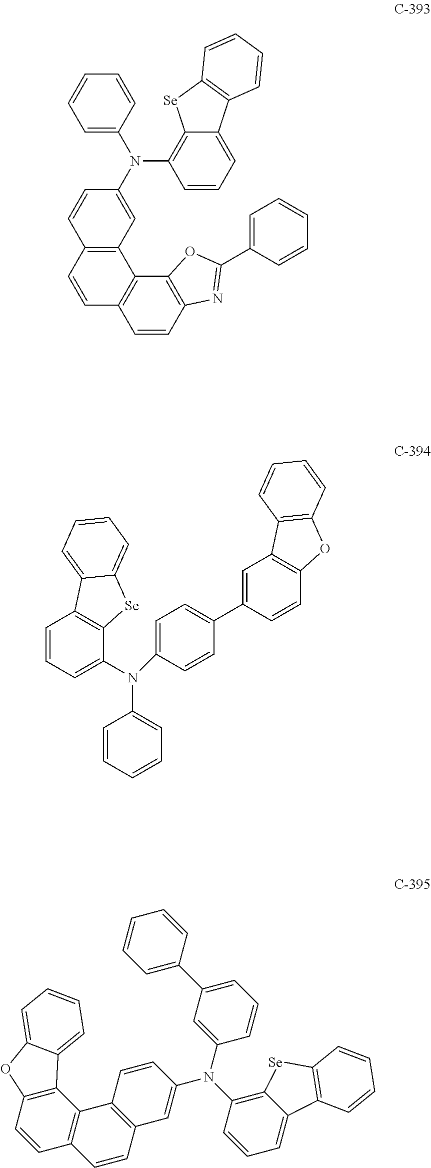 [SMILES: c1ccc(-c2nc3ccc4ccc5ccc(N(c6ccccc6)c6cccc7c6[se]c6ccccc67)cc5c4c3o2)cc1 ; c1ccc(N(c2ccc(-c3ccc4oc5ccccc5c4c3)cc2)c2cccc3c2[se]c2ccccc23)cc1 ; c1ccc(-c2cccc(N(c3ccc4c(ccc5ccc6oc7ccccc7c6c54)c3)c3cccc4c3[se]c3ccccc34)c2)cc1]

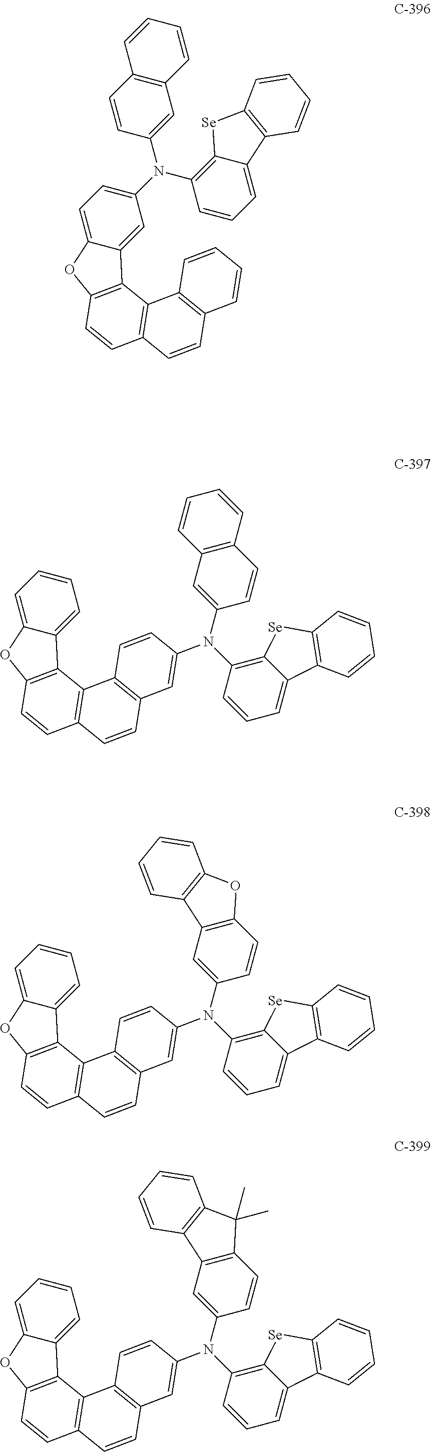 [SMILES: c1ccc2cc(N(c3ccc4oc5ccc6ccc7ccccc7c6c5c4c3)c3cccc4c3[se]c3ccccc34)ccc2c1 ; c1ccc2cc(N(c3ccc4c(ccc5ccc6oc7ccccc7c6c54)c3)c3cccc4c3[se]c3ccccc34)ccc2c1 ; c1ccc2c(c1)oc1ccc(N(c3ccc4c(ccc5ccc6oc7ccccc7c6c54)c3)c3cccc4c3[se]c3ccccc34)cc12 ; CC1(C)c2ccccc2-c2cc(N(c3ccc4c(ccc5ccc6oc7ccccc7c6c54)c3)c3cccc4c3[se]c3ccccc34)ccc21]

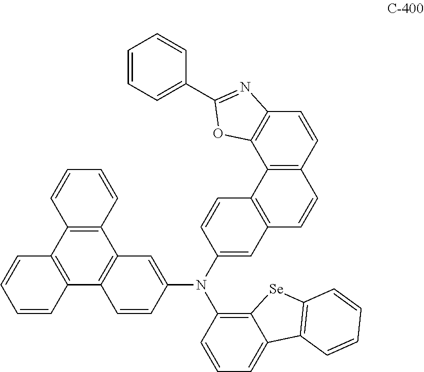 [SMILES: c1ccc(-c2nc3ccc4ccc5cc(N(c6ccc7c8ccccc8c8ccccc8c7c6)c6cccc7c6[se]c6ccccc67)ccc5c4c3o2)cc1]

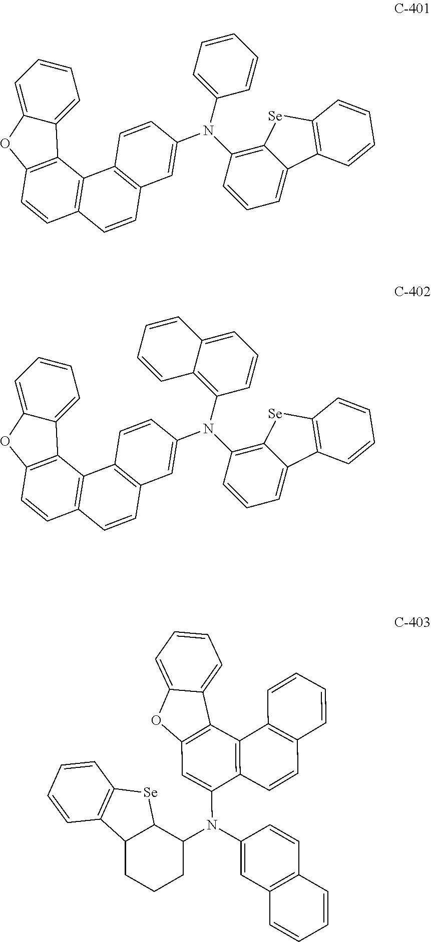 [SMILES: c1ccc(N(c2ccc3c(ccc4ccc5oc6ccccc6c5c43)c2)c2cccc3c2[se]c2ccccc23)cc1 ; c1ccc2c(N(c3ccc4c(ccc5ccc6oc7ccccc7c6c54)c3)c3cccc4c3[se]c3ccccc34)cccc2c1 ; c1ccc2c(c1)[Se]C1C2CCCC1N(c1ccc2ccccc2c1)c1cc2oc3ccccc3c2c2c1ccc1ccccc12]

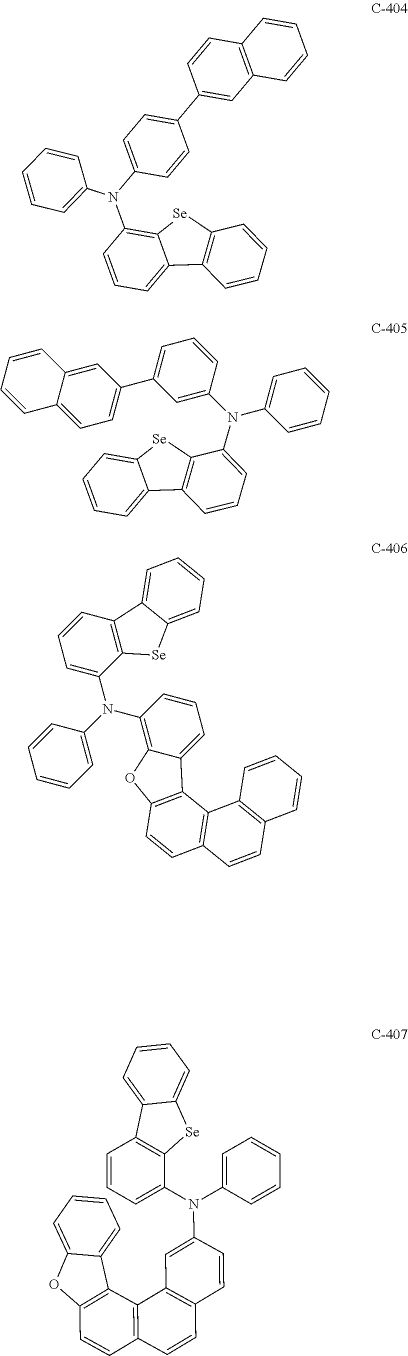 [SMILES: c1ccc(N(c2ccc(-c3ccc4ccccc4c3)cc2)c2cccc3c2[se]c2ccccc23)cc1 ; c1ccc(N(c2cccc(-c3ccc4ccccc4c3)c2)c2cccc3c2[se]c2ccccc23)cc1 ; c1ccc(N(c2cccc3c2oc2ccc4ccc5ccccc5c4c23)c2cccc3c2[se]c2ccccc23)cc1 ; c1ccc(N(c2ccc3ccc4ccc5oc6ccccc6c5c4c3c2)c2cccc3c2[se]c2ccccc23)cc1]

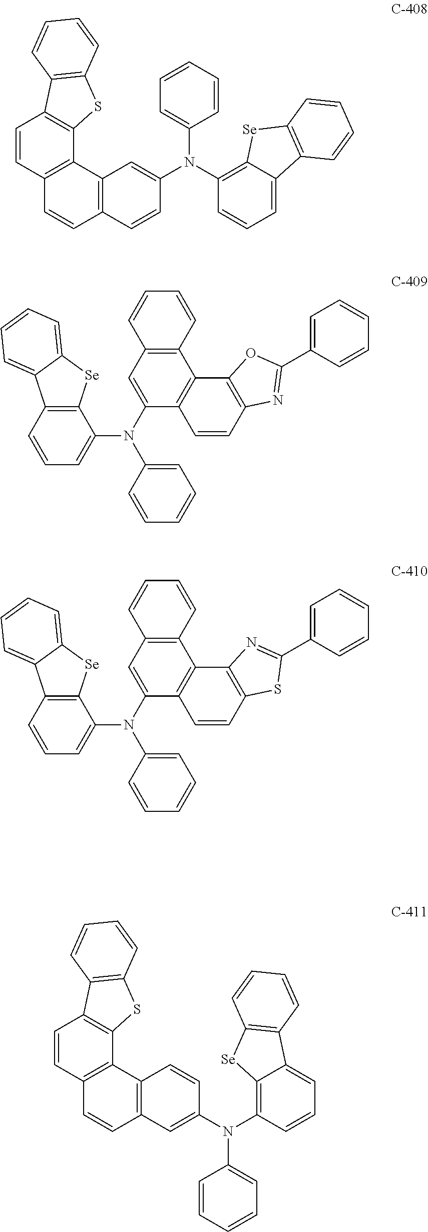 [SMILES: c1ccc(N(c2ccc3ccc4ccc5c6ccccc6sc5c4c3c2)c2cccc3c2[se]c2ccccc23)cc1 ; c1ccc(-c2nc3ccc4c(N(c5ccccc5)c5cccc6c5[se]c5ccccc56)cc5ccccc5c4c3o2)cc1 ; c1ccc(-c2nc3c(ccc4c(N(c5ccccc5)c5cccc6c5[se]c5ccccc56)cc5ccccc5c43)s2)cc1 ; c1ccc(N(c2ccc3c(ccc4ccc5c6ccccc6sc5c43)c2)c2cccc3c2[se]c2ccccc23)cc1]

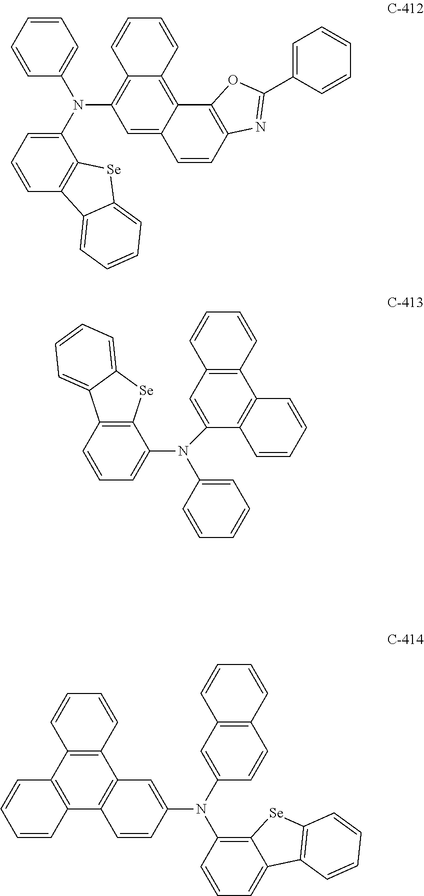 [SMILES: c1ccc(-c2nc3ccc4cc(N(c5ccccc5)c5cccc6c5[se]c5ccccc56)c5ccccc5c4c3o2)cc1 ; c1ccc(N(c2cc3ccccc3c3ccccc23)c2cccc3c2[se]c2ccccc23)cc1 ; c1ccc2cc(N(c3ccc4c5ccccc5c5ccccc5c4c3)c3cccc4c3[se]c3ccccc34)ccc2c1]

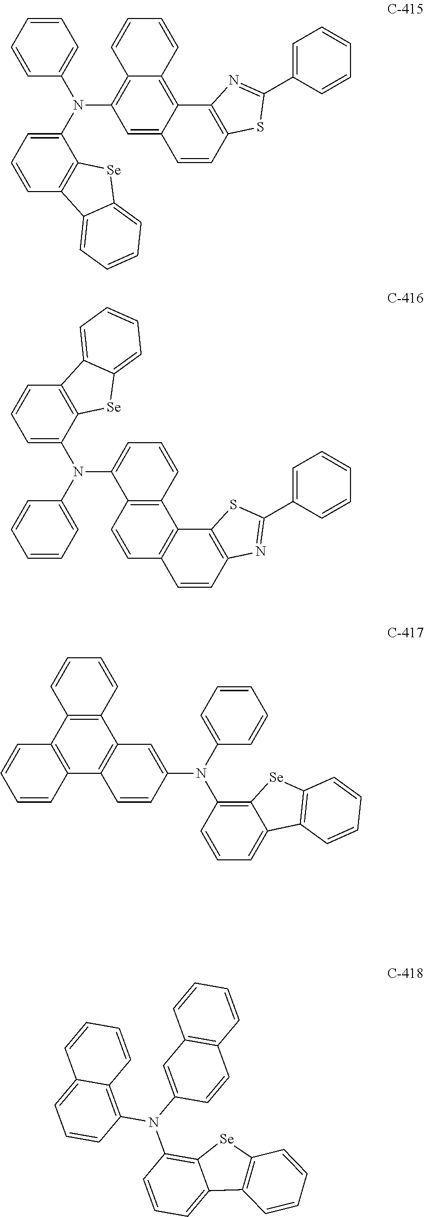

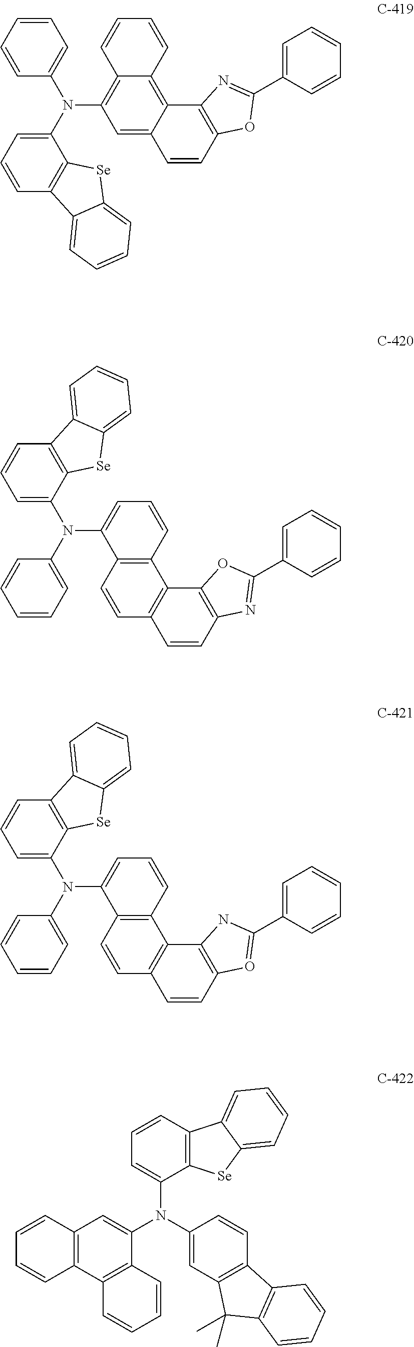

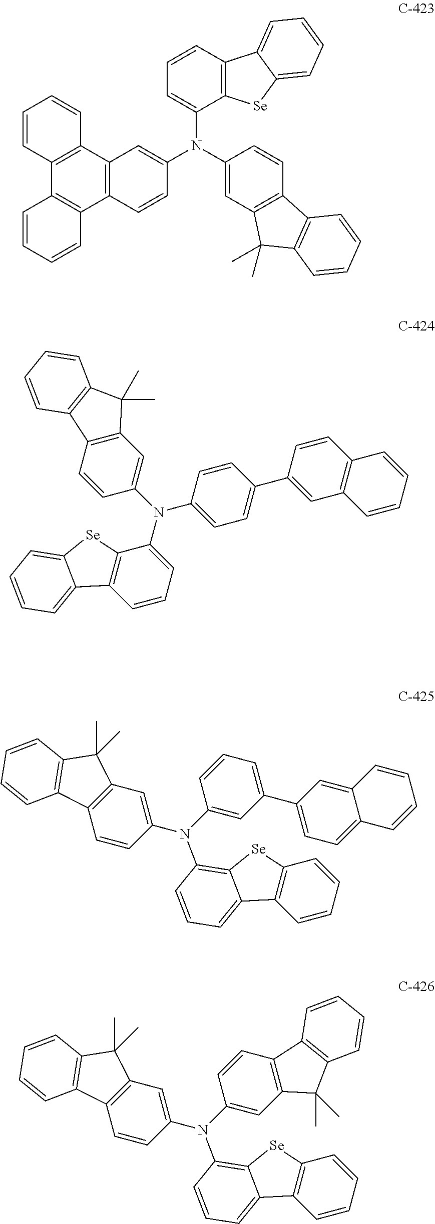 [SMILES: CC1(C)c2ccccc2-c2ccc(N(c3ccc4c5ccccc5c5ccccc5c4c3)c3cccc4c3[se]c3ccccc34)cc21 ; CC1(C)c2ccccc2-c2ccc(N(c3ccc(-c4ccc5ccccc5c4)cc3)c3cccc4c3[se]c3ccccc34)cc21 ; CC1(C)c2ccccc2-c2ccc(N(c3cccc(-c4ccc5ccccc5c4)c3)c3cccc4c3[se]c3ccccc34)cc21 ; CC1(C)c2ccccc2-c2ccc(N(c3ccc4c(c3)C(C)(C)c3ccccc3-4)c3cccc4c3[se]c3ccccc34)cc21]

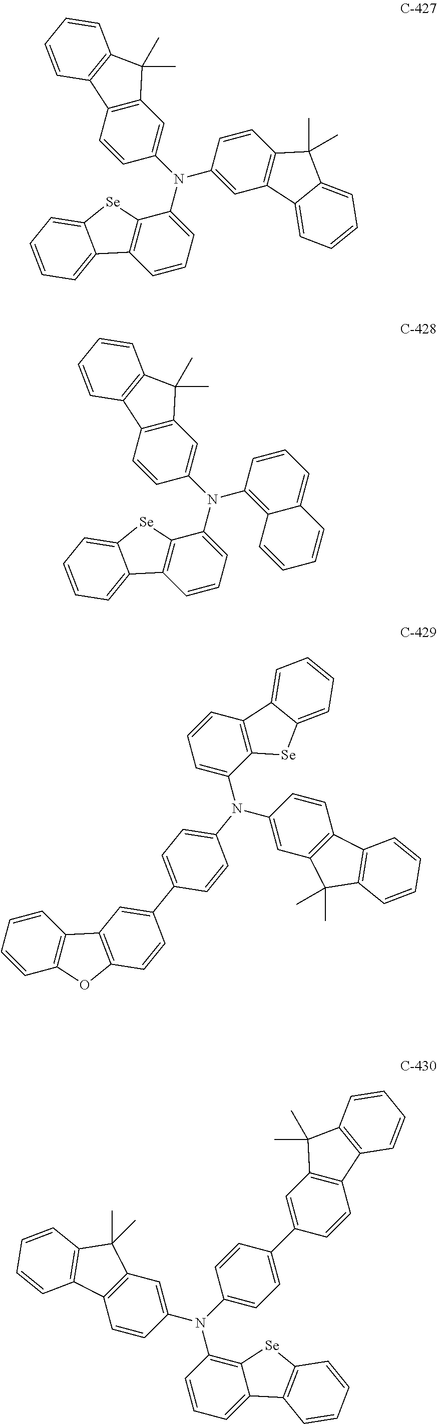 [SMILES: CC1(C)c2ccccc2-c2cc(N(c3ccc4c(c3)C(C)(C)c3ccccc3-4)c3cccc4c3[se]c3ccccc34)ccc21 ; CC1(C)c2ccccc2-c2ccc(N(c3cccc4ccccc34)c3cccc4c3[se]c3ccccc34)cc21 ; CC1(C)c2ccccc2-c2ccc(N(c3ccc(-c4ccc5oc6ccccc6c5c4)cc3)c3cccc4c3[se]c3ccccc34)cc21 ; CC1(C)c2ccccc2-c2ccc(-c3ccc(N(c4ccc5c(c4)C(C)(C)c4ccccc4-5)c4cccc5c4[se]c4ccccc45)cc3)cc21]

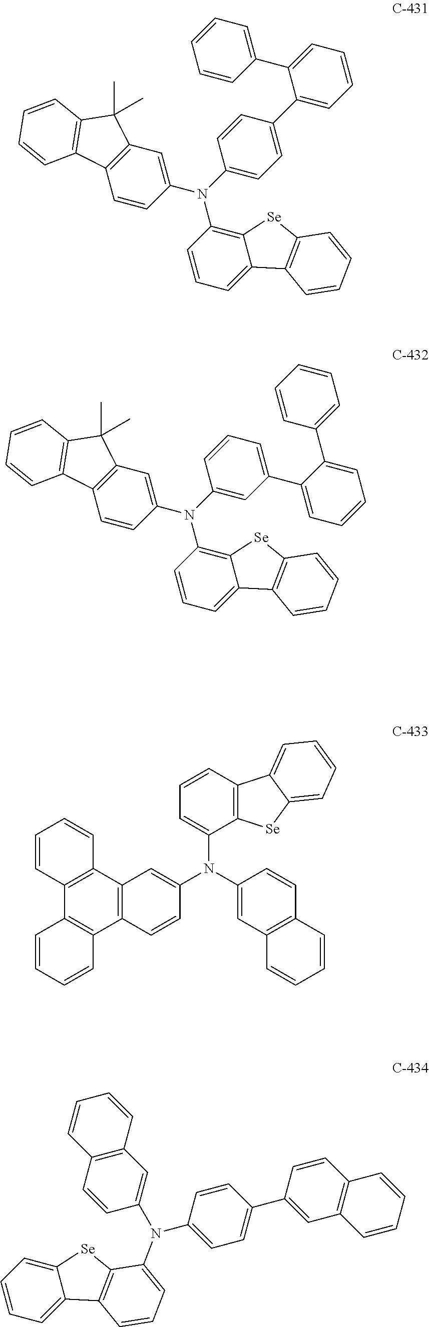 [SMILES: CC1(C)c2ccccc2-c2ccc(N(c3ccc(-c4ccccc4-c4ccccc4)cc3)c3cccc4c3[se]c3ccccc34)cc21 ; CC1(C)c2ccccc2-c2ccc(N(c3cccc(-c4ccccc4-c4ccccc4)c3)c3cccc4c3[se]c3ccccc34)cc21 ; c1ccc2cc(N(c3ccc4c5ccccc5c5ccccc5c4c3)c3cccc4c3[se]c3ccccc34)ccc2c1 ; c1ccc2cc(-c3ccc(N(c4ccc5ccccc5c4)c4cccc5c4[se]c4ccccc45)cc3)ccc2c1]

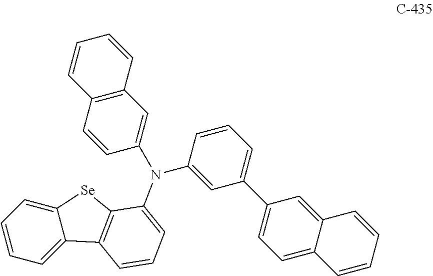 [SMILES: c1cc(-c2ccc3ccccc3c2)cc(N(c2ccc3ccccc3c2)c2cccc3c2[se]c2ccccc23)c1]

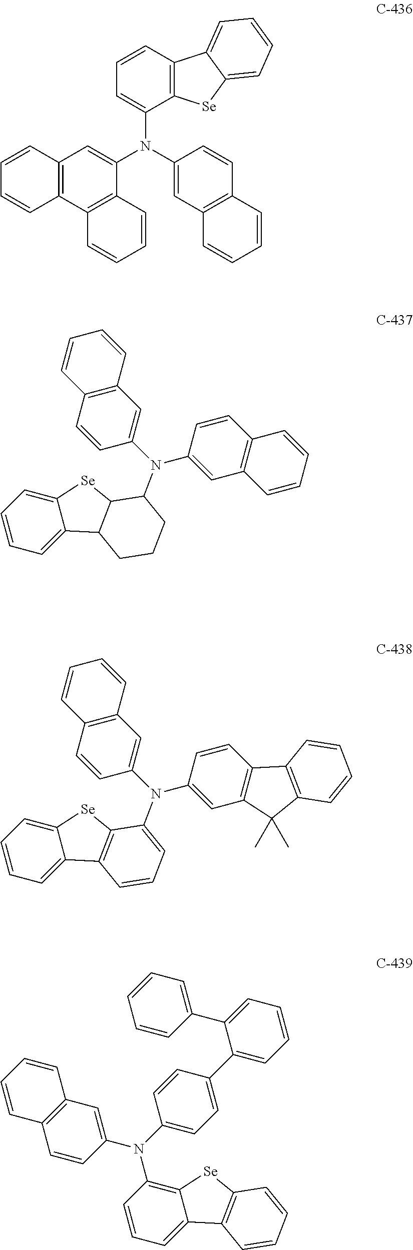 [SMILES: c1ccc2cc(N(c3cc4ccccc4c4ccccc34)c3cccc4c3[se]c3ccccc34)ccc2c1 ; c1ccc2c(c1)[Se]C1C2CCCC1N(c1ccc2ccccc2c1)c1ccc2ccccc2c1 ; CC1(C)c2ccccc2-c2ccc(N(c3ccc4ccccc4c3)c3cccc4c3[se]c3ccccc34)cc21 ; c1ccc(-c2ccccc2-c2ccc(N(c3ccc4ccccc4c3)c3cccc4c3[se]c3ccccc34)cc2)cc1]

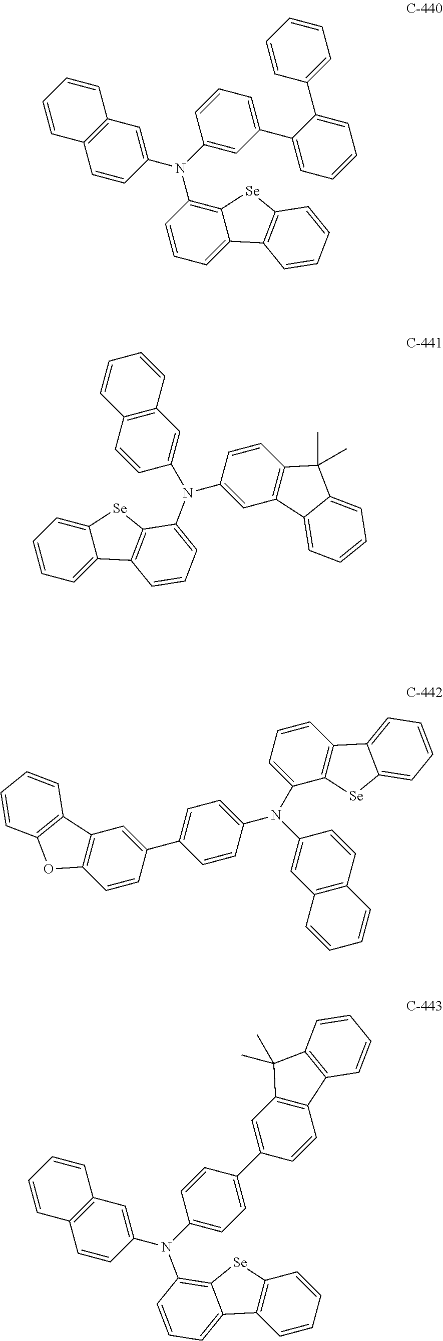 [SMILES: c1ccc(-c2ccccc2-c2cccc(N(c3ccc4ccccc4c3)c3cccc4c3[se]c3ccccc34)c2)cc1 ; CC1(C)c2ccccc2-c2cc(N(c3ccc4ccccc4c3)c3cccc4c3[se]c3ccccc34)ccc21 ; c1ccc2cc(N(c3ccc(-c4ccc5oc6ccccc6c5c4)cc3)c3cccc4c3[se]c3ccccc34)ccc2c1 ; CC1(C)c2ccccc2-c2ccc(-c3ccc(N(c4ccc5ccccc5c4)c4cccc5c4[se]c4ccccc45)cc3)cc21]

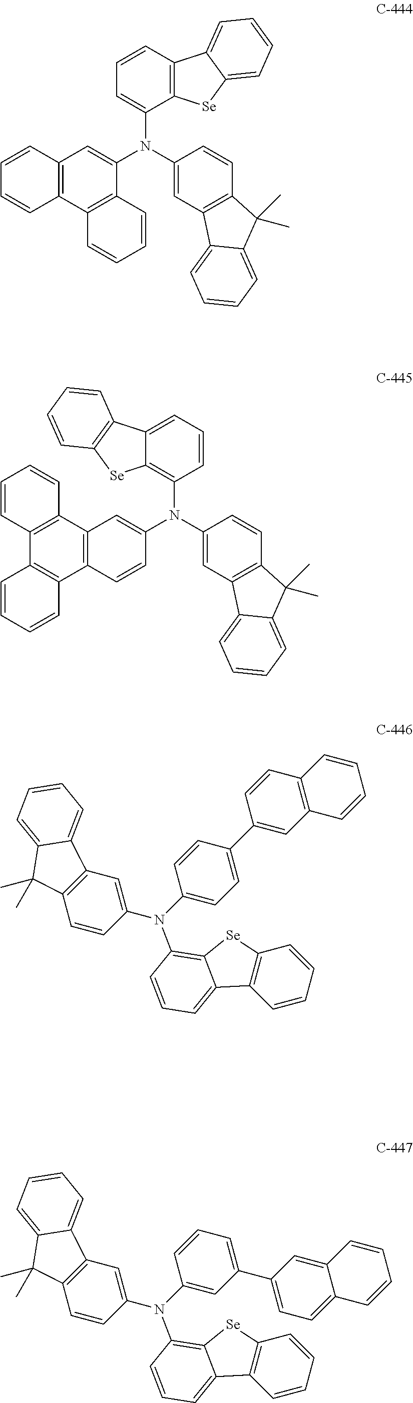

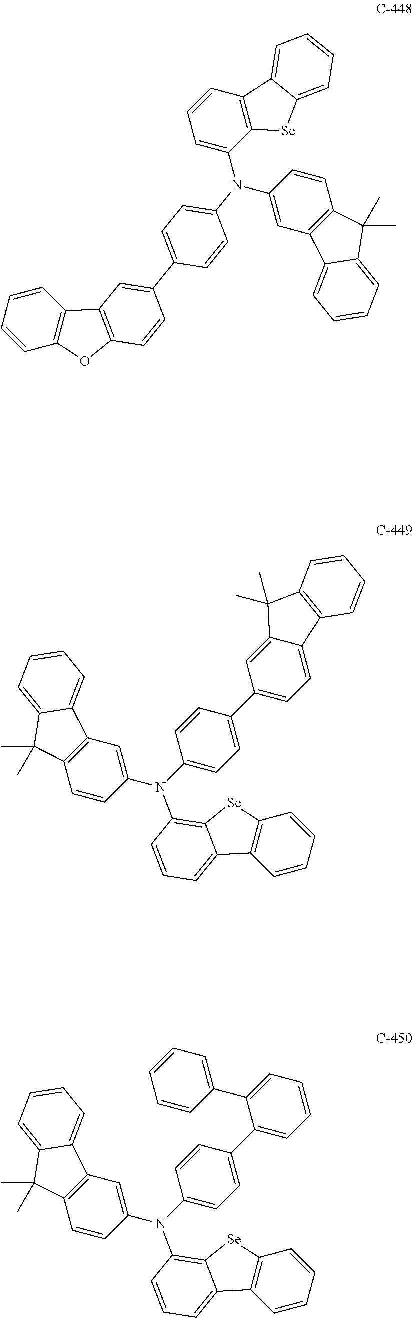

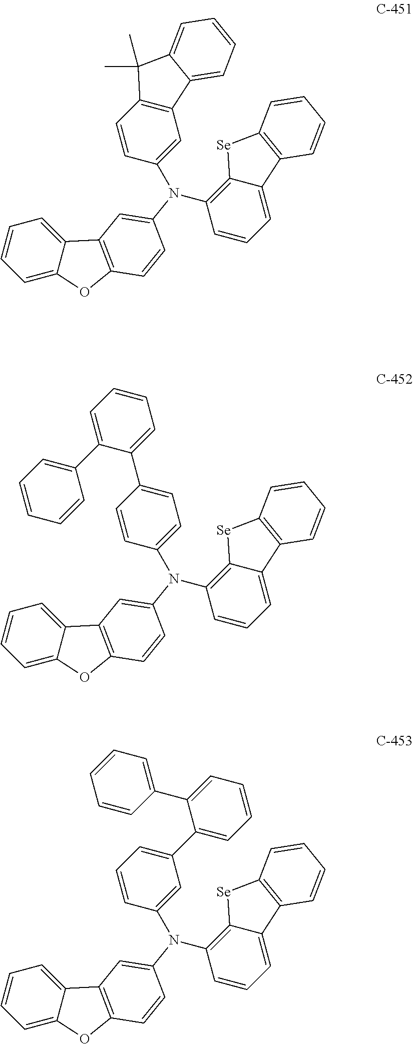 [SMILES: CC1(C)c2ccccc2-c2cc(N(c3ccc4oc5ccccc5c4c3)c3cccc4c3[se]c3ccccc34)ccc21 ; c1ccc(-c2ccccc2-c2ccc(N(c3ccc4oc5ccccc5c4c3)c3cccc4c3[se]c3ccccc34)cc2)cc1 ; c1ccc(-c2ccccc2-c2cccc(N(c3ccc4oc5ccccc5c4c3)c3cccc4c3[se]c3ccccc34)c2)cc1]

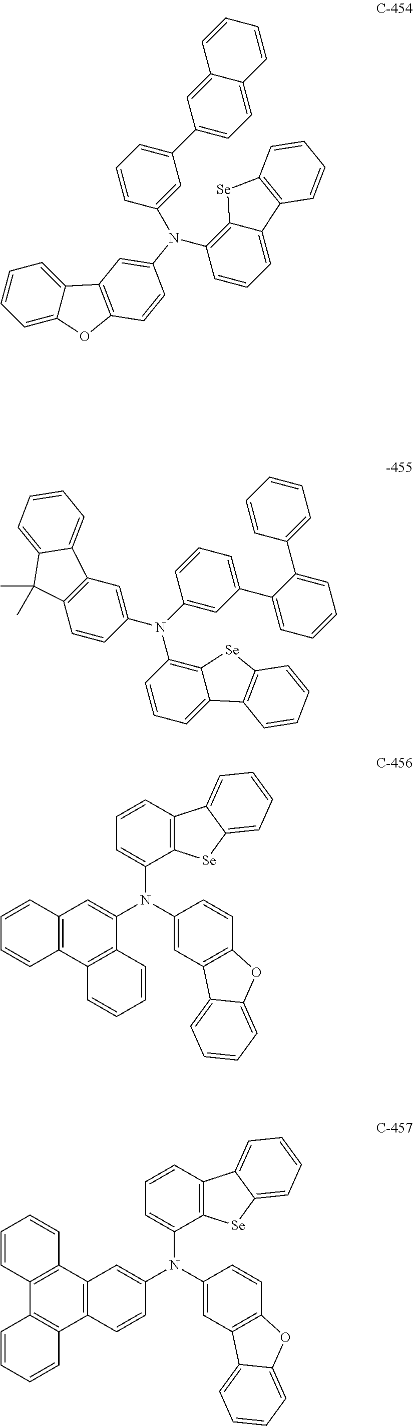

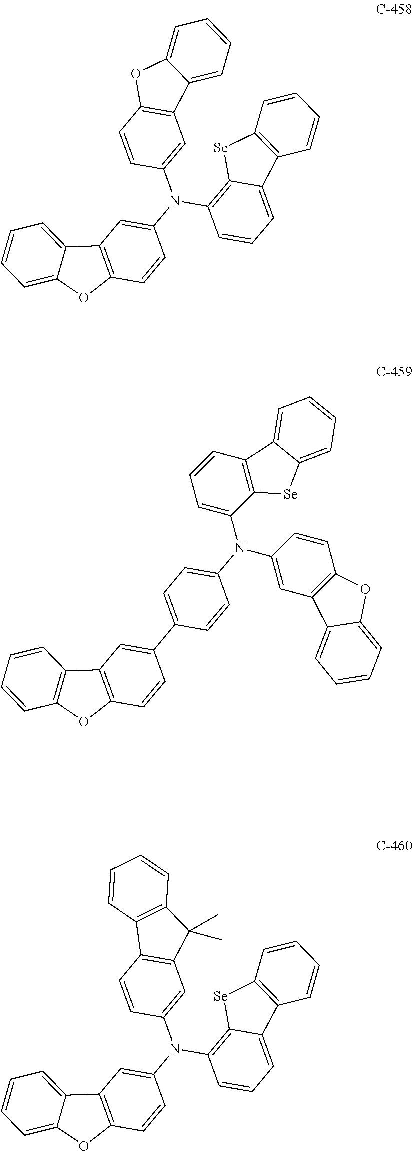 [SMILES: c1ccc2c(c1)oc1ccc(N(c3ccc4oc5ccccc5c4c3)c3cccc4c3[se]c3ccccc34)cc12 ; c1ccc2c(c1)oc1ccc(-c3ccc(N(c4ccc5oc6ccccc6c5c4)c4cccc5c4[se]c4ccccc45)cc3)cc12 ; CC1(C)c2ccccc2-c2ccc(N(c3ccc4oc5ccccc5c4c3)c3cccc4c3[se]c3ccccc34)cc21]

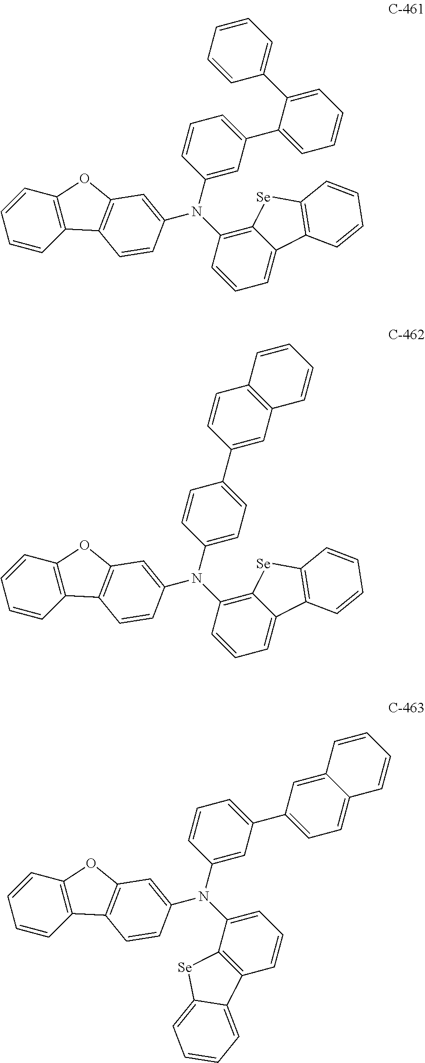 [SMILES: c1ccc(-c2ccccc2-c2cccc(N(c3ccc4c(c3)oc3ccccc34)c3cccc4c3[se]c3ccccc34)c2)cc1 ; c1ccc2cc(-c3ccc(N(c4ccc5c(c4)oc4ccccc45)c4cccc5c4[se]c4ccccc45)cc3)ccc2c1 ; c1cc(-c2ccc3ccccc3c2)cc(N(c2ccc3c(c2)oc2ccccc23)c2cccc3c2[se]c2ccccc23)c1]

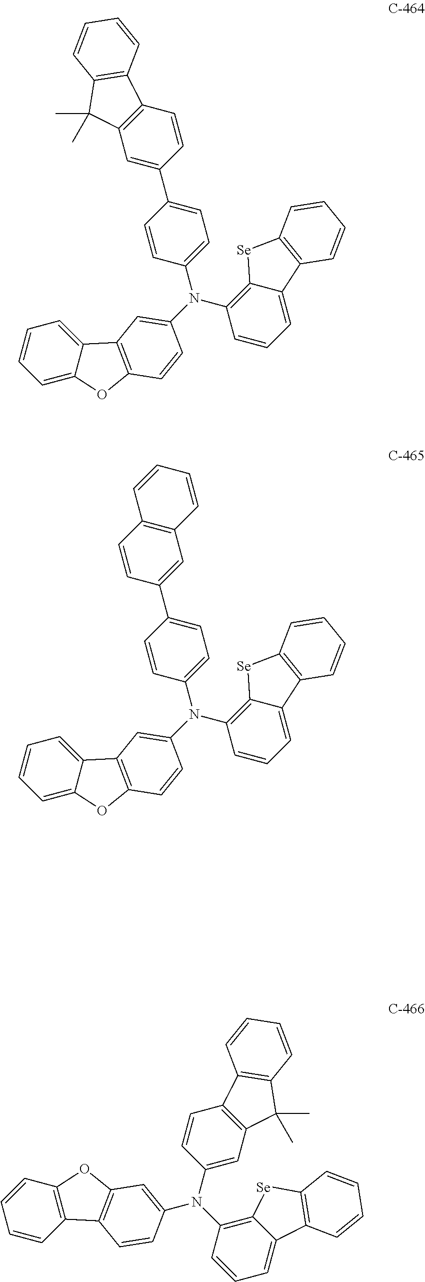 [SMILES: CC1(C)c2ccccc2-c2ccc(-c3ccc(N(c4ccc5oc6ccccc6c5c4)c4cccc5c4[se]c4ccccc45)cc3)cc21 ; c1ccc2cc(-c3ccc(N(c4ccc5oc6ccccc6c5c4)c4cccc5c4[se]c4ccccc45)cc3)ccc2c1 ; CC1(C)c2ccccc2-c2ccc(N(c3ccc4c(c3)oc3ccccc34)c3cccc4c3[se]c3ccccc34)cc21]

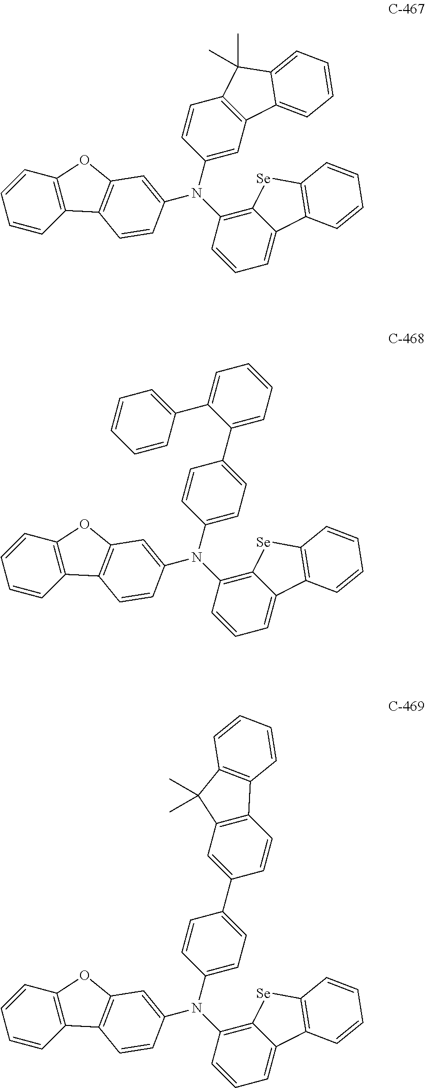 [SMILES: CC1(C)c2ccccc2-c2cc(N(c3ccc4c(c3)oc3ccccc34)c3cccc4c3[se]c3ccccc34)ccc21 ; c1ccc(-c2ccccc2-c2ccc(N(c3ccc4c(c3)oc3ccccc34)c3cccc4c3[se]c3ccccc34)cc2)cc1 ; CC1(C)c2ccccc2-c2ccc(-c3ccc(N(c4ccc5c(c4)oc4ccccc45)c4cccc5c4[se]c4ccccc45)cc3)cc21]

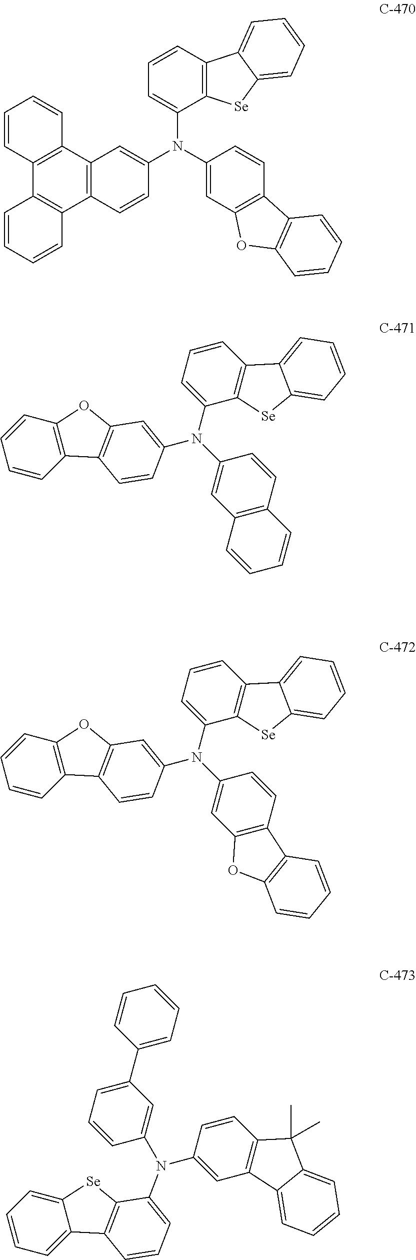 [SMILES: c1ccc2c(c1)oc1cc(N(c3ccc4c5ccccc5c5ccccc5c4c3)c3cccc4c3[se]c3ccccc34)ccc12 ; c1ccc2cc(N(c3ccc4c(c3)oc3ccccc34)c3cccc4c3[se]c3ccccc34)ccc2c1 ; c1ccc2c(c1)oc1cc(N(c3ccc4c(c3)oc3ccccc34)c3cccc4c3[se]c3ccccc34)ccc12 ; CC1(C)c2ccccc2-c2cc(N(c3cccc(-c4ccccc4)c3)c3cccc4c3[se]c3ccccc34)ccc21]

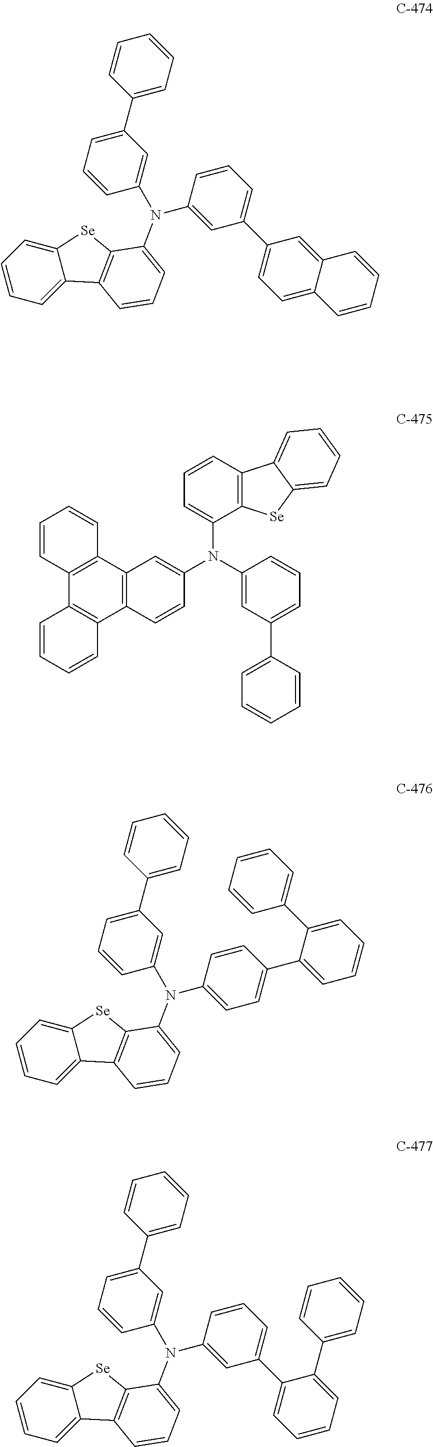 [SMILES: c1ccc(-c2cccc(N(c3cccc(-c4ccc5ccccc5c4)c3)c3cccc4c3[se]c3ccccc34)c2)cc1 ; c1ccc(-c2cccc(N(c3ccc4c5ccccc5c5ccccc5c4c3)c3cccc4c3[se]c3ccccc34)c2)cc1 ; c1ccc(-c2cccc(N(c3ccc(-c4ccccc4-c4ccccc4)cc3)c3cccc4c3[se]c3ccccc34)c2)cc1 ; c1ccc(-c2cccc(N(c3cccc(-c4ccccc4-c4ccccc4)c3)c3cccc4c3[se]c3ccccc34)c2)cc1]

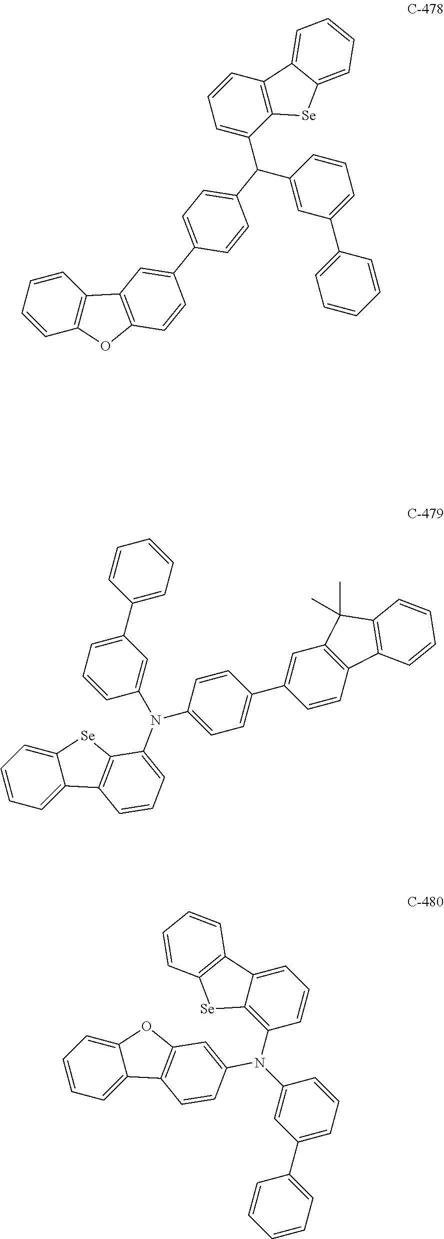 [SMILES: c1ccc(-c2cccc(C(c3ccc(-c4ccc5oc6ccccc6c5c4)cc3)c3cccc4c3[se]c3ccccc34)c2)cc1 ; CC1(C)c2ccccc2-c2ccc(-c3ccc(N(c4cccc(-c5ccccc5)c4)c4cccc5c4[se]c4ccccc45)cc3)cc21 ; c1ccc(-c2cccc(N(c3ccc4c(c3)oc3ccccc34)c3cccc4c3[se]c3ccccc34)c2)cc1]

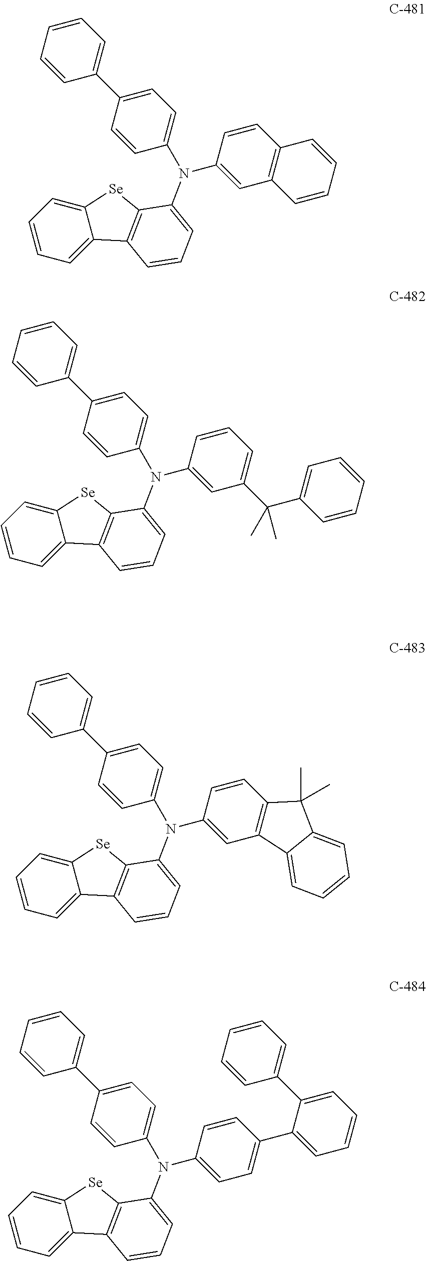 [SMILES: c1ccc(-c2ccc(N(c3ccc4ccccc4c3)c3cccc4c3[se]c3ccccc34)cc2)cc1 ; CC(C)(c1ccccc1)c1cccc(N(c2ccc(-c3ccccc3)cc2)c2cccc3c2[se]c2ccccc23)c1 ; CC1(C)c2ccccc2-c2cc(N(c3ccc(-c4ccccc4)cc3)c3cccc4c3[se]c3ccccc34)ccc21 ; c1ccc(-c2ccc(N(c3ccc(-c4ccccc4-c4ccccc4)cc3)c3cccc4c3[se]c3ccccc34)cc2)cc1]

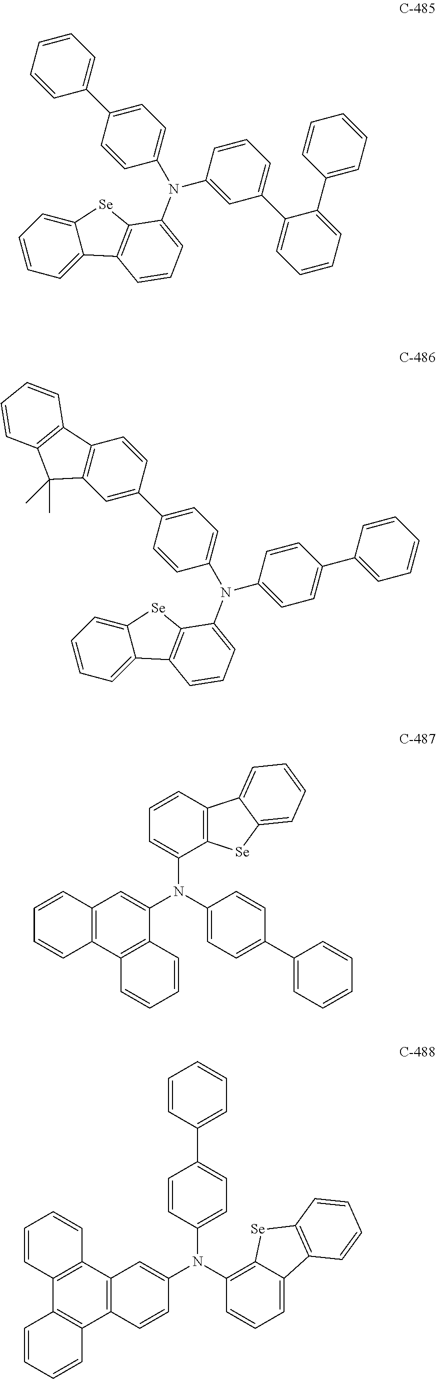 [SMILES: c1ccc(-c2ccc(N(c3cccc(-c4ccccc4-c4ccccc4)c3)c3cccc4c3[se]c3ccccc34)cc2)cc1 ; CC1(C)c2ccccc2-c2ccc(-c3ccc(N(c4ccc(-c5ccccc5)cc4)c4cccc5c4[se]c4ccccc45)cc3)cc21 ; c1ccc(-c2ccc(N(c3cc4ccccc4c4ccccc34)c3cccc4c3[se]c3ccccc34)cc2)cc1 ; c1ccc(-c2ccc(N(c3ccc4c5ccccc5c5ccccc5c4c3)c3cccc4c3[se]c3ccccc34)cc2)cc1]

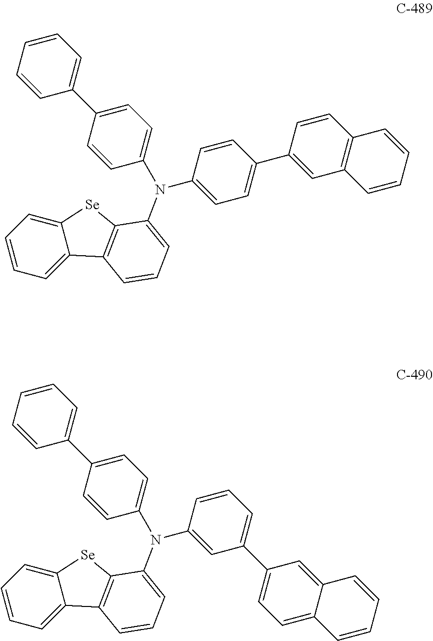 [SMILES: c1ccc(-c2ccc(N(c3ccc(-c4ccc5ccccc5c4)cc3)c3cccc4c3[se]c3ccccc34)cc2)cc1 ; c1ccc(-c2ccc(N(c3cccc(-c4ccc5ccccc5c4)c3)c3cccc4c3[se]c3ccccc34)cc2)cc1]

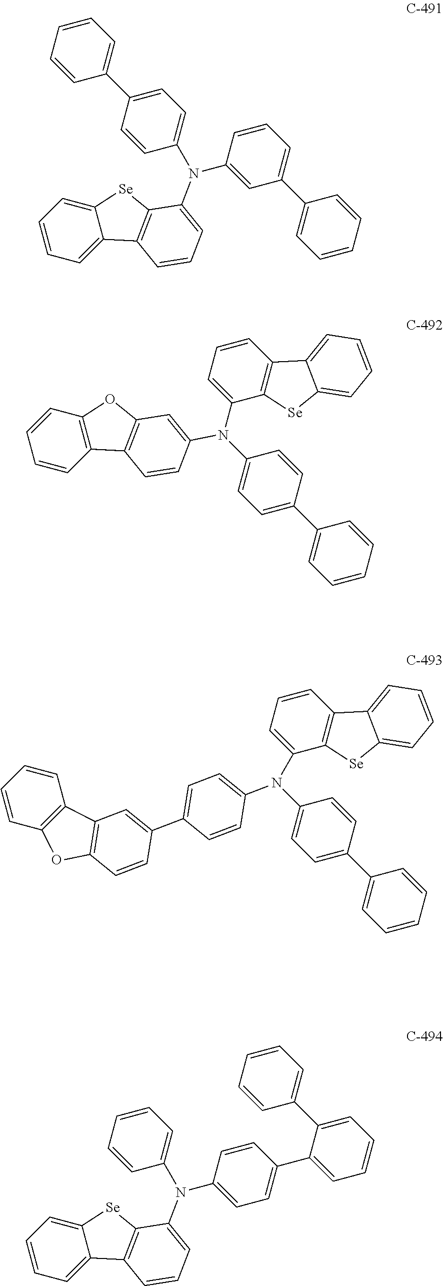 [SMILES: c1ccc(-c2ccc(N(c3cccc(-c4ccccc4)c3)c3cccc4c3[se]c3ccccc34)cc2)cc1 ; c1ccc(-c2ccc(N(c3ccc4c(c3)oc3ccccc34)c3cccc4c3[se]c3ccccc34)cc2)cc1 ; c1ccc(-c2ccc(N(c3ccc(-c4ccc5oc6ccccc6c5c4)cc3)c3cccc4c3[se]c3ccccc34)cc2)cc1 ; c1ccc(-c2ccccc2-c2ccc(N(c3ccccc3)c3cccc4c3[se]c3ccccc34)cc2)cc1]

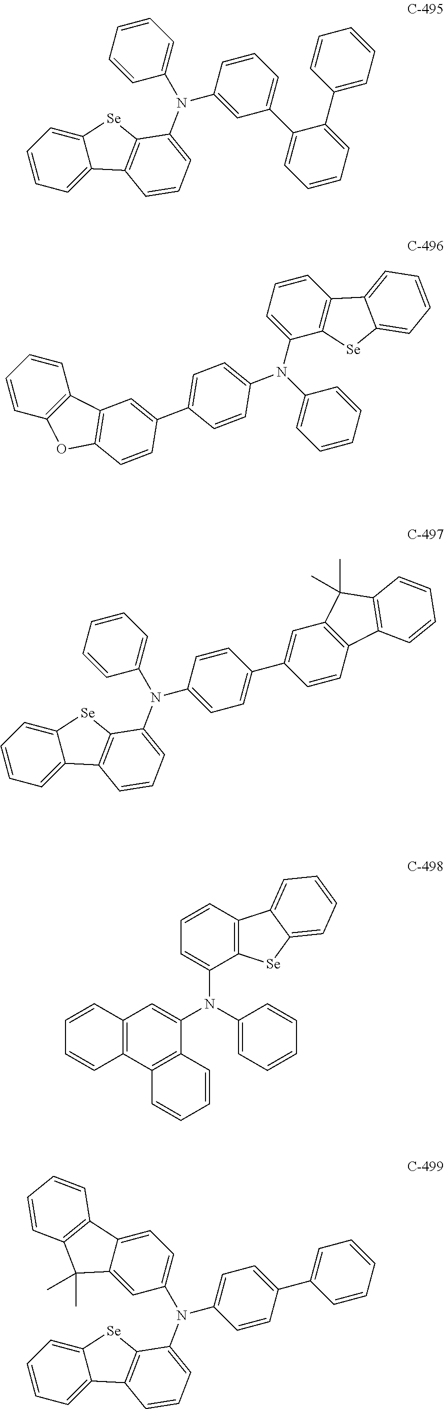 [SMILES: c1ccc(-c2ccccc2-c2cccc(N(c3ccccc3)c3cccc4c3[se]c3ccccc34)c2)cc1 ; c1ccc(N(c2ccc(-c3ccc4oc5ccccc5c4c3)cc2)c2cccc3c2[se]c2ccccc23)cc1 ; CC1(C)c2ccccc2-c2ccc(-c3ccc(N(c4ccccc4)c4cccc5c4[se]c4ccccc45)cc3)cc21 ; c1ccc(N(c2cc3ccccc3c3ccccc23)c2cccc3c2[se]c2ccccc23)cc1 ; CC1(C)c2ccccc2-c2ccc(N(c3ccc(-c4ccccc4)cc3)c3cccc4c3[se]c3ccccc34)cc21]

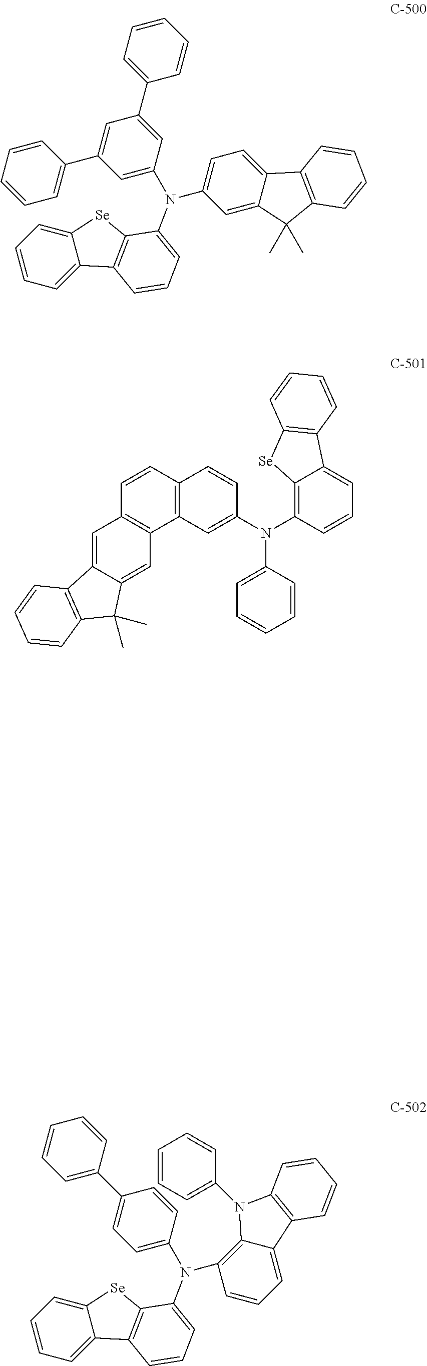 [SMILES: CC1(C)c2ccccc2-c2ccc(N(c3cc(-c4ccccc4)cc(-c4ccccc4)c3)c3cccc4c3[se]c3ccccc34)cc21 ; CC1(C)c2ccccc2-c2cc3ccc4ccc(N(c5ccccc5)c5cccc6c5[se]c5ccccc56)cc4c3cc21 ; c1ccc(-c2ccc(N(c3cccc4c3[se]c3ccccc34)c3cccc4c5ccccc5n(-c5ccccc5)c34)cc2)cc1]

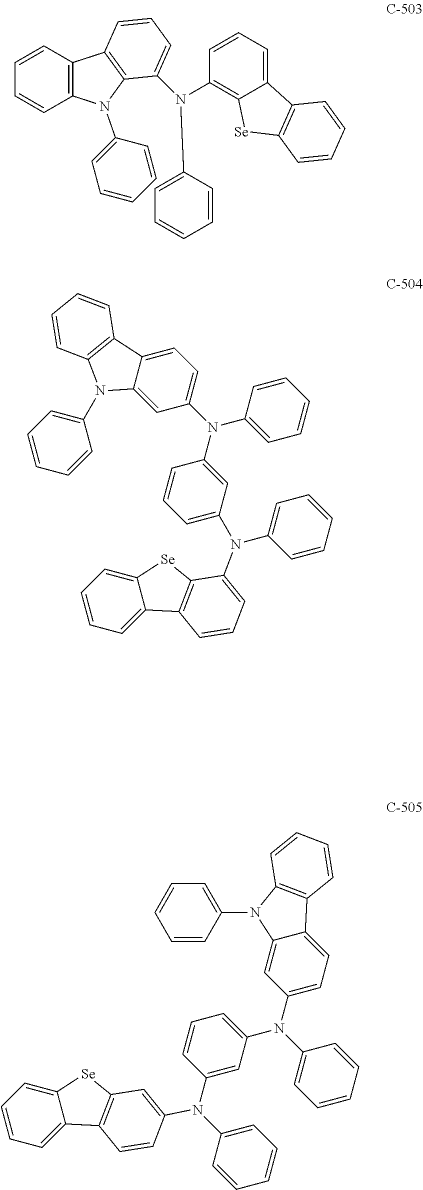 [SMILES: c1ccc(N(c2cccc3c2[se]c2ccccc23)c2cccc3c4ccccc4n(-c4ccccc4)c23)cc1 ; c1ccc(N(c2cccc(N(c3ccccc3)c3cccc4c3[se]c3ccccc34)c2)c2ccc3c4ccccc4n(-c4ccccc4)c3c2)cc1 ; c1ccc(N(c2cccc(N(c3ccccc3)c3ccc4c5ccccc5n(-c5ccccc5)c4c3)c2)c2ccc3c(c2)[se]c2ccccc23)cc1]

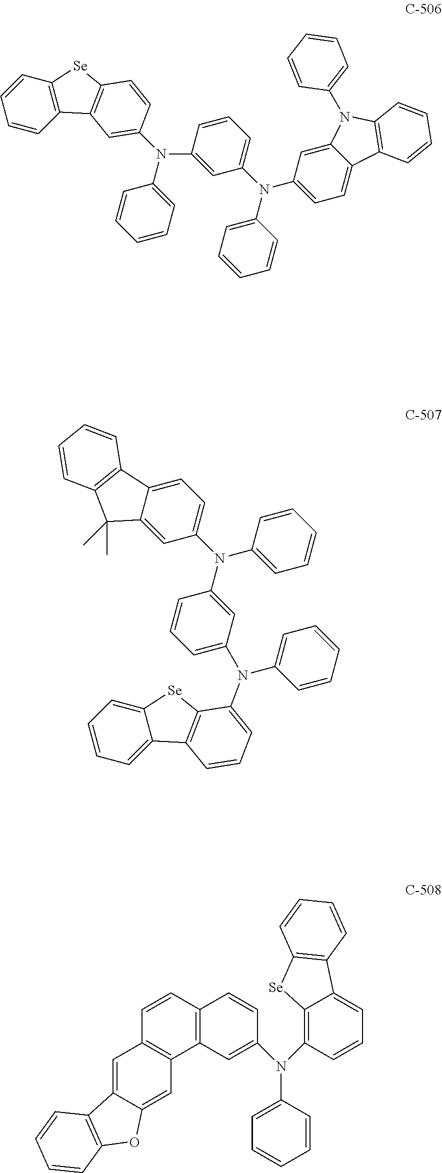 [SMILES: c1ccc(N(c2cccc(N(c3ccccc3)c3ccc4c5ccccc5n(-c5ccccc5)c4c3)c2)c2ccc3[se]c4ccccc4c3c2)cc1 ; CC1(C)c2ccccc2-c2ccc(N(c3ccccc3)c3cccc(N(c4ccccc4)c4cccc5c4[se]c4ccccc45)c3)cc21 ; c1ccc(N(c2ccc3ccc4cc5c(cc4c3c2)oc2ccccc25)c2cccc3c2[se]c2ccccc23)cc1]

The compound represented by formula 1 according to the present disclosure may be prepared by a synthetic method known to one skilled in the art. For example, the compound represented by formula 1 according to the present disclosure may be prepared by referring to the following reaction scheme 1, but is not limited thereto.

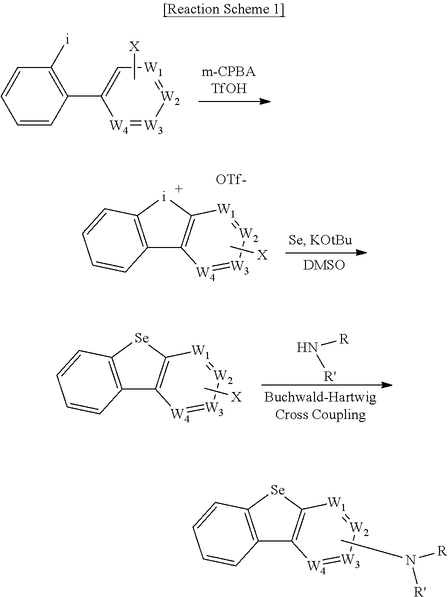

In reaction scheme 1, X represents a halogen, W₁ to W₄ represent CR₅ to CR₈, respectively, provided that any one of W₁ to W₄ represents X. In addition, in reaction scheme 1, R and R′ represent L₁-Ar₁ and L₂-Ar₂, respectively. R₅ to R₈, L₁, L₂, Ar₁ and Ar₂ are as defined in formula 1.

Although illustrative synthesis examples of the compounds represented by formula 1 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formula 1 above, but are not specified in the specific synthesis examples, are bonded.

The present disclosure provides an organic electroluminescent material comprising the organic electroluminescent compound of formula 1, and an organic electroluminescent device comprising the organic electroluminescent material.

The organic electroluminescent material may be a hole transport material, a hole auxiliary material or a light-emitting auxiliary material, and specifically, a hole transport material, a hole auxiliary material or a light-emitting auxiliary material of a blue light-emitting organic electroluminescent device. When the hole transport layer is two or more layers, the organic electroluminescent material may be a hole transport material (a hole auxiliary material) included in the hole transport layer adjacent to a light-emitting layer.

The organic electroluminescent material may be comprised solely of the organic electroluminescent compound of the present disclosure, or may further comprise conventional materials included in the organic electroluminescent material.

The hole transport zone of the present disclosure may be comprised of one or more layers from the group consisting of a hole transport layer, a hole injection layer, an electron blocking layer and a hole auxiliary layer, and each of the layers may consist of one or more layers.

According to one embodiment of the present disclosure, the hole transport zone includes a hole transport layer. In addition, the hole transport zone may include a hole transport layer, and further include at least one of a hole injection layer, an electron blocking layer, and a hole auxiliary layer.

The organic electroluminescent device according to the present disclosure includes a first electrode; a second electrode; and at least one organic layer interposed between the first electrode and the second electrode. The organic layer may comprise at least one organic electroluminescent compound represented by formula 1. One of the first electrode and the second electrode may be an anode and the other may be a cathode. The organic layer may comprise a light-emitting layer, and may further include at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The organic electroluminescent compound represented by formula 1 of the present disclosure may be included in any one layer of the light-emitting layer, the hole injection layer, the hole transport layer, the hole auxiliary layer, the light-emitting auxiliary layer, the electron transport layer, the electron buffer layer, the electron injection layer, the interlayer, the hole blocking layer, and the electron blocking layer. In some cases, preferably, it may be included in at least one layer of the hole transport layer, the hole auxiliary layer, the light-emitting auxiliary layer, and the light-emitting layer. When the hole transport layer is two or more layers, the organic electroluminescent compound represented by formula 1 of the present disclosure may be used in at least one of the hole transport layers. For example, when used in the hole transport layer, the organic electroluminescent compound of the present disclosure may be comprised as a hole transport material. In addition, when used in the light-emitting layer, the organic electroluminescent compound of the present disclosure may be comprised as a host material.

The light-emitting layer may include at least one host and at least one dopant. If necessary, the light-emitting layer may include a co-host material, i.e., two or more host materials. The organic electroluminescent compound of the present disclosure may be used as a co-host material.

The host used in the present disclosure may be a phosphorescent host compound or a fluorescent host compound, and these host compounds are not particularly limited.

As a dopant included in the organic electroluminescent device of the present disclosure, at least one phosphorescent or fluorescent dopant may be used, and a fluorescent dopant may be preferred.

The fluorescent host material included in the organic electroluminescent device of the present disclosure is not particularly limited, but a compound represented by the following formula 90 may be used.

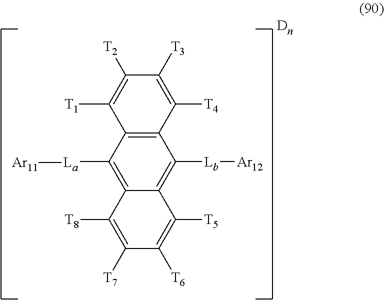

In formula 90,

Lₐ and L_{b}, each independently, represent a single bond, a substituted or unsubstituted (C6-C30)arylene, or a substituted or unsubstituted (5- to 30-membered)heteroarylene;

Ar₁₁ and Ar₁₂, each independently, represent a substituted or unsubstituted (C6-C30)aryl, or a substituted or unsubstituted (5- to 30-membered)heteroaryl;

T₁ to T₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L_{c}-N—(Ar₁₃)(Ar₁₄);

L_{c}, each independently, represents a single bond, a substituted or unsubstituted (C6-C30)arylene, or a substituted or unsubstituted (5- to 30-membered)heteroarylene;

Ar₁₃ and Ar₁₄, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (5- to 30-membered)heteroaryl;

Dₙ represents that n number of hydrogens are replaced with deuterium; and

n represents 0, or an integer of 8 or more.

Specifically, the examples of the fluorescent host compound include the following compounds, but are not limited thereto.

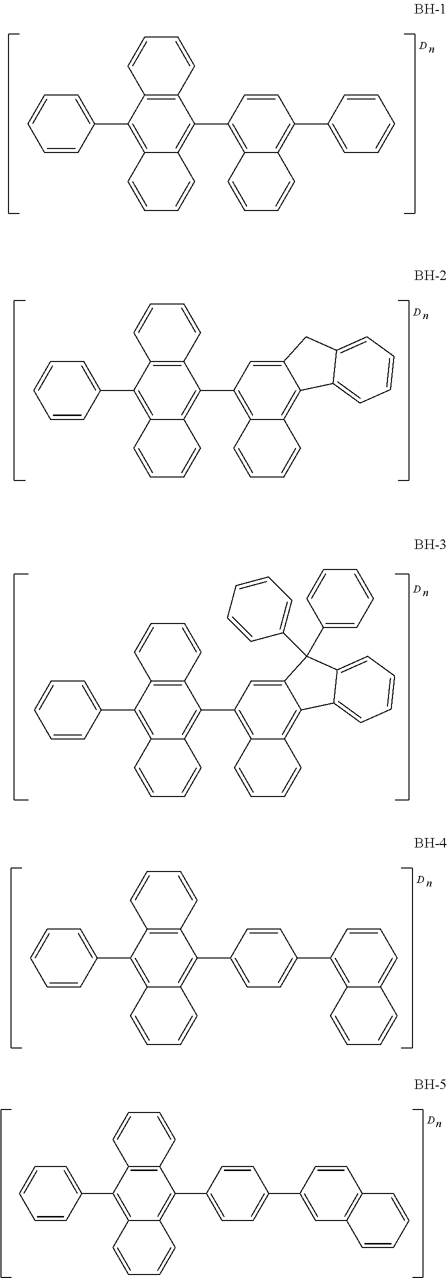

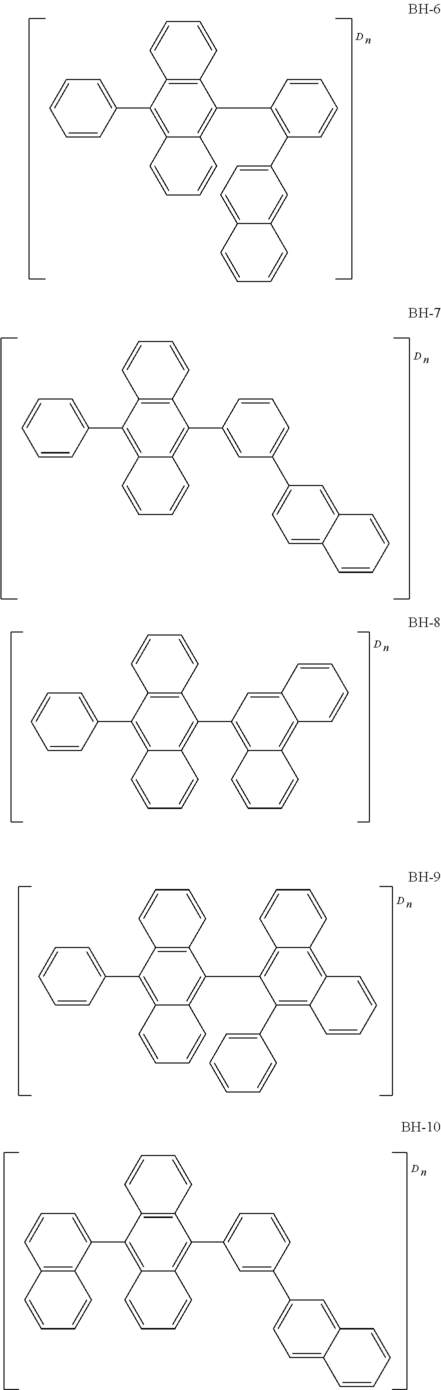

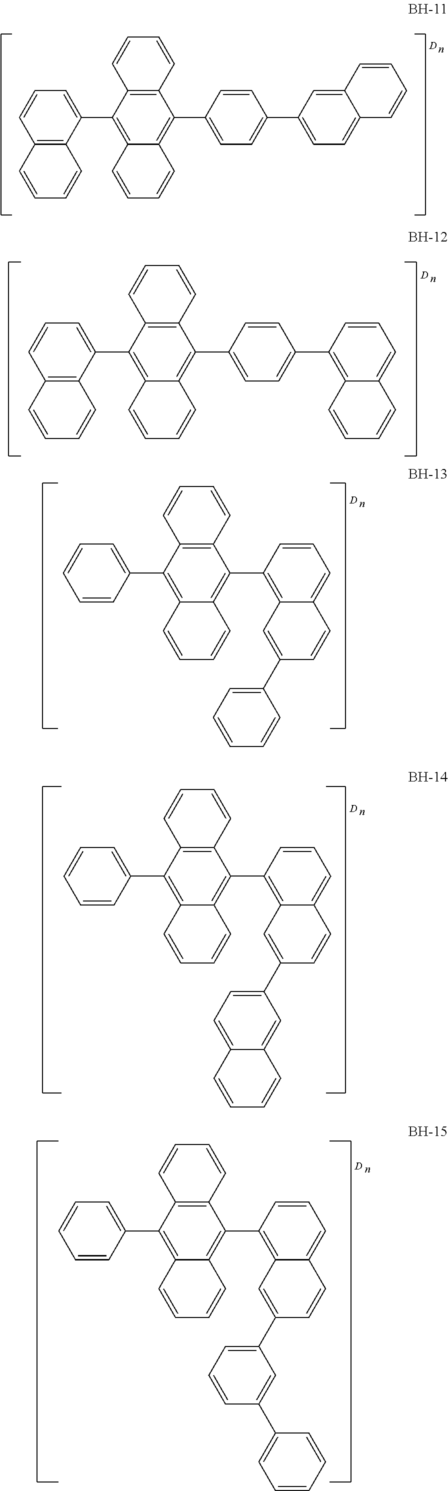

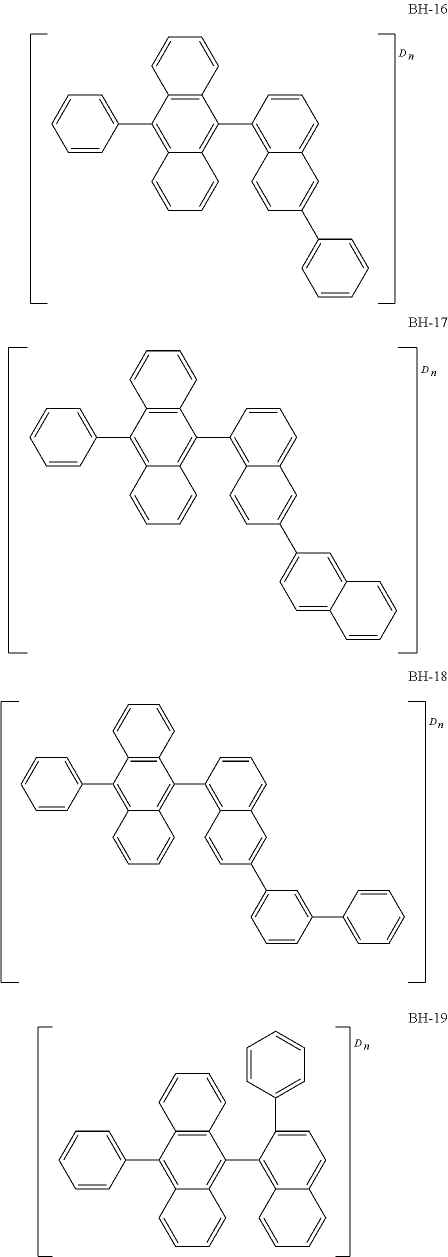

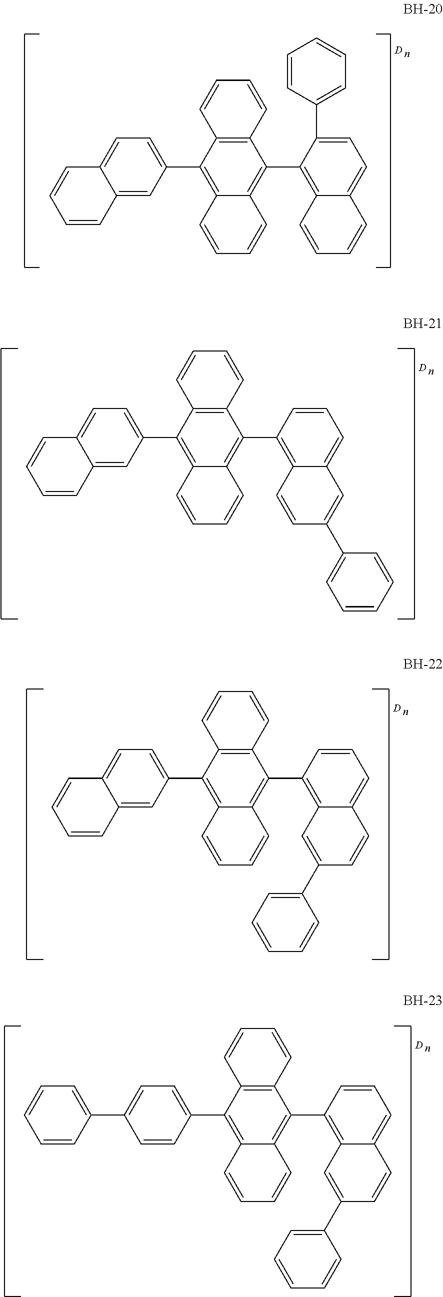

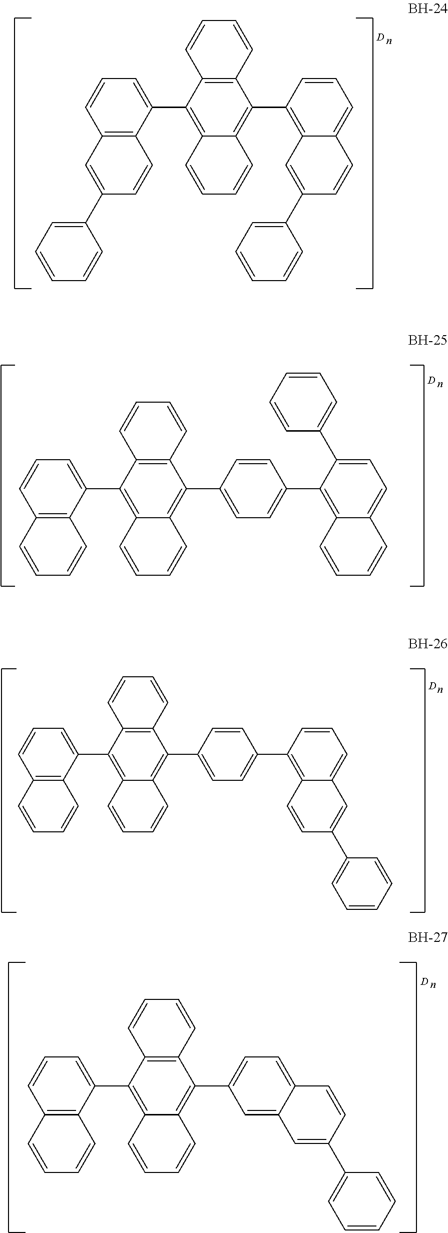

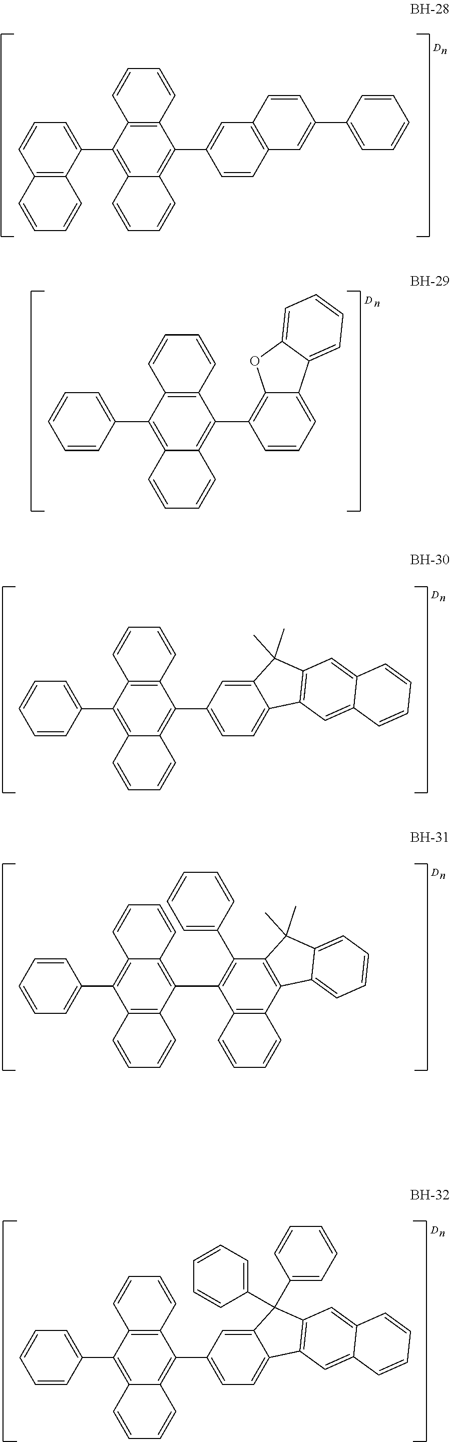

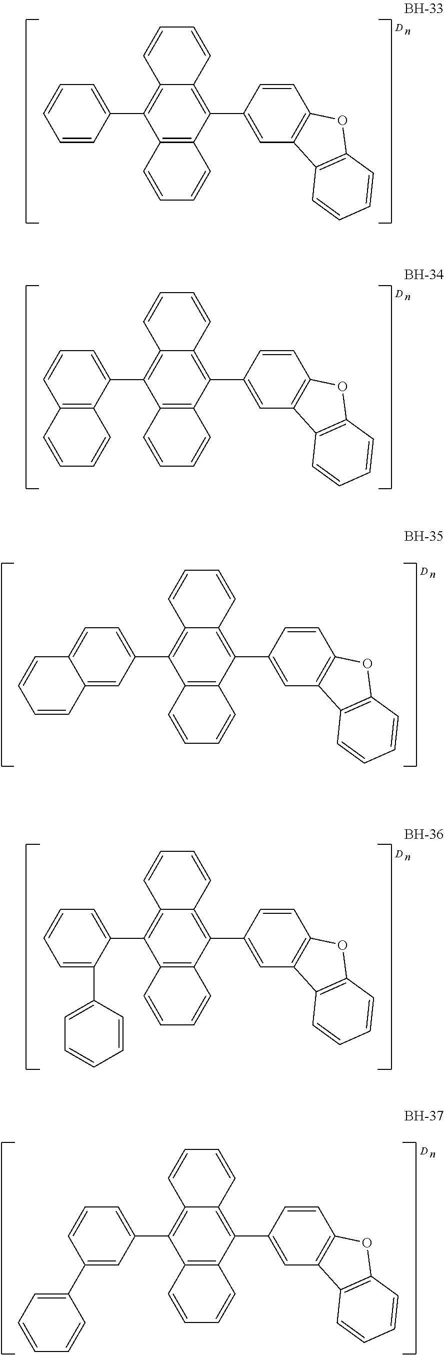

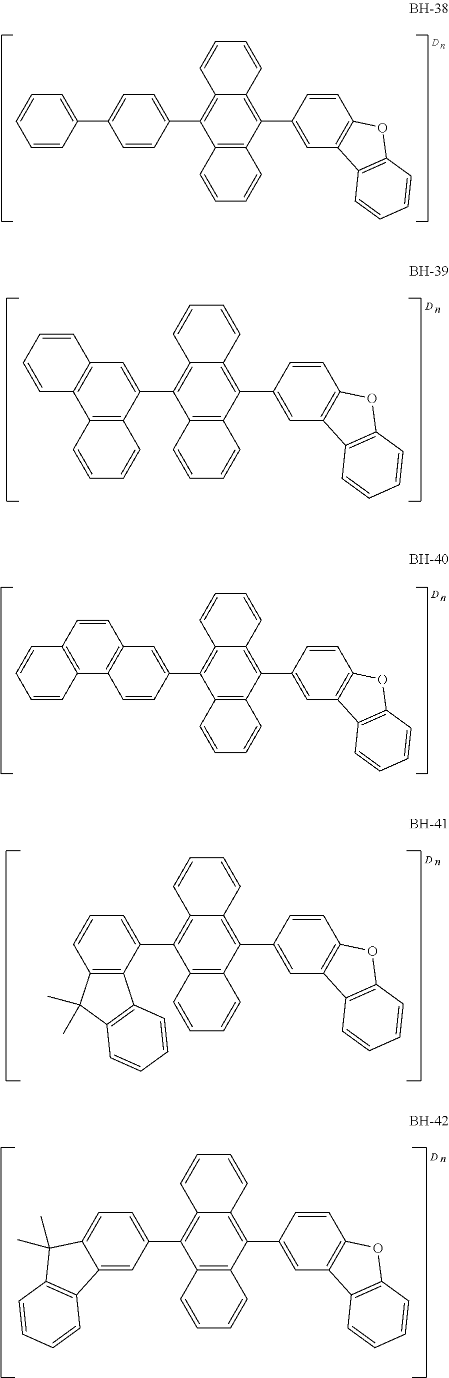

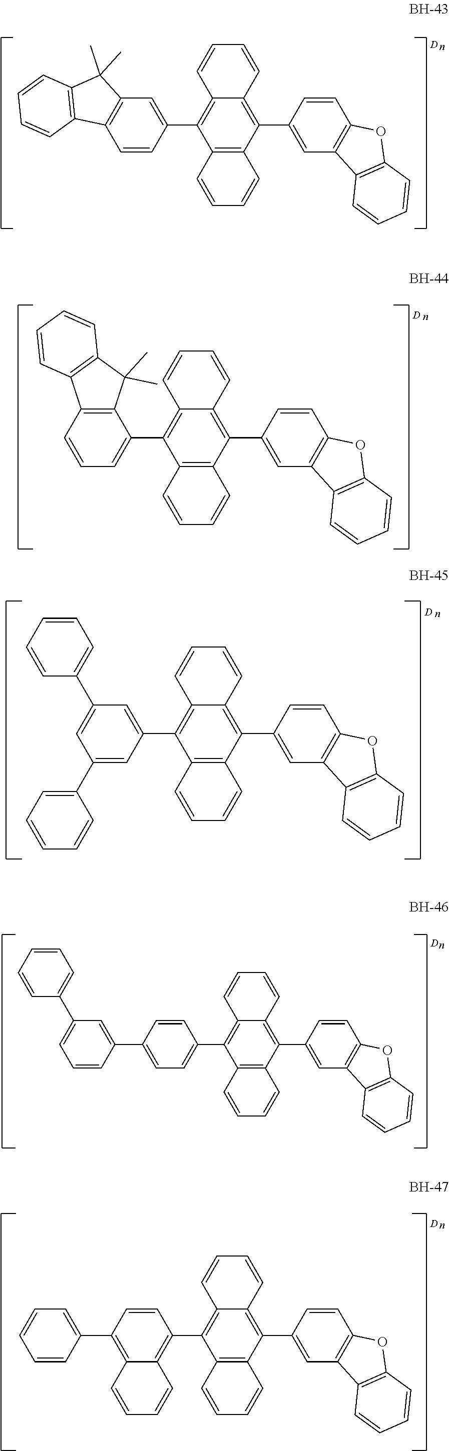

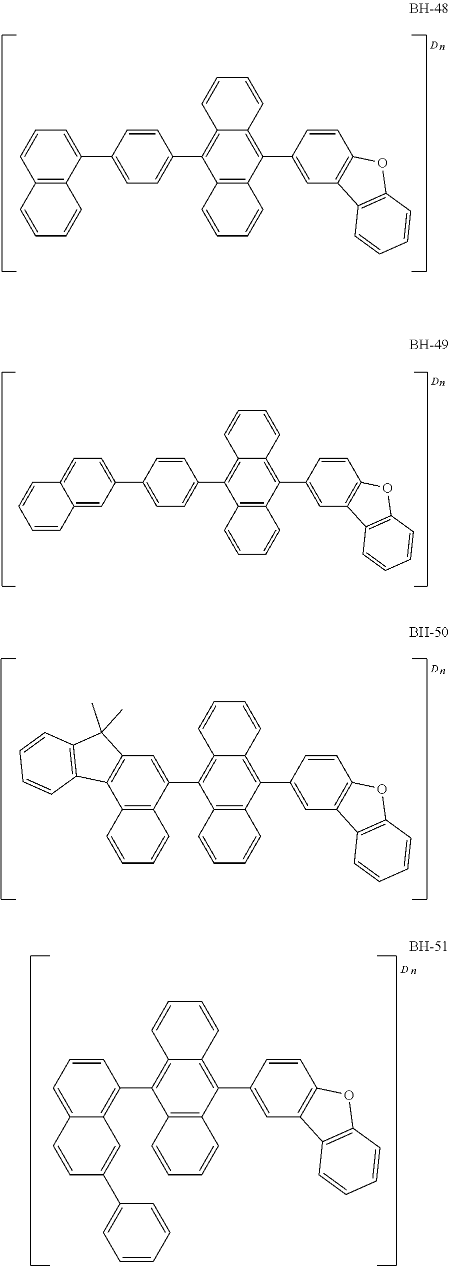

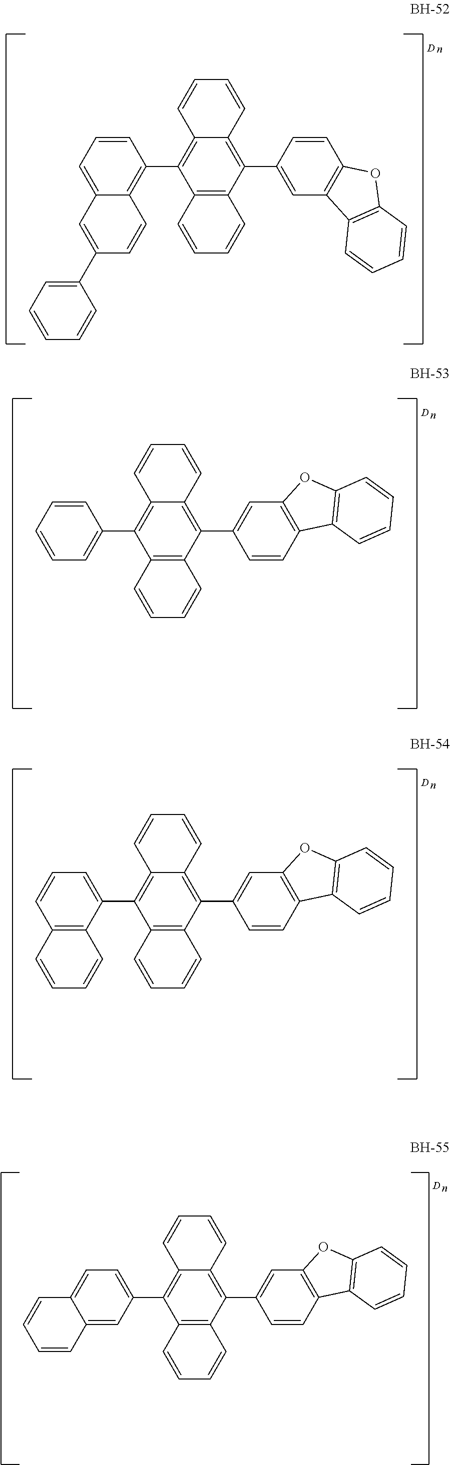

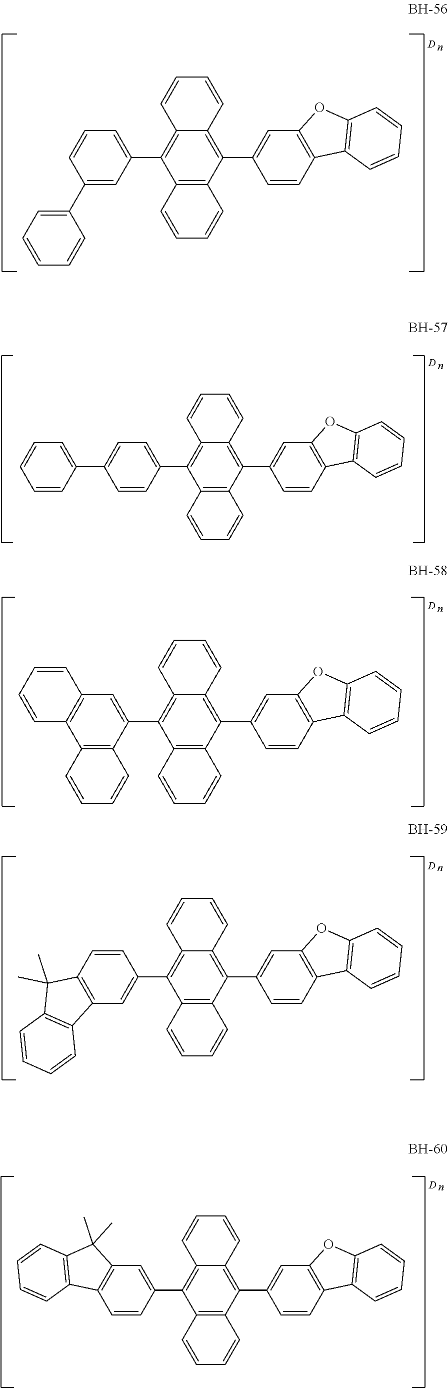

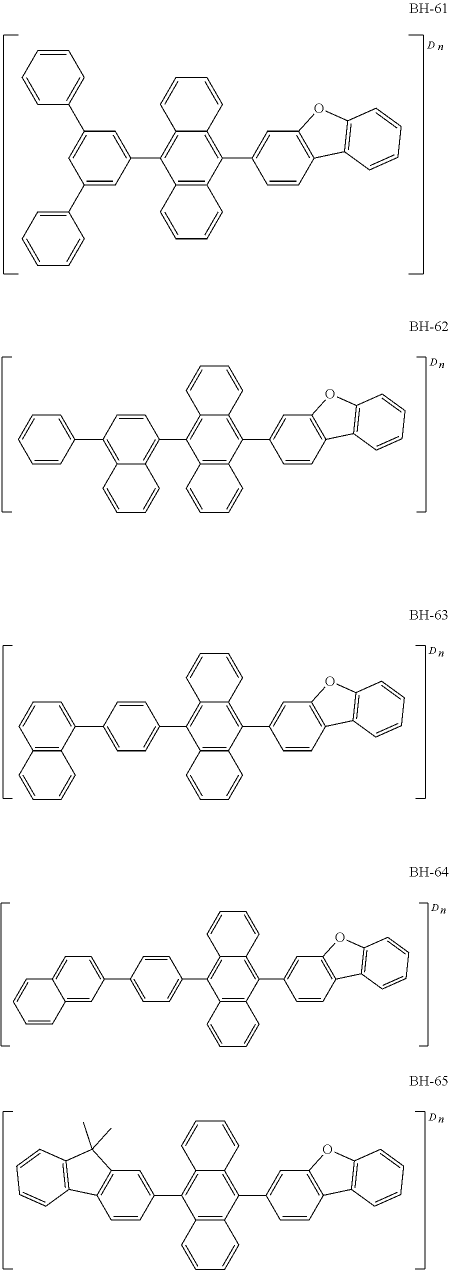

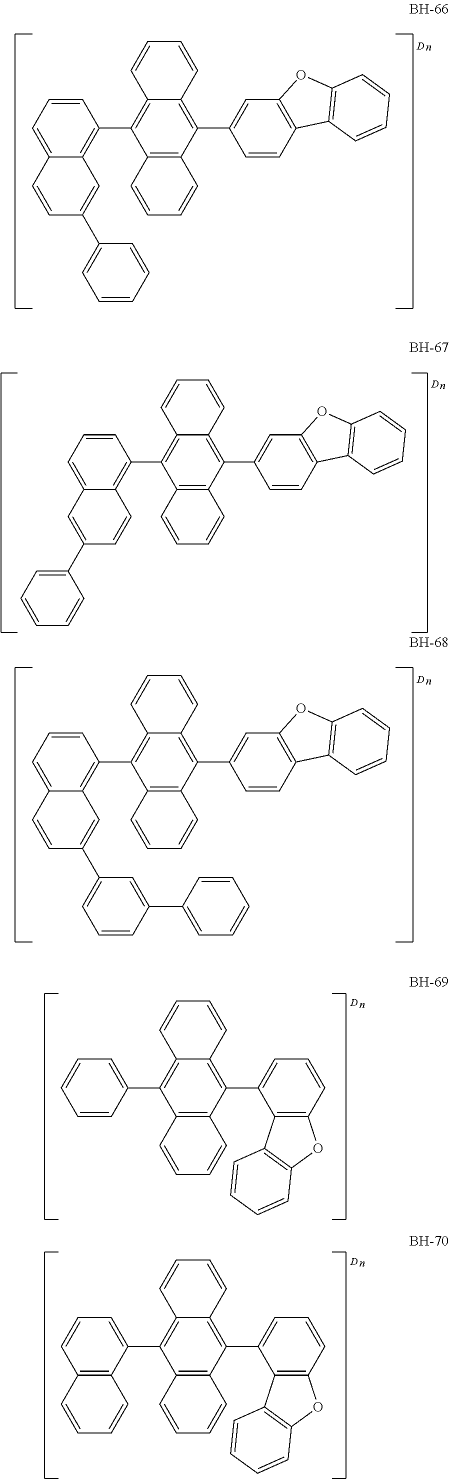

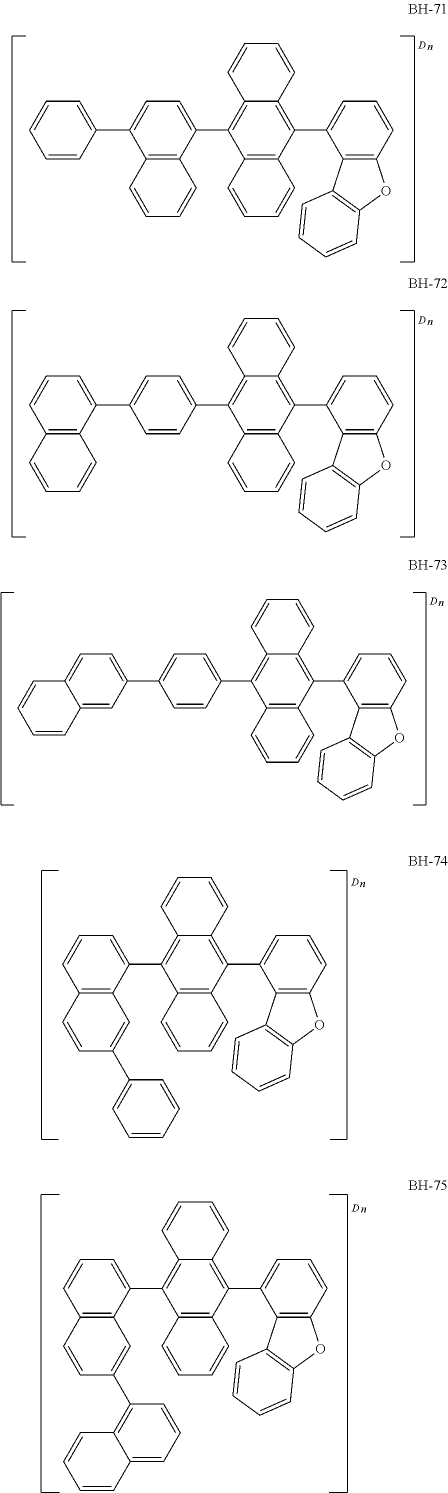

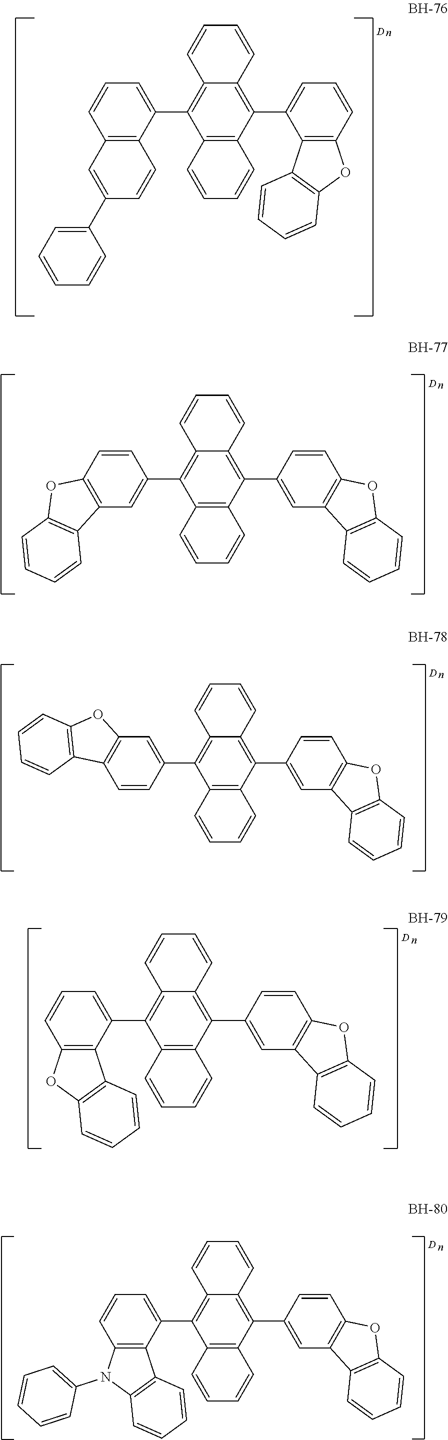

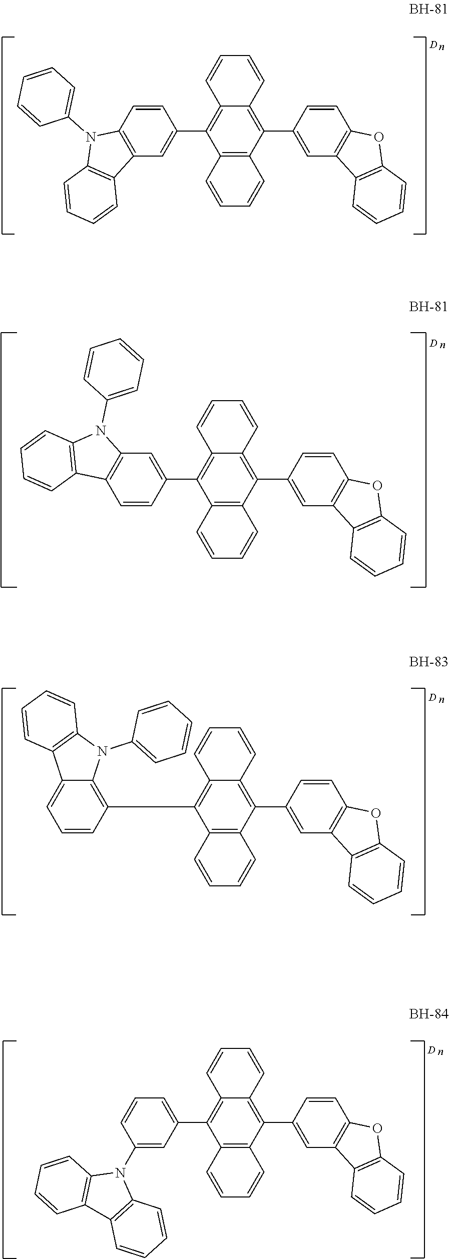

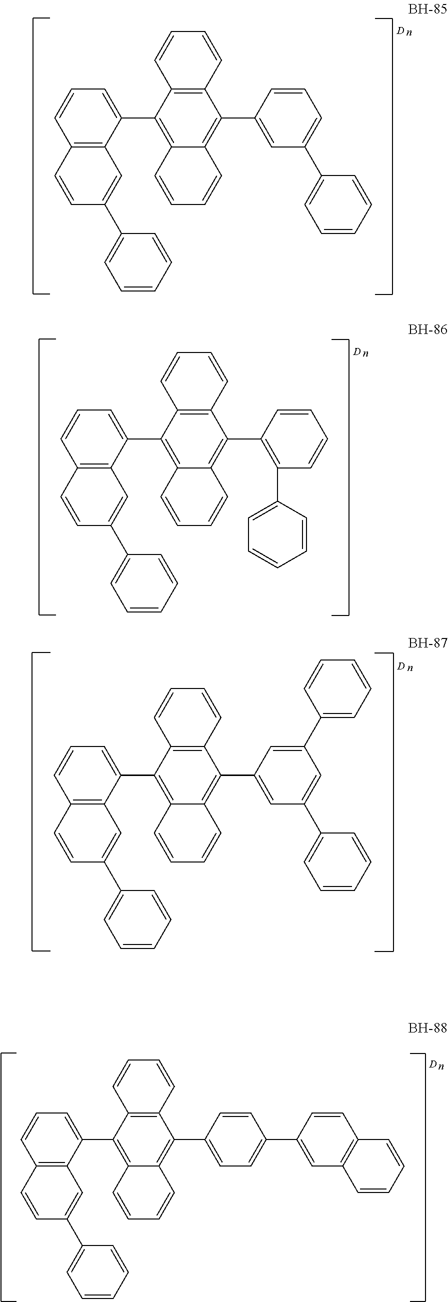

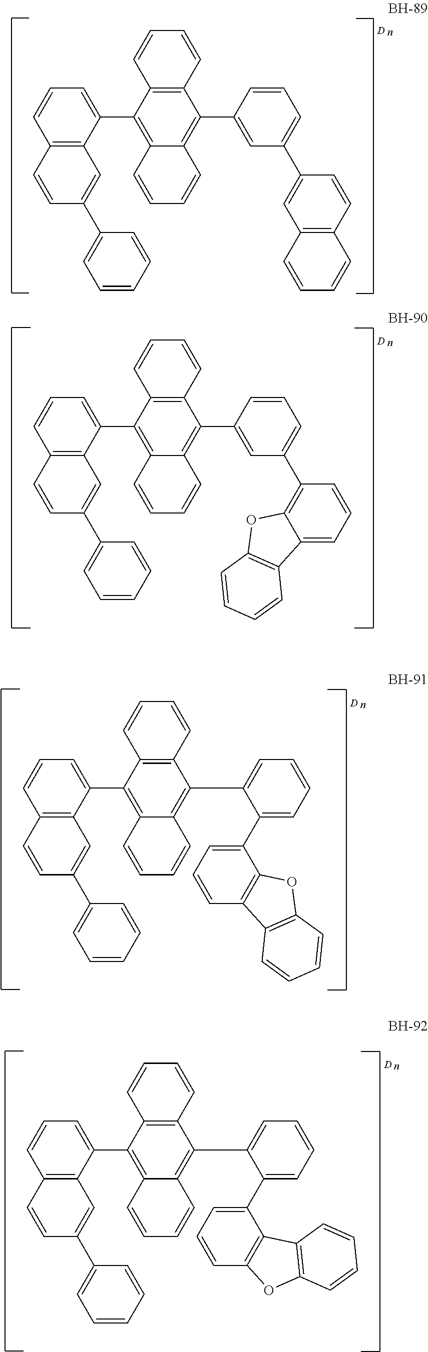

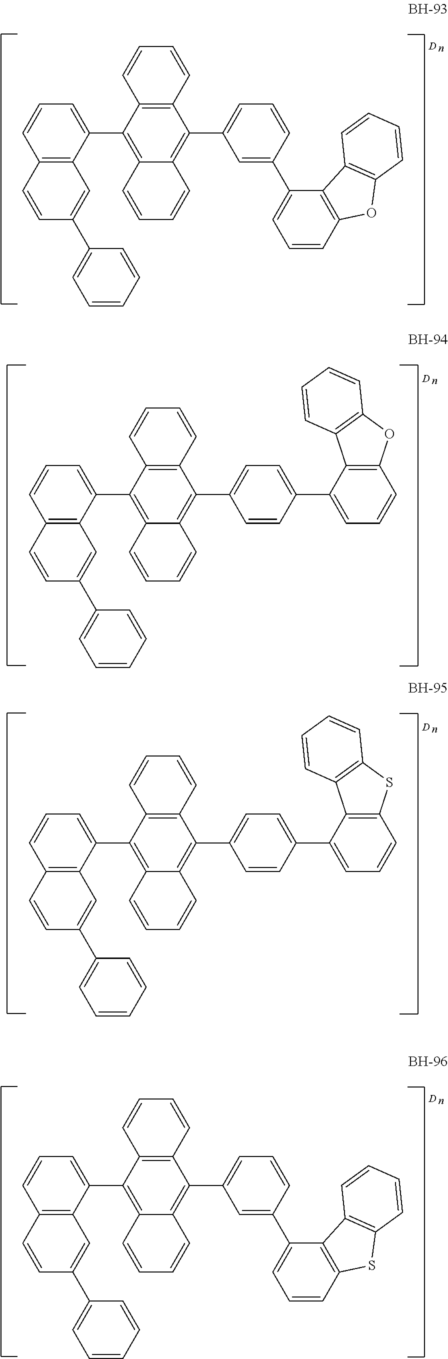

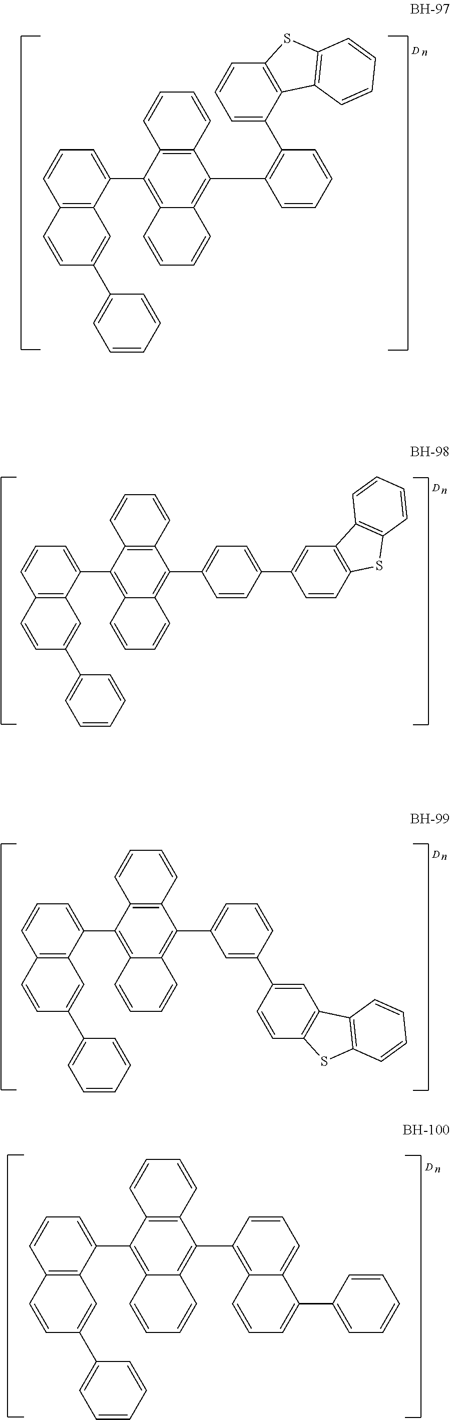

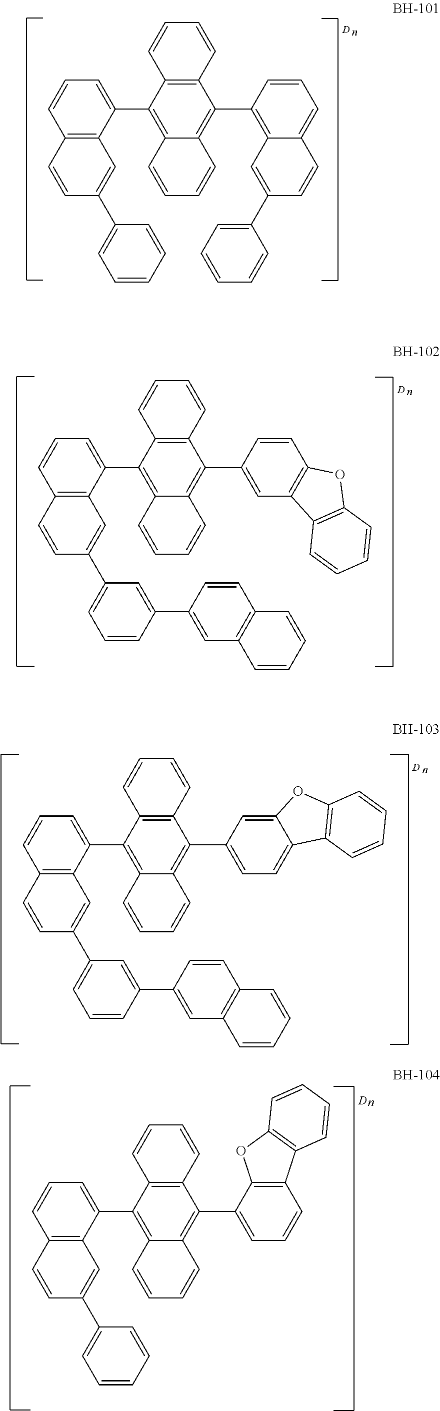

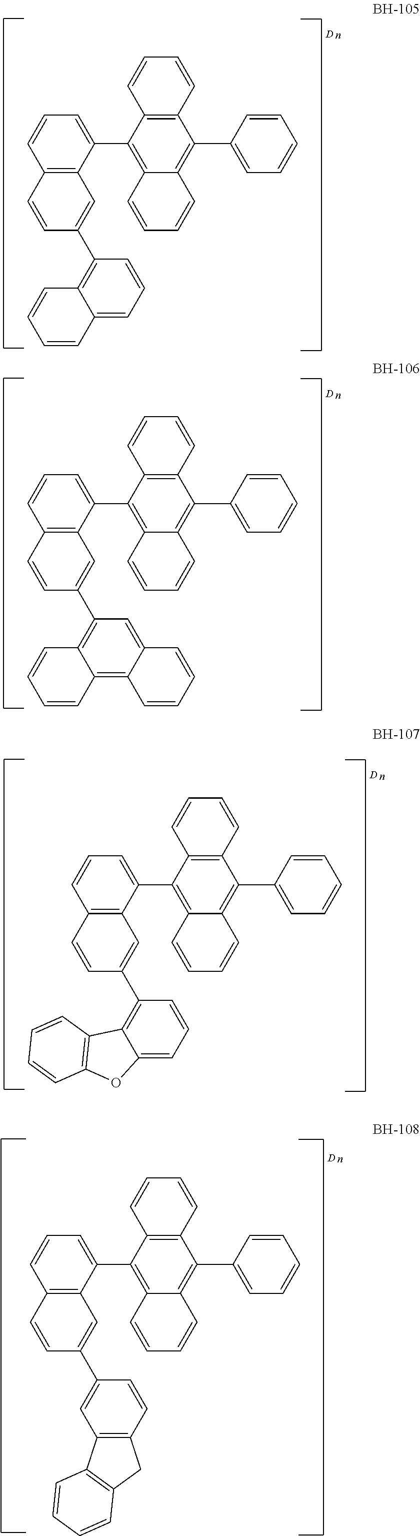

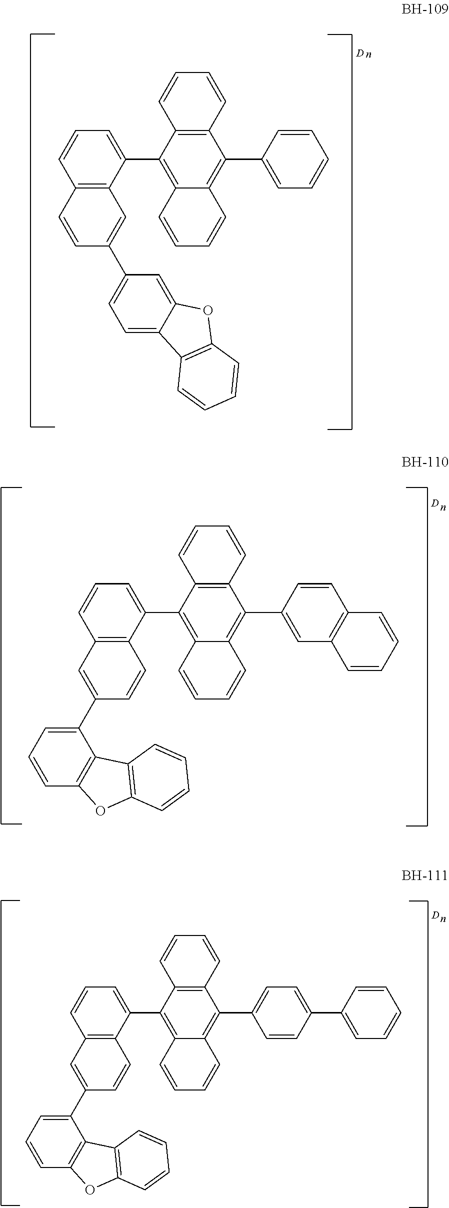

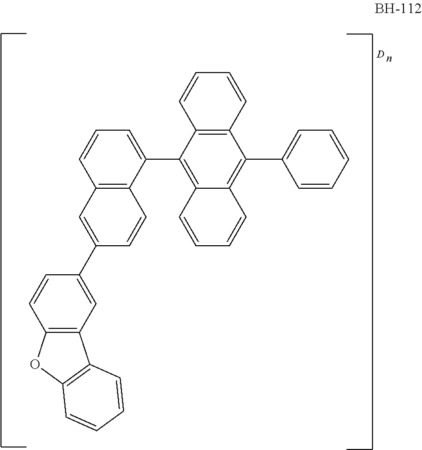

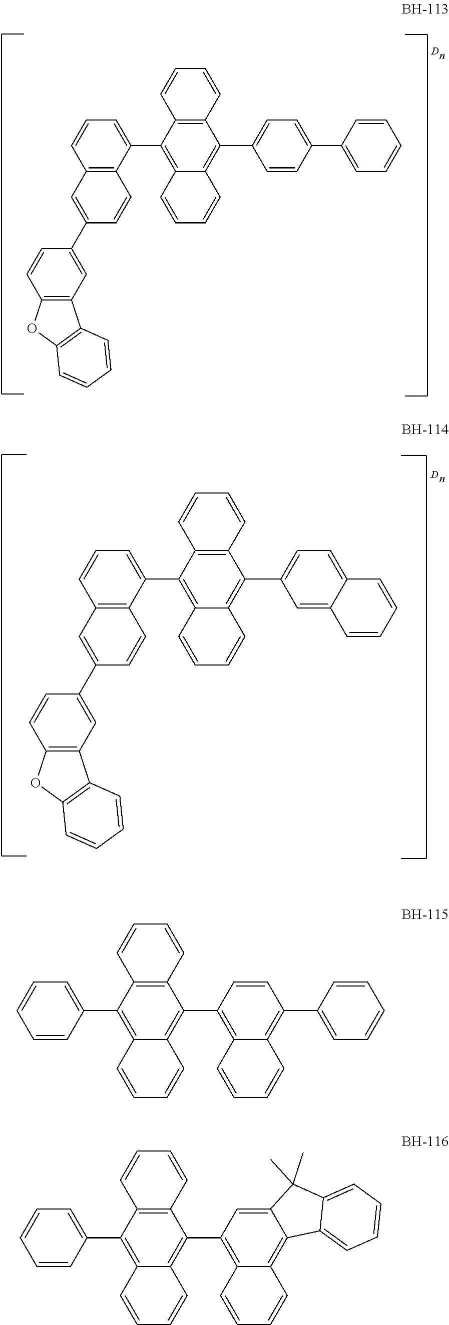

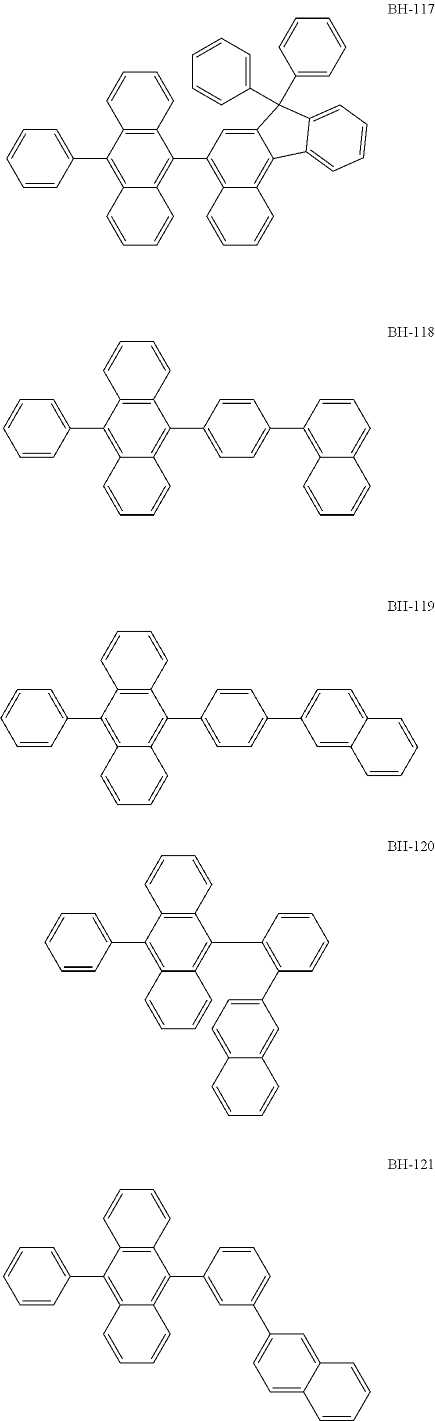

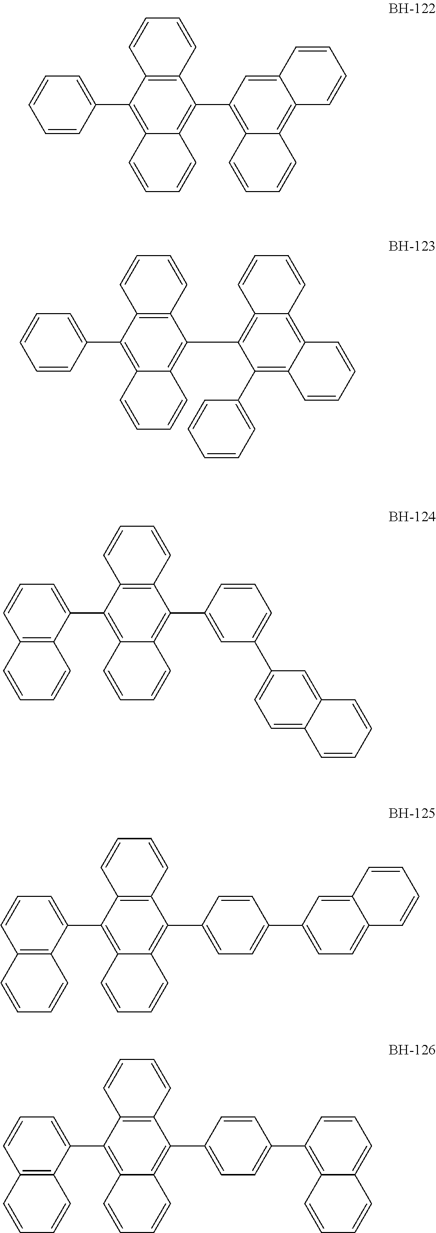

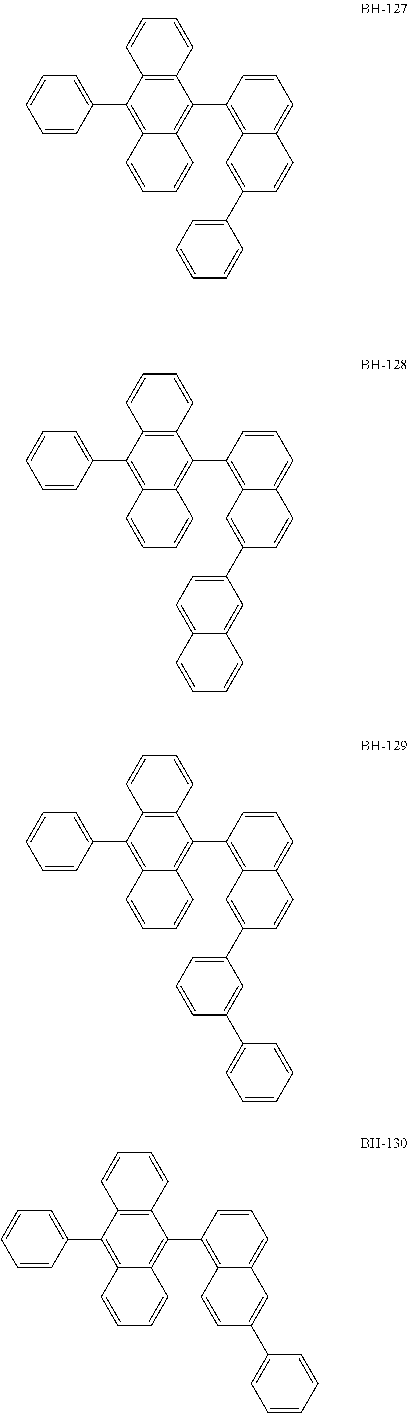

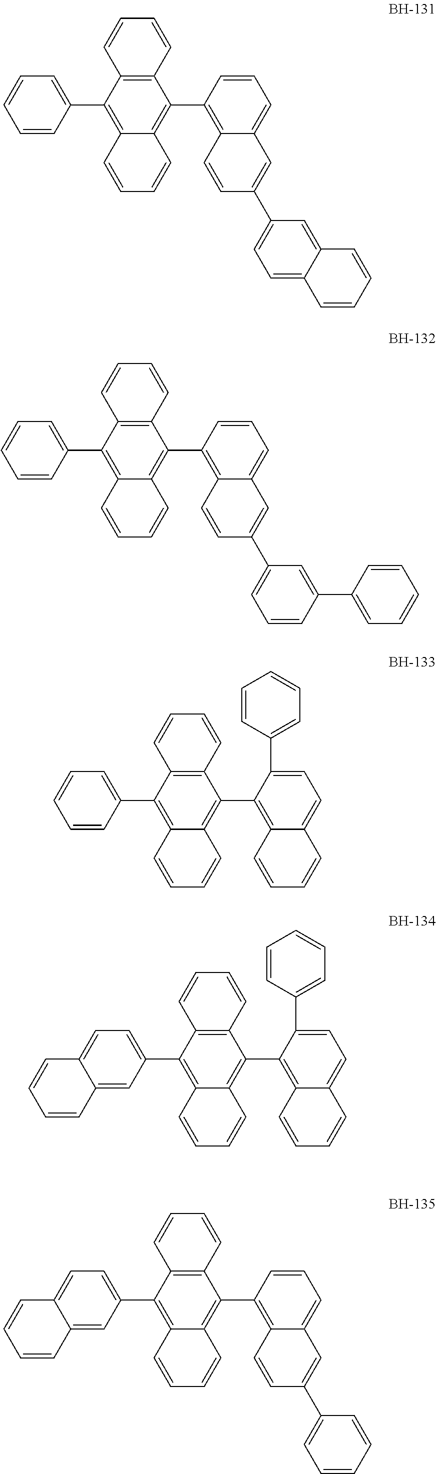

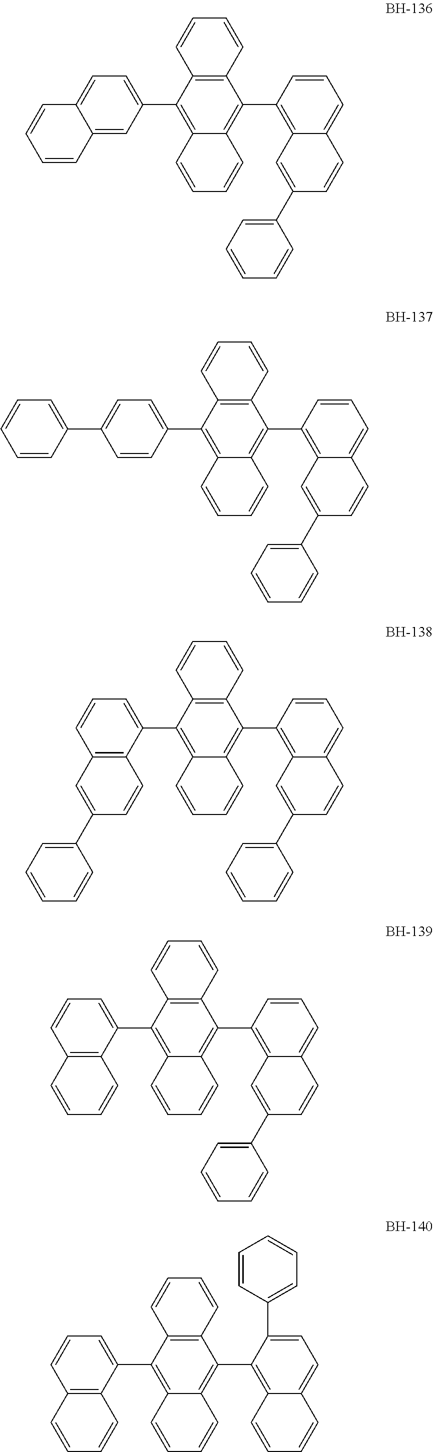

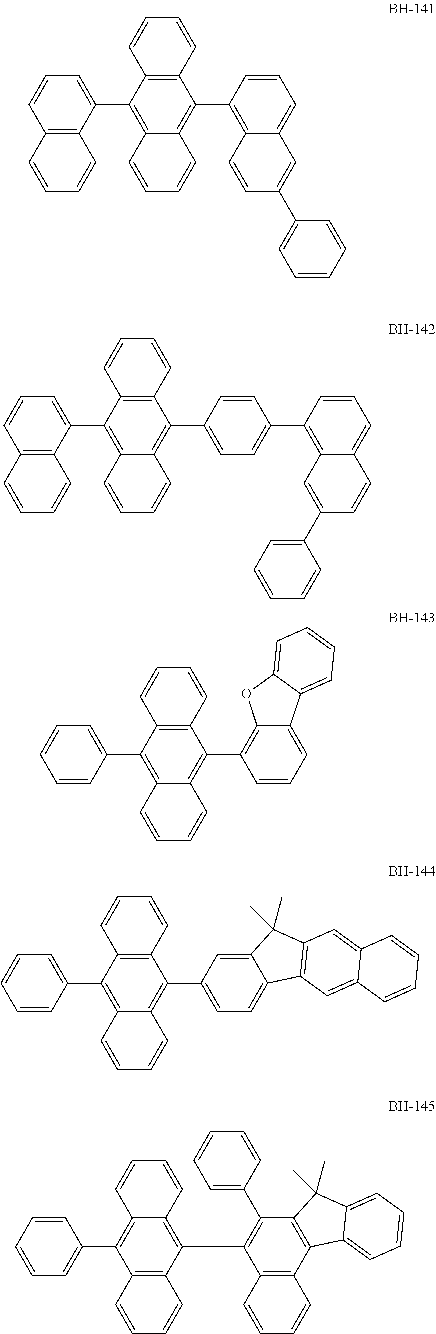

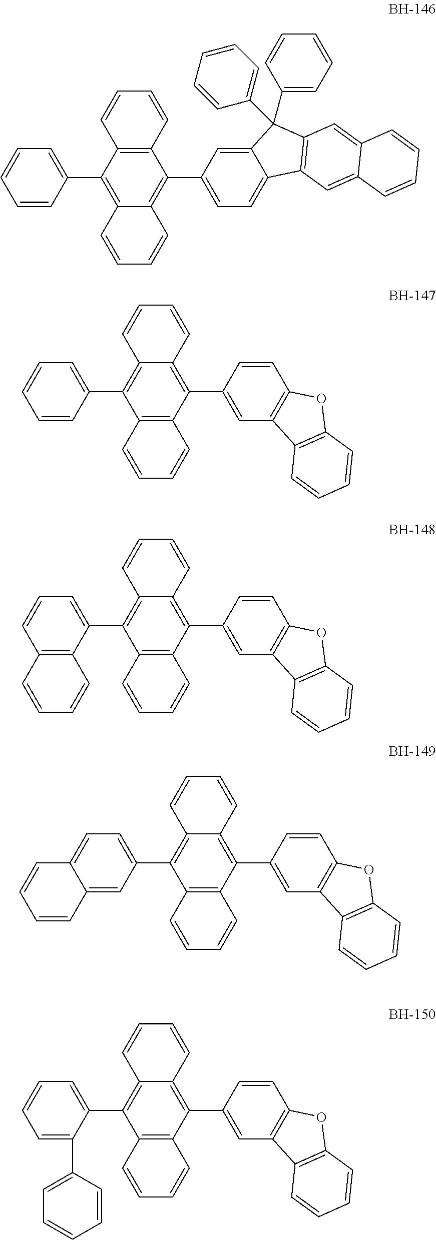

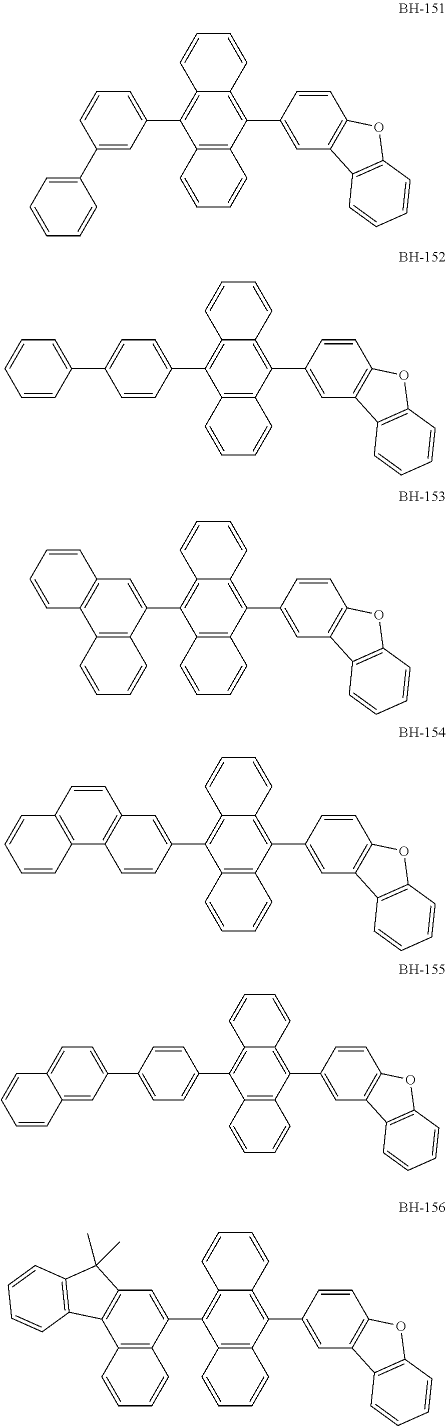 [SMILES: c1ccc(-c2cccc(-c3c4ccccc4c(-c4ccc5oc6ccccc6c5c4)c4ccccc34)c2)cc1 ; c1ccc(-c2ccc(-c3c4ccccc4c(-c4ccc5oc6ccccc6c5c4)c4ccccc34)cc2)cc1 ; c1ccc2c(c1)cc(-c1c3ccccc3c(-c3ccc4oc5ccccc5c4c3)c3ccccc13)c1ccccc12 ; c1ccc2c(c1)ccc1cc(-c3c4ccccc4c(-c4ccc5oc6ccccc6c5c4)c4ccccc34)ccc12 ; c1ccc2cc(-c3ccc(-c4c5ccccc5c(-c5ccc6oc7ccccc7c6c5)c5ccccc45)cc3)ccc2c1 ; CC1(C)c2ccccc2-c2c1cc(-c1c3ccccc3c(-c3ccc4oc5ccccc5c4c3)c3ccccc13)c1ccccc21]

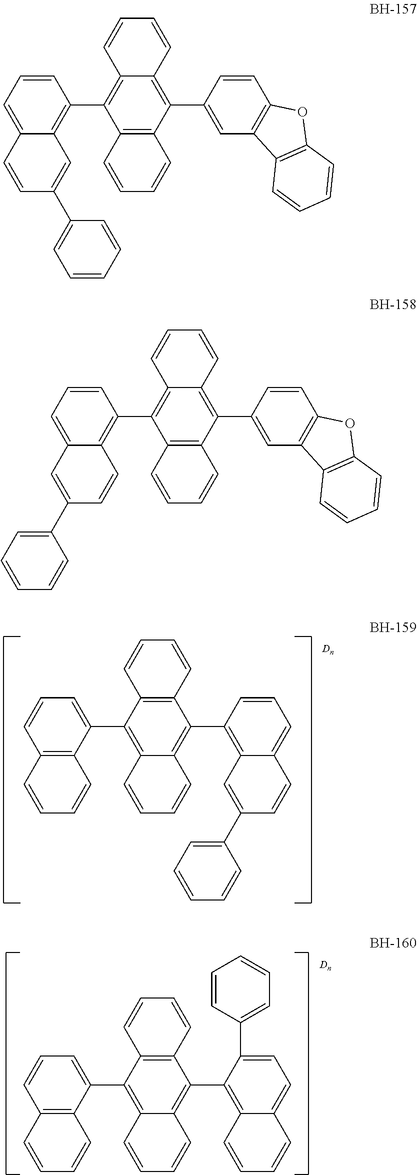

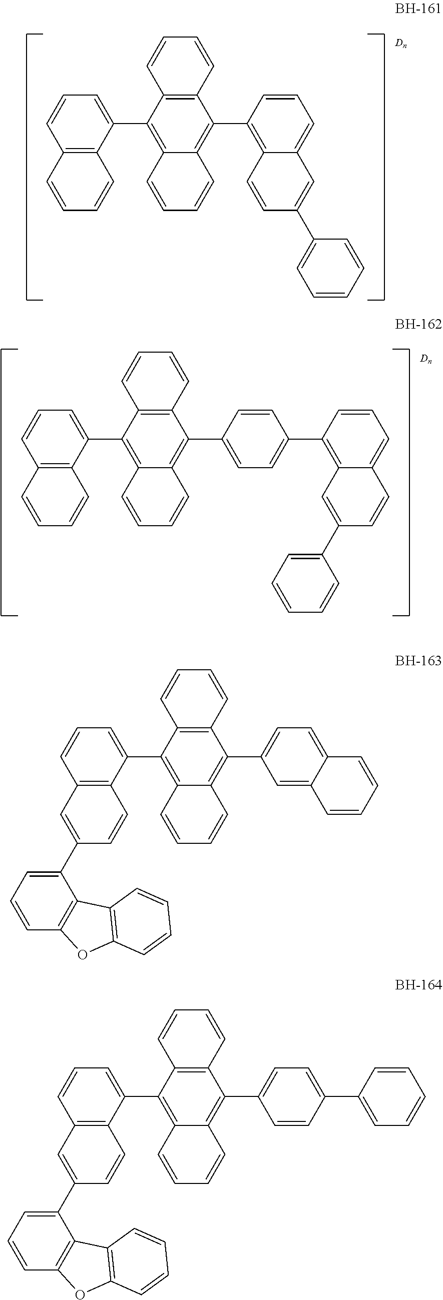

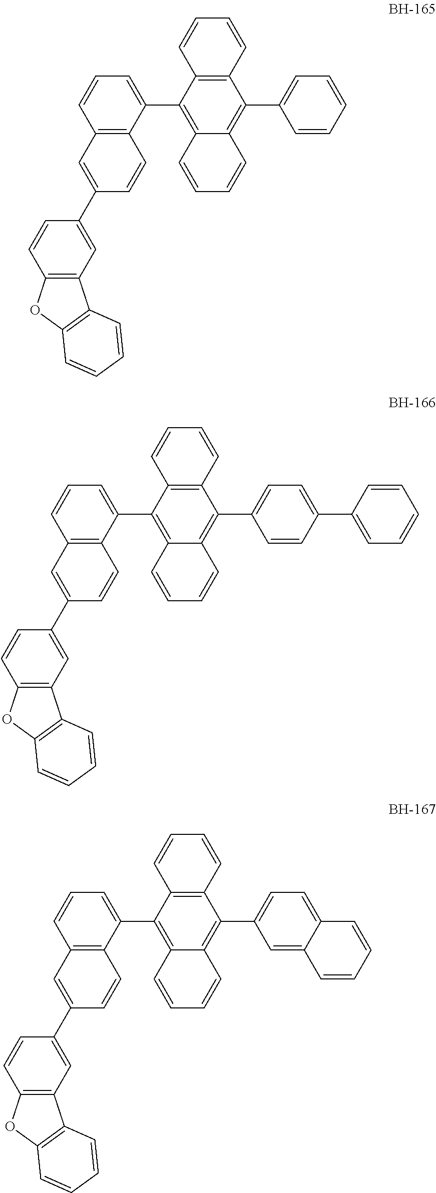 [SMILES: c1ccc(-c2c3ccccc3c(-c3cccc4cc(-c5ccc6oc7ccccc7c6c5)ccc34)c3ccccc23)cc1 ; c1ccc(-c2ccc(-c3c4ccccc4c(-c4cccc5cc(-c6ccc7oc8ccccc8c7c6)ccc45)c4ccccc34)cc2)cc1 ; c1ccc2cc(-c3c4ccccc4c(-c4cccc5cc(-c6ccc7oc8ccccc8c7c6)ccc45)c4ccccc34)ccc2c1]

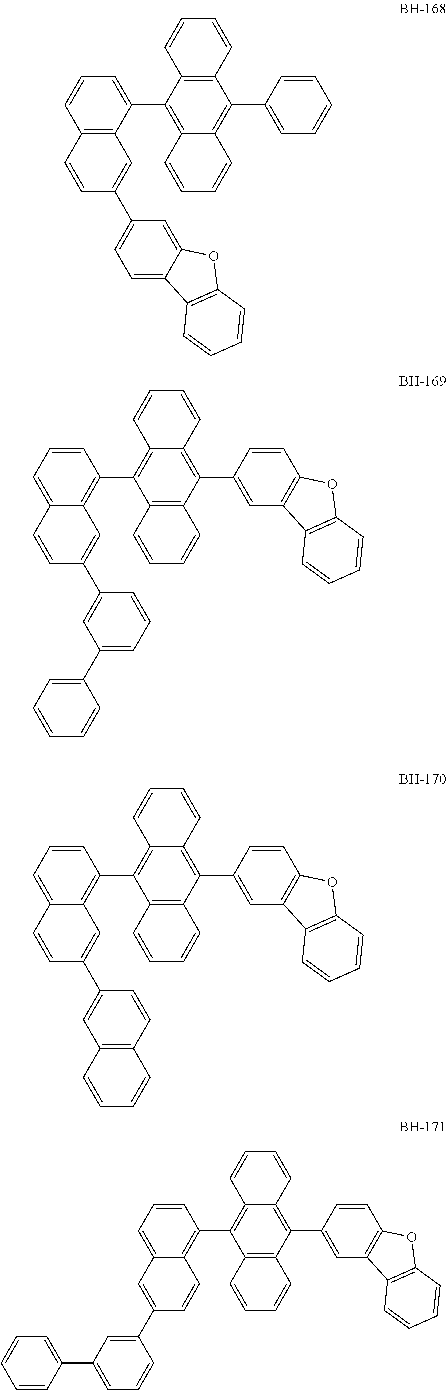

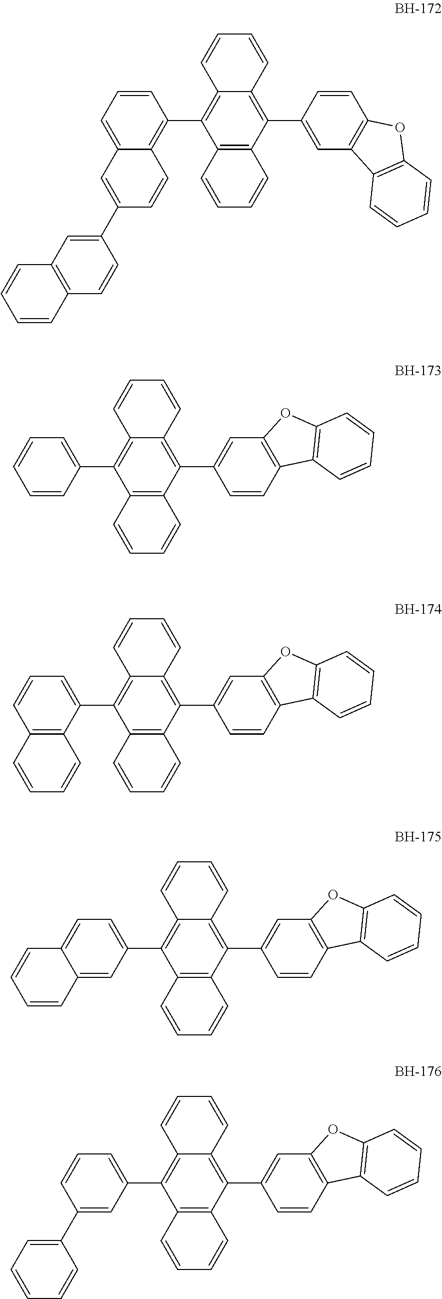 [SMILES: c1ccc2cc(-c3ccc4c(-c5c6ccccc6c(-c6ccc7oc8ccccc8c7c6)c6ccccc56)cccc4c3)ccc2c1 ; c1ccc(-c2c3ccccc3c(-c3ccc4c(c3)oc3ccccc34)c3ccccc23)cc1 ; c1ccc2c(-c3c4ccccc4c(-c4ccc5c(c4)oc4ccccc45)c4ccccc34)cccc2c1 ; c1ccc2cc(-c3c4ccccc4c(-c4ccc5c(c4)oc4ccccc45)c4ccccc34)ccc2c1 ; c1ccc(-c2cccc(-c3c4ccccc4c(-c4ccc5c(c4)oc4ccccc45)c4ccccc34)c2)cc1]

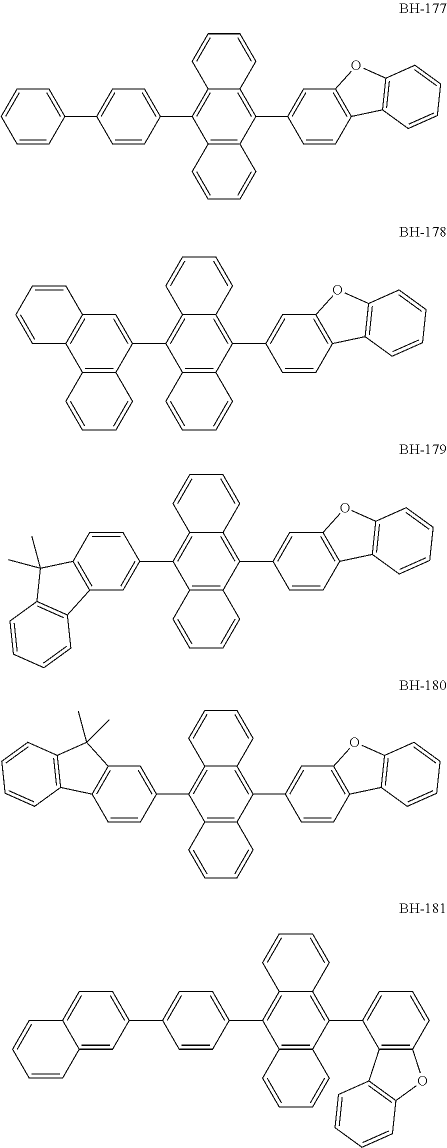

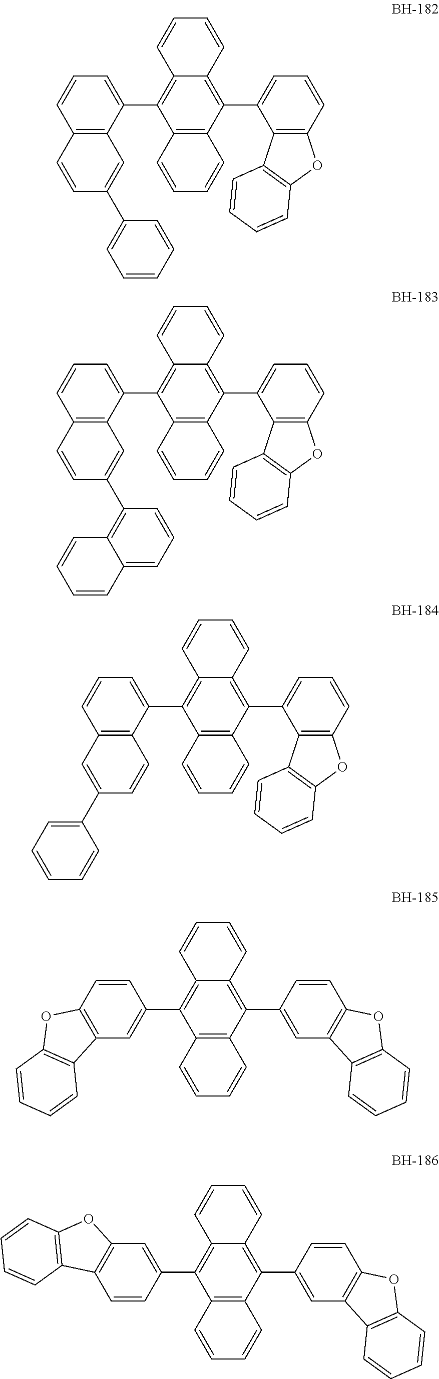

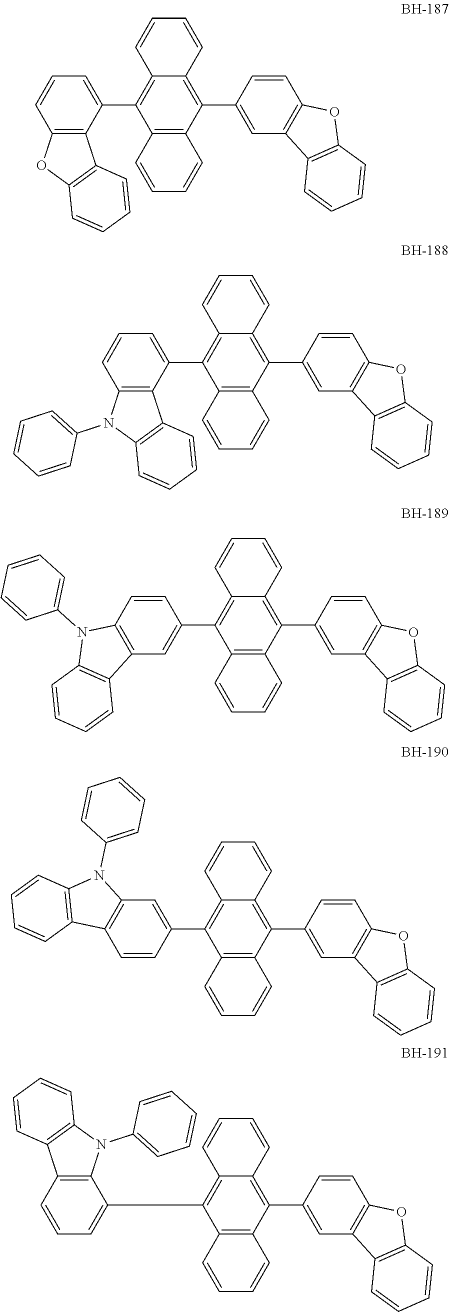 [SMILES: c1ccc2c(c1)oc1ccc(-c3c4ccccc4c(-c4cccc5oc6ccccc6c45)c4ccccc34)cc12 ; c1ccc(-n2c3ccccc3c3c(-c4c5ccccc5c(-c5ccc6oc7ccccc7c6c5)c5ccccc45)cccc32)cc1 ; c1ccc(-n2c3ccccc3c3cc(-c4c5ccccc5c(-c5ccc6oc7ccccc7c6c5)c5ccccc45)ccc32)cc1 ; c1ccc(-n2c3ccccc3c3ccc(-c4c5ccccc5c(-c5ccc6oc7ccccc7c6c5)c5ccccc45)cc32)cc1 ; c1ccc(-n2c3ccccc3c3cccc(-c4c5ccccc5c(-c5ccc6oc7ccccc7c6c5)c5ccccc45)c32)cc1]

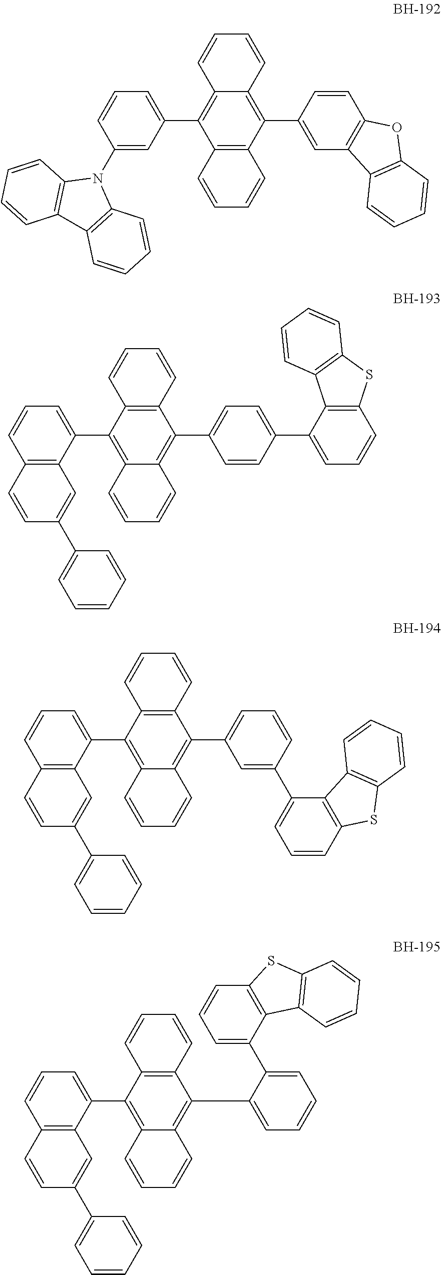 [SMILES: c1cc(-c2c3ccccc3c(-c3ccc4oc5ccccc5c4c3)c3ccccc23)cc(-n2c3ccccc3c3ccccc32)c1 ; c1ccc(-c2ccc3cccc(-c4c5ccccc5c(-c5ccc(-c6cccc7sc8ccccc8c67)cc5)c5ccccc45)c3c2)cc1 ; c1ccc(-c2ccc3cccc(-c4c5ccccc5c(-c5cccc(-c6cccc7sc8ccccc8c67)c5)c5ccccc45)c3c2)cc1 ; c1ccc(-c2ccc3cccc(-c4c5ccccc5c(-c5ccccc5-c5cccc6sc7ccccc7c56)c5ccccc45)c3c2)cc1]

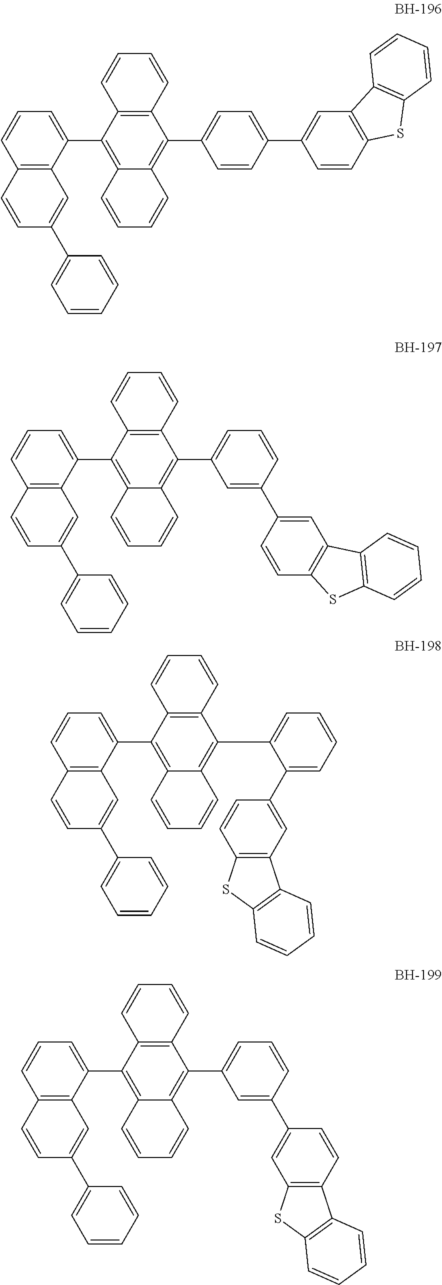 [SMILES: c1ccc(-c2ccc3cccc(-c4c5ccccc5c(-c5ccc(-c6ccc7sc8ccccc8c7c6)cc5)c5ccccc45)c3c2)cc1 ; c1ccc(-c2ccc3cccc(-c4c5ccccc5c(-c5cccc(-c6ccc7sc8ccccc8c7c6)c5)c5ccccc45)c3c2)cc1 ; c1ccc(-c2ccc3cccc(-c4c5ccccc5c(-c5ccccc5-c5ccc6sc7ccccc7c6c5)c5ccccc45)c3c2)cc1 ; c1ccc(-c2ccc3cccc(-c4c5ccccc5c(-c5cccc(-c6ccc7c(c6)sc6ccccc67)c5)c5ccccc45)c3c2)cc1]

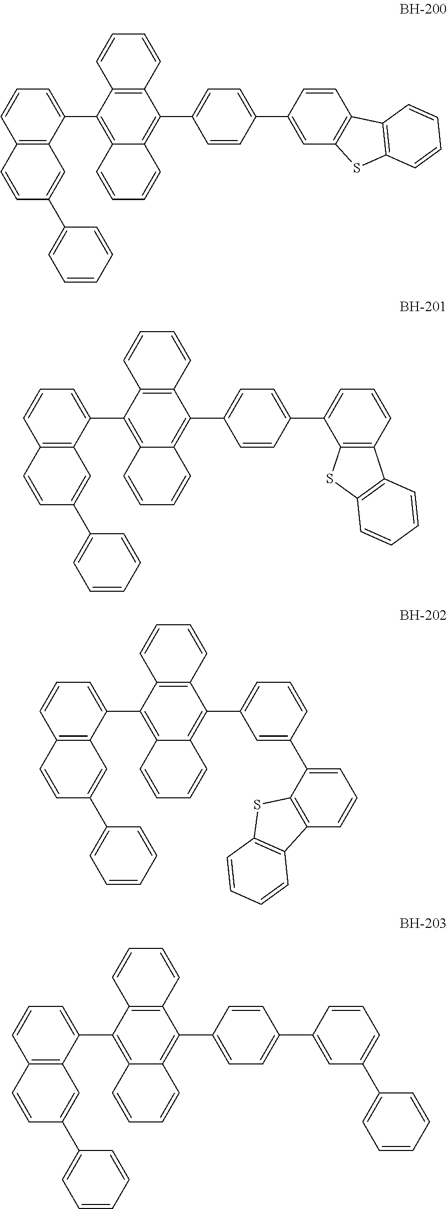

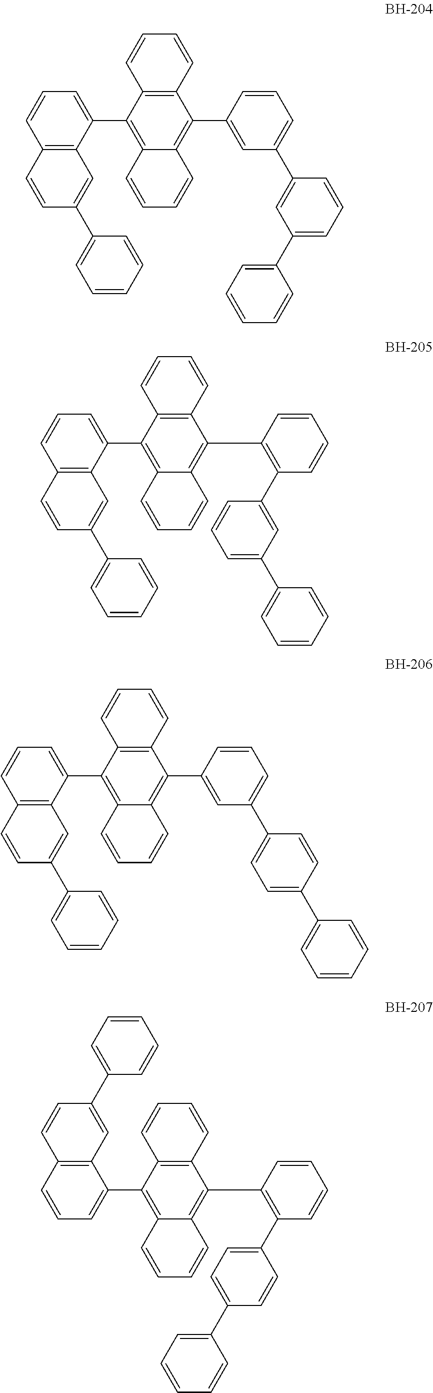

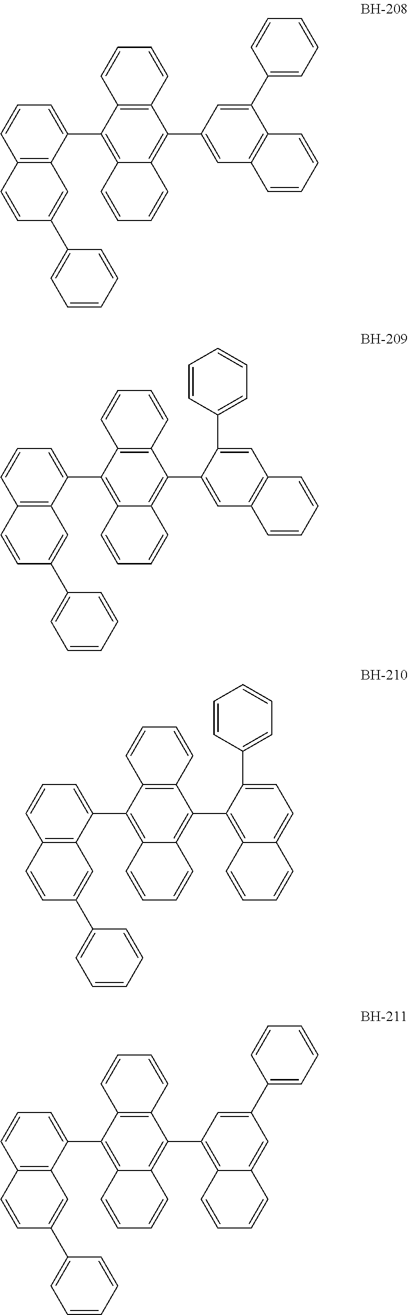

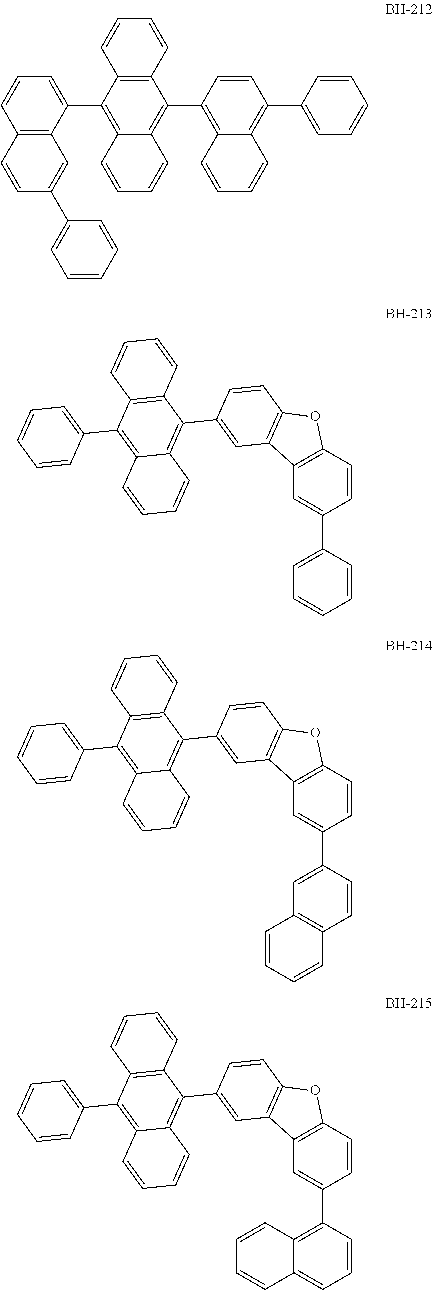

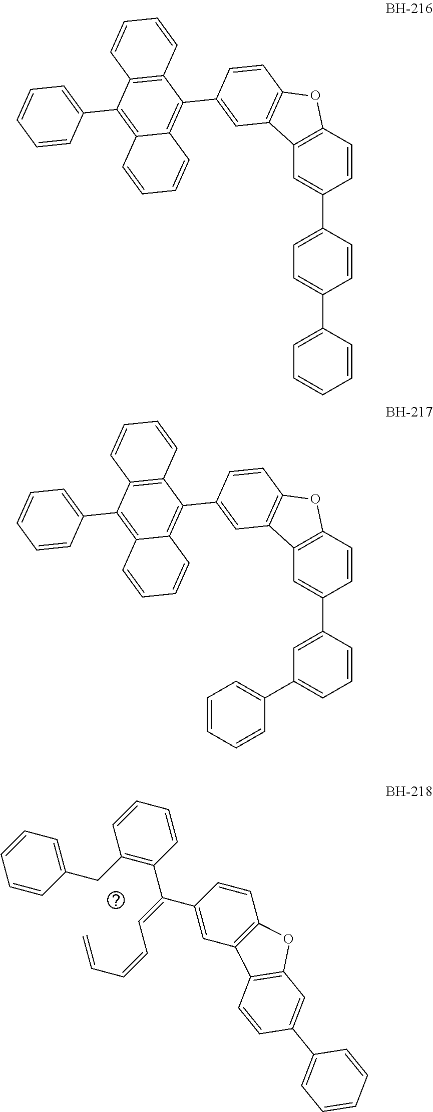 [SMILES: c1ccc(-c2ccc(-c3ccc4oc5ccc(-c6c7ccccc7c(-c7ccccc7)c7ccccc67)cc5c4c3)cc2)cc1 ; c1ccc(-c2cccc(-c3ccc4oc5ccc(-c6c7ccccc7c(-c7ccccc7)c7ccccc67)cc5c4c3)c2)cc1 ; C=C/C=C\C=C(/c1ccc2oc3cc(-c4ccccc4)ccc3c2c1)c1ccccc1Cc1ccccc1]

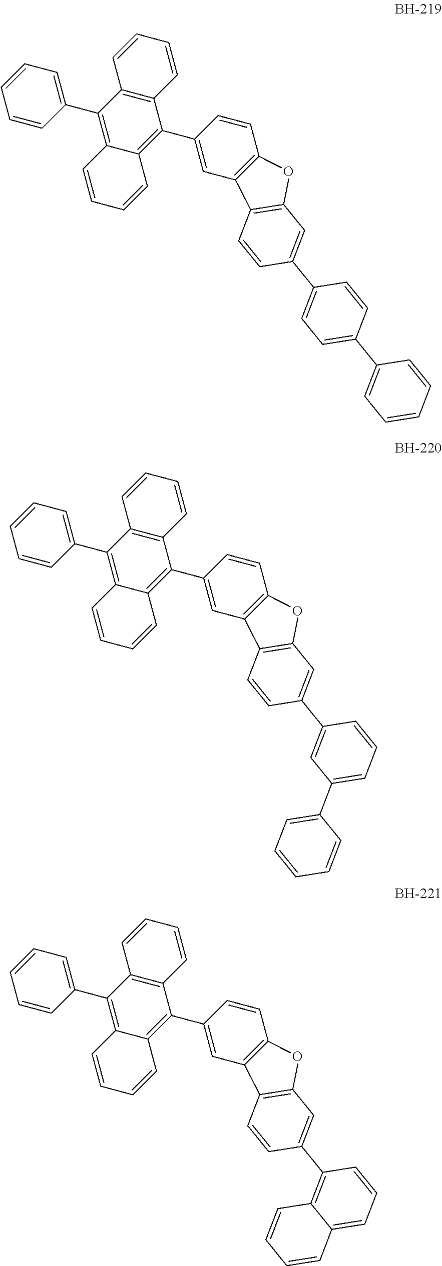 [SMILES: c1ccc(-c2ccc(-c3ccc4c(c3)oc3ccc(-c5c6ccccc6c(-c6ccccc6)c6ccccc56)cc34)cc2)cc1 ; c1ccc(-c2cccc(-c3ccc4c(c3)oc3ccc(-c5c6ccccc6c(-c6ccccc6)c6ccccc56)cc34)c2)cc1 ; c1ccc(-c2c3ccccc3c(-c3ccc4oc5cc(-c6cccc7ccccc67)ccc5c4c3)c3ccccc23)cc1]

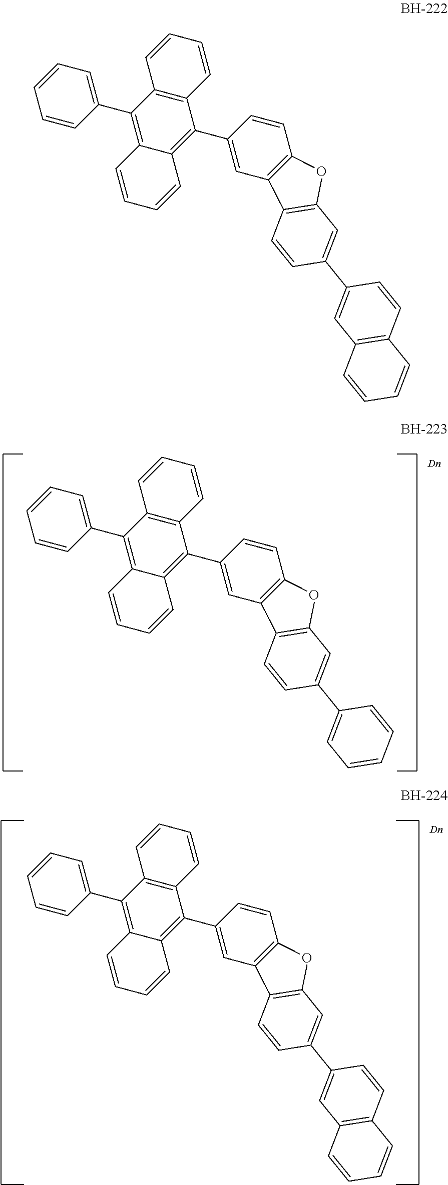

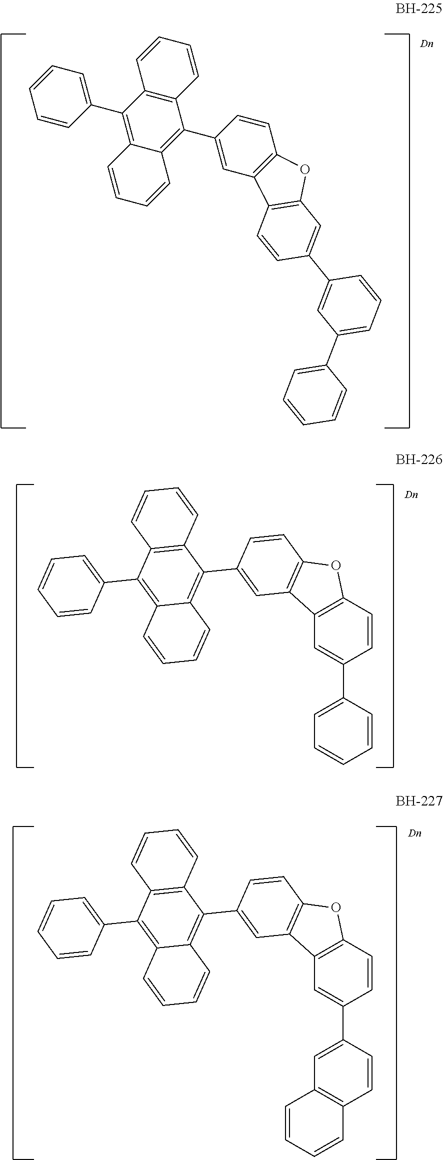

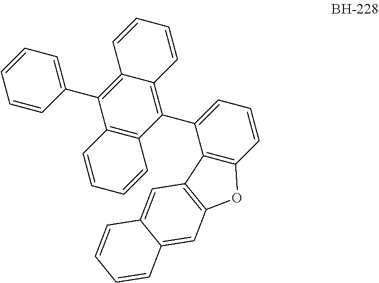 [SMILES: c1ccc(-c2c3ccccc3c(-c3cccc4oc5cc6ccccc6cc5c34)c3ccccc23)cc1]

The fluorescent dopant material included in the organic electroluminescent device of the present disclosure is not particularly limited, but a compound represented by the following formula 100 may be used.

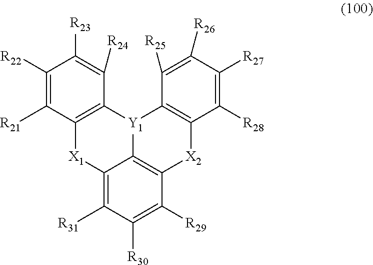

In formula 100,

Y₁ represents B:

X₁ and X₂, each independently, represent NR or O;

R, and R₂₁ to R₃₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N(Ar₄)(Ar₅); or may be linked to an adjacent substituent(s) to form a ring(s);

L₄, each independently, represents a single bond, a substituted or unsubstituted (C6-C30)arylene, or a substituted or unsubstituted (5- to 30-membered)heteroarylene; and

Ar₄ and Ar₅, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (5- to 30-membered)heteroaryl.

Specifically, the examples of the fluorescent dopant compound include the following compounds, but are not limited thereto.

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

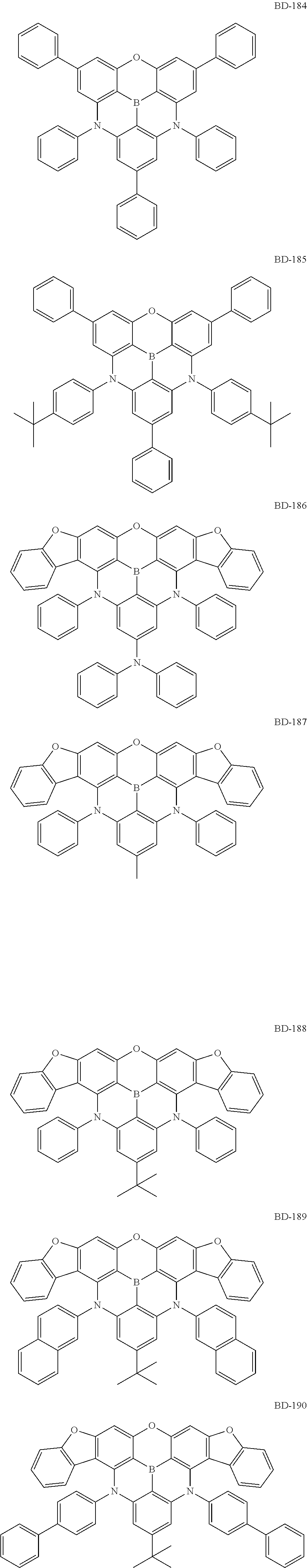

In the above compounds, D2 to D5 represent that 2 to 5 hydrogens are replaced with deuterium, respectively,

According to further embodiment of the present disclosure, the present disclosure provides a composition for manufacturing an organic electroluminescent device. The composition is preferably a composition for manufacturing a hole transport layer, a hole auxiliary layer or a light-emitting auxiliary layer of an organic electroluminescent device, and includes the compound of the present disclosure. When the hole transport layer is two or more layers, the compound of the present disclosure may be included in the composition for manufacturing a hole transport layer (a hole auxiliary layer) adjacent to the light-emitting layer.

An organic electroluminescent device according to the present disclosure has an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

Further, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from a chalcogenide layer, a metal halide layer, and a metal oxide layer (hereinafter, “a surface layer”) may be placed on an inner surface(s) of one or both electrode(s). Specifically, a chalcogenide (including oxides) layer of silicon and aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ (1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, preferably, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars, or a lighting system, for example an outdoor or indoor lighting system, by using the organic electroluminescent device of the present disclosure.

Hereinafter, the preparation method of the organic electroluminescent compound according to the present disclosure, the properties thereof, and light-emitting characteristics of the organic electroluminescent device comprising the same will be explained in detail with reference to the representative compounds of the present disclosure. However, the present disclosure is not limited by the following examples.

### Example 1: Preparation of Compound C-311

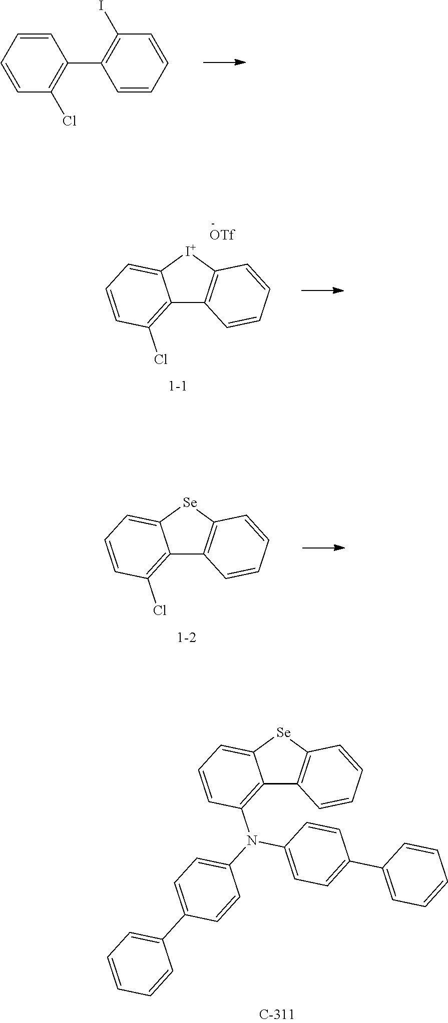 [SMILES: Clc1ccccc1-c1ccccc1I ; Clc1cccc2c1-c1ccccc1[I+]2 ; Clc1cccc2[se]c3ccccc3c12 ; c1ccc(-c2ccc(N(c3ccc(-c4ccccc4)cc3)c3cccc4[se]c5ccccc5c34)cc2)cc1]

Synthesis of Compound 1-1

2-chloro-2′-iodo-1,1′-biphenyl (20 g, 63.5 mmol), 3-chloro peroxybenzoic acid (21.3 g, 95.3 mmol), 16 mL of triflic acid, and 320 mL of methylene chloride were added to a reaction vessel, and then reacted for an hour. After completion of the reaction, the organic solvent was evaporated and the residue was washed with ethyl acetate to obtain compound 1-1 (25 g).

Synthesis of Compound 1-2

Compound 1-1 (19.7 g), potassium tert-butoxide (20.6 g, 184 mmol), selenium (10.9 g, 138 mmol), and 460 mL of dimethyl sulfoxide were added to a reaction vessel and stirred at 80° C. for 2 hours. After completion of the reaction, the mixture was washed with distilled water, and the organic layer was extracted with ethyl acetate. After removing residual moisture with magnesium sulfate, the organic layer was dried and separated by column chromatography to obtain compound 1-2 (8.6 g, yield of step 2: 71%).

Synthesis of Compound C-311

Compound 1-2 (5 g, 18.8 mmol), bis(4-biphenyl)amine (6 g, 18.8 mmol), tris(dibenzylideneacetone)dipalladium (0.86 g, 0.94 mmol), tri-tert-butylphosphine (0.8 mL, 1.88 mmol), sodium tert-butoxide (4.5 g, 47 mmol), and 95 mL of toluene were added to a reaction vessel, and then stirred under reflux for 16 hours. After completion of the reaction, the mixture was washed with distilled water, and the organic layer was extracted with ethyl acetate. After removing residual moisture with magnesium sulfate, the organic layer was dried and separated by column chromatography to obtain compound C-311 (6.1 g, yield: 62%).

| | MW | M.P. |
|---|---|---|
| C-311 | 550.56 | 221° C. |

### Example 2: Preparation of Compound C-312

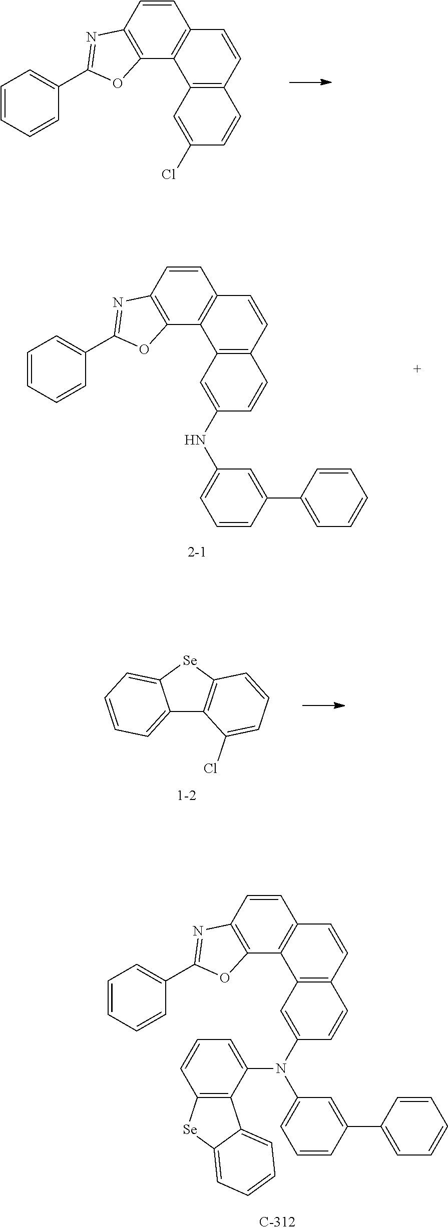 [SMILES: Clc1ccc2ccc3ccc4nc(-c5ccccc5)oc4c3c2c1 ; c1ccc(-c2cccc(Nc3ccc4ccc5ccc6nc(-c7ccccc7)oc6c5c4c3)c2)cc1 ; Clc1cccc2[se]c3ccccc3c12 ; c1ccc(-c2cccc(N(c3ccc4ccc5ccc6nc(-c7ccccc7)oc6c5c4c3)c3cccc4[se]c5ccccc5c34)c2)cc1]

Synthesis of Compound 2-1

10-chloro-2-phenyl-phenanthro[3,4-d]oxazole (10 g, 30.3 mmol), [1,1′-biphenyl]-3-amine (6.7 g, 39.4 mmol) palladium acetate (0.34 g, 1.5 mmol), tri-tert-butylphosphine (0.75 mL, 3.03 mmol), sodium tert-butoxide (7.3 g, 75.7 mmol), and 150 mL of o-xylene were added to a reaction vessel, and then reacted for 16 hours. After completion of the reaction, the mixture was washed with distilled water, and the organic layer was extracted with ethyl acetate. After removing residual moisture with magnesium sulfate, the organic layer was dried and separated by column chromatography to obtain compound 2-1 (11 g, yield: 80%).

Synthesis of Compound C-312

Compound 2-1 (4 g, 8.65 mmol), compound 1-2 (2.4 g, 9.08 mmol), tris(dibenzylideneacetone)dipalladium (0.39 g, 0.43 mmol), 2-dichlorohexylphosphine-2′,6′-dimethoxybiphenyl (0.35 g, 0.86 mmol), sodium teart-butoxide (2 g, 21.6 mmol), and 45 mL of toluene were added to a reaction vessel, and then reacted for 3 hours. After completion of the reaction, the mixture was washed with distilled water, and the organic layer was extracted with ethyl acetate. After removing residual moisture with magnesium sulfate, the organic layer was dried and then separated by column chromatography to obtain compound C-312 (5.3 g, yield: 90%).

| | MW | M.P. |
|---|---|---|
| C-312 | 691.69 | 183° C. |

### Example 3: Preparation of Compound C-304

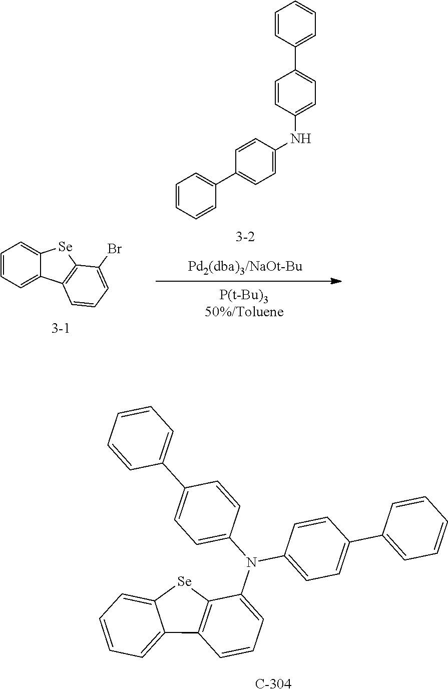 [SMILES: c1ccc(-c2ccc(Nc3ccc(-c4ccccc4)cc3)cc2)cc1 ; Brc1cccc2c1[se]c1ccccc12 ; c1ccc(-c2ccc(N(c3ccc(-c4ccccc4)cc3)c3cccc4c3[se]c3ccccc34)cc2)cc1]

Compound 3-1 (5.8 g, 18.68 mmol), compound 3-2 (5 g, 15.57 mmol), Pd₂(dba)₃ (0.71 g, 0.778 mmol), P(t-Bu)₃ 50% (0.77 mL, 1.557 mmol), NaOt-Bu (2.2 g, 23.35 mmol), and 80 mL of toluene were added to a flask, dissolved, and then stirred under reflux for 3 hours. After completion of the reaction, the mixture was cooled to room temperature, filtered with celite, dissolved in methylene chloride (MC), and then separated by column chromatography to obtain compound C-304 (4.4 g, yield: 51%).

| | MW | M.P. |
|---|---|---|
| C-304 | 550.5 | 395.9° C. |

### Example 4: Preparation of Compound C-426

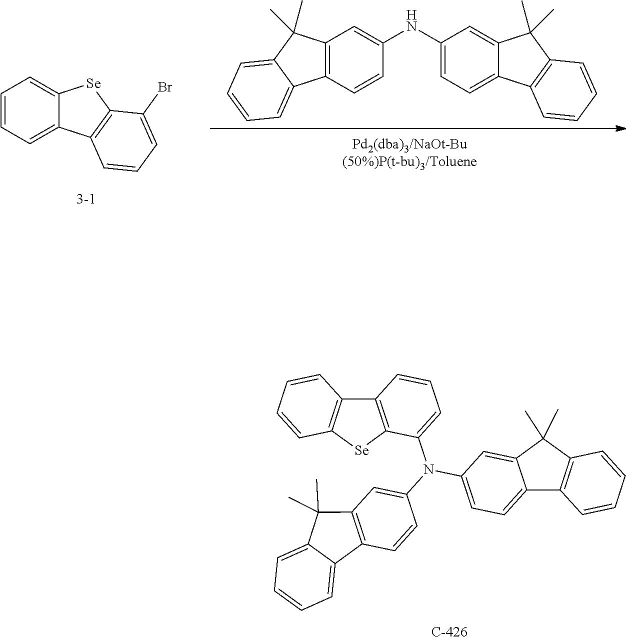 [SMILES: Brc1cccc2c1[se]c1ccccc12 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3cccc4c3[se]c3ccccc34)cc21]

Compound 3-1 (8.5 g, 27.4 mmol), bis(9,9-dimethyl-9H-fluorene-2-yl)amine (10.0 g, 24.9 mmol), Pd₂(dba)₃ (1.14 g, 1.24 mmol), P(t-bu)₃ 50% (1.23 mL, 2.5 mmol), NaOt-bu (4.78 g, 49.8 mmol), and 125 mL of toluene were added to a flask, and then stirred at 150° C. After completion of the reaction, the mixture was cooled to room temperature, filtered with celite, dissolved in MC, and then separated by column chromatography. Next, methanol was added thereto and the resulting solid was filtered under reduced pressure to obtain compound C-426 (13.1 g, yield: 83.4%).

| | MW | M.P. |
|---|---|---|
| C-426 | 630.7 | 210° C. |

### Example 5: Preparation of Compound C-499

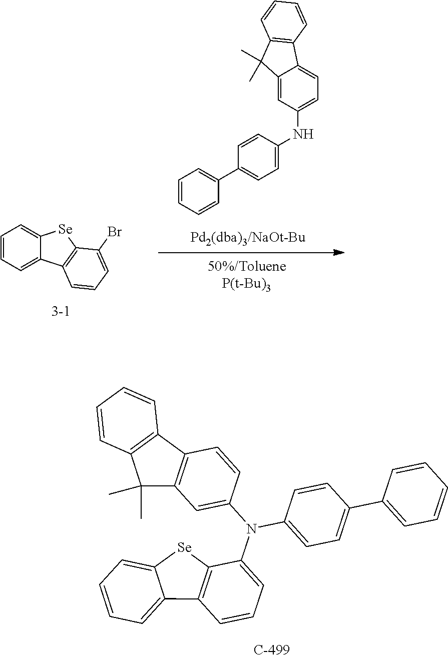 [SMILES: CC1(C)c2ccccc2-c2ccc(Nc3ccc(-c4ccccc4)cc3)cc21 ; Brc1cccc2c1[se]c1ccccc12 ; CC1(C)c2ccccc2-c2ccc(N(c3ccc(-c4ccccc4)cc3)c3cccc4c3[se]c3ccccc34)cc21]

Compound 3-1 (9.4 g, 30.43 mmol), N-([1,1′-biphenyl]-4-yl)-9,9-dimethyl-9H-fluorene-2-amine (10 g, 27.66 mmol), Pd₂(dba)₃ (1.27 g, 1.38 mmol), P(t-Bu)₃ 50% (1.2 mL, 2.7 mmol), NaOt-Bu (7.9 g, 82.98 mmol), and 140 mL of toluene were added to a flask, dissolved, and then stirred under reflux for 3 hours. After completion of the reaction, the mixture was cooled to room temperature, filtered with celite, dissolved in MC, and then filtered with silica to obtain compound C-499 (10.7 g, yield: 65%).

| | MW | M.P. |
|---|---|---|
| C-499 | 591.1 | 231.7° C. |

### Example 6: Preparation of Compound C-500

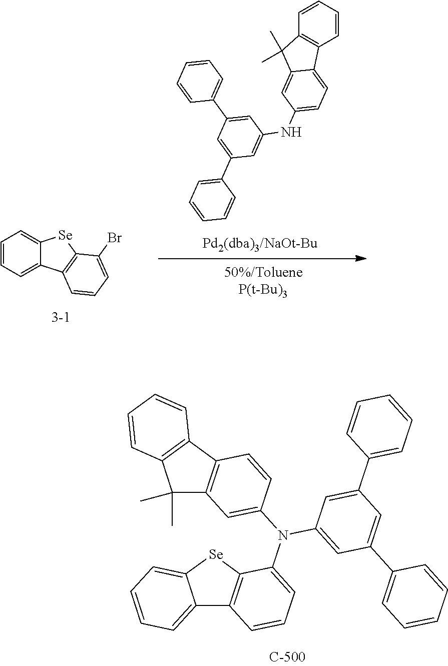 [SMILES: CC1(C)c2ccccc2-c2ccc(Nc3cc(-c4ccccc4)cc(-c4ccccc4)c3)cc21 ; Brc1cccc2c1[se]c1ccccc12 ; CC1(C)c2ccccc2-c2ccc(N(c3cc(-c4ccccc4)cc(-c4ccccc4)c3)c3cccc4c3[se]c3ccccc34)cc21]

Compound 3-1 (7.8 g, 25.14 mmol), N-([1,1′:3′,1″-terphenyl]-5′-yl)-9,9-dimethyl-9H-fluorene-2-amine (10 g, 22.85 mmol), Pd₂(dba)₃ (1.05 g, 1.14 mmol), P(t-Bu)₃ 50% (1 mL, 2.28 mmol), NaOt-Bu (6.6 g, 68.55 mmol), and 114 mL of toluene were added to a flask, dissolved, and then stirred under reflux for 3 hours. After completion of the reaction, the mixture was cooled to room temperature, filtered with celite, dissolved in MC, and then filtered with silica to obtain compound C-500 (11.9 g, yield: 78%).

| | MW | M.P. |
|---|---|---|
| C-500 | 667.1 | 139.4° C. |

### Example 7: Preparation of Compound C-501

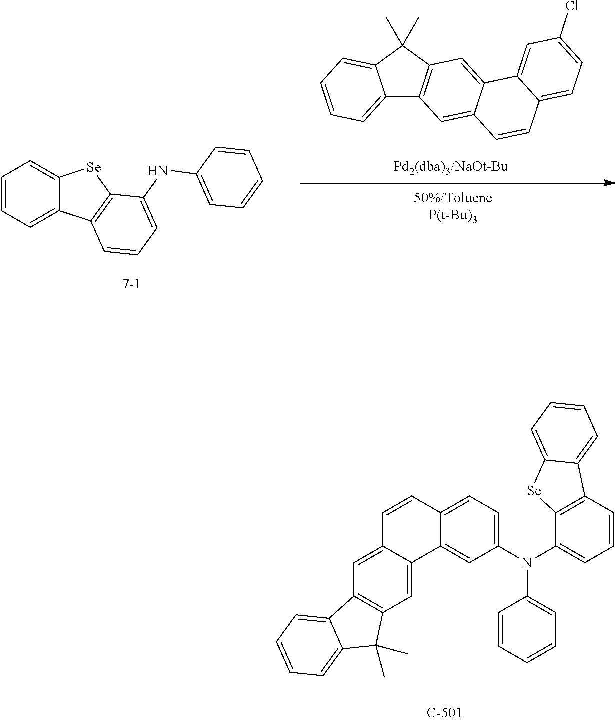 [SMILES: CC1(C)c2ccccc2-c2cc3ccc4ccc(Cl)cc4c3cc21 ; c1ccc(Nc2cccc3c2[se]c2ccccc23)cc1 ; CC1(C)c2ccccc2-c2cc3ccc4ccc(N(c5ccccc5)c5cccc6c5[se]c5ccccc56)cc4c3cc21]

2-chloro-12,12-dimethyl-12H-indeno[1,2-b]phenanthrene (6.04 g, 18.4 mmol), compound 7-1 (5.64 g, 17.5 mmol), Pd₂(dba)₃ (801 mg, 0.875 mmol), P(t-Bu)₃ 50% (0.850 mL, 1.75 mmol), and NaOt-Bu (5.05, 52.5 mmol) were dissolved in 88 mL of toluene, and then refluxed at 145° C. for 2 hours. After completion of the reaction, the mixture was distilled under reduced pressure and separated by column chromatography to obtain compound C-501 (9.0 g, yield: 83.6%).

| | MW | M.P. |
|---|---|---|
| C-501 | 639.66 | 247.1° C. |

### Example 8: Preparation of Compound C-502

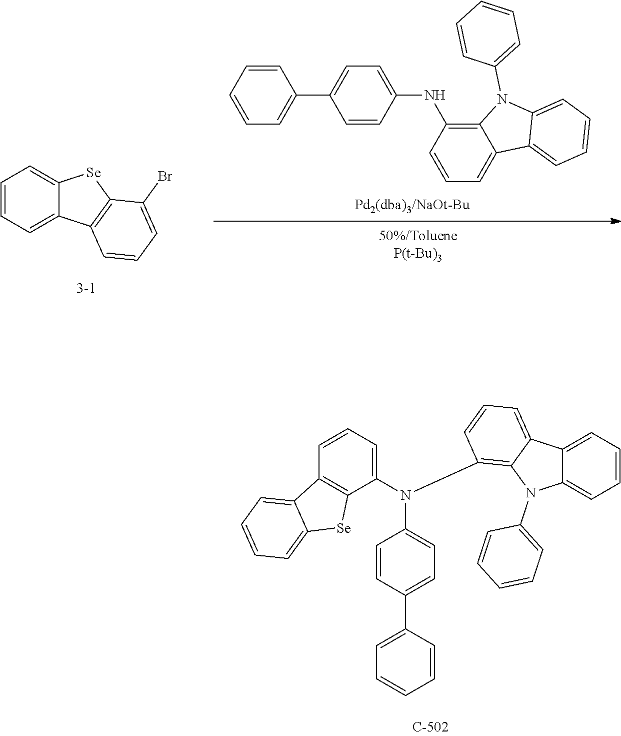 [SMILES: Brc1cccc2c1[se]c1ccccc12 ; c1ccc(-c2ccc(Nc3cccc4c5ccccc5n(-c5ccccc5)c34)cc2)cc1 ; c1ccc(-c2ccc(N(c3cccc4c3[se]c3ccccc34)c3cccc4c5ccccc5n(-c5ccccc5)c34)cc2)cc1]

Compound 3-1 (8.79 g, 21.4 mmol), N-([1,1′-biphenyl]-4-yl)-9-phenyl-9H-carbazole-1-amine (6.20 g, 20.0 mmol), Pd₂(dba)₃ (0.92 g, 1.00 mmol), P(t-Bu)₃ 50% (0.97 mL, 2.00 mmcl), NaOt-Bu (5.77 g, 60.0 mmol), and 100 mL of toluene were added to a flask, dissolved, and then stirred under reflux for 2 hours. After completion of the reaction, the mixture was cooled to room temperature, filtered with celite, dissolved in MC, and then filtered with silica to obtain compound C-502 (7.0 g, yield: 54.7%).

| | MW | M.P. |
|---|---|---|
| C-502 | 614.65 | 170.7° C. |

### Example 9: Preparation of Compound C-503

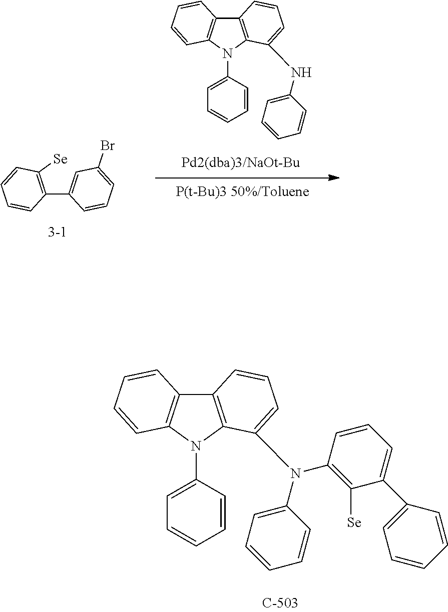

Compound 3-1 (20.9 g, 67.28 mmcl), N,9-diphenyl-9H-carbazole-1-amine (15 g, 44.85 mmol), Pd₂(dba)₃ (2.05 g, 2.24 mmol), P(t-Bu)₃ 50% (2.2 mL, 4.49 mmol), NaOt-Bu (6.5 g, 67.28 mmcl), and 225 mL of toluene were added to a flask, dissolved, and then stirred under reflux. After completion of the reaction, the mixture was cooled to room temperature, filtered with celite, dissolved with MC, and then filtered with silica to obtain compound C-503 (3.4 g, yield: 13.5%).

| | MW | M.P. |
|---|---|---|
| C-503 | 563.6 | 265.3° C. |

### Device Example 1: Producing an OLED Comprising an Organic Electroluminescent Compound According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. Thereafter, the ITO substrate was mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates and compound HI-1 was deposited in a doping amount of 3 wt % based on the total amount of the compound HI-1 and compound HT-1 to form a hole injection layer having a thickness of 10 nm. Next, compound HT-1 was deposited on the hole injection layer to form a first hole transport layer having a thickness of 75 nm. Compound C-311 was then deposited on the first hole transport layer to form a second hole transport layer having a thickness of 5 nm. After forming the hole injection layer and the hole transport layers, a light-emitting layer was formed thereon as follows:

Compound BH-228 was introduced into two cells of the vacuum vapor deposition apparatus as host, and compound BD-96 was introduced into another cell as a dopant. The two materials were evaporated at a different rate and the dopant was doped in a doping amount of 2 wt % based on the total amount of the host and dopant to form a light-emitting layer having a thickness of 20 nm on the hole transport layer. Next, compound HBL was deposited as an electron buffer material on the light-emitting layer to form an electron buffer layer having a thickness of 5 nm. Next, compounds ETL-1 and EIL-1 were deposited at a weight ratio of 5:5 to form an electron transport layer having a thickness of 30 nm. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Device Example 2: Producing an OLED Comprising an Organic Electroluminescent Compound According to the Present Disclosure

An OLED was produced in the same manner as in Device Example 1, except that compound C-312 was used as the second hole transport layer.

### Device Example 3: Producing an OLED Comprising an Organic Electroluminescent Compound According to the Present Disclosure

An OLED was produced in the same manner as in Device Example 1, except that compound C-304 was used as the second hole transport layer.

### Device Example 4: Producing an OLED Comprising an Organic Electroluminescent Compound According to the Resent Disclosure

An OLED was produced in the same manner as in Device Example 1, except that compound C-499 was used as the second hole transport layer.

### Device Example 5: Producing an OLED Comprising an Organic Electroluminescent Compound According to the Present Disclosure

An OLED was produced in the same manner as in Device Example 1, except that compound C-500 was used as the second hole transport layer.

### Device Example 6: Producing an OLED Comprising an Organic Electroluminescent Compound According to the Present Disclosure

An OLED was produced in the same manner as in Device Example 1, except that compound C-426 was used as the second hole transport layer.

### Comparative Example 1: Producing an OLED not Comprising an Organic Electroluminescent Compound According to the Present Disclosure

An OLED was produced in the same manner as in Device Example 1, except that the first hole transport layer having a thickness of 80 nm was deposited without the second hole transport layer

### Comparative Example 2: Producing an OLED not Comprising an Organic Electroluminescent Compound According to the Resent Disclosure

An OLED was produced in the same manner as in Device Example 1, except that compound A-1 was used as the second hole transport layer.

The driving voltage, luminous efficiency, and CIE 1931 color coordinate at a luminance of 1,000 nit of the OLEDs produced in Device Examples 1 and 2, and Comparative Examples 1 and 2 are provided in Table 1 below. In addition, the driving voltage, luminous efficiency, and CIE 1931 color coordinate at a luminance of 1,000 nit, and the time taken for luminance to decrease from 100% to 95% at a luminance of 20006 nit (lifetime: T95) of the OLEDs produced in Device Examples 3 to 6, and Comparative Examples 1 and 2 are provided in Table 2 below.

TABLE 1

| | Second | | Luminous | |
|---|---|---|---|---|
| | Hole | Driving | Effi- | CIE Color |
| | Transport | Voltage | ciency | Coordinate |
| | Layer | [V] | [cd/A] | (x, y) |
| Device | C-311 | 3.3 | 7.6 | (0.129, 0.090) |
| Example 1 | | | | |
| Device | C-312 | 3.3 | 7.2 | (0.130, 0.088) |
| Example 2 | | | | |
| Comparative | — | 3.3 | 6.6 | (0 130, 0 089) |
| Example 1 | | | | |
| Comparative | A-1 | 3.4 | 6.9 | (0.130. 0.089) |
| Example 2 | | | | |

TABLE 2

| | Second | | Luminous | | Life- |
|---|---|---|---|---|---|
| | Hole | Driving | Effi- | CIE Color | time |
| | Transport | Voltage | ciency | Coordinate | (T95) |
| | Layer | [V] | [cd/A] | (x, y) | [hr] |
| Device | C-304 | 3.3 | 7.5 | (0.129, 0.090) | 30 |
| Example 3 | | | | | |
| Device | C-499 | 3.3 | 7.2 | (0.130, 0.086) | 27 |
| Example 4 | | | | | |
| Device | C-500 | 3.3 | 7.3 | (0.131, 0.084) | 15 |
| Example 5 | | | | | |
| Device | C-426 | 3.2 | 6.6 | 0.131, 0.083) | 18 |
| Example 6 | | | | | |
| Comparative | — | 3.3 | 6.6 | (0.130, 0.089) | 5 |
| Example 1 | | | | | |
| Comparative | A-1 | 3.4 | 6.9 | (0.130. 0.089) | 1 |
| Example 2 | | | | | |

From Tables 1 and 2 above, it can be confirmed that the OLEDs comprising the hole transport compounds developed in the present disclosure as the second hole transport material (Device Examples 1 to 6) exhibited significantly improved luminous efficiency and/or lifetime properties compared to the OLED using the single hole transport material (Comparative Example 1), and the OLED using compound A-1 not according to the present disclosure (Comparative Example 2) as the second hole transport material.

Referring to Table 3 below, it was confirmed that compound A-1 had a low HOMO (high occupied molecular orbital) of −5.1 eV or less compared to compound C-304 comprising one dibenzoselenophene since compound A-1 comprises two dibenzoselenophenes. A preferred HOMO level for use as a hole transport layer is about −4.7 to −5.0 eV, and a compound comprising two dibenzoselenophenes has an energy level that is not suitable for use as a hole transport layer. In particular, when compound A-1 is used in the hole transport layer, it does not have an appropriate energy level with the adjacent layer and affects the injection and mobility of holes and electrons. Therefore, as shown in Table 2 above, it can be confirmed that luminous efficiency and lifetime of the OLEDs comprising compound A-1 were decreased.

TABLE 3

| | Compound C-304 | Compound A-1 |
|---|---|---|
| Structure | 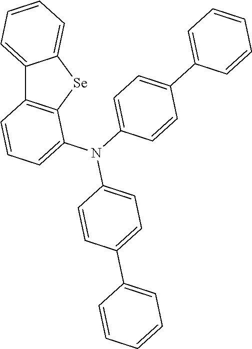 [SMILES: c1ccc(-c2ccc(N(c3ccc(-c4ccccc4)cc3)c3cccc4c3[se]c3ccccc34)cc2)cc1] | 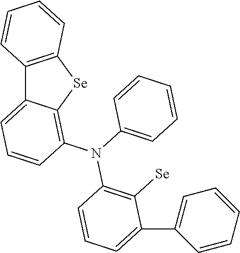 |
| LUMO | −1.024 | −1.023 |
| HOMO | −4.962 | −5.183 |
| Triplet Energy | 2.799 | 2.973 |

* With Gaussian 16, which is Gaussian's quantum chemistry calculation program, the structure was optimized by applying the background sets of B3LYP and 6-31G(d), which are hybrid Density Functional Theory (hybrid DFT), and TD-DFT (time dependent DFT) was used to calculate the triplet state.

The compounds used in the Device Examples and the Comparative Examples are shown in Table 4 below.

TABLE 4

| | |
|---|---|
| Hole Injection Layer/ Hole Transport Layer | 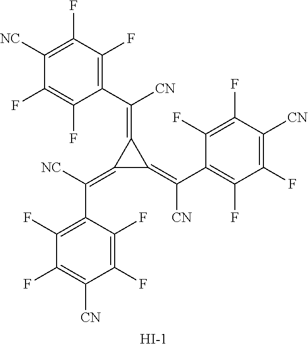 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 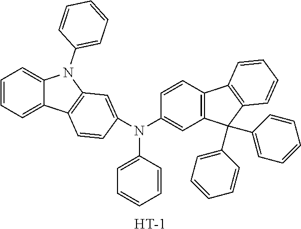 |
| | 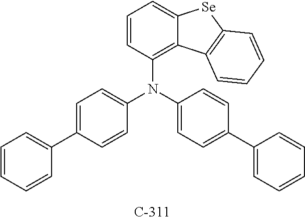 [SMILES: c1ccc(-c2ccc(N(c3ccc(-c4ccccc4)cc3)c3cccc4[se]c5ccccc5c34)cc2)cc1] |
| | 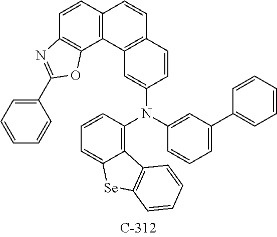 [SMILES: c1ccc(-c2cccc(N(c3ccc4ccc5ccc6nc(-c7ccccc7)oc6c5c4c3)c3cccc4[se]c5ccccc5c34)c2)cc1] |
| | 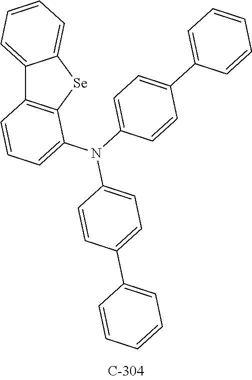 [SMILES: c1ccc(-c2ccc(N(c3ccc(-c4ccccc4)cc3)c3cccc4c3[se]c3ccccc34)cc2)cc1] |
| | 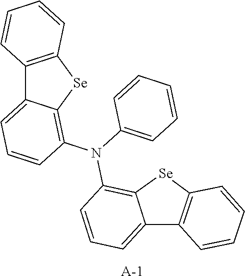 |
| | 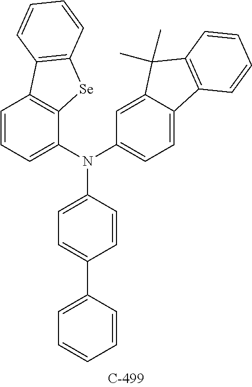 [SMILES: CC1(C)c2ccccc2-c2ccc(N(c3ccc(-c4ccccc4)cc3)c3cccc4c3[se]c3ccccc34)cc21] |
| | 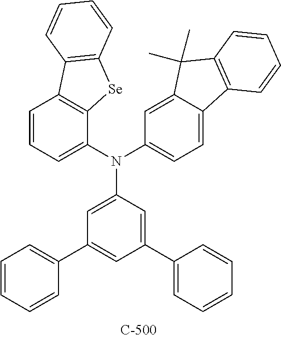 [SMILES: CC1(C)c2ccccc2-c2ccc(N(c3cc(-c4ccccc4)cc(-c4ccccc4)c3)c3cccc4c3[se]c3ccccc34)cc21] |
| | 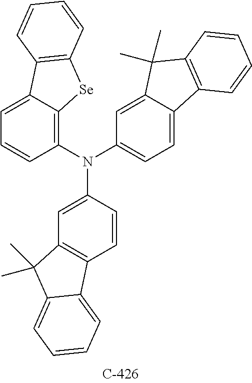 [SMILES: CC1(C)c2ccccc2-c2ccc(N(c3ccc4c(c3)C(C)(C)c3ccccc3-4)c3cccc4c3[se]c3ccccc34)cc21] |
| Light- Emitting Layer | 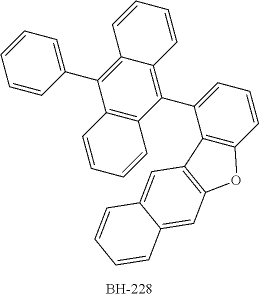 [SMILES: c1ccc(-c2c3ccccc3c(-c3cccc4oc5cc6ccccc6cc5c34)c3ccccc23)cc1] |
| | 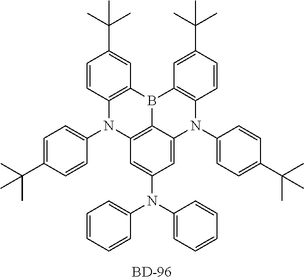 |
| Electron Buffer Layer | 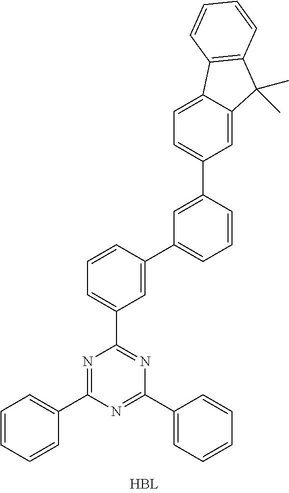 [SMILES: CC1(C)c2ccccc2-c2ccc(-c3cccc(-c4cccc(-c5nc(-c6ccccc6)nc(-c6ccccc6)n5)c4)c3)cc21] |
| Electron Transport Layer/ Electron Injection Layer | 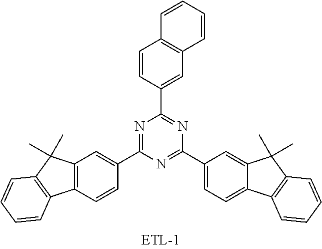 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 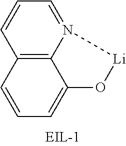 |

1. An organic electroluminescent compound represented by the following formula 1: 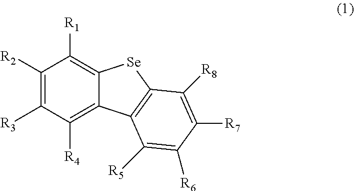
in formula 1,
R₁ to R₄, each independently, represent hydrogen or deuterium;
R₅ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or 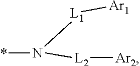
with a proviso that at least one of R₅ to R₈ represents 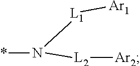
L₁ and L₂, each independently, represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ and Ar₂, each independently, represent hydrogen, deuterium, a halogen, a cyano, a substituted or unsubstituted (C1-C30)alkyl, a (C6-C30)aryl unsubstituted or substituted with a (C1-C30)alkyl(s), a substituted or unsubstituted (3- to 30-membered)heteroaryl, a substituted or unsubstituted (C3-C30)cycloalkyl, a substituted or unsubstituted mono- or di-(C1-C30)alkylamino, a substituted or unsubstituted mono- or di-(C2-C30)alkenylamino, a mono- or di-(C6-C30) arylamino substituted with a (C1-C30)alkyl(s), a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3-to 30-membered)heteroarylamino, or a substituted or unsubstituted (C6-C30)aryl(3- to 30-membered)heteroarylamino; with a proviso that Ar₁ and Ar₂ do not comprise dibenzoselenophene; and
* represents a bonding position;
with a proviso that the following structures are excluded.

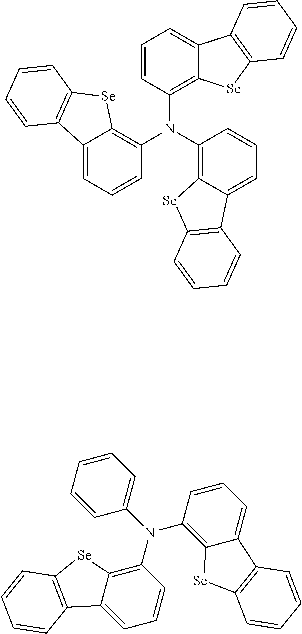 [SMILES: c1ccc2c(c1)[se]c1c(N(c3cccc4c3[se]c3ccccc34)c3cccc4c3[se]c3ccccc34)cccc12 ; c1ccc(N(c2cccc3c2[se]c2ccccc23)c2cccc3c2[se]c2ccccc23)cc1]

2. The organic electroluminescent compound according to claim 1, wherein formula 1 is represented by any one of the following formulas 1-1 to 1-4: 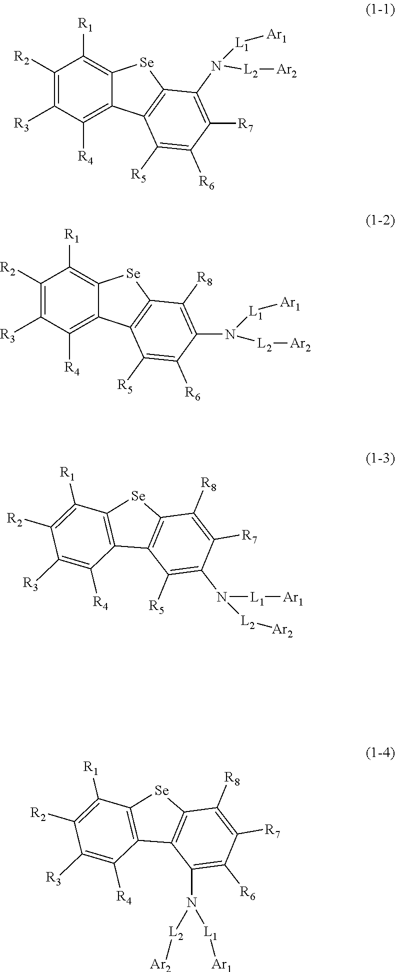
in formulas 1-1 to 1-4,
R₅ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and
R₁ to R₄, L₁, L₂, Ar₁, and Ar₂ are as defined in claim 1.

3. The organic electroluminescent compound according to claim 1, wherein Ar₁ or Ar₂ is represented by any one of the following formulas 1-5 to 1-8: 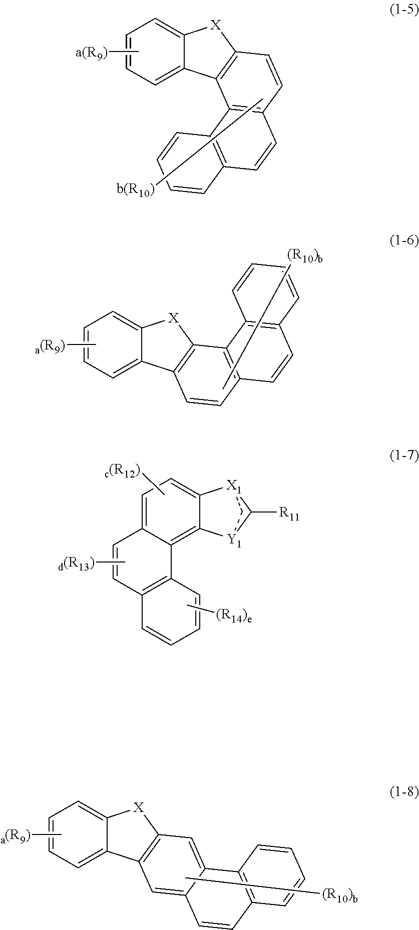
in formulas 1-5 to 1-8,
X₁ each independently, represents —O—, —S—, or CR′R″;
R′ and R″, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s);
X₁ and Y₁, each independently, represent —N═, —NR₁₅—, —O— or —S—, with the proviso that any one of X₁ and Y₁ represents —N═, and the other of X₁ and Y₁ represents —NR₁₅—, —O— or —S—;
R₁₁ represents a substituted or unsubstituted (C6-C30)aryl, or a substituted or unsubstituted (3- to 30-membered)heteroaryl, or a bonding position with L₁ or L₂;
R₉, R₁₀, and R₁₂ to R₁₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3-to 30-membered)heteroarylamino, a substituted or unsubstituted (C2-C30)alkenyl(C6-C30)arylamino, a substituted or unsubstituted (C2-C30)alkenyl(3-to 30-membered)heteroarylamino, or a substituted or unsubstituted (C6-C30)aryl(3- to 30-membered)heteroarylamino, or may be linked to an adjacent substituent(s) to form a ring(s); with a proviso that any one of R₉ and R₁₀ is linked to L₁ or L₂, and any one of R₁₁ to R₁₄ is linked to L₁ or L₂; and
a, each independently, represents an integer of 1 to 4, b, each independently, represents an integer of 1 to 3, c and d, each independently, represent an integer of 1 or 2, and e represents an integer of 1 to 4, in which if a to e are an integer of 2 or more, each of R₉, each of R₁₀, and each of R₁₂ to each of R₁₄ may be the same as or different from each other.

4. The organic electroluminescent compound according to claim 1, wherein the substituent(s) of the substituted alkyl, the substituted alkylene, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino, each independently, are at least one selected from the group consisting of deuterium: a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3-to 30-membered)heteroaryl unsubstituted or substituted with a (C6-C30)aryl(s); a (C6-C30)aryl unsubstituted or substituted with at least one of a (C1-C30)alkyl(s), a (C6-C30)aryl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The organic electroluminescent compound according to claim 1, wherein
R₁ to R₄, each independently, represent hydrogen or deuterium;
R₅ to R₈, each independently, represent hydrogen or deuterium, with a proviso that at least one of R₅ to R₈ represents 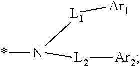
L₁ and L₂, each independently, represent a single bond, a substituted or unsubstituted (C6-C30)arylene, or a substituted or unsubstituted (5- to 30-membered)heteroarylene; and
Ar₁ and Ar₂, each independently, represent hydrogen, deuterium, a substituted or unsubstituted (C1-C6)alkyl, a (C6-C18)aryl unsubstituted or substituted with a (C1-C6)alkyl(s), a substituted or unsubstituted (5- to 20-membered)heteroaryl, a substituted or unsubstituted (C6-C18)aryl(5- to 20-membered)heteroarylamino, or a di(C6-C25)arylamino substituted with a (C1-C10)alkyl(s).

6. The organic electroluminescent compound according to claim 1, wherein
R₁ to R₄, each independently, represent hydrogen or deuterium;
R₅ to R₈, each independently, represent hydrogen or deuterium, with a proviso that at least one of R₅ to R₈ represents 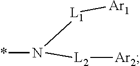
L₁ and L₂, each independently, represent a single bond, a substituted or unsubstituted (C6-C18)arylene, or a substituted or unsubstituted (5- to 20-membered)heteroarylene; and
Ar₁ and Ar₂, each independently, represent hydrogen, an unsubstituted (C1-C6)alkyl, a (C6-C18)aryl unsubstituted or substituted with a (C1-C6)alkyl(s), a (5-to 20-membered)heteroaryl unsubstituted or substituted with a (C6-C15)aryl(s), a (C6-C18)aryl(5- to 20-membered)heteroarylamino unsubstituted or substituted with a (C6-C12)aryl(s), or a di(C6-C18)arylamino substituted with a (C1-C6)alkyl(s).

7. The organic electroluminescent compound according to claim 1, wherein the compound represented by formula 1 is selected from the group consisting of the following compounds:

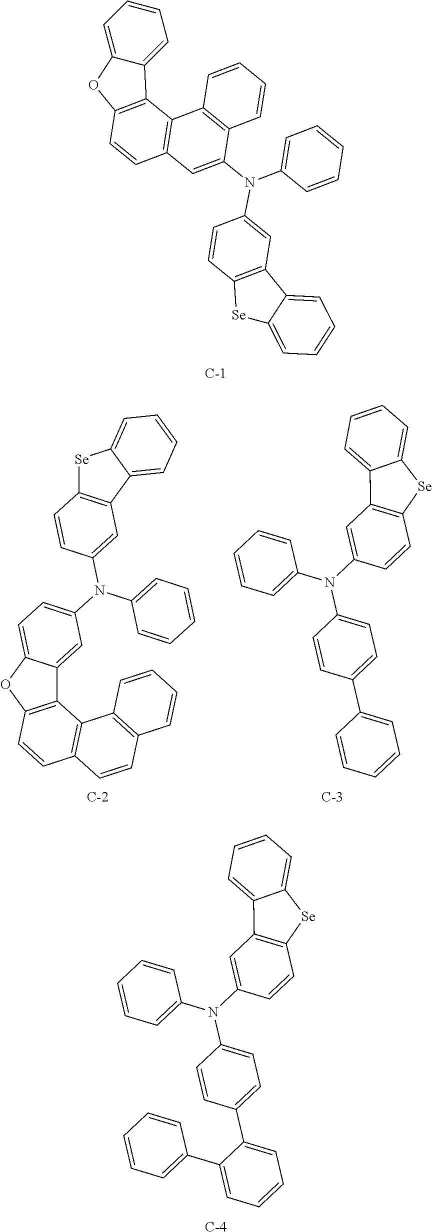

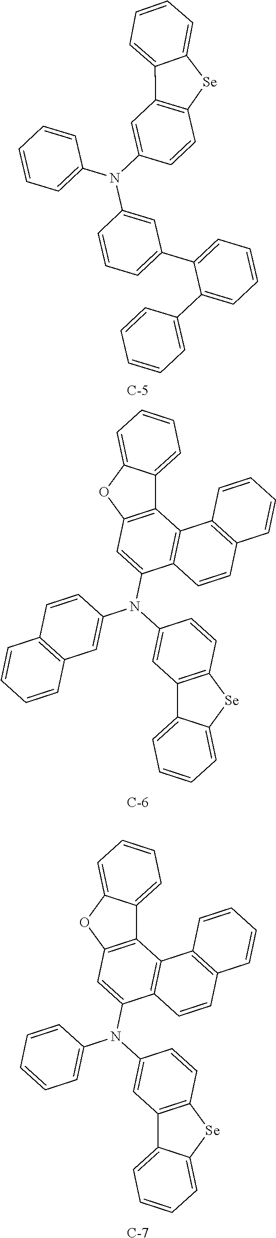

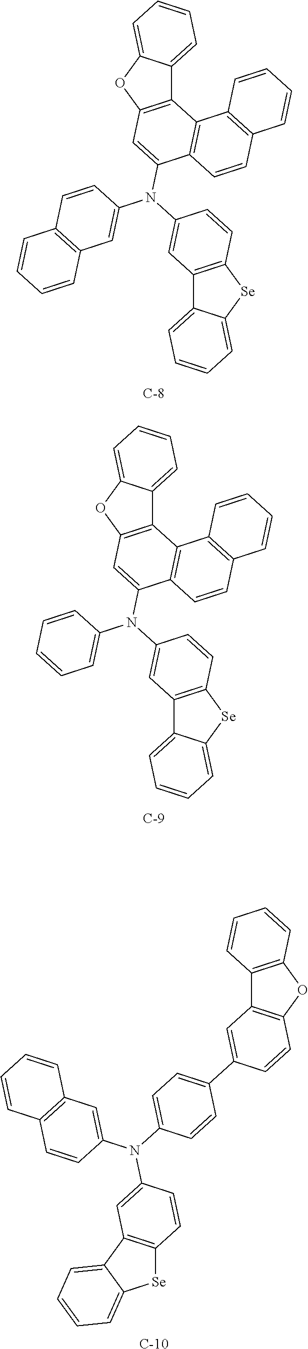

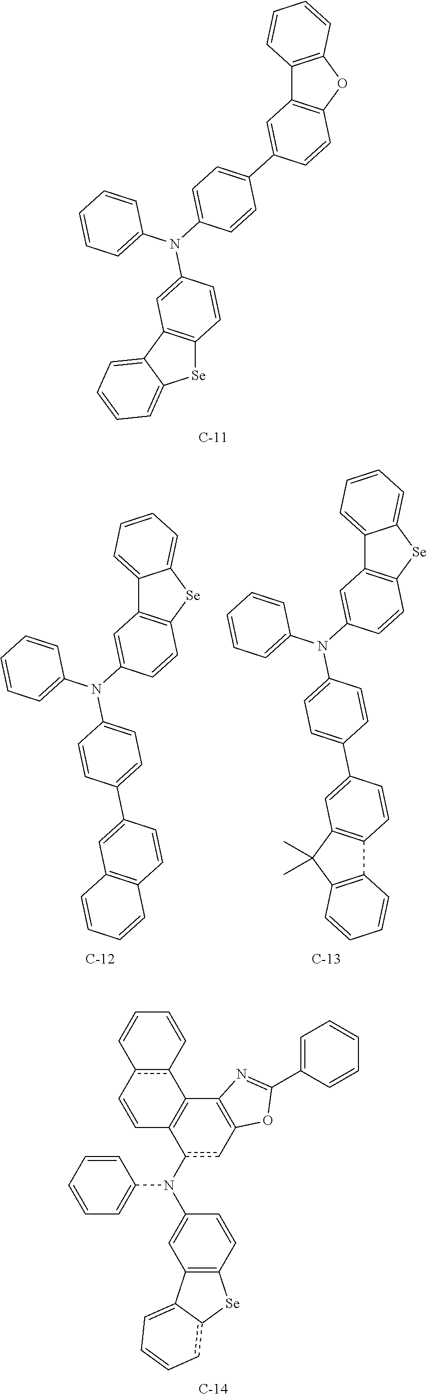

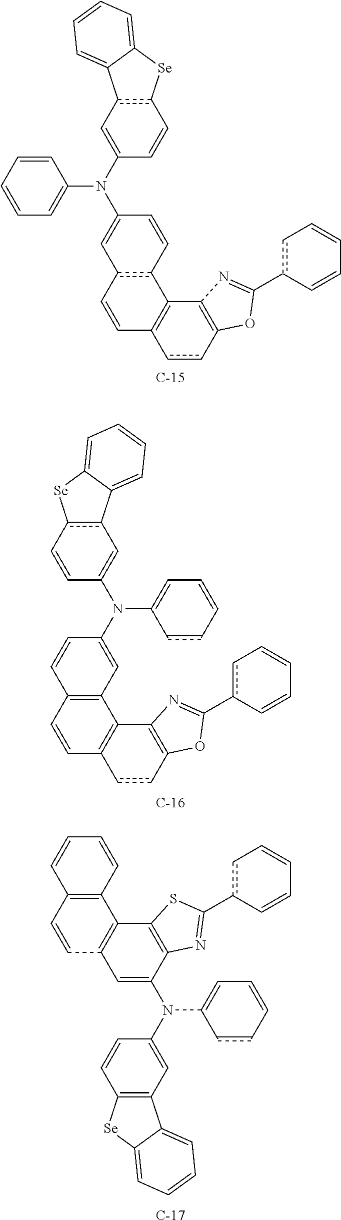

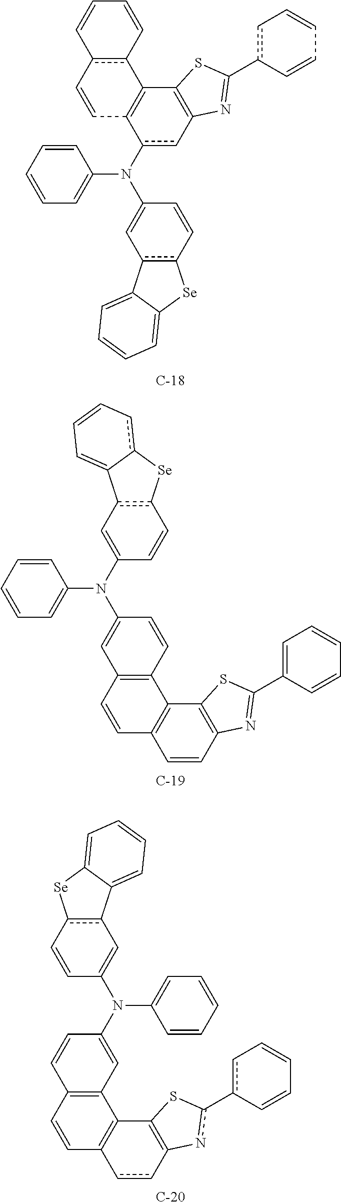

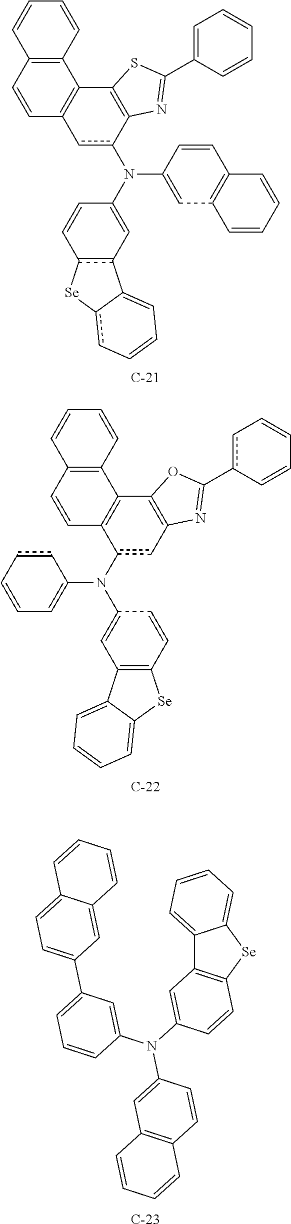

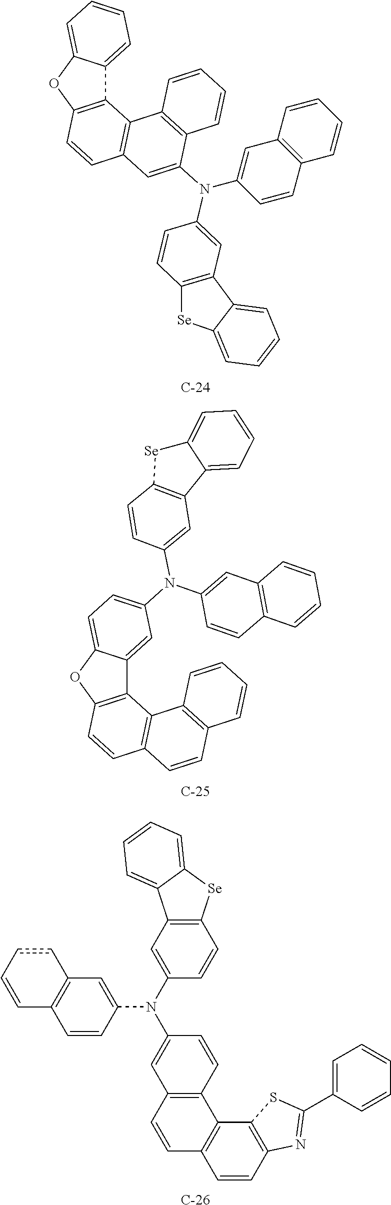

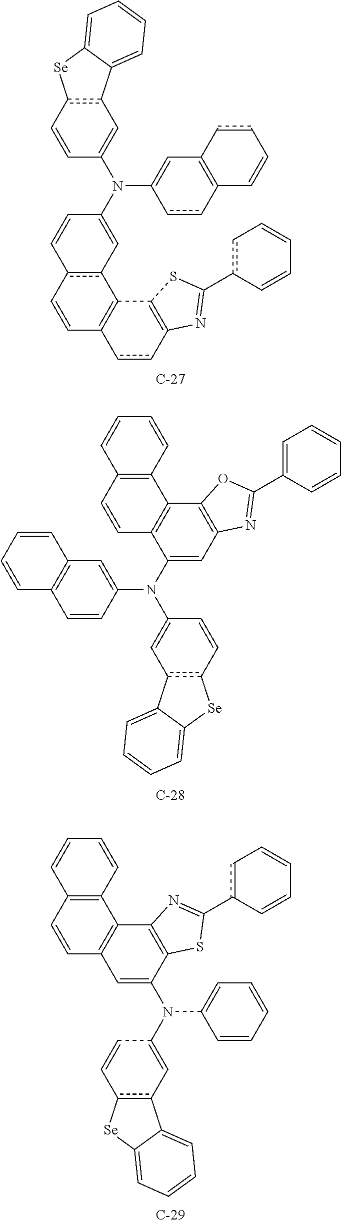

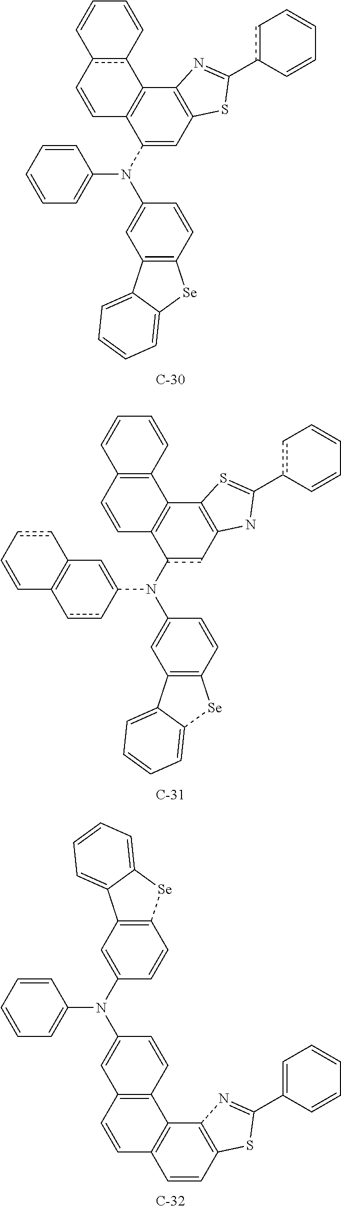

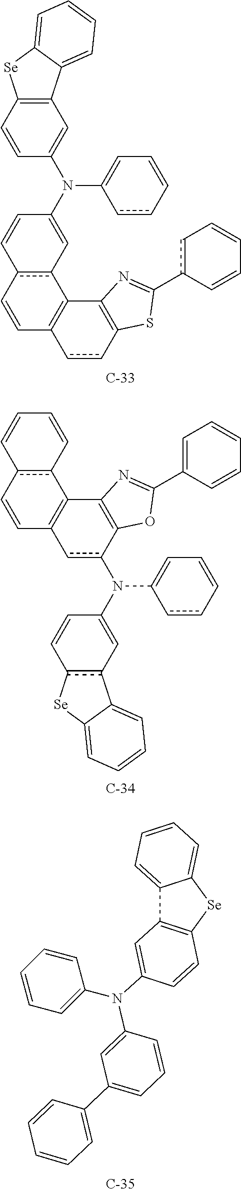

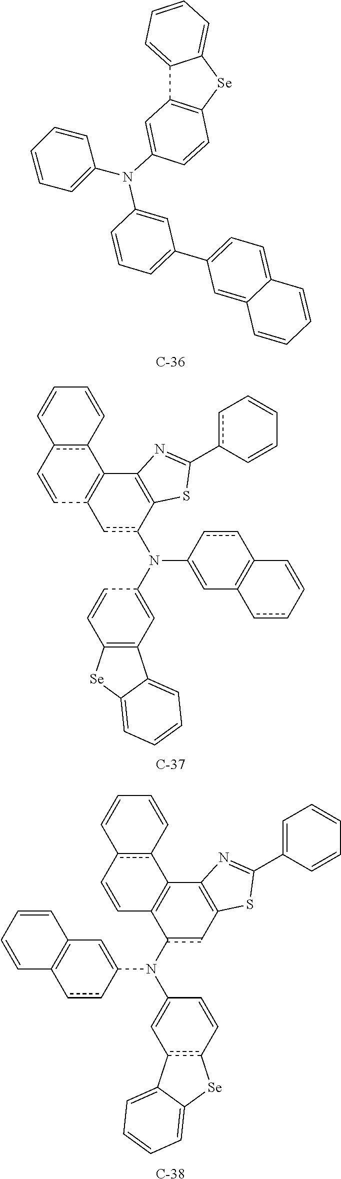

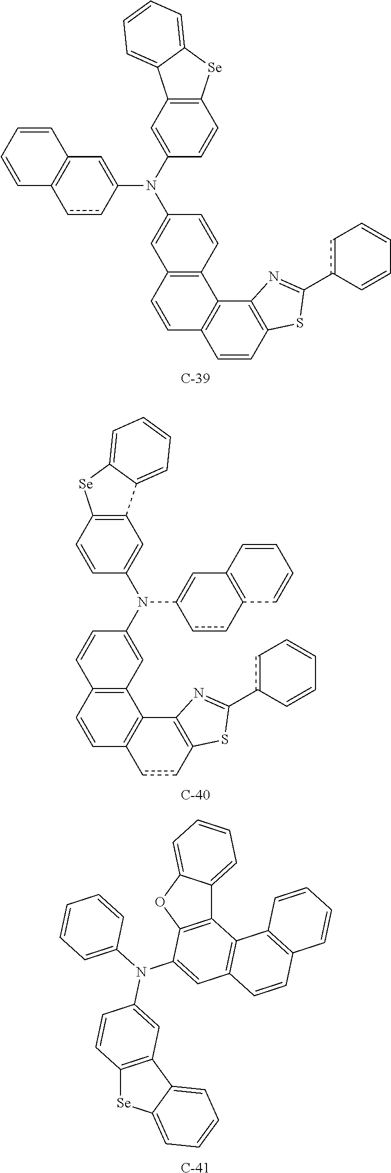

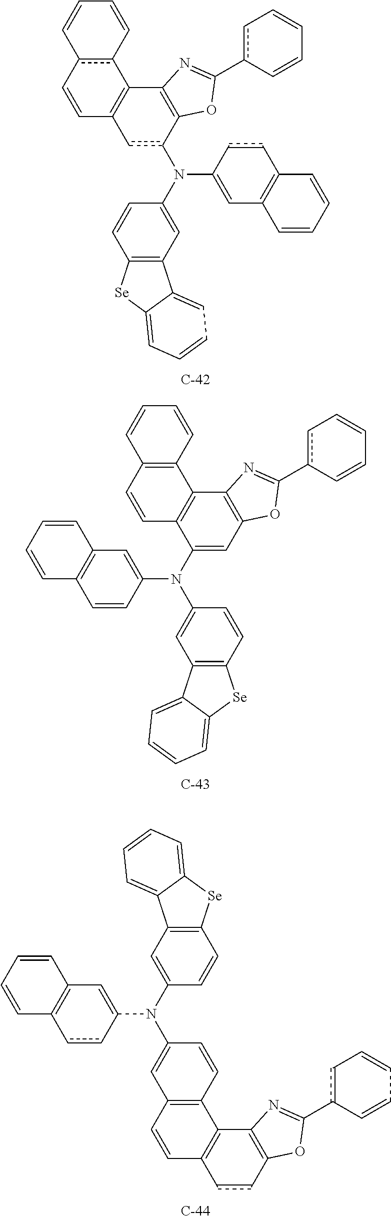

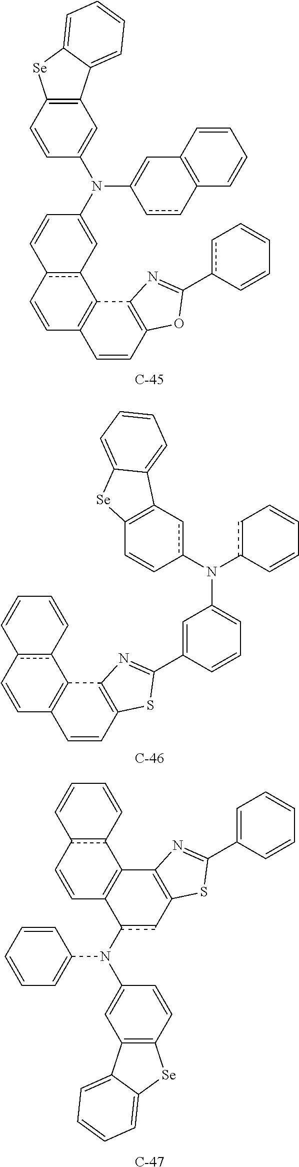

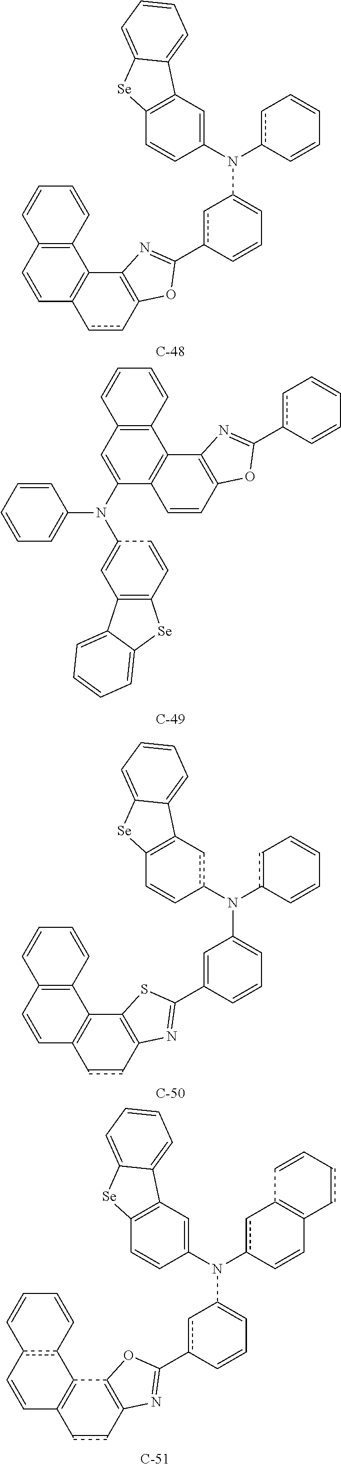

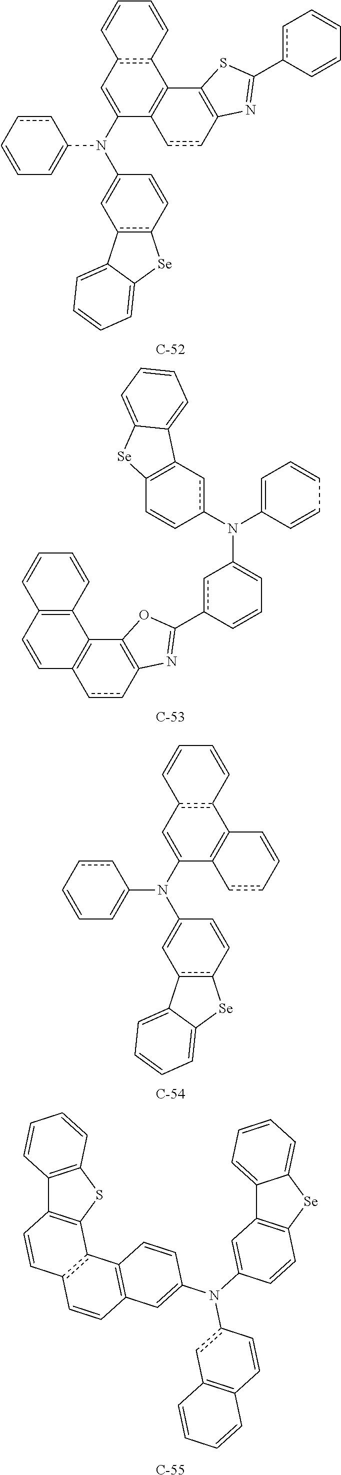

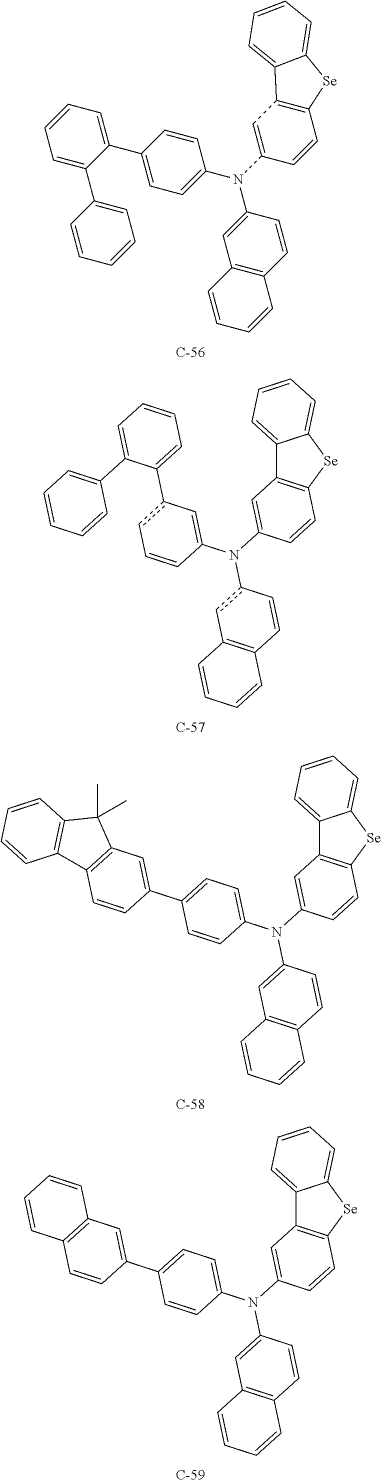

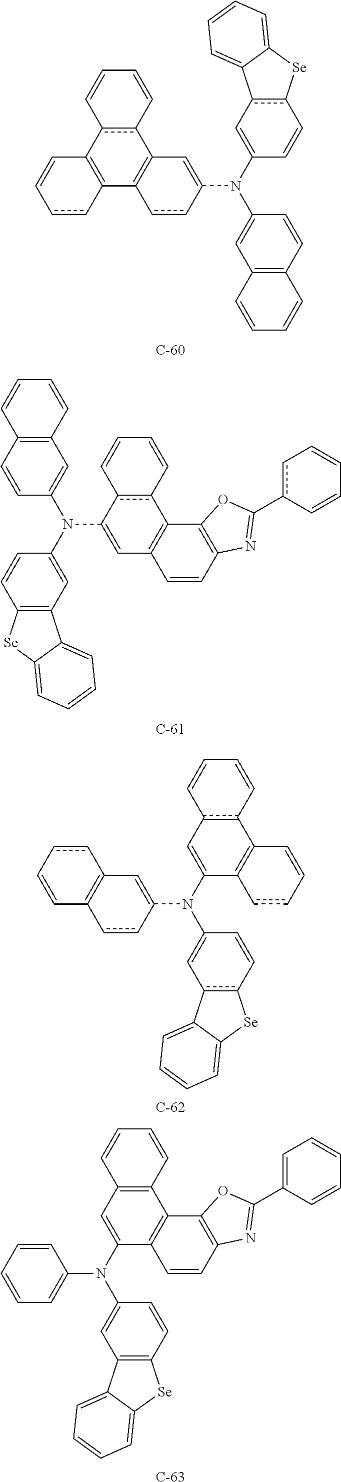

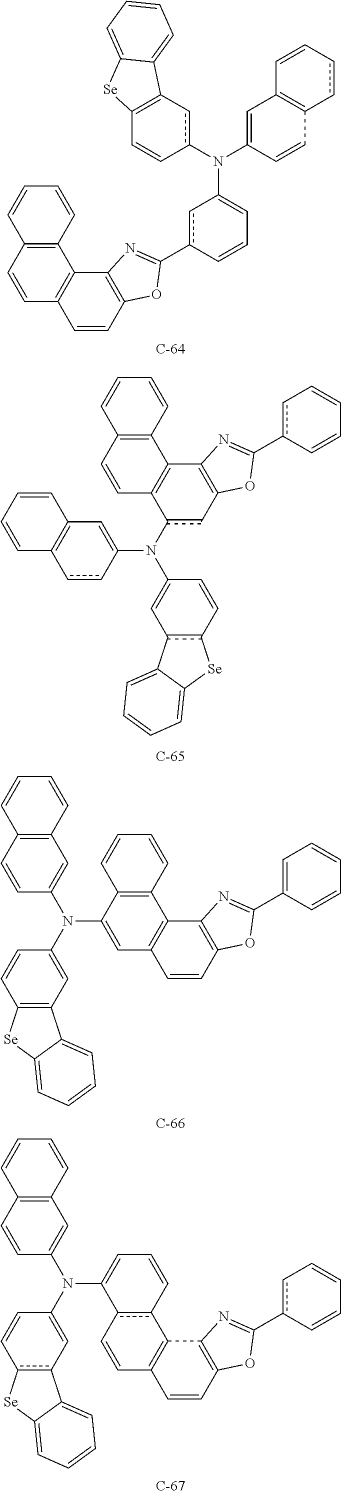

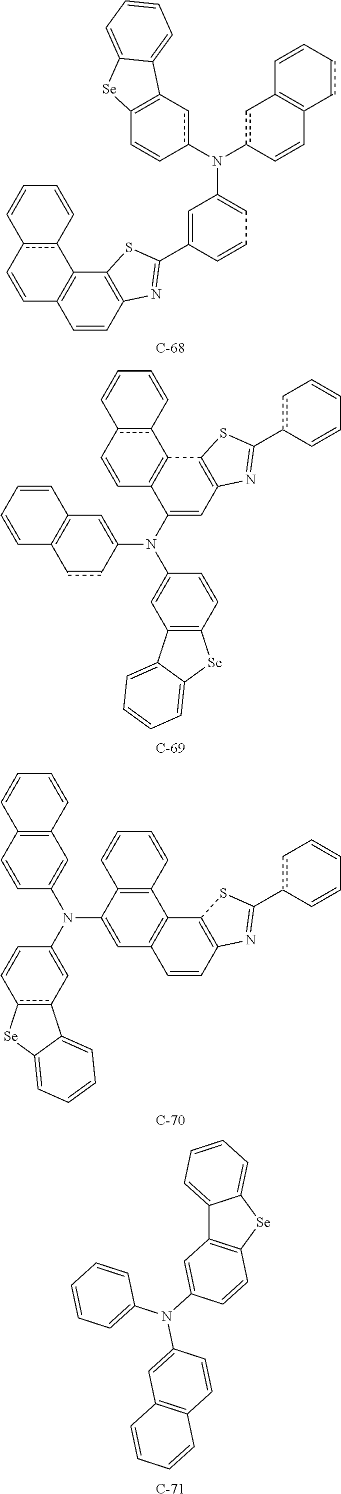

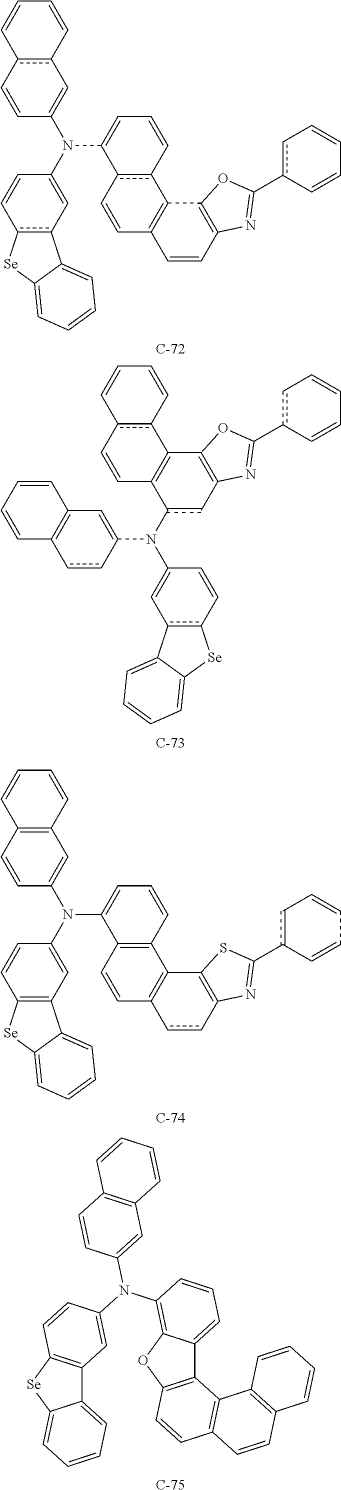

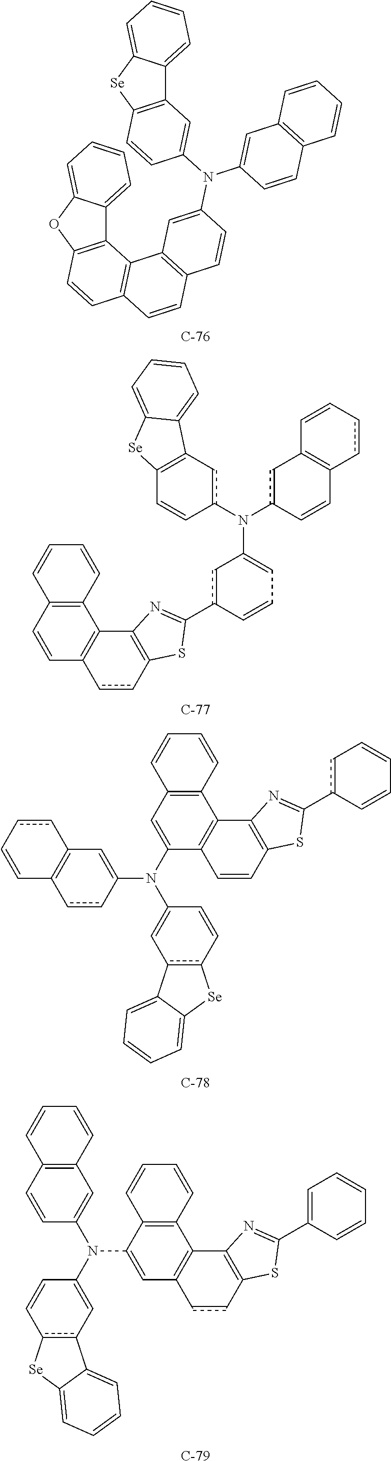

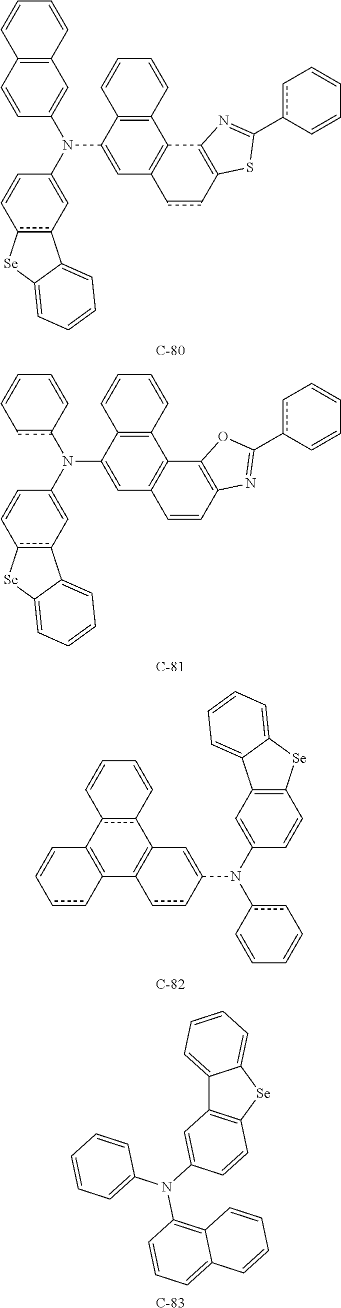

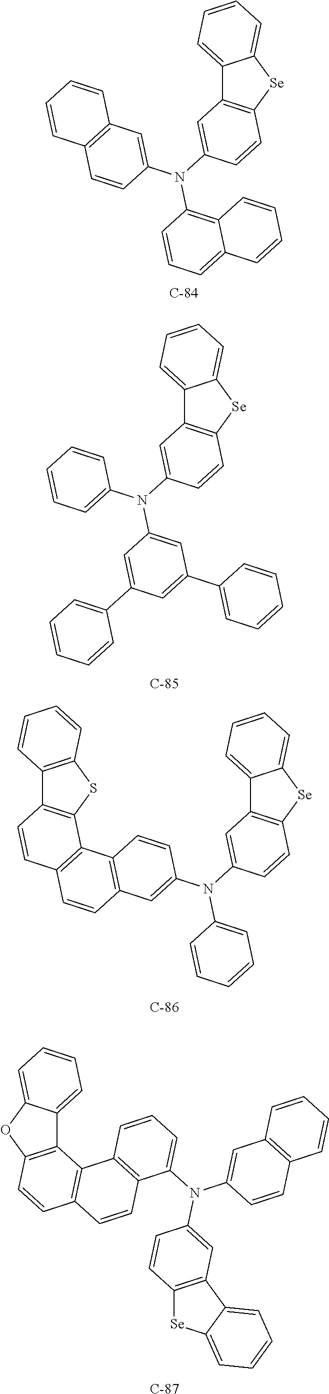

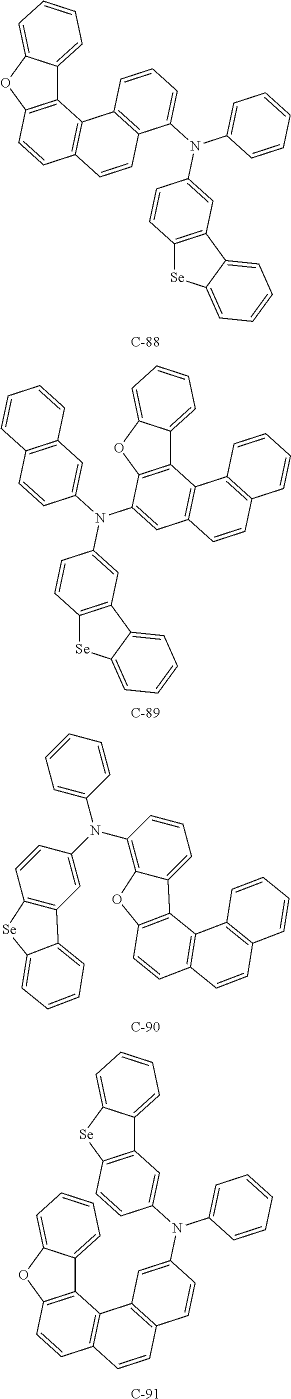

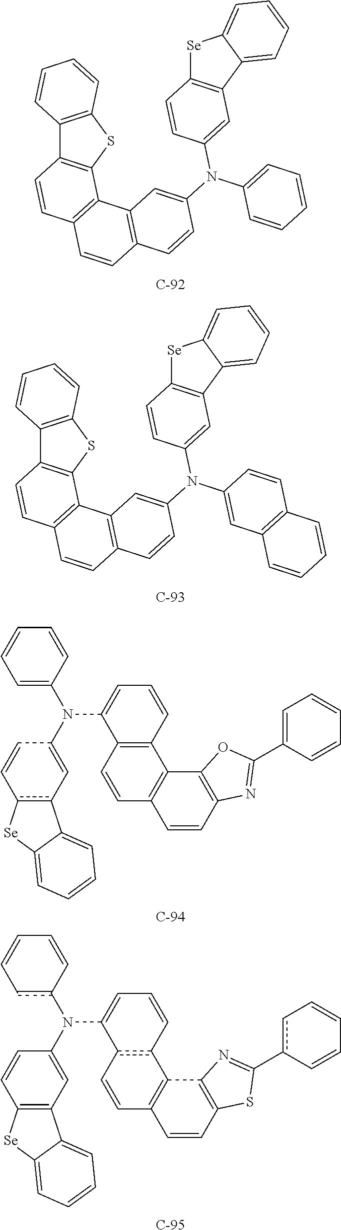

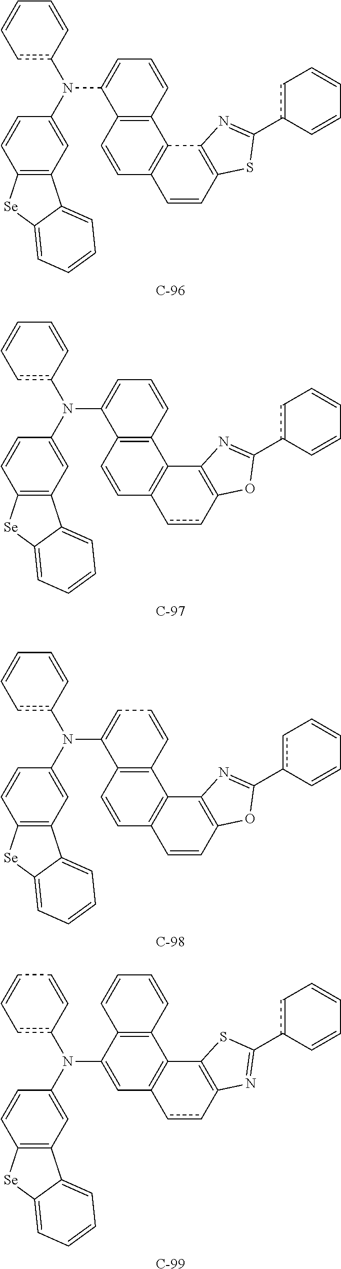

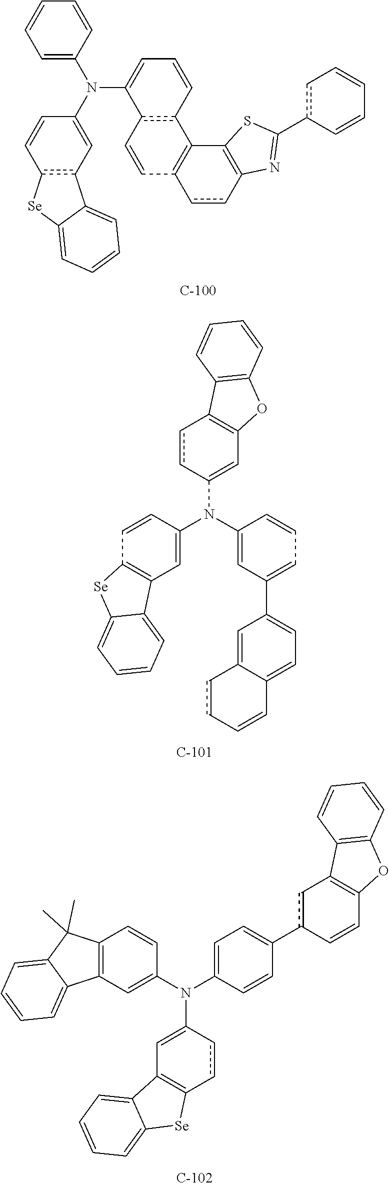

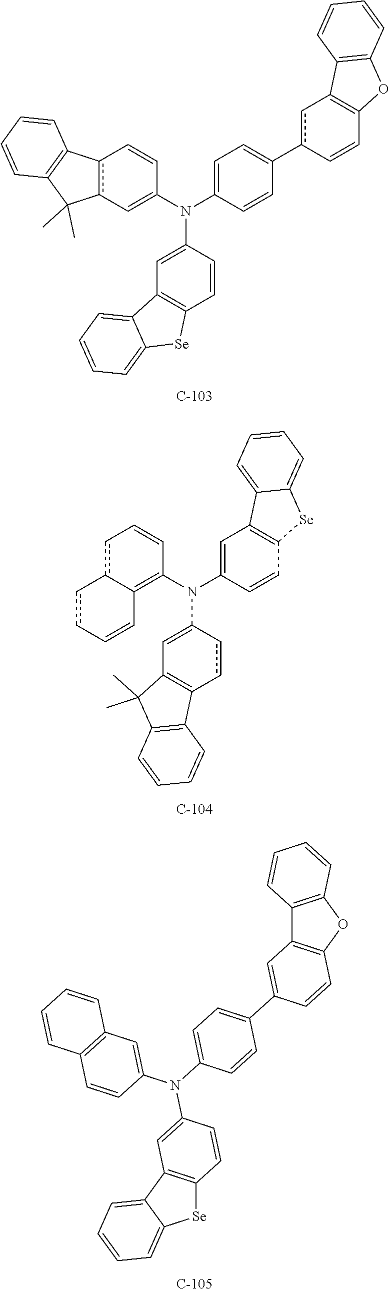

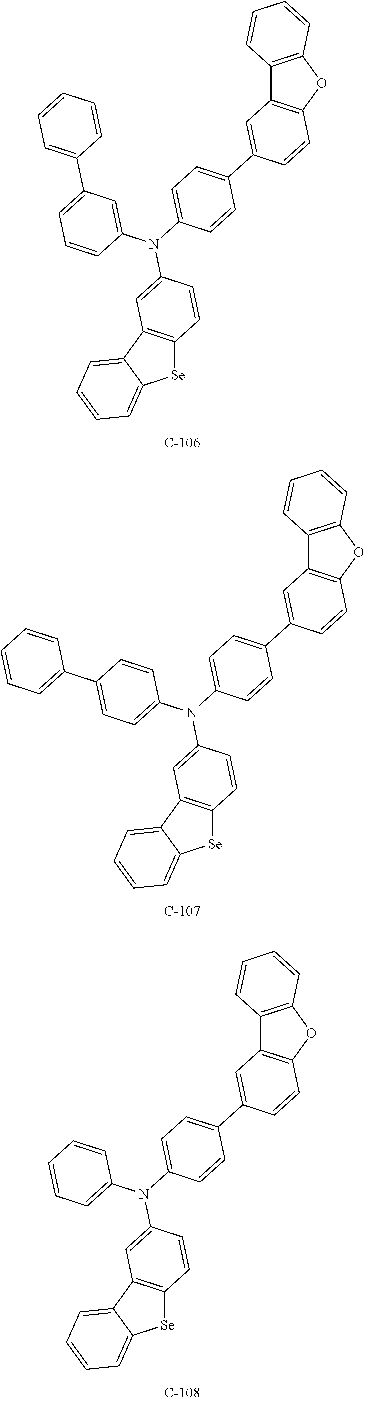 [SMILES: c1ccc(-c2cccc(N(c3ccc(-c4ccc5oc6ccccc6c5c4)cc3)c3ccc4[se]c5ccccc5c4c3)c2)cc1 ; c1ccc(-c2ccc(N(c3ccc(-c4ccc5oc6ccccc6c5c4)cc3)c3ccc4[se]c5ccccc5c4c3)cc2)cc1 ; c1ccc(N(c2ccc(-c3ccc4oc5ccccc5c4c3)cc2)c2ccc3[se]c4ccccc4c3c2)cc1]

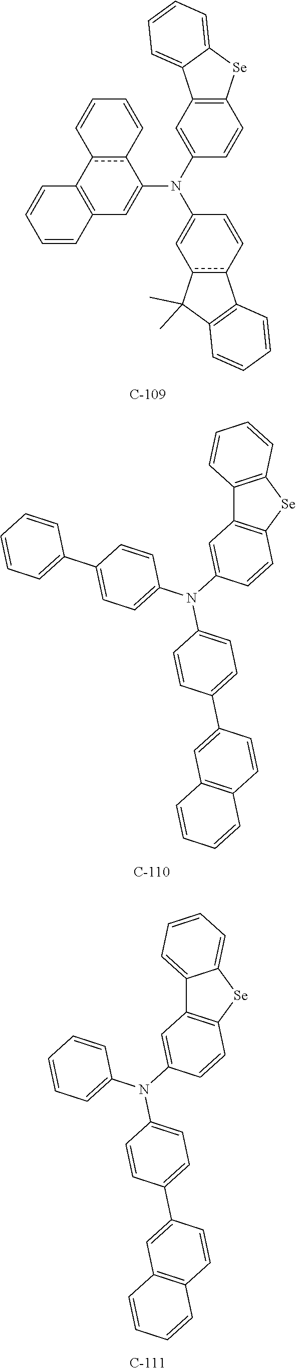

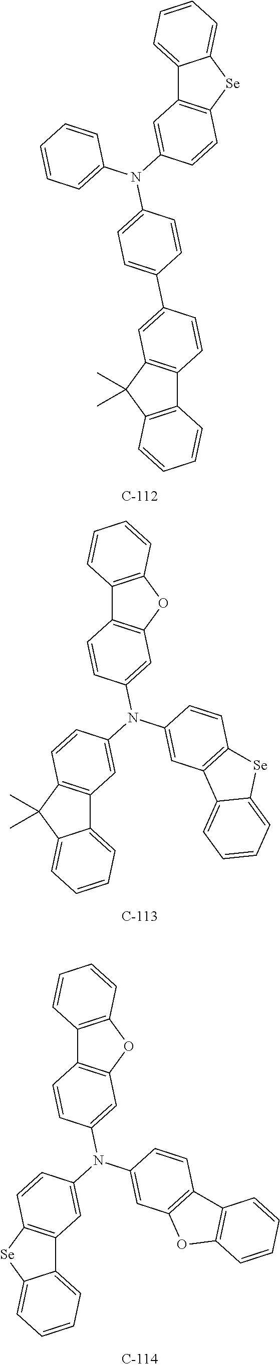 [SMILES: CC1(C)c2ccccc2-c2ccc(-c3ccc(N(c4ccccc4)c4ccc5[se]c6ccccc6c5c4)cc3)cc21 ; CC1(C)c2ccccc2-c2cc(N(c3ccc4c(c3)oc3ccccc34)c3ccc4[se]c5ccccc5c4c3)ccc21 ; c1ccc2c(c1)oc1cc(N(c3ccc4c(c3)oc3ccccc34)c3ccc4[se]c5ccccc5c4c3)ccc12]

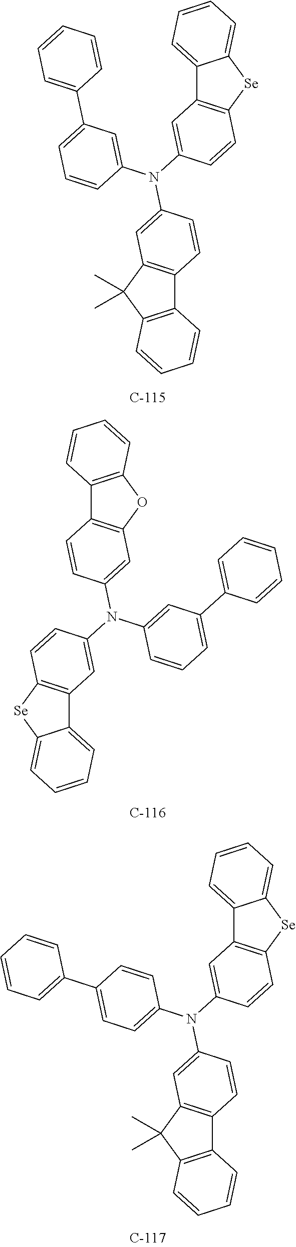 [SMILES: CC1(C)c2ccccc2-c2ccc(N(c3cccc(-c4ccccc4)c3)c3ccc4[se]c5ccccc5c4c3)cc21 ; c1ccc(-c2cccc(N(c3ccc4c(c3)oc3ccccc34)c3ccc4[se]c5ccccc5c4c3)c2)cc1 ; CC1(C)c2ccccc2-c2ccc(N(c3ccc(-c4ccccc4)cc3)c3ccc4[se]c5ccccc5c4c3)cc21]

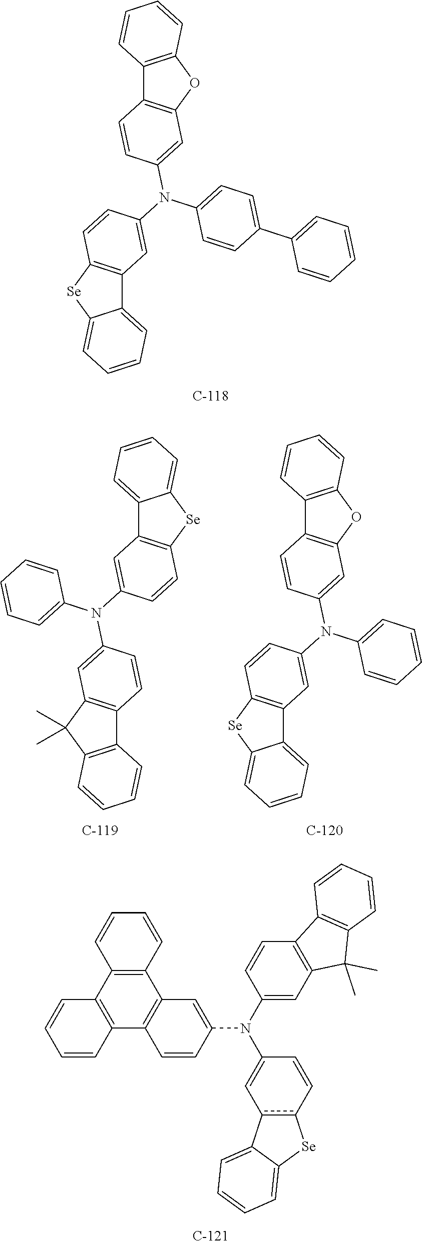

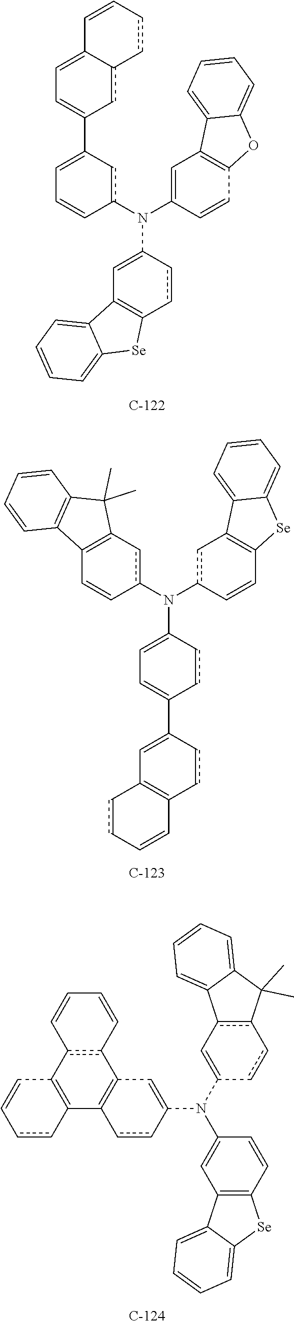

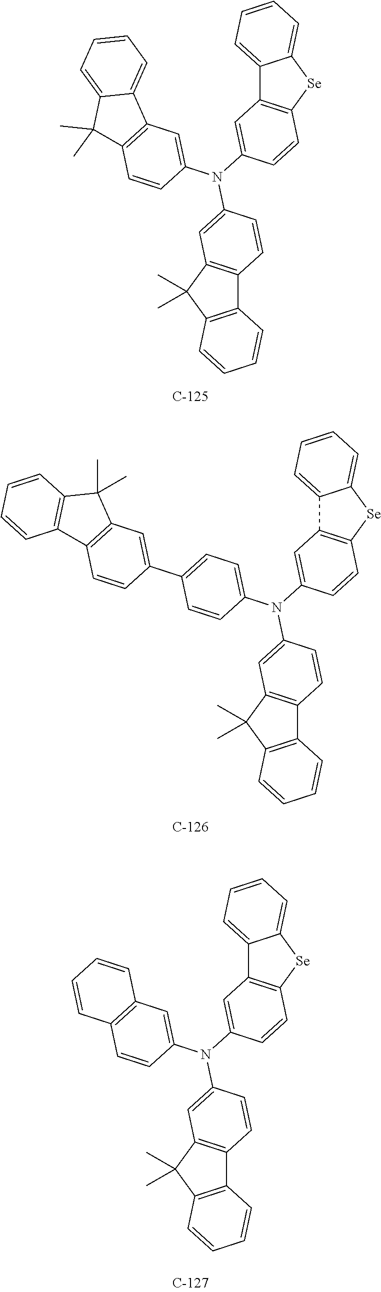

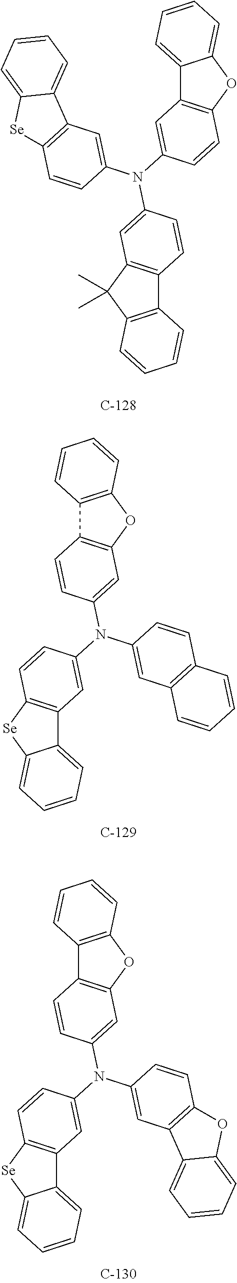

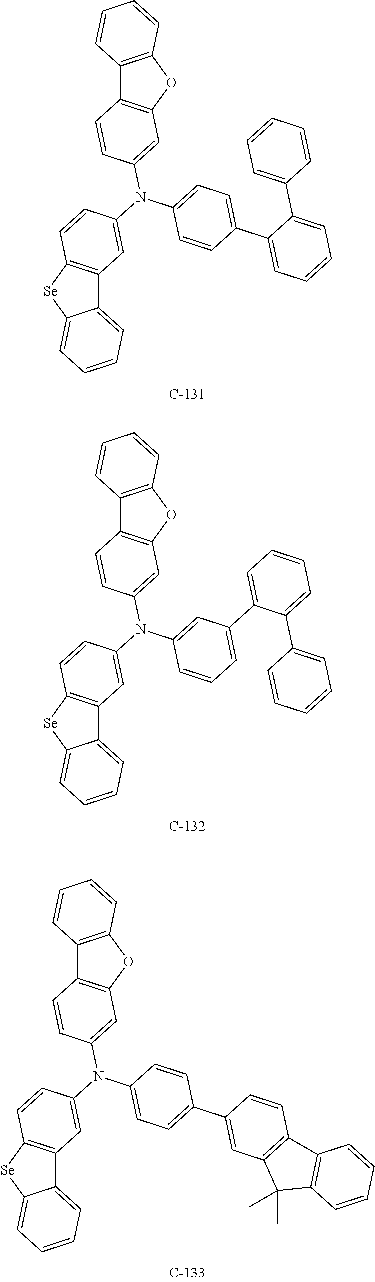 [SMILES: c1ccc(-c2ccccc2-c2ccc(N(c3ccc4c(c3)oc3ccccc34)c3ccc4[se]c5ccccc5c4c3)cc2)cc1 ; c1ccc(-c2ccccc2-c2cccc(N(c3ccc4c(c3)oc3ccccc34)c3ccc4[se]c5ccccc5c4c3)c2)cc1 ; CC1(C)c2ccccc2-c2ccc(-c3ccc(N(c4ccc5c(c4)oc4ccccc45)c4ccc5[se]c6ccccc6c5c4)cc3)cc21]

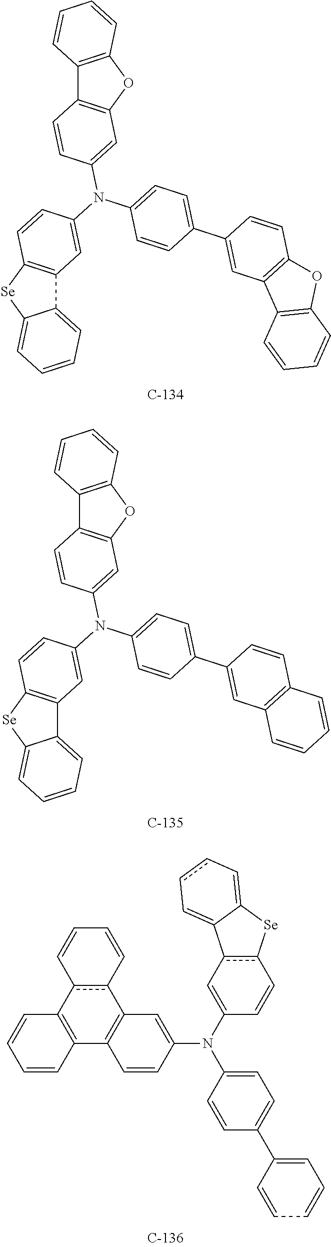

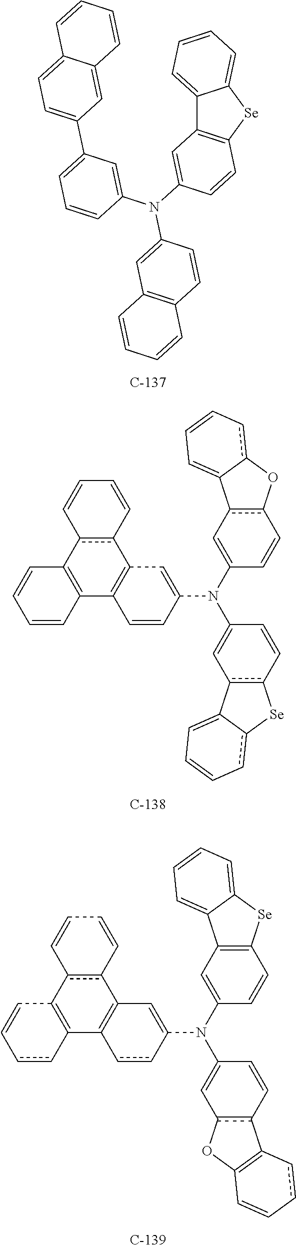

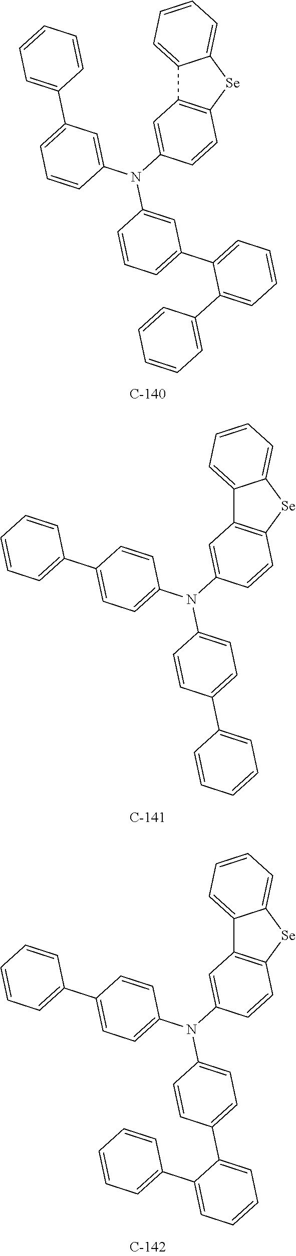

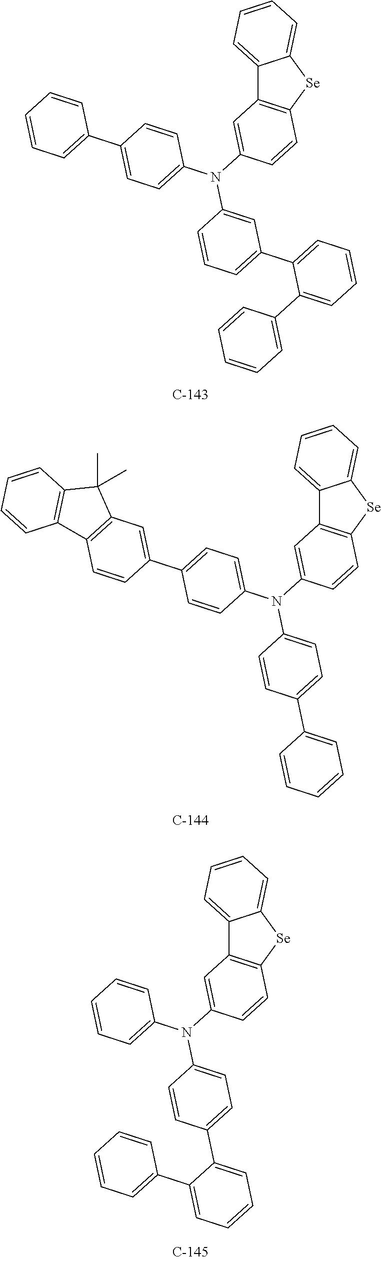 [SMILES: c1ccc(-c2ccc(N(c3cccc(-c4ccccc4-c4ccccc4)c3)c3ccc4[se]c5ccccc5c4c3)cc2)cc1 ; CC1(C)c2ccccc2-c2ccc(-c3ccc(N(c4ccc(-c5ccccc5)cc4)c4ccc5[se]c6ccccc6c5c4)cc3)cc21 ; c1ccc(-c2ccccc2-c2ccc(N(c3ccccc3)c3ccc4[se]c5ccccc5c4c3)cc2)cc1]

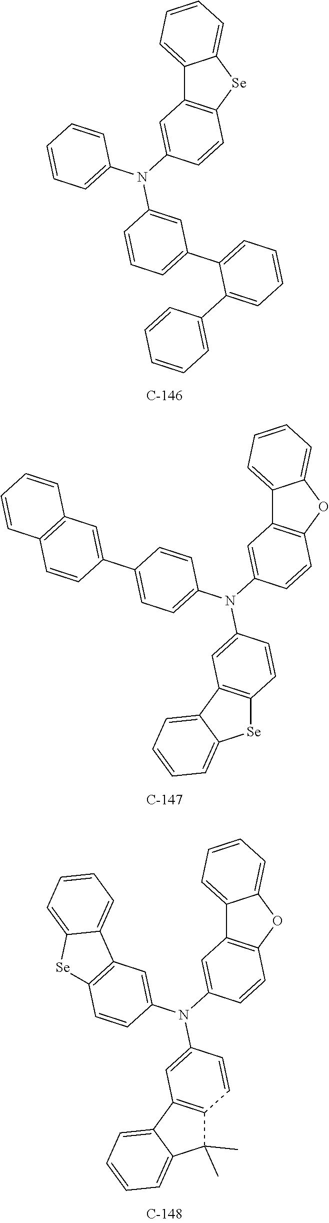

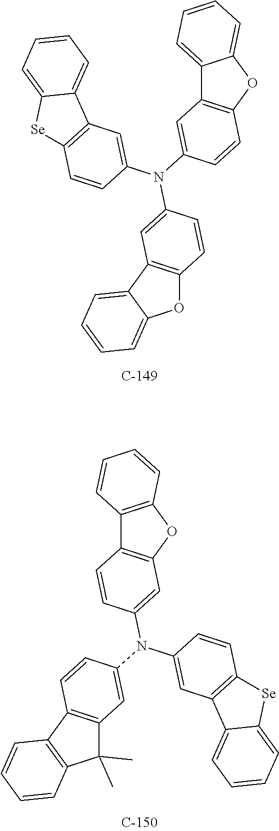

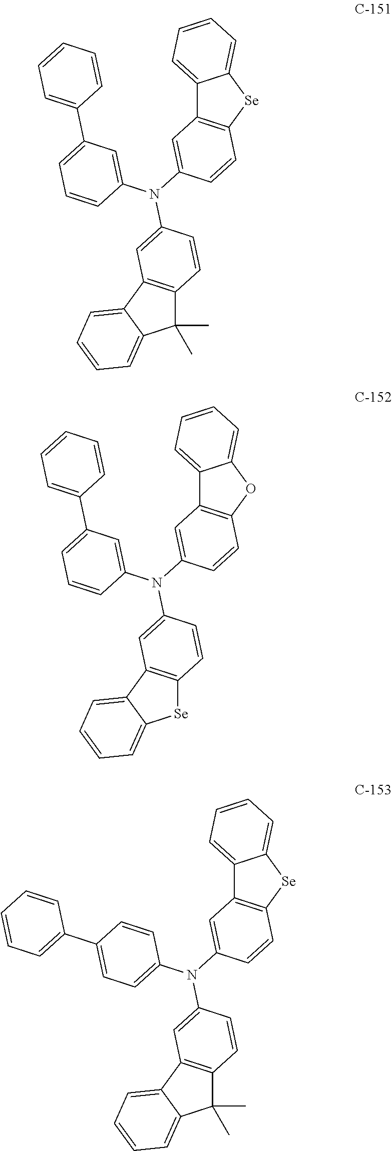 [SMILES: CC1(C)c2ccccc2-c2cc(N(c3cccc(-c4ccccc4)c3)c3ccc4[se]c5ccccc5c4c3)ccc21 ; c1ccc(-c2cccc(N(c3ccc4oc5ccccc5c4c3)c3ccc4[se]c5ccccc5c4c3)c2)cc1 ; CC1(C)c2ccccc2-c2cc(N(c3ccc(-c4ccccc4)cc3)c3ccc4[se]c5ccccc5c4c3)ccc21]

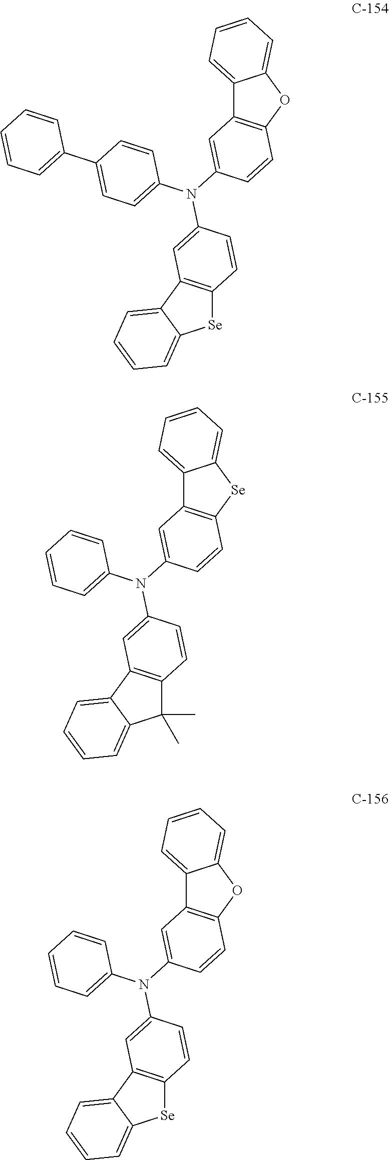 [SMILES: c1ccc(-c2ccc(N(c3ccc4oc5ccccc5c4c3)c3ccc4[se]c5ccccc5c4c3)cc2)cc1 ; CC1(C)c2ccccc2-c2cc(N(c3ccccc3)c3ccc4[se]c5ccccc5c4c3)ccc21 ; c1ccc(N(c2ccc3oc4ccccc4c3c2)c2ccc3[se]c4ccccc4c3c2)cc1]

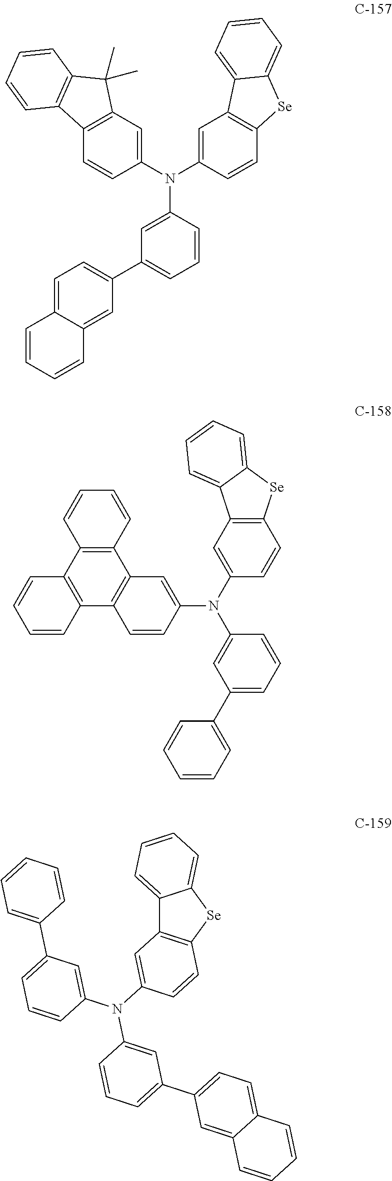 [SMILES: CC1(C)c2ccccc2-c2ccc(N(c3cccc(-c4ccc5ccccc5c4)c3)c3ccc4[se]c5ccccc5c4c3)cc21 ; c1ccc(-c2cccc(N(c3ccc4[se]c5ccccc5c4c3)c3ccc4c5ccccc5c5ccccc5c4c3)c2)cc1 ; c1ccc(-c2cccc(N(c3cccc(-c4ccc5ccccc5c4)c3)c3ccc4[se]c5ccccc5c4c3)c2)cc1]

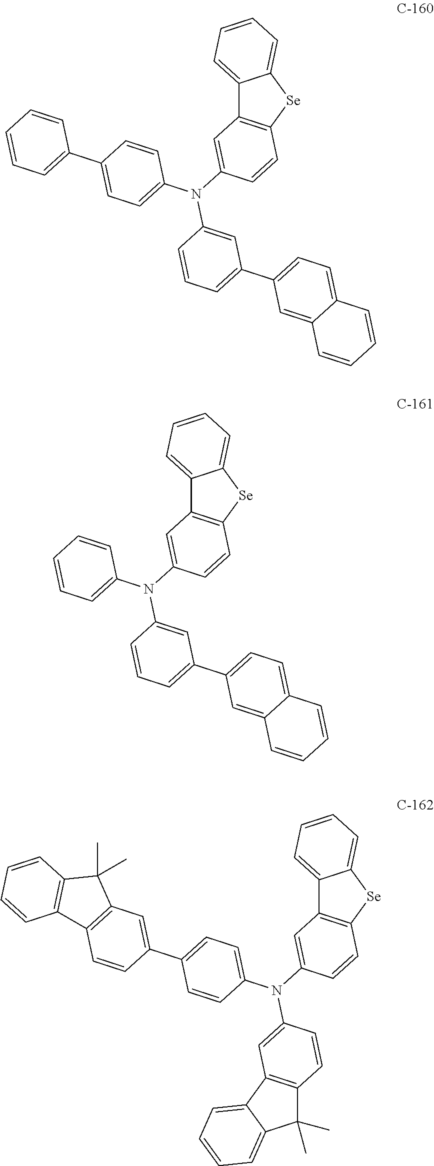 [SMILES: c1ccc(-c2ccc(N(c3cccc(-c4ccc5ccccc5c4)c3)c3ccc4[se]c5ccccc5c4c3)cc2)cc1 ; c1ccc(N(c2cccc(-c3ccc4ccccc4c3)c2)c2ccc3[se]c4ccccc4c3c2)cc1 ; CC1(C)c2ccccc2-c2cc(N(c3ccc(-c4ccc5c(c4)C(C)(C)c4ccccc4-5)cc3)c3ccc4[se]c5ccccc5c4c3)ccc21]

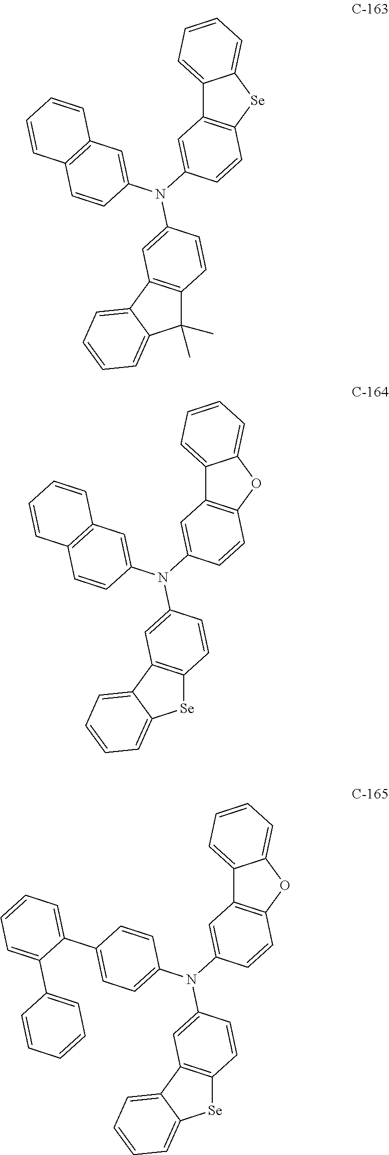 [SMILES: CC1(C)c2ccccc2-c2cc(N(c3ccc4ccccc4c3)c3ccc4[se]c5ccccc5c4c3)ccc21 ; c1ccc2cc(N(c3ccc4oc5ccccc5c4c3)c3ccc4[se]c5ccccc5c4c3)ccc2c1 ; c1ccc(-c2ccccc2-c2ccc(N(c3ccc4oc5ccccc5c4c3)c3ccc4[se]c5ccccc5c4c3)cc2)cc1]

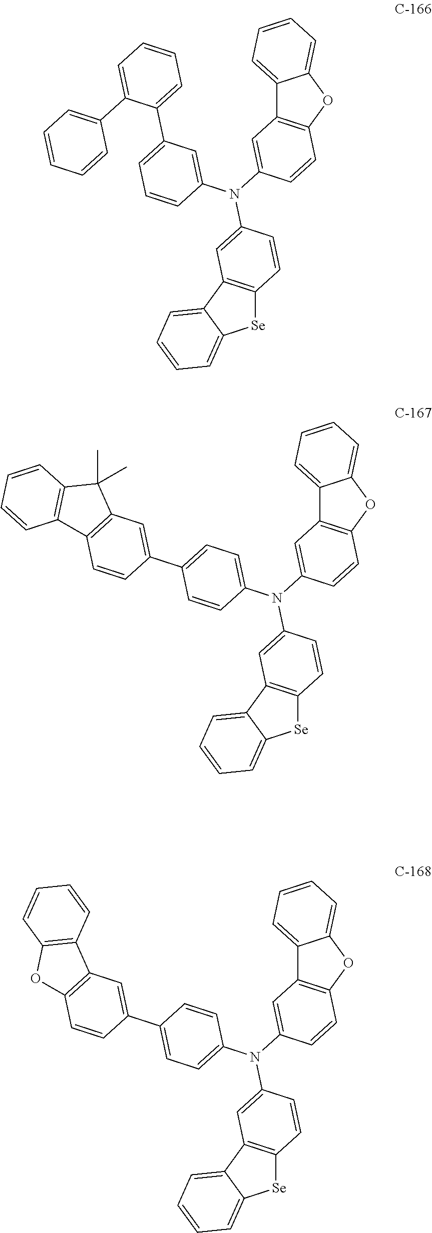 [SMILES: c1ccc(-c2ccccc2-c2cccc(N(c3ccc4oc5ccccc5c4c3)c3ccc4[se]c5ccccc5c4c3)c2)cc1 ; CC1(C)c2ccccc2-c2ccc(-c3ccc(N(c4ccc5oc6ccccc6c5c4)c4ccc5[se]c6ccccc6c5c4)cc3)cc21 ; c1ccc2c(c1)oc1ccc(-c3ccc(N(c4ccc5oc6ccccc6c5c4)c4ccc5[se]c6ccccc6c5c4)cc3)cc12]

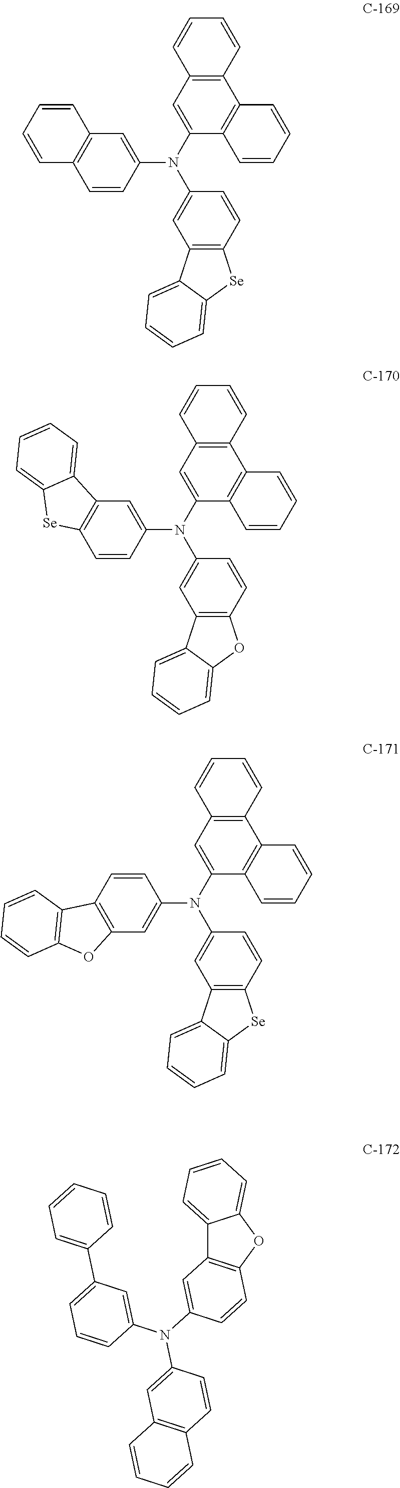

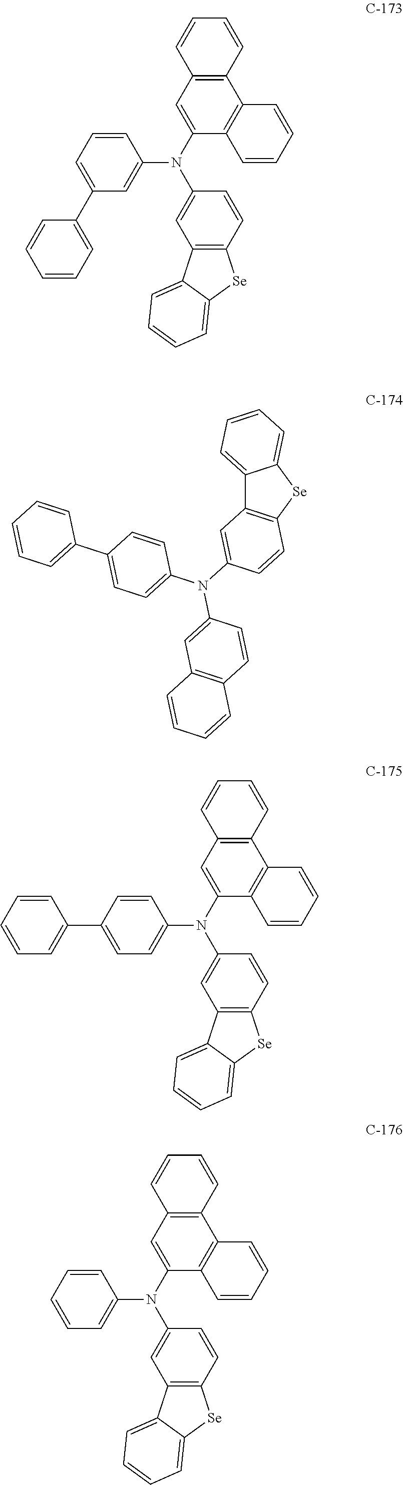

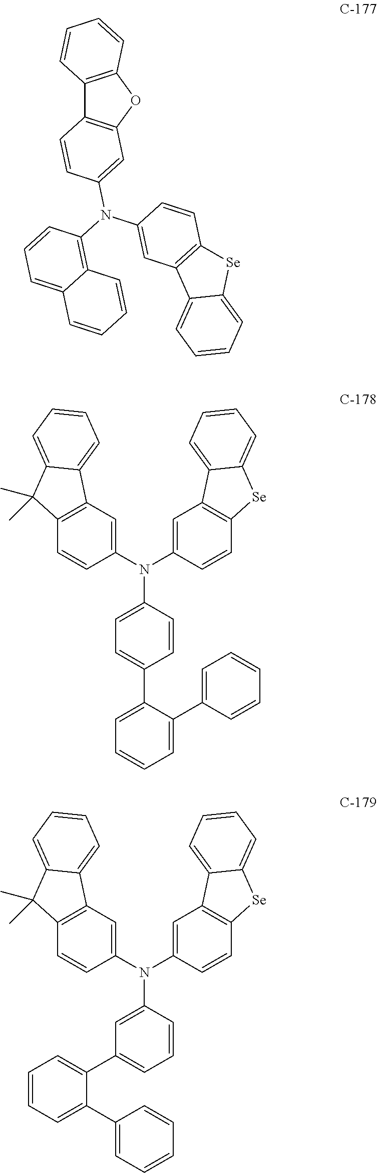 [SMILES: c1ccc2c(N(c3ccc4c(c3)oc3ccccc34)c3ccc4[se]c5ccccc5c4c3)cccc2c1 ; CC1(C)c2ccccc2-c2cc(N(c3ccc(-c4ccccc4-c4ccccc4)cc3)c3ccc4[se]c5ccccc5c4c3)ccc21 ; CC1(C)c2ccccc2-c2cc(N(c3cccc(-c4ccccc4-c4ccccc4)c3)c3ccc4[se]c5ccccc5c4c3)ccc21]

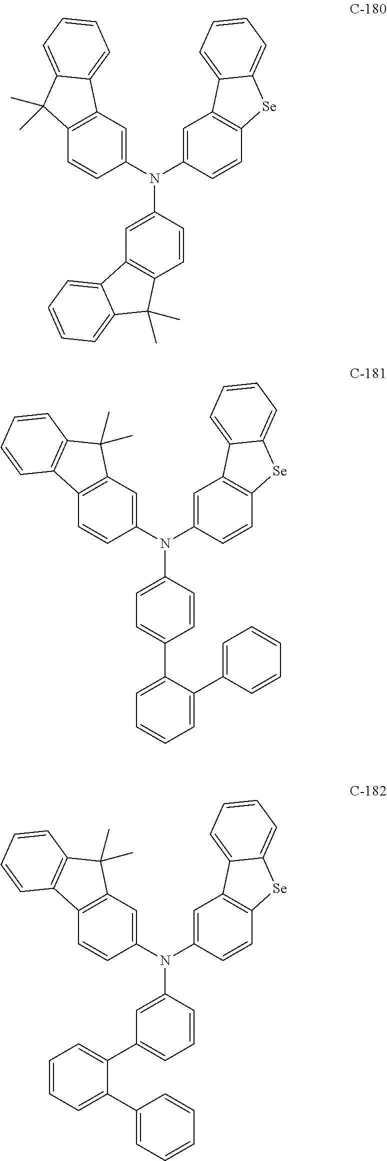 [SMILES: CC1(C)c2ccccc2-c2cc(N(c3ccc4c(c3)-c3ccccc3C4(C)C)c3ccc4[se]c5ccccc5c4c3)ccc21 ; CC1(C)c2ccccc2-c2ccc(N(c3ccc(-c4ccccc4-c4ccccc4)cc3)c3ccc4[se]c5ccccc5c4c3)cc21 ; CC1(C)c2ccccc2-c2ccc(N(c3cccc(-c4ccccc4-c4ccccc4)c3)c3ccc4[se]c5ccccc5c4c3)cc21]

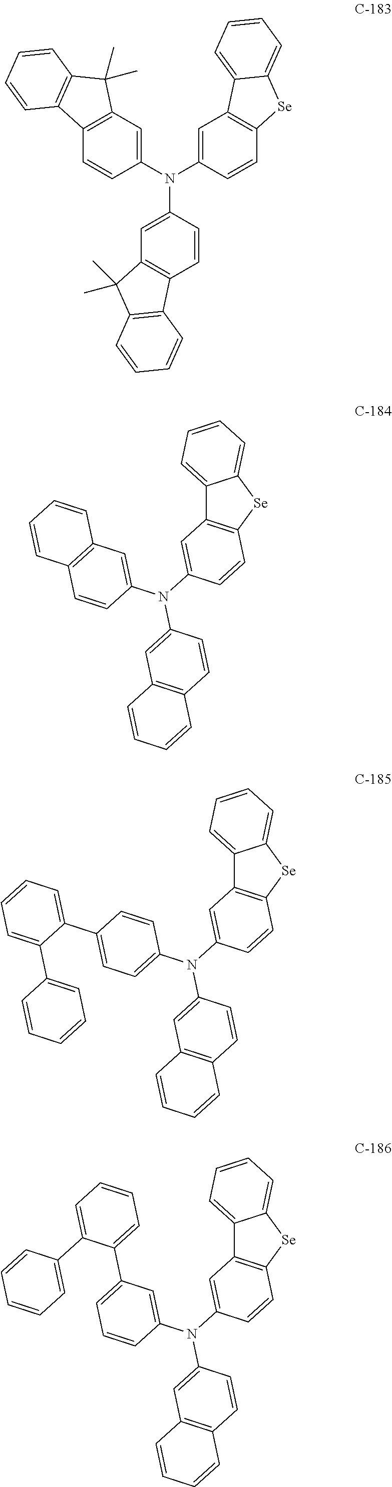 [SMILES: CC1(C)c2ccccc2-c2ccc(N(c3ccc4c(c3)C(C)(C)c3ccccc3-4)c3ccc4[se]c5ccccc5c4c3)cc21 ; c1ccc2cc(N(c3ccc4ccccc4c3)c3ccc4[se]c5ccccc5c4c3)ccc2c1 ; c1ccc(-c2ccccc2-c2ccc(N(c3ccc4ccccc4c3)c3ccc4[se]c5ccccc5c4c3)cc2)cc1 ; c1ccc(-c2ccccc2-c2cccc(N(c3ccc4ccccc4c3)c3ccc4[se]c5ccccc5c4c3)c2)cc1]

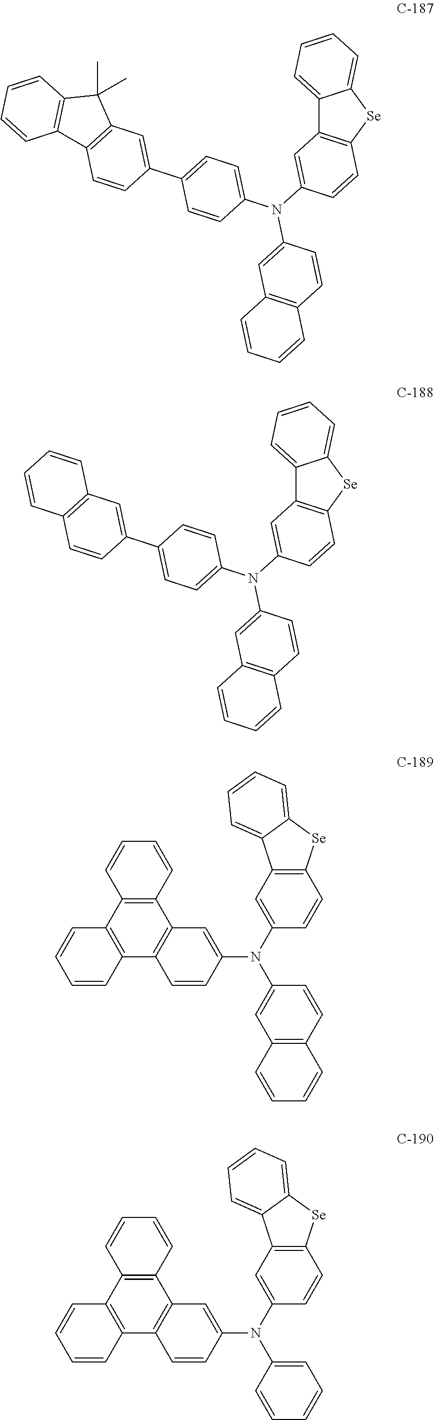

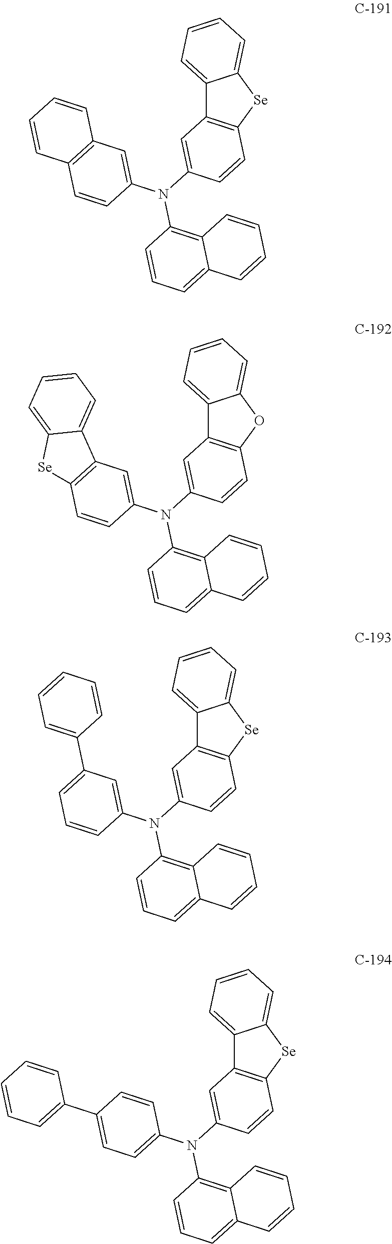

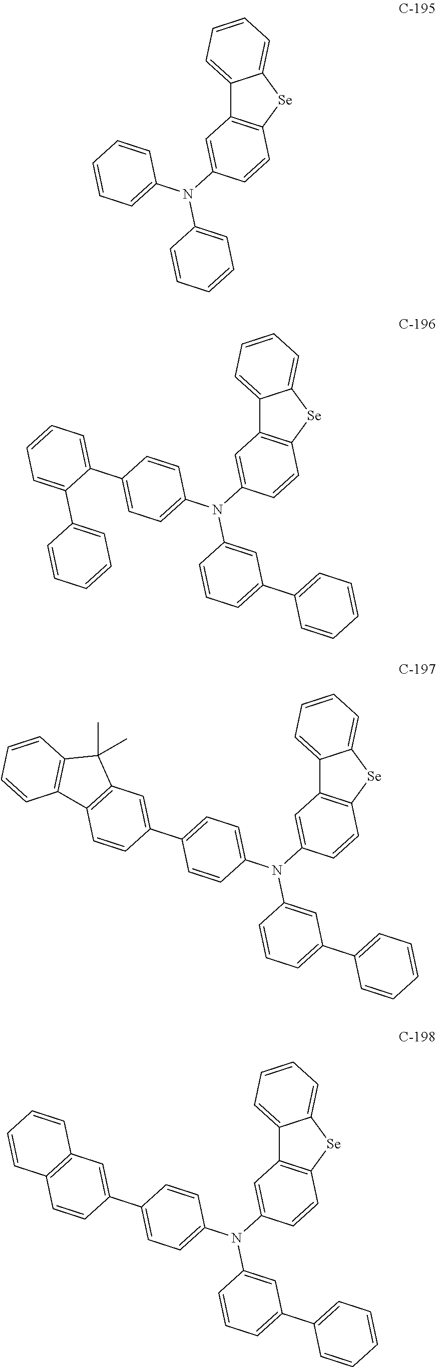 [SMILES: c1ccc(N(c2ccccc2)c2ccc3[se]c4ccccc4c3c2)cc1 ; c1ccc(-c2cccc(N(c3ccc(-c4ccccc4-c4ccccc4)cc3)c3ccc4[se]c5ccccc5c4c3)c2)cc1 ; CC1(C)c2ccccc2-c2ccc(-c3ccc(N(c4cccc(-c5ccccc5)c4)c4ccc5[se]c6ccccc6c5c4)cc3)cc21 ; c1ccc(-c2cccc(N(c3ccc(-c4ccc5ccccc5c4)cc3)c3ccc4[se]c5ccccc5c4c3)c2)cc1]

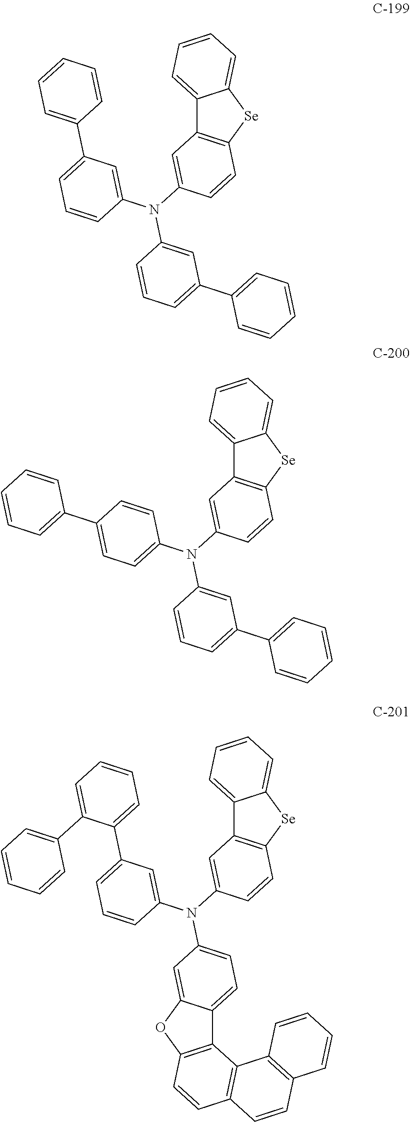

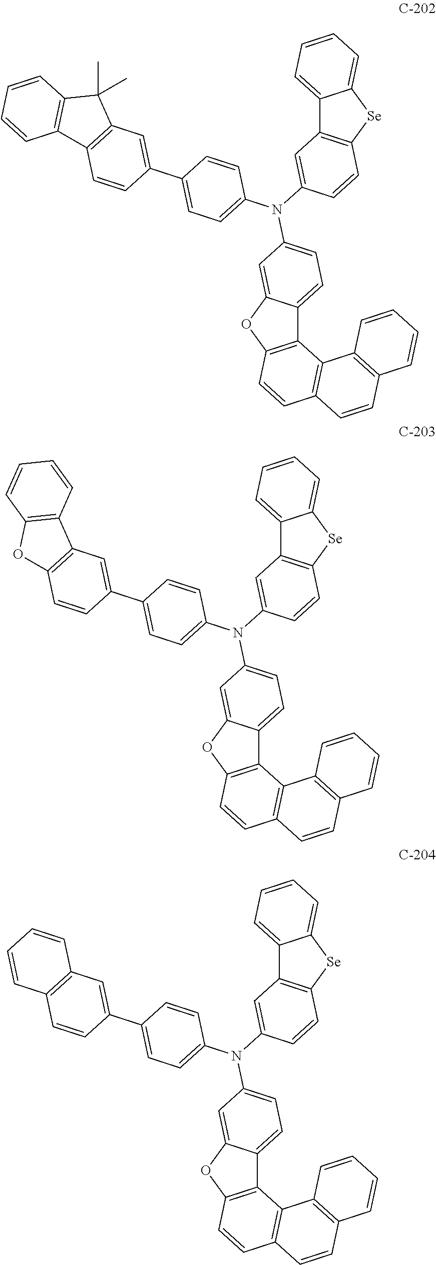

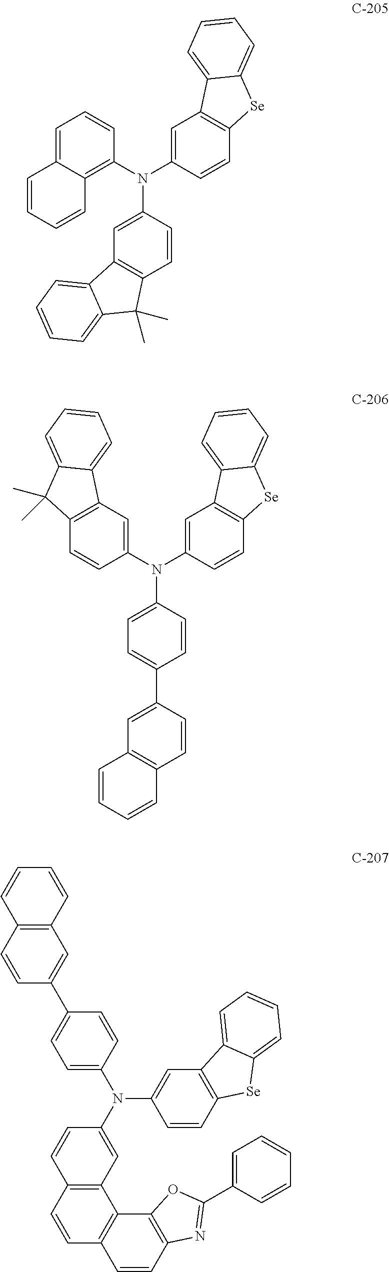 [SMILES: CC1(C)c2ccccc2-c2cc(N(c3ccc4[se]c5ccccc5c4c3)c3cccc4ccccc34)ccc21 ; CC1(C)c2ccccc2-c2cc(N(c3ccc(-c4ccc5ccccc5c4)cc3)c3ccc4[se]c5ccccc5c4c3)ccc21 ; c1ccc(-c2nc3ccc4ccc5ccc(N(c6ccc(-c7ccc8ccccc8c7)cc6)c6ccc7[se]c8ccccc8c7c6)cc5c4c3o2)cc1]

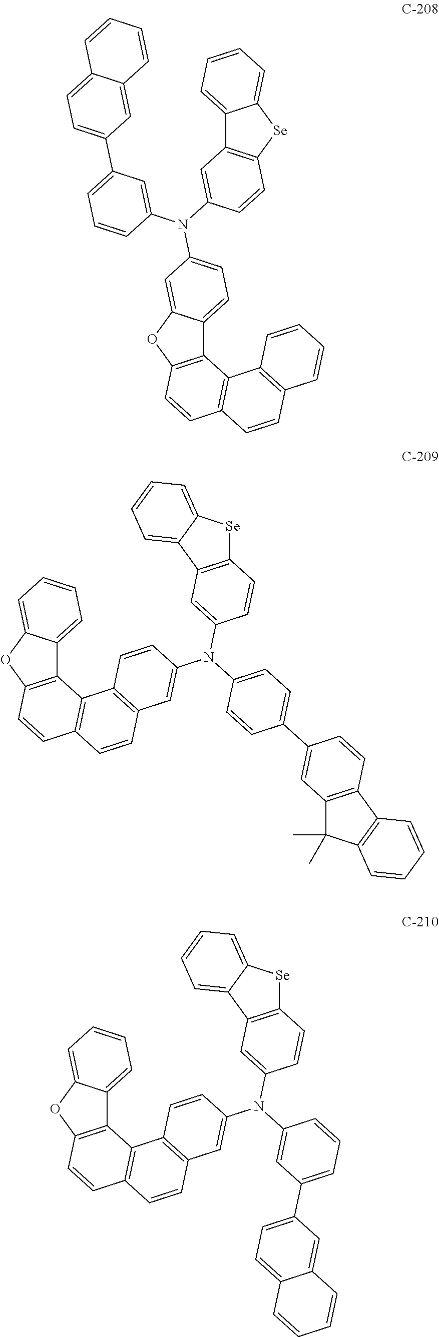 [SMILES: c1cc(-c2ccc3ccccc3c2)cc(N(c2ccc3c(c2)oc2ccc4ccc5ccccc5c4c23)c2ccc3[se]c4ccccc4c3c2)c1 ; CC1(C)c2ccccc2-c2ccc(-c3ccc(N(c4ccc5c(ccc6ccc7oc8ccccc8c7c65)c4)c4ccc5[se]c6ccccc6c5c4)cc3)cc21 ; c1cc(-c2ccc3ccccc3c2)cc(N(c2ccc3c(ccc4ccc5oc6ccccc6c5c43)c2)c2ccc3[se]c4ccccc4c3c2)c1]

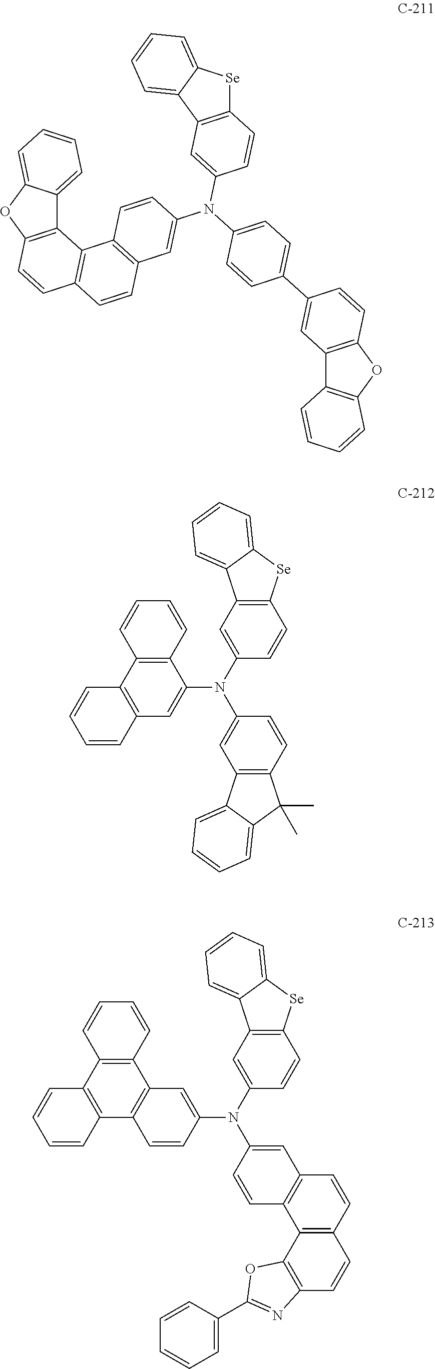 [SMILES: c1ccc2c(c1)oc1ccc(-c3ccc(N(c4ccc5c(ccc6ccc7oc8ccccc8c7c65)c4)c4ccc5[se]c6ccccc6c5c4)cc3)cc12 ; CC1(C)c2ccccc2-c2cc(N(c3ccc4[se]c5ccccc5c4c3)c3cc4ccccc4c4ccccc34)ccc21 ; c1ccc(-c2nc3ccc4ccc5cc(N(c6ccc7[se]c8ccccc8c7c6)c6ccc7c8ccccc8c8ccccc8c7c6)ccc5c4c3o2)cc1]

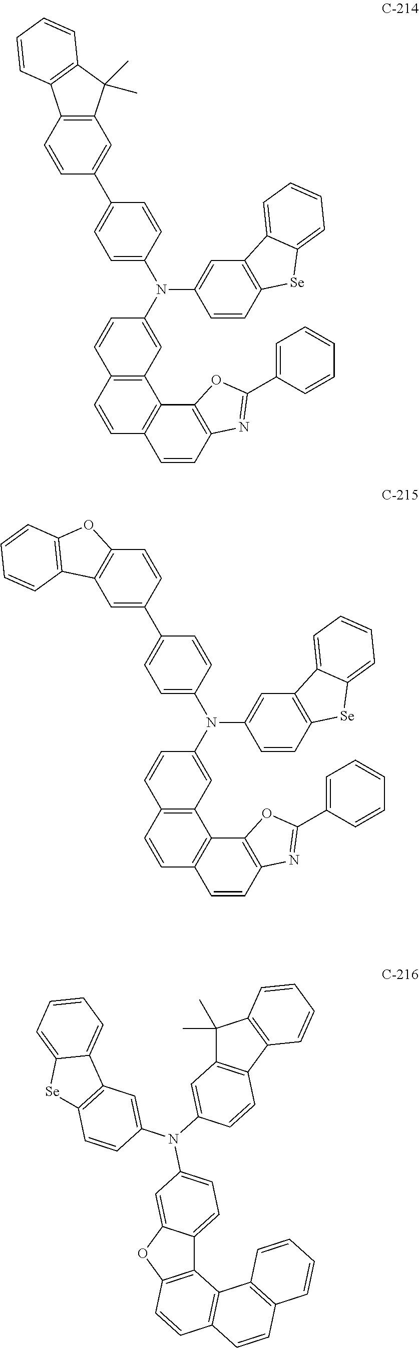 [SMILES: CC1(C)c2ccccc2-c2ccc(-c3ccc(N(c4ccc5[se]c6ccccc6c5c4)c4ccc5ccc6ccc7nc(-c8ccccc8)oc7c6c5c4)cc3)cc21 ; c1ccc(-c2nc3ccc4ccc5ccc(N(c6ccc(-c7ccc8oc9ccccc9c8c7)cc6)c6ccc7[se]c8ccccc8c7c6)cc5c4c3o2)cc1 ; CC1(C)c2ccccc2-c2ccc(N(c3ccc4c(c3)oc3ccc5ccc6ccccc6c5c34)c3ccc4[se]c5ccccc5c4c3)cc21]

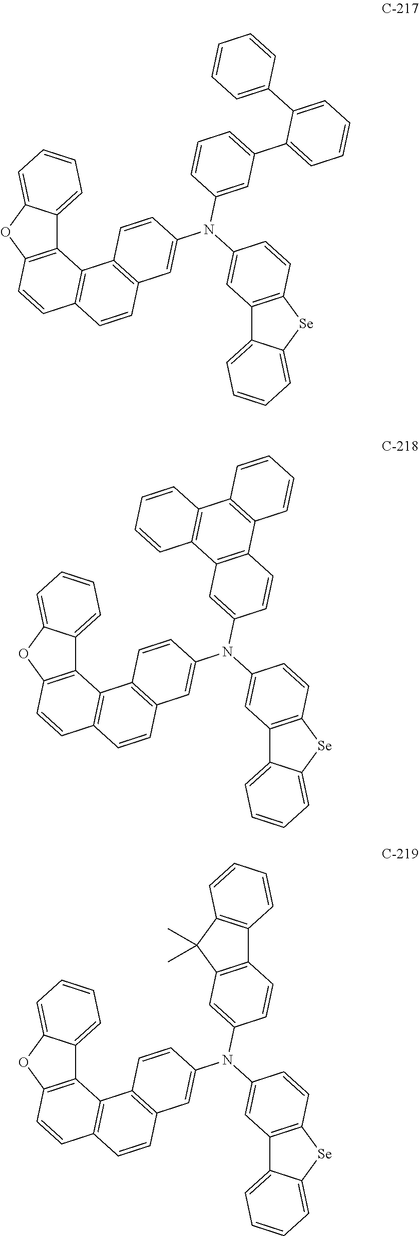 [SMILES: c1ccc(-c2ccccc2-c2cccc(N(c3ccc4c(ccc5ccc6oc7ccccc7c6c54)c3)c3ccc4[se]c5ccccc5c4c3)c2)cc1 ; c1ccc2c(c1)oc1ccc3ccc4cc(N(c5ccc6[se]c7ccccc7c6c5)c5ccc6c7ccccc7c7ccccc7c6c5)ccc4c3c12 ; CC1(C)c2ccccc2-c2ccc(N(c3ccc4c(ccc5ccc6oc7ccccc7c6c54)c3)c3ccc4[se]c5ccccc5c4c3)cc21]

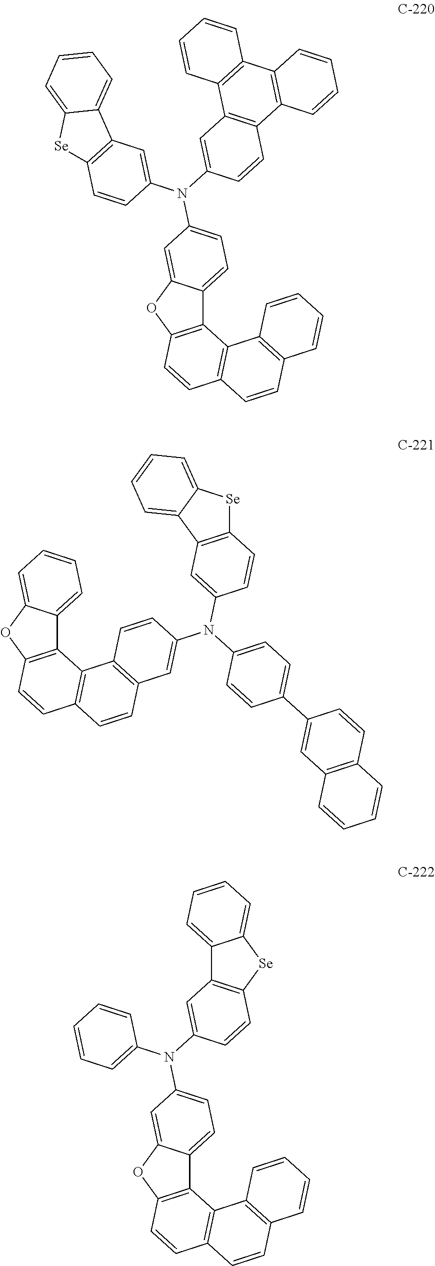 [SMILES: c1ccc2c(c1)ccc1ccc3oc4cc(N(c5ccc6[se]c7ccccc7c6c5)c5ccc6c7ccccc7c7ccccc7c6c5)ccc4c3c12 ; c1ccc2cc(-c3ccc(N(c4ccc5c(ccc6ccc7oc8ccccc8c7c65)c4)c4ccc5[se]c6ccccc6c5c4)cc3)ccc2c1 ; c1ccc(N(c2ccc3c(c2)oc2ccc4ccc5ccccc5c4c23)c2ccc3[se]c4ccccc4c3c2)cc1]

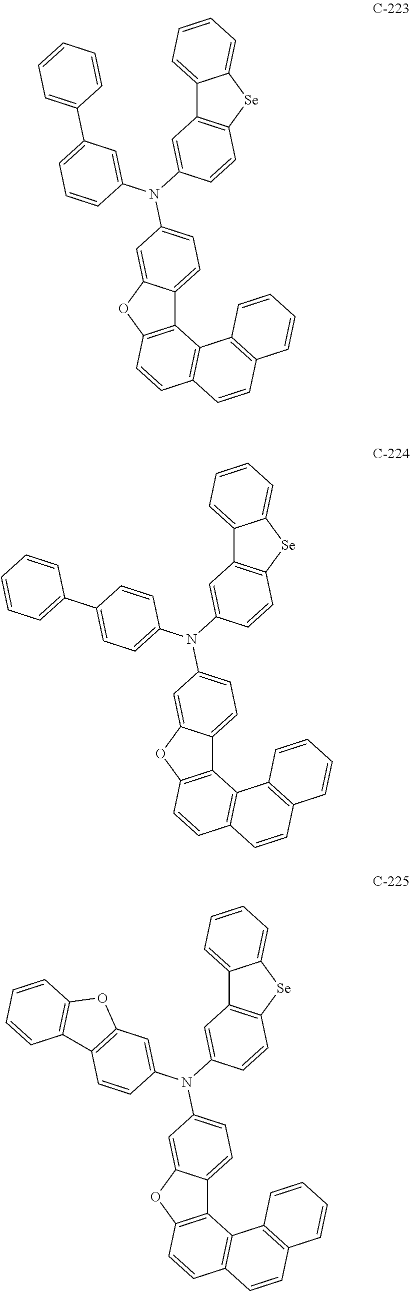 [SMILES: c1ccc(-c2cccc(N(c3ccc4c(c3)oc3ccc5ccc6ccccc6c5c34)c3ccc4[se]c5ccccc5c4c3)c2)cc1 ; c1ccc(-c2ccc(N(c3ccc4c(c3)oc3ccc5ccc6ccccc6c5c34)c3ccc4[se]c5ccccc5c4c3)cc2)cc1 ; c1ccc2c(c1)ccc1ccc3oc4cc(N(c5ccc6c(c5)oc5ccccc56)c5ccc6[se]c7ccccc7c6c5)ccc4c3c12]

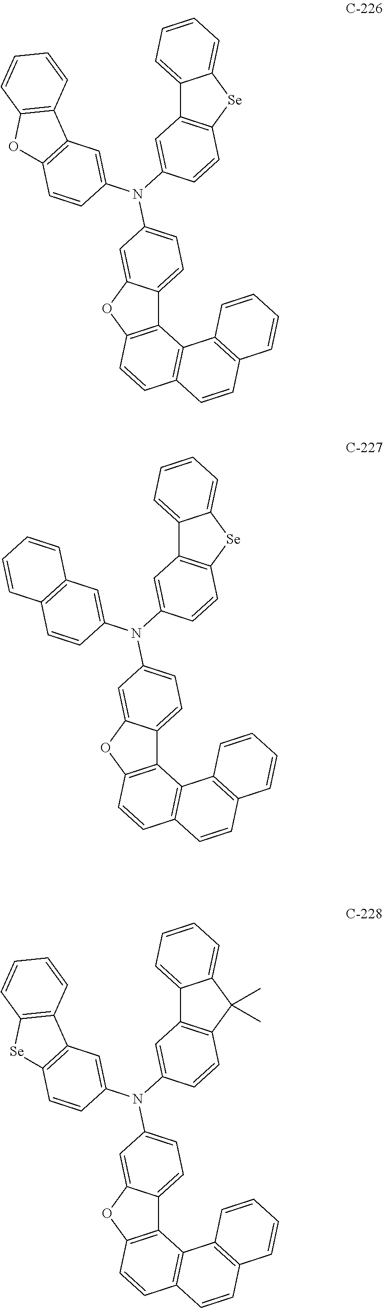 [SMILES: c1ccc2c(c1)ccc1ccc3oc4cc(N(c5ccc6oc7ccccc7c6c5)c5ccc6[se]c7ccccc7c6c5)ccc4c3c12 ; c1ccc2cc(N(c3ccc4c(c3)oc3ccc5ccc6ccccc6c5c34)c3ccc4[se]c5ccccc5c4c3)ccc2c1 ; CC1(C)c2ccccc2-c2cc(N(c3ccc4c(c3)oc3ccc5ccc6ccccc6c5c34)c3ccc4[se]c5ccccc5c4c3)ccc21]

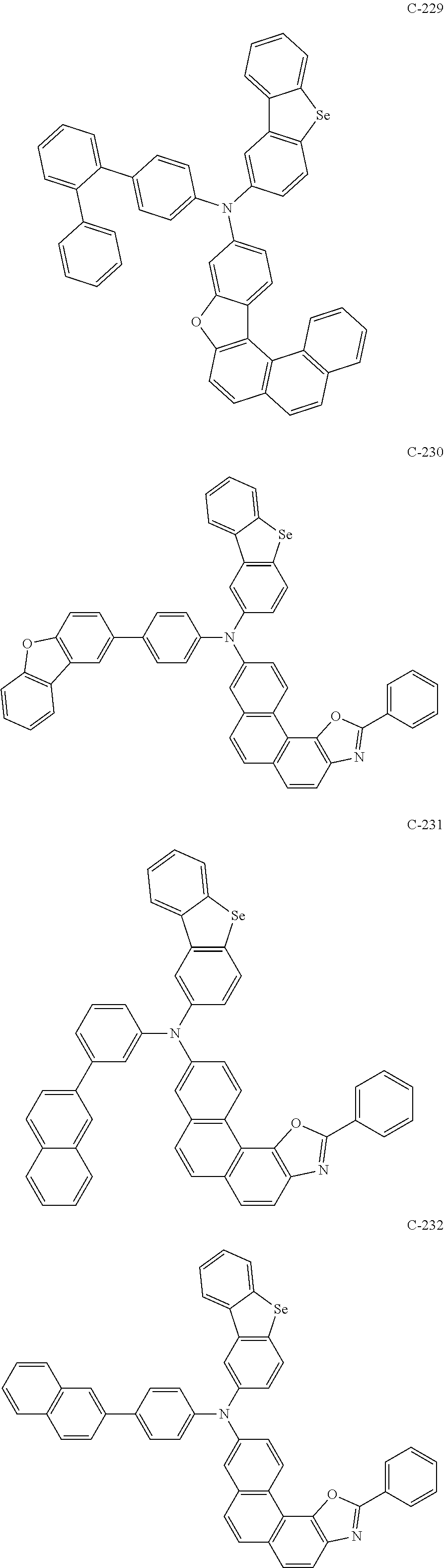 [SMILES: c1ccc(-c2ccccc2-c2ccc(N(c3ccc4c(c3)oc3ccc5ccc6ccccc6c5c34)c3ccc4[se]c5ccccc5c4c3)cc2)cc1 ; c1ccc(-c2nc3ccc4ccc5cc(N(c6ccc(-c7ccc8oc9ccccc9c8c7)cc6)c6ccc7[se]c8ccccc8c7c6)ccc5c4c3o2)cc1 ; c1ccc(-c2nc3ccc4ccc5cc(N(c6cccc(-c7ccc8ccccc8c7)c6)c6ccc7[se]c8ccccc8c7c6)ccc5c4c3o2)cc1 ; c1ccc(-c2nc3ccc4ccc5cc(N(c6ccc(-c7ccc8ccccc8c7)cc6)c6ccc7[se]c8ccccc8c7c6)ccc5c4c3o2)cc1]

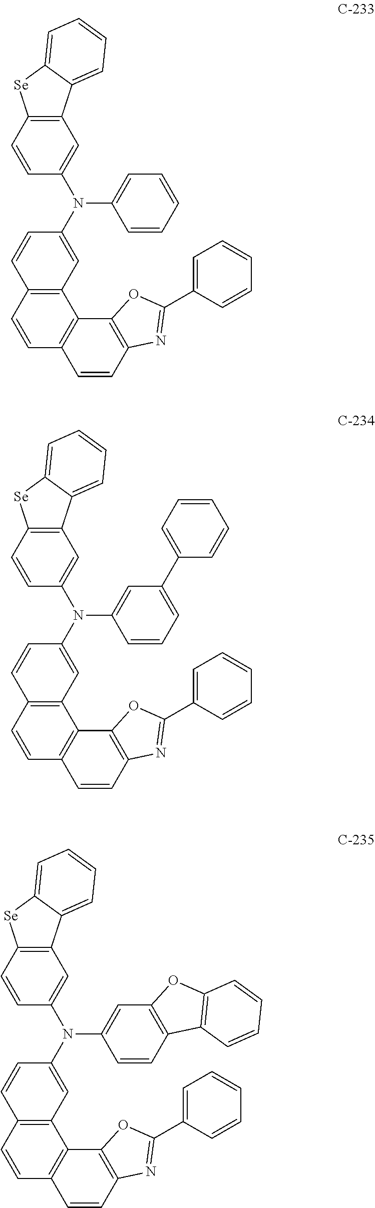 [SMILES: c1ccc(-c2nc3ccc4ccc5ccc(N(c6ccccc6)c6ccc7[se]c8ccccc8c7c6)cc5c4c3o2)cc1 ; c1ccc(-c2cccc(N(c3ccc4[se]c5ccccc5c4c3)c3ccc4ccc5ccc6nc(-c7ccccc7)oc6c5c4c3)c2)cc1 ; c1ccc(-c2nc3ccc4ccc5ccc(N(c6ccc7c(c6)oc6ccccc67)c6ccc7[se]c8ccccc8c7c6)cc5c4c3o2)cc1]

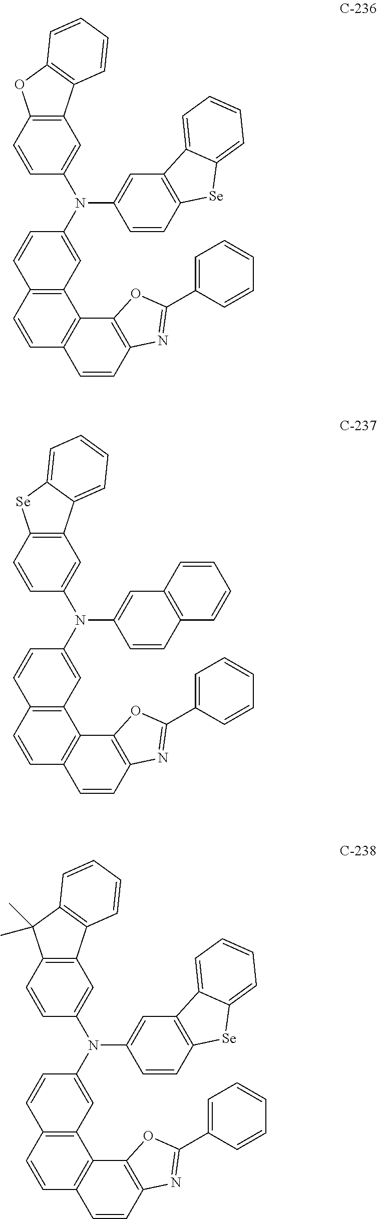 [SMILES: c1ccc(-c2nc3ccc4ccc5ccc(N(c6ccc7oc8ccccc8c7c6)c6ccc7[se]c8ccccc8c7c6)cc5c4c3o2)cc1 ; c1ccc(-c2nc3ccc4ccc5ccc(N(c6ccc7ccccc7c6)c6ccc7[se]c8ccccc8c7c6)cc5c4c3o2)cc1 ; CC1(C)c2ccccc2-c2cc(N(c3ccc4[se]c5ccccc5c4c3)c3ccc4ccc5ccc6nc(-c7ccccc7)oc6c5c4c3)ccc21]

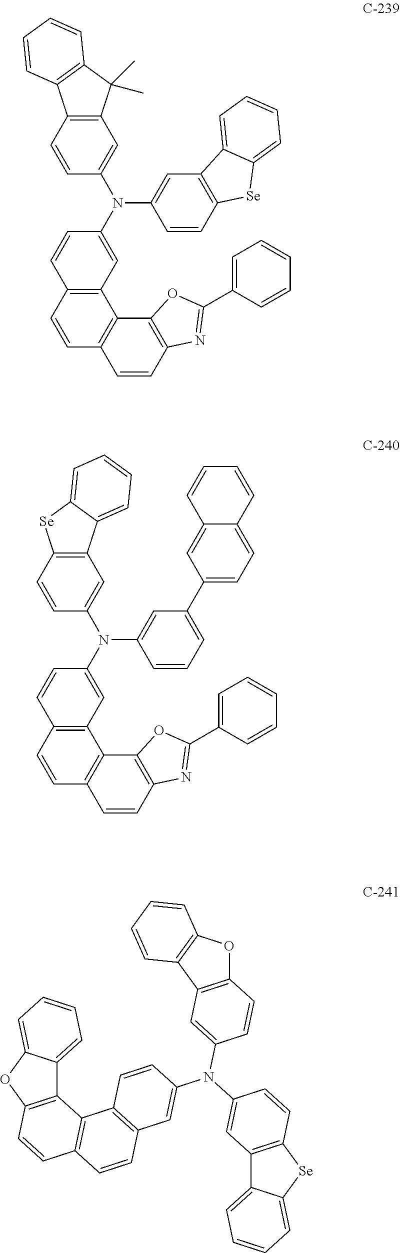 [SMILES: CC1(C)c2ccccc2-c2ccc(N(c3ccc4[se]c5ccccc5c4c3)c3ccc4ccc5ccc6nc(-c7ccccc7)oc6c5c4c3)cc21 ; c1ccc(-c2nc3ccc4ccc5ccc(N(c6cccc(-c7ccc8ccccc8c7)c6)c6ccc7[se]c8ccccc8c7c6)cc5c4c3o2)cc1 ; c1ccc2c(c1)oc1ccc(N(c3ccc4c(ccc5ccc6oc7ccccc7c6c54)c3)c3ccc4[se]c5ccccc5c4c3)cc12]

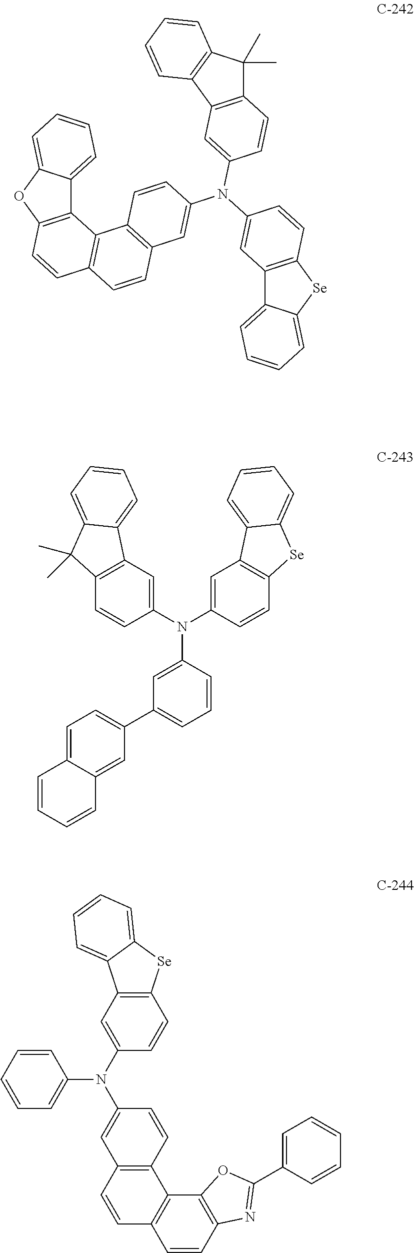 [SMILES: CC1(C)c2ccccc2-c2cc(N(c3ccc4c(ccc5ccc6oc7ccccc7c6c54)c3)c3ccc4[se]c5ccccc5c4c3)ccc21 ; CC1(C)c2ccccc2-c2cc(N(c3cccc(-c4ccc5ccccc5c4)c3)c3ccc4[se]c5ccccc5c4c3)ccc21 ; c1ccc(-c2nc3ccc4ccc5cc(N(c6ccccc6)c6ccc7[se]c8ccccc8c7c6)ccc5c4c3o2)cc1]

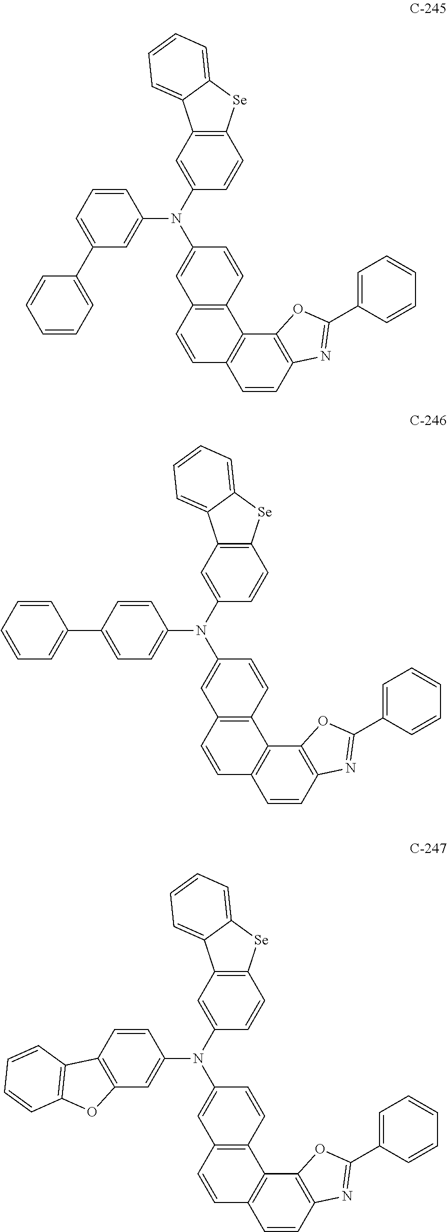 [SMILES: c1ccc(-c2cccc(N(c3ccc4c(ccc5ccc6nc(-c7ccccc7)oc6c54)c3)c3ccc4[se]c5ccccc5c4c3)c2)cc1 ; c1ccc(-c2ccc(N(c3ccc4c(ccc5ccc6nc(-c7ccccc7)oc6c54)c3)c3ccc4[se]c5ccccc5c4c3)cc2)cc1 ; c1ccc(-c2nc3ccc4ccc5cc(N(c6ccc7c(c6)oc6ccccc67)c6ccc7[se]c8ccccc8c7c6)ccc5c4c3o2)cc1]

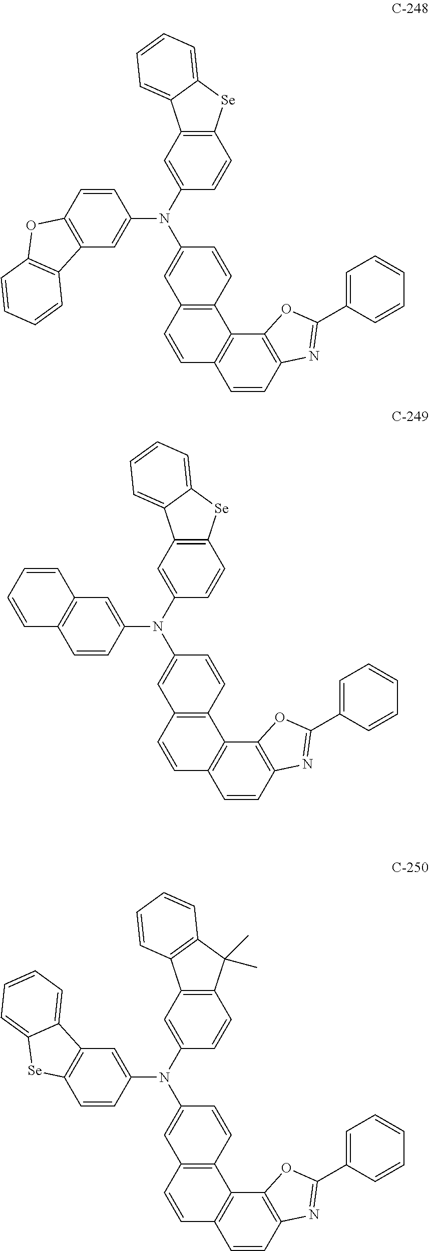 [SMILES: c1ccc(-c2nc3ccc4ccc5cc(N(c6ccc7oc8ccccc8c7c6)c6ccc7[se]c8ccccc8c7c6)ccc5c4c3o2)cc1 ; c1ccc(-c2nc3ccc4ccc5cc(N(c6ccc7ccccc7c6)c6ccc7[se]c8ccccc8c7c6)ccc5c4c3o2)cc1 ; CC1(C)c2ccccc2-c2cc(N(c3ccc4c(ccc5ccc6nc(-c7ccccc7)oc6c54)c3)c3ccc4[se]c5ccccc5c4c3)ccc21]

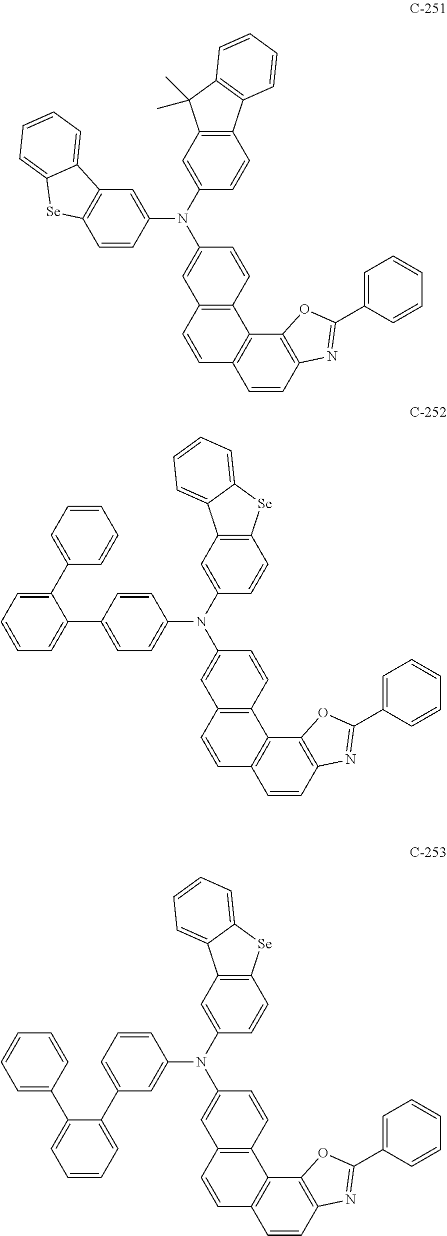 [SMILES: CC1(C)c2ccccc2-c2ccc(N(c3ccc4c(ccc5ccc6nc(-c7ccccc7)oc6c54)c3)c3ccc4[se]c5ccccc5c4c3)cc21 ; c1ccc(-c2nc3ccc4ccc5cc(N(c6ccc(-c7ccccc7-c7ccccc7)cc6)c6ccc7[se]c8ccccc8c7c6)ccc5c4c3o2)cc1 ; c1ccc(-c2nc3ccc4ccc5cc(N(c6cccc(-c7ccccc7-c7ccccc7)c6)c6ccc7[se]c8ccccc8c7c6)ccc5c4c3o2)cc1]

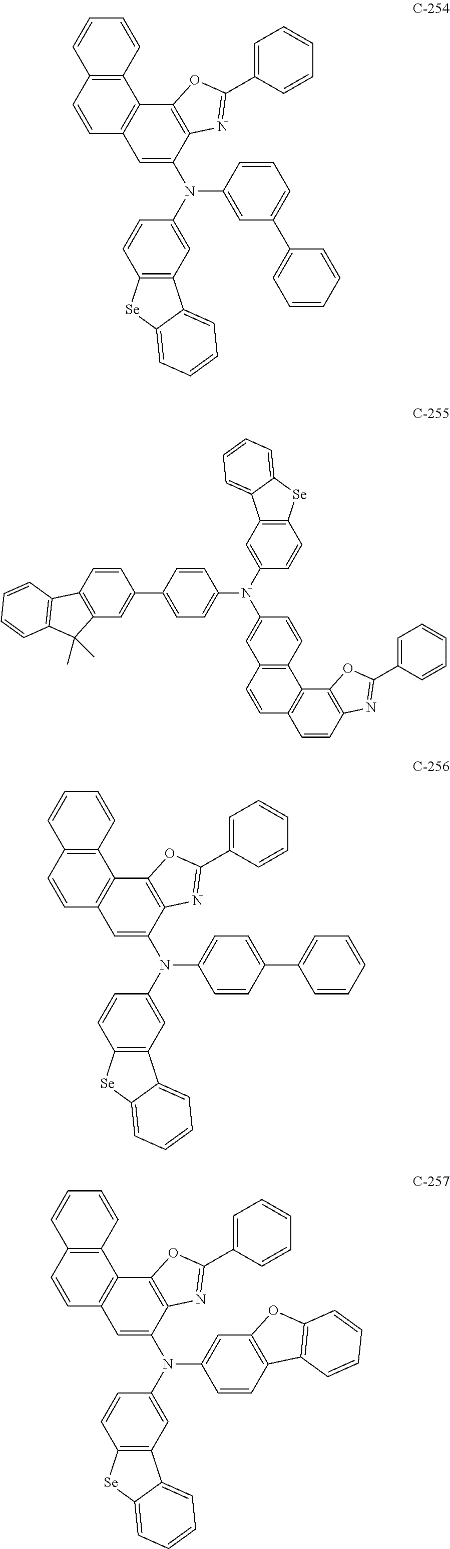 [SMILES: c1ccc(-c2cccc(N(c3ccc4[se]c5ccccc5c4c3)c3cc4ccc5ccccc5c4c4oc(-c5ccccc5)nc34)c2)cc1 ; CC1(C)c2ccccc2-c2ccc(-c3ccc(N(c4ccc5c(ccc6ccc7nc(-c8ccccc8)oc7c65)c4)c4ccc5[se]c6ccccc6c5c4)cc3)cc21 ; c1ccc(-c2ccc(N(c3ccc4[se]c5ccccc5c4c3)c3cc4ccc5ccccc5c4c4oc(-c5ccccc5)nc34)cc2)cc1 ; c1ccc(-c2nc3c(N(c4ccc5c(c4)oc4ccccc45)c4ccc5[se]c6ccccc6c5c4)cc4ccc5ccccc5c4c3o2)cc1]

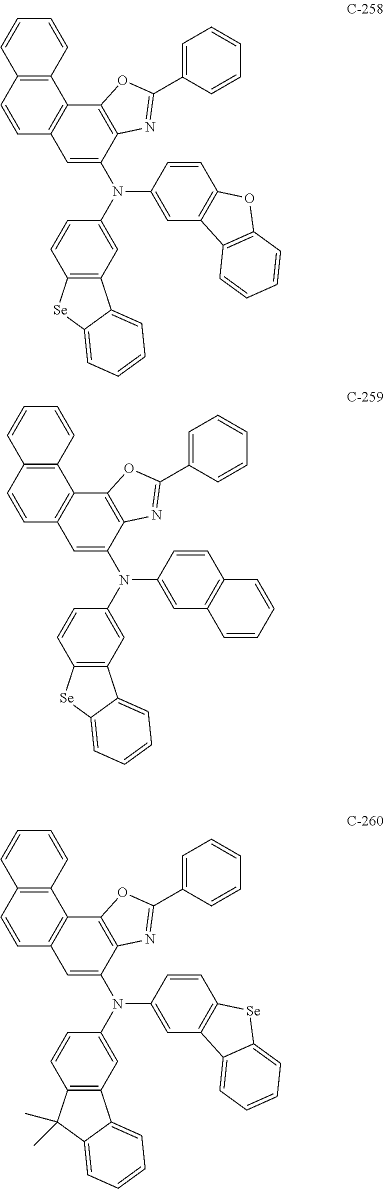 [SMILES: c1ccc(-c2nc3c(N(c4ccc5oc6ccccc6c5c4)c4ccc5[se]c6ccccc6c5c4)cc4ccc5ccccc5c4c3o2)cc1 ; c1ccc(-c2nc3c(N(c4ccc5ccccc5c4)c4ccc5[se]c6ccccc6c5c4)cc4ccc5ccccc5c4c3o2)cc1 ; CC1(C)c2ccccc2-c2cc(N(c3ccc4[se]c5ccccc5c4c3)c3cc4ccc5ccccc5c4c4oc(-c5ccccc5)nc34)ccc21]

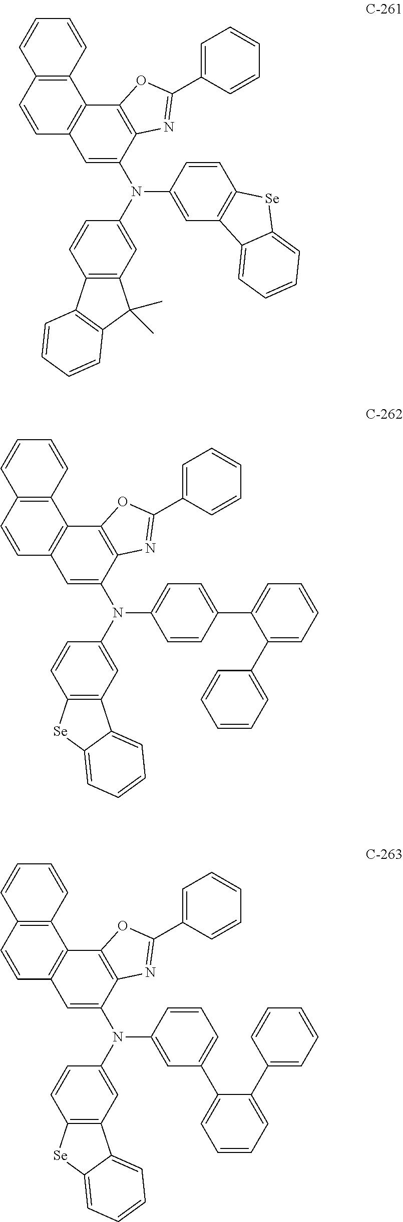 [SMILES: CC1(C)c2ccccc2-c2ccc(N(c3ccc4[se]c5ccccc5c4c3)c3cc4ccc5ccccc5c4c4oc(-c5ccccc5)nc34)cc21 ; c1ccc(-c2nc3c(N(c4ccc(-c5ccccc5-c5ccccc5)cc4)c4ccc5[se]c6ccccc6c5c4)cc4ccc5ccccc5c4c3o2)cc1 ; c1ccc(-c2nc3c(N(c4cccc(-c5ccccc5-c5ccccc5)c4)c4ccc5[se]c6ccccc6c5c4)cc4ccc5ccccc5c4c3o2)cc1]

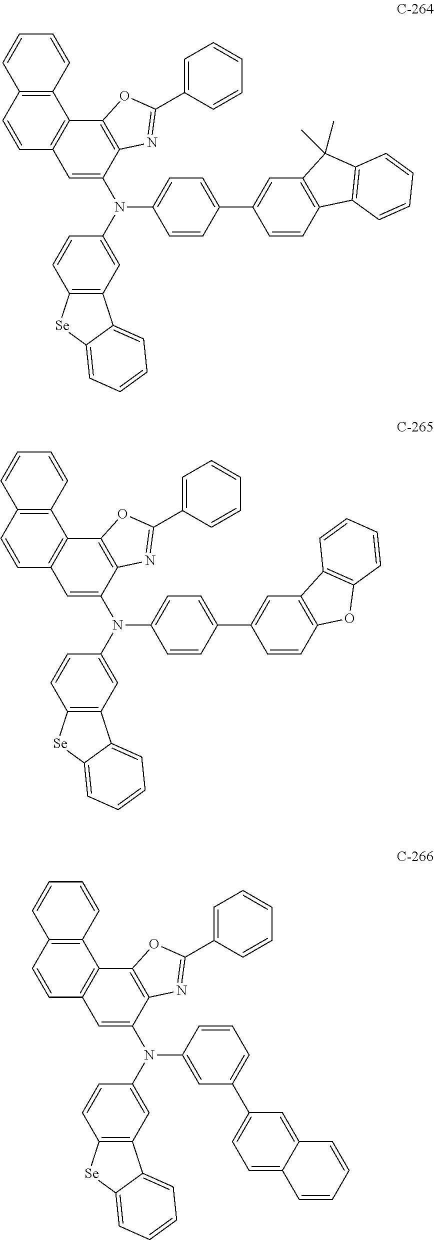 [SMILES: CC1(C)c2ccccc2-c2ccc(-c3ccc(N(c4ccc5[se]c6ccccc6c5c4)c4cc5ccc6ccccc6c5c5oc(-c6ccccc6)nc45)cc3)cc21 ; c1ccc(-c2nc3c(N(c4ccc(-c5ccc6oc7ccccc7c6c5)cc4)c4ccc5[se]c6ccccc6c5c4)cc4ccc5ccccc5c4c3o2)cc1 ; c1ccc(-c2nc3c(N(c4cccc(-c5ccc6ccccc6c5)c4)c4ccc5[se]c6ccccc6c5c4)cc4ccc5ccccc5c4c3o2)cc1]

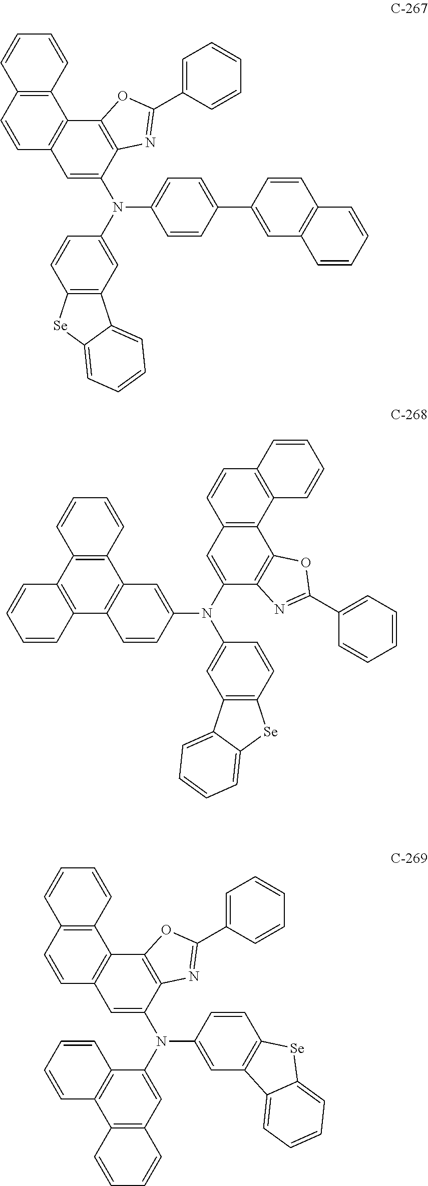 [SMILES: c1ccc(-c2nc3c(N(c4ccc(-c5ccc6ccccc6c5)cc4)c4ccc5[se]c6ccccc6c5c4)cc4ccc5ccccc5c4c3o2)cc1 ; c1ccc(-c2nc3c(N(c4ccc5[se]c6ccccc6c5c4)c4ccc5c6ccccc6c6ccccc6c5c4)cc4ccc5ccccc5c4c3o2)cc1 ; c1ccc(-c2nc3c(N(c4ccc5[se]c6ccccc6c5c4)c4cc5ccccc5c5ccccc45)cc4ccc5ccccc5c4c3o2)cc1]

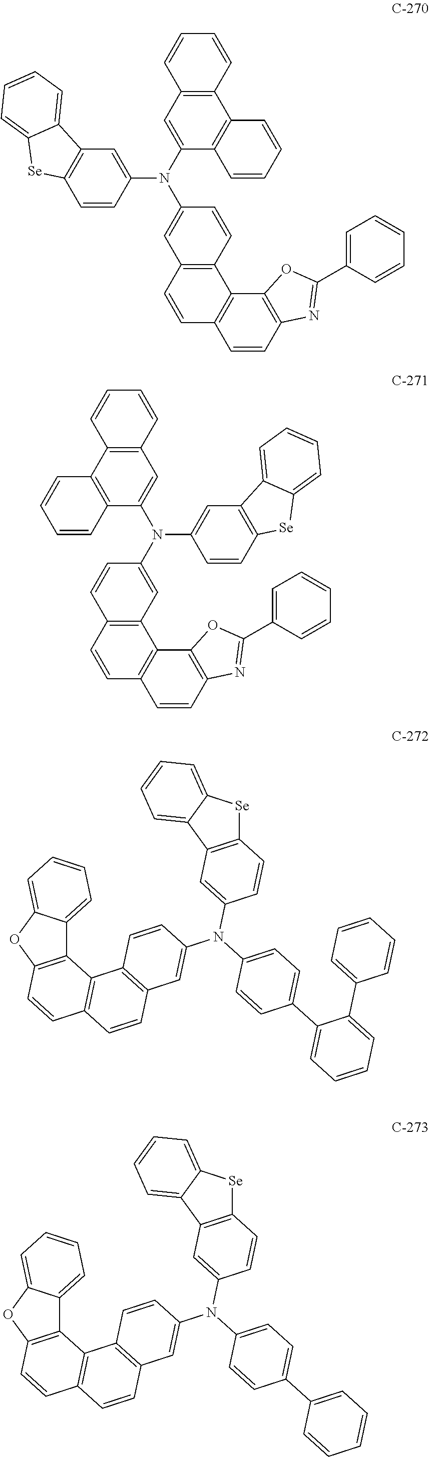 [SMILES: c1ccc(-c2nc3ccc4ccc5cc(N(c6ccc7[se]c8ccccc8c7c6)c6cc7ccccc7c7ccccc67)ccc5c4c3o2)cc1 ; c1ccc(-c2nc3ccc4ccc5ccc(N(c6ccc7[se]c8ccccc8c7c6)c6cc7ccccc7c7ccccc67)cc5c4c3o2)cc1 ; c1ccc(-c2ccccc2-c2ccc(N(c3ccc4c(ccc5ccc6oc7ccccc7c6c54)c3)c3ccc4[se]c5ccccc5c4c3)cc2)cc1 ; c1ccc(-c2ccc(N(c3ccc4c(ccc5ccc6oc7ccccc7c6c54)c3)c3ccc4[se]c5ccccc5c4c3)cc2)cc1]

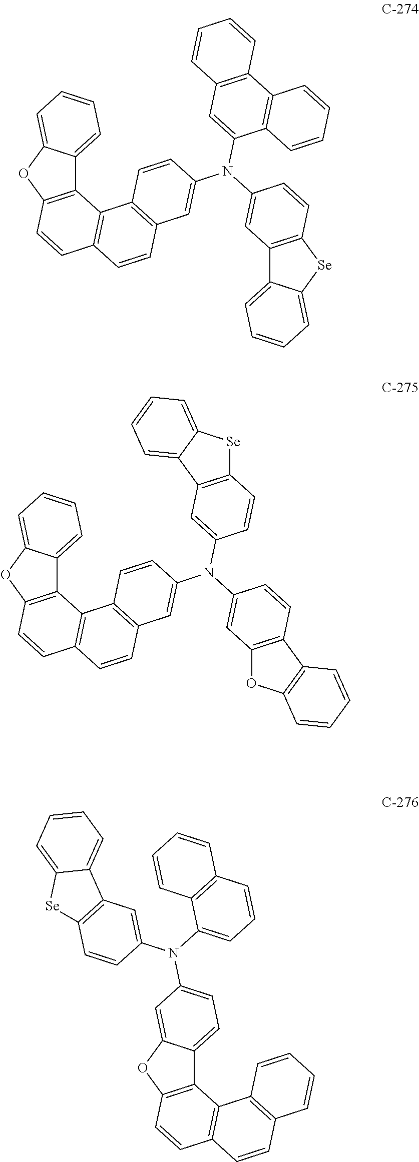 [SMILES: c1ccc2c(c1)cc(N(c1ccc3c(ccc4ccc5oc6ccccc6c5c43)c1)c1ccc3[se]c4ccccc4c3c1)c1ccccc12 ; c1ccc2c(c1)oc1cc(N(c3ccc4c(ccc5ccc6oc7ccccc7c6c54)c3)c3ccc4[se]c5ccccc5c4c3)ccc12 ; c1ccc2c(N(c3ccc4c(c3)oc3ccc5ccc6ccccc6c5c34)c3ccc4[se]c5ccccc5c4c3)cccc2c1]

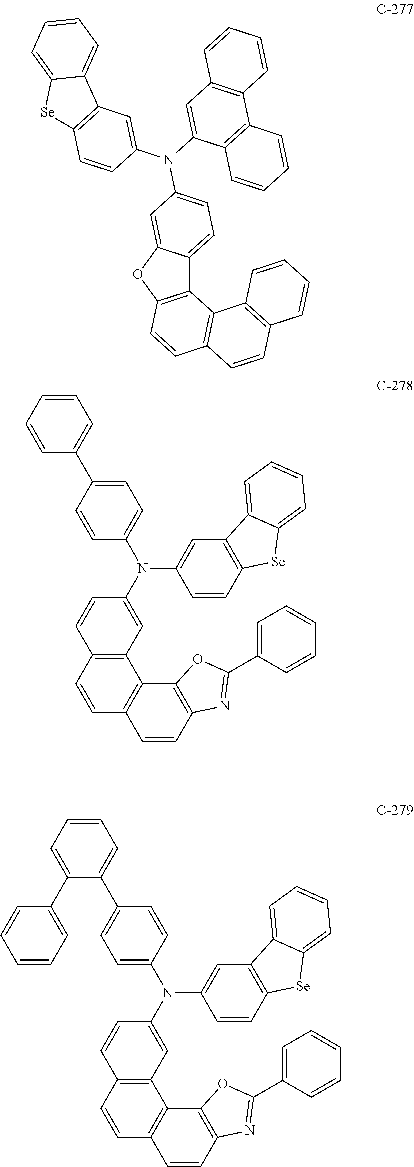 [SMILES: c1ccc2c(c1)cc(N(c1ccc3c(c1)oc1ccc4ccc5ccccc5c4c13)c1ccc3[se]c4ccccc4c3c1)c1ccccc12 ; c1ccc(-c2ccc(N(c3ccc4[se]c5ccccc5c4c3)c3ccc4ccc5ccc6nc(-c7ccccc7)oc6c5c4c3)cc2)cc1 ; c1ccc(-c2nc3ccc4ccc5ccc(N(c6ccc(-c7ccccc7-c7ccccc7)cc6)c6ccc7[se]c8ccccc8c7c6)cc5c4c3o2)cc1]

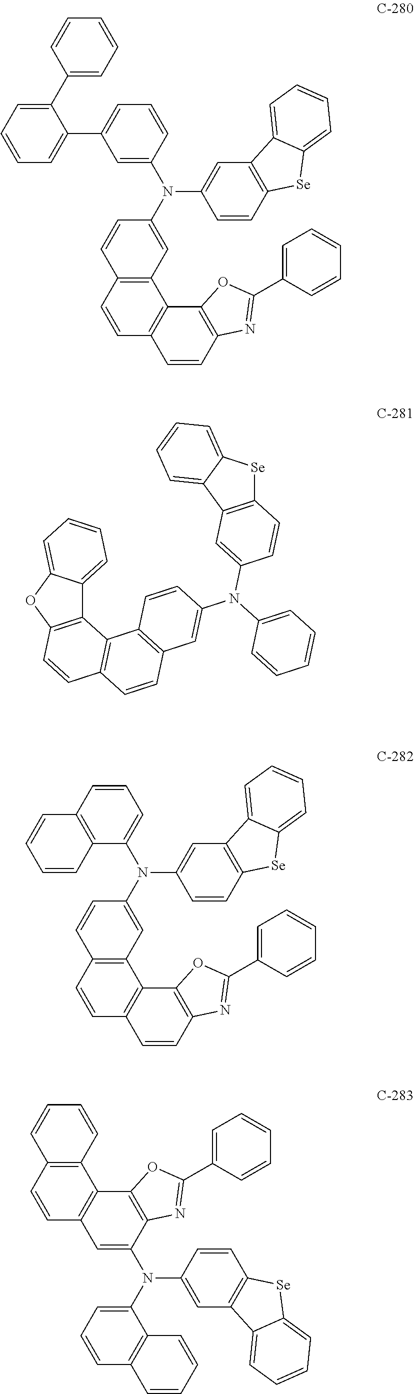 [SMILES: c1ccc(-c2nc3ccc4ccc5ccc(N(c6cccc(-c7ccccc7-c7ccccc7)c6)c6ccc7[se]c8ccccc8c7c6)cc5c4c3o2)cc1 ; c1ccc(N(c2ccc3c(ccc4ccc5oc6ccccc6c5c43)c2)c2ccc3[se]c4ccccc4c3c2)cc1 ; c1ccc(-c2nc3ccc4ccc5ccc(N(c6ccc7[se]c8ccccc8c7c6)c6cccc7ccccc67)cc5c4c3o2)cc1 ; c1ccc(-c2nc3c(N(c4ccc5[se]c6ccccc6c5c4)c4cccc5ccccc45)cc4ccc5ccccc5c4c3o2)cc1]

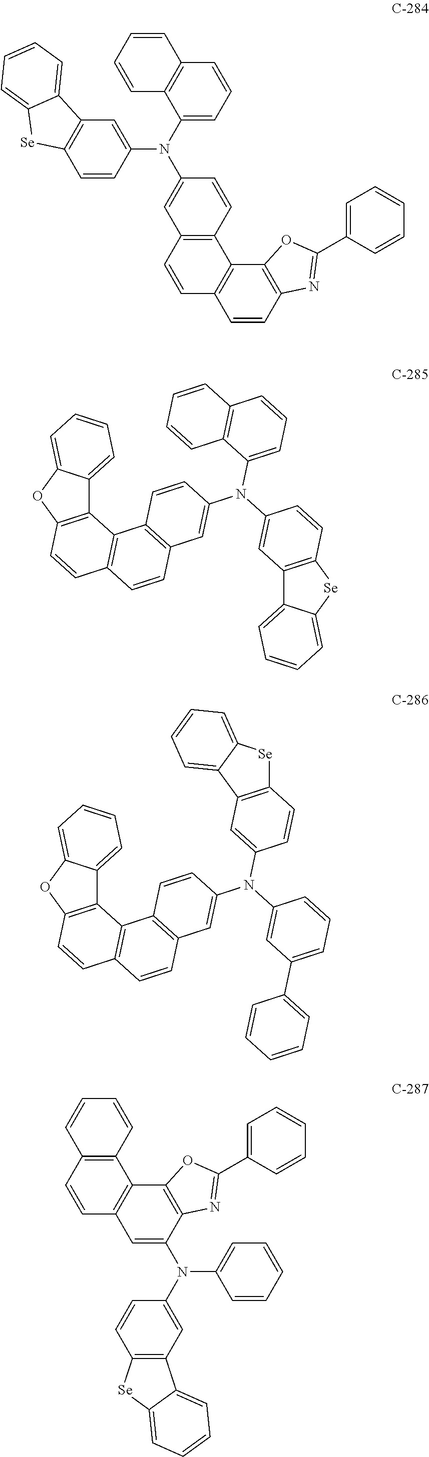 [SMILES: c1ccc(-c2nc3ccc4ccc5cc(N(c6ccc7[se]c8ccccc8c7c6)c6cccc7ccccc67)ccc5c4c3o2)cc1 ; c1ccc2c(N(c3ccc4c(ccc5ccc6oc7ccccc7c6c54)c3)c3ccc4[se]c5ccccc5c4c3)cccc2c1 ; c1ccc(-c2cccc(N(c3ccc4c(ccc5ccc6oc7ccccc7c6c54)c3)c3ccc4[se]c5ccccc5c4c3)c2)cc1 ; c1ccc(-c2nc3c(N(c4ccccc4)c4ccc5[se]c6ccccc6c5c4)cc4ccc5ccccc5c4c3o2)cc1]

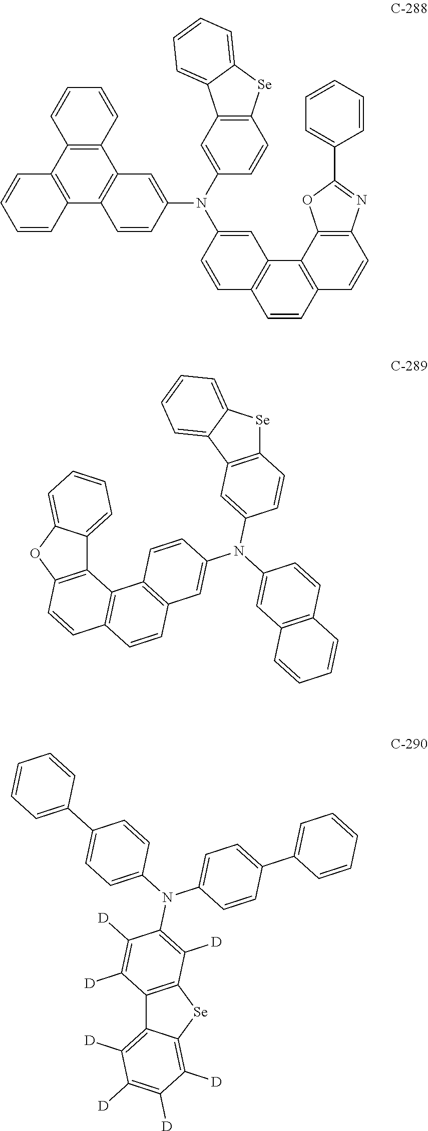 [SMILES: c1ccc(-c2nc3ccc4ccc5ccc(N(c6ccc7[se]c8ccccc8c7c6)c6ccc7c8ccccc8c8ccccc8c7c6)cc5c4c3o2)cc1 ; c1ccc2cc(N(c3ccc4c(ccc5ccc6oc7ccccc7c6c54)c3)c3ccc4[se]c5ccccc5c4c3)ccc2c1 ; [2H]c1c([2H])c([2H])c2c([se]c3c([2H])c(N(c4ccc(-c5ccccc5)cc4)c4ccc(-c5ccccc5)cc4)c([2H])c([2H])c32)c1[2H]]

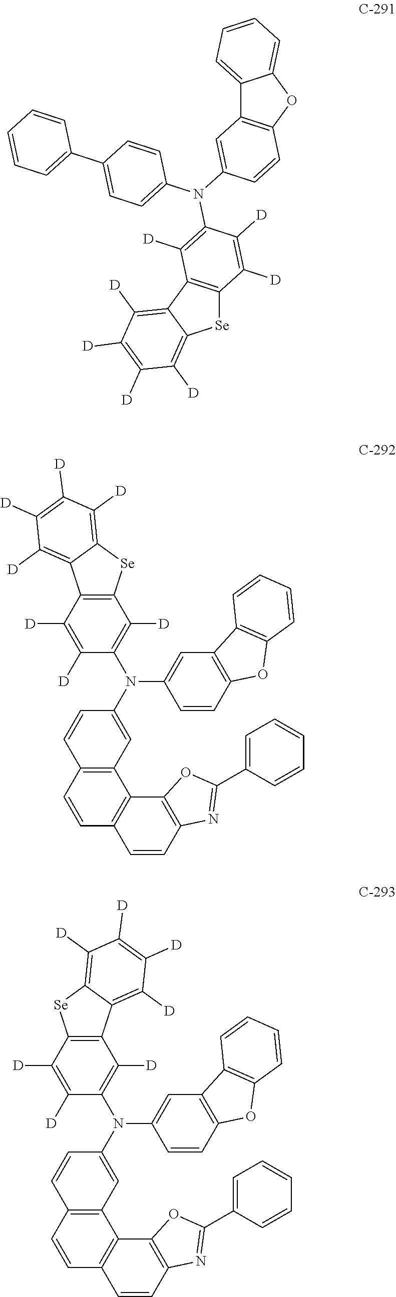 [SMILES: [2H]c1c([2H])c([2H])c2c([se]c3c([2H])c([2H])c(N(c4ccc(-c5ccccc5)cc4)c4ccc5oc6ccccc6c5c4)c([2H])c32)c1[2H] ; [2H]c1c([2H])c([2H])c2c([se]c3c([2H])c(N(c4ccc5oc6ccccc6c5c4)c4ccc5ccc6ccc7nc(-c8ccccc8)oc7c6c5c4)c([2H])c([2H])c32)c1[2H] ; [2H]c1c([2H])c([2H])c2c([se]c3c([2H])c([2H])c(N(c4ccc5oc6ccccc6c5c4)c4ccc5ccc6ccc7nc(-c8ccccc8)oc7c6c5c4)c([2H])c32)c1[2H]]

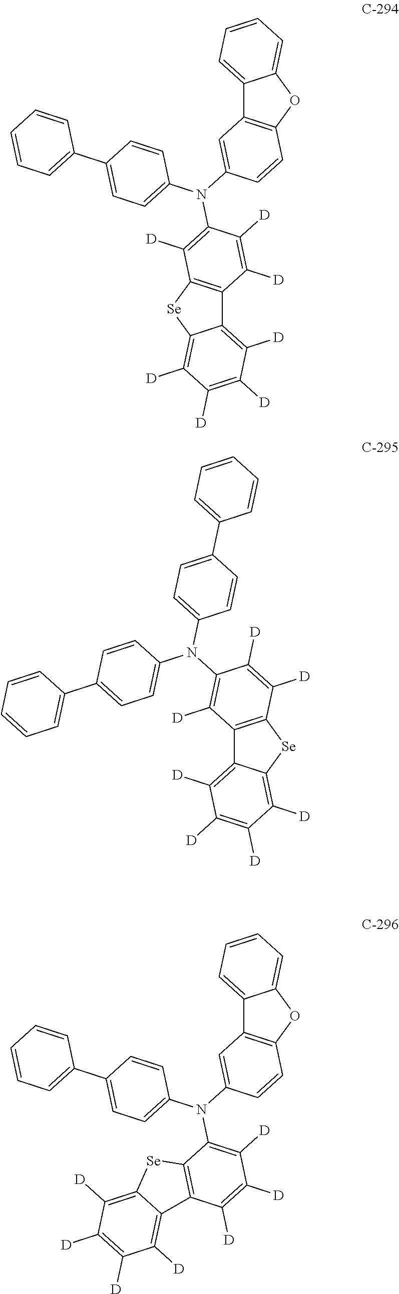

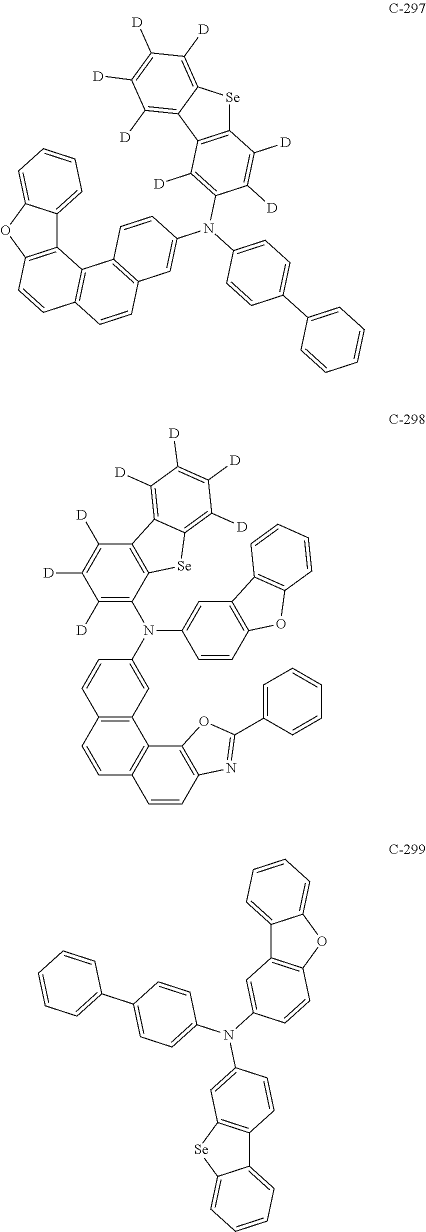 [SMILES: [2H]c1c([2H])c([2H])c2c([se]c3c([2H])c([2H])c(N(c4ccc(-c5ccccc5)cc4)c4ccc5c(ccc6ccc7oc8ccccc8c7c65)c4)c([2H])c32)c1[2H] ; [2H]c1c([2H])c([2H])c2c([se]c3c(N(c4ccc5oc6ccccc6c5c4)c4ccc5ccc6ccc7nc(-c8ccccc8)oc7c6c5c4)c([2H])c([2H])c([2H])c32)c1[2H] ; c1ccc(-c2ccc(N(c3ccc4c(c3)[se]c3ccccc34)c3ccc4oc5ccccc5c4c3)cc2)cc1]

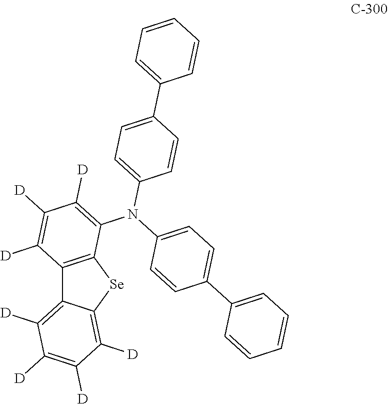 [SMILES: [2H]c1c([2H])c([2H])c2c([se]c3c(N(c4ccc(-c5ccccc5)cc4)c4ccc(-c5ccccc5)cc4)c([2H])c([2H])c([2H])c32)c1[2H]]

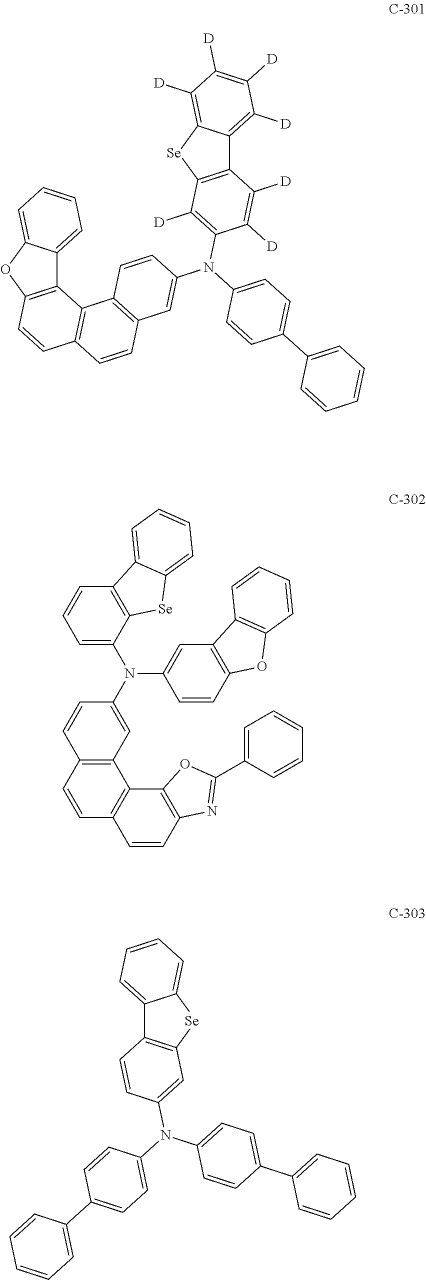 [SMILES: [2H]c1c([2H])c([2H])c2c([se]c3c([2H])c(N(c4ccc(-c5ccccc5)cc4)c4ccc5c(ccc6ccc7oc8ccccc8c7c65)c4)c([2H])c([2H])c32)c1[2H] ; c1ccc(-c2nc3ccc4ccc5ccc(N(c6ccc7oc8ccccc8c7c6)c6cccc7c6[se]c6ccccc67)cc5c4c3o2)cc1 ; c1ccc(-c2ccc(N(c3ccc(-c4ccccc4)cc3)c3ccc4c(c3)[se]c3ccccc34)cc2)cc1]

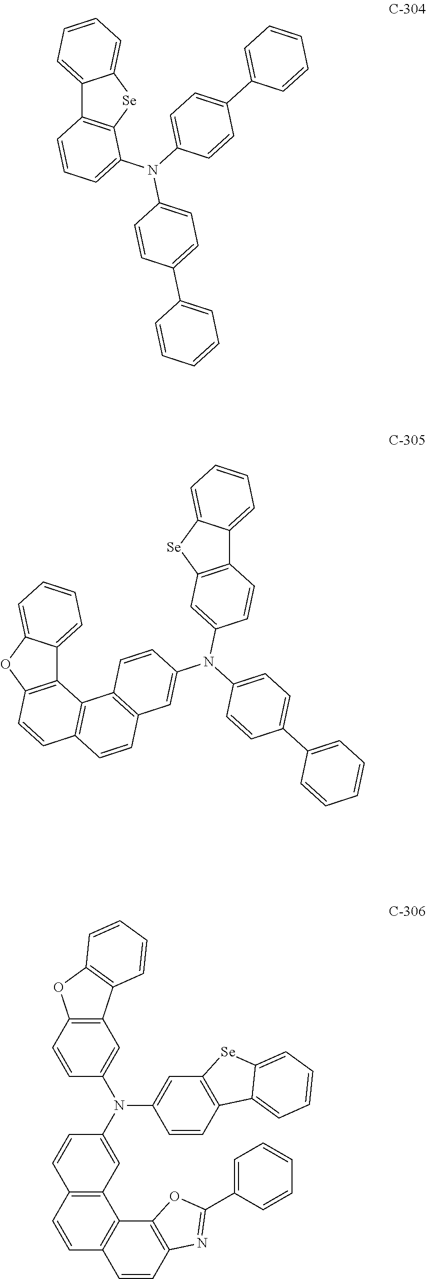 [SMILES: c1ccc(-c2ccc(N(c3ccc(-c4ccccc4)cc3)c3cccc4c3[se]c3ccccc34)cc2)cc1 ; c1ccc(-c2ccc(N(c3ccc4c(ccc5ccc6oc7ccccc7c6c54)c3)c3ccc4c(c3)[se]c3ccccc34)cc2)cc1 ; c1ccc(-c2nc3ccc4ccc5ccc(N(c6ccc7c(c6)[se]c6ccccc67)c6ccc7oc8ccccc8c7c6)cc5c4c3o2)cc1]

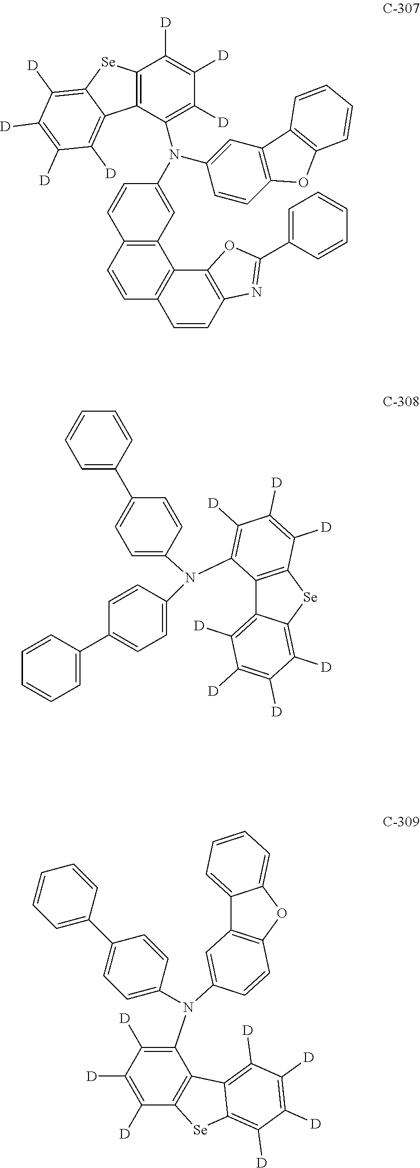 [SMILES: [2H]c1c([2H])c([2H])c2c([se]c3c([2H])c([2H])c([2H])c(N(c4ccc5oc6ccccc6c5c4)c4ccc5ccc6ccc7nc(-c8ccccc8)oc7c6c5c4)c32)c1[2H] ; [2H]c1c([2H])c([2H])c2c([se]c3c([2H])c([2H])c([2H])c(N(c4ccc(-c5ccccc5)cc4)c4ccc(-c5ccccc5)cc4)c32)c1[2H] ; [2H]c1c([2H])c([2H])c2c([se]c3c([2H])c([2H])c([2H])c(N(c4ccc(-c5ccccc5)cc4)c4ccc5oc6ccccc6c5c4)c32)c1[2H]]

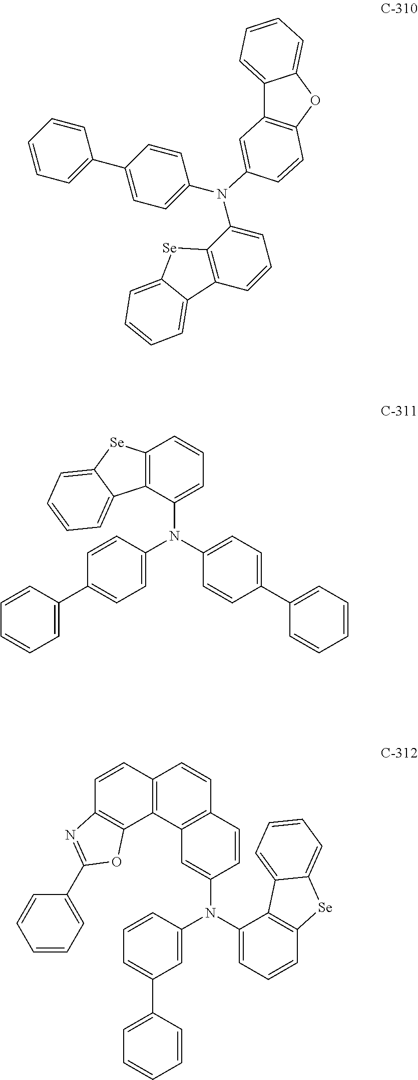 [SMILES: c1ccc(-c2ccc(N(c3ccc4oc5ccccc5c4c3)c3cccc4c3[se]c3ccccc34)cc2)cc1 ; c1ccc(-c2ccc(N(c3ccc(-c4ccccc4)cc3)c3cccc4[se]c5ccccc5c34)cc2)cc1 ; c1ccc(-c2cccc(N(c3ccc4ccc5ccc6nc(-c7ccccc7)oc6c5c4c3)c3cccc4[se]c5ccccc5c34)c2)cc1]

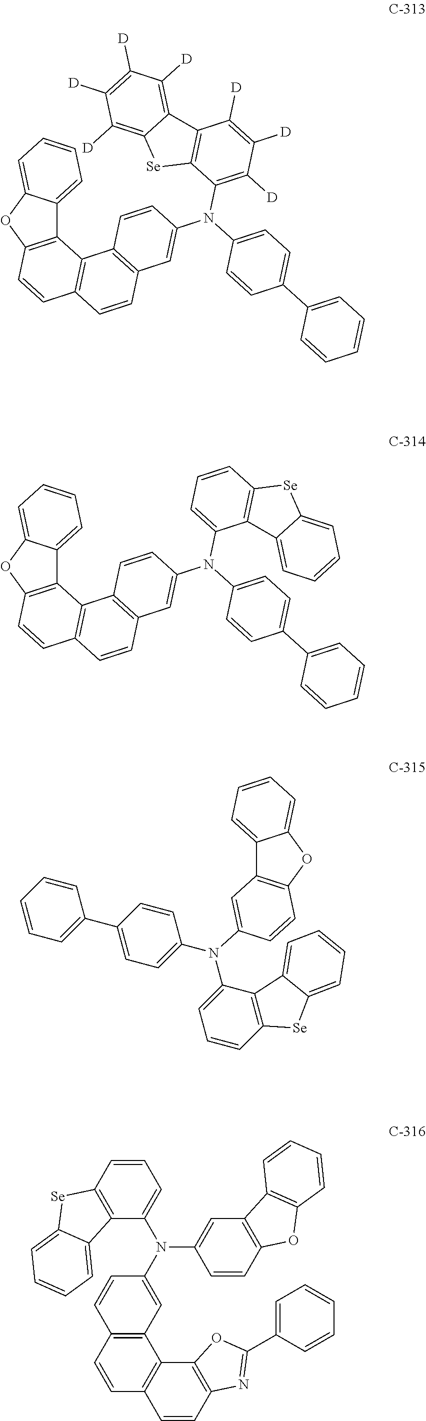 [SMILES: [2H]c1c([2H])c([2H])c2c([se]c3c(N(c4ccc(-c5ccccc5)cc4)c4ccc5c(ccc6ccc7oc8ccccc8c7c65)c4)c([2H])c([2H])c([2H])c32)c1[2H] ; c1ccc(-c2ccc(N(c3ccc4c(ccc5ccc6oc7ccccc7c6c54)c3)c3cccc4[se]c5ccccc5c34)cc2)cc1 ; c1ccc(-c2ccc(N(c3ccc4oc5ccccc5c4c3)c3cccc4[se]c5ccccc5c34)cc2)cc1 ; c1ccc(-c2nc3ccc4ccc5ccc(N(c6ccc7oc8ccccc8c7c6)c6cccc7[se]c8ccccc8c67)cc5c4c3o2)cc1]

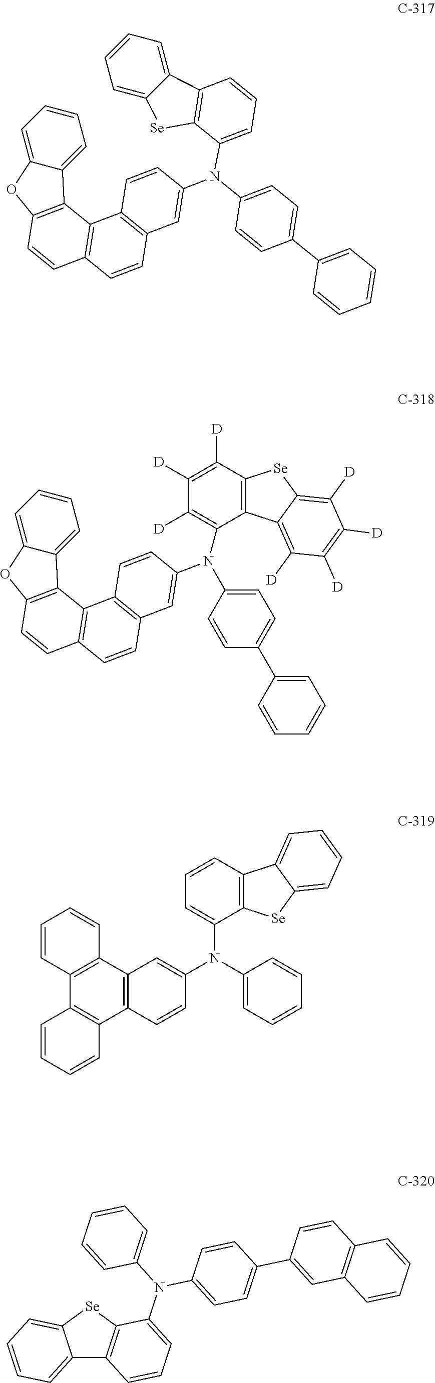 [SMILES: c1ccc(-c2ccc(N(c3ccc4c(ccc5ccc6oc7ccccc7c6c54)c3)c3cccc4c3[se]c3ccccc34)cc2)cc1 ; [2H]c1c([2H])c([2H])c2c([se]c3c([2H])c([2H])c([2H])c(N(c4ccc(-c5ccccc5)cc4)c4ccc5c(ccc6ccc7oc8ccccc8c7c65)c4)c32)c1[2H] ; c1ccc(N(c2ccc3c4ccccc4c4ccccc4c3c2)c2cccc3c2[se]c2ccccc23)cc1 ; c1ccc(N(c2ccc(-c3ccc4ccccc4c3)cc2)c2cccc3c2[se]c2ccccc23)cc1]

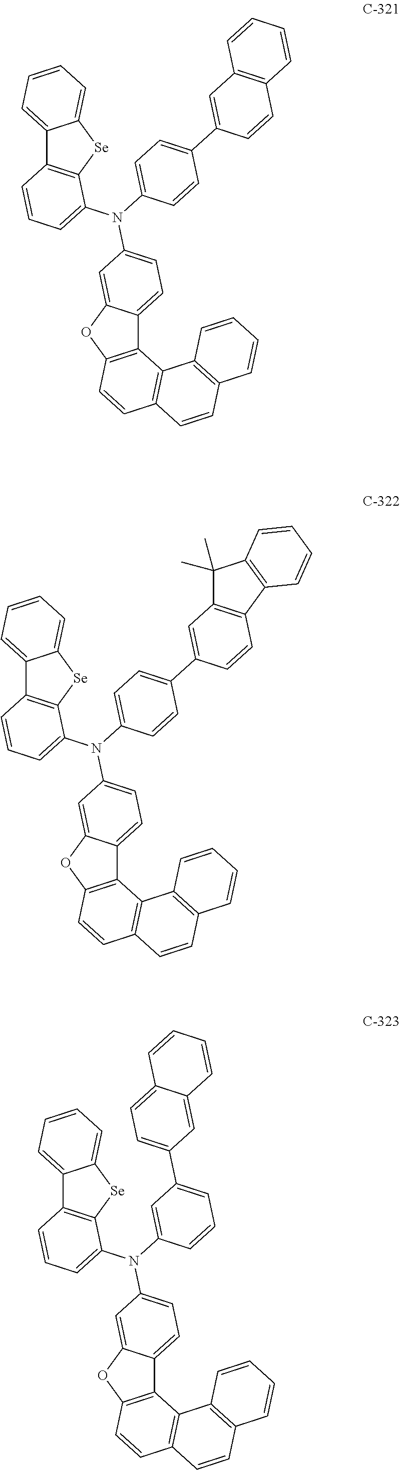 [SMILES: c1ccc2cc(-c3ccc(N(c4ccc5c(c4)oc4ccc6ccc7ccccc7c6c45)c4cccc5c4[se]c4ccccc45)cc3)ccc2c1 ; CC1(C)c2ccccc2-c2ccc(-c3ccc(N(c4ccc5c(c4)oc4ccc6ccc7ccccc7c6c45)c4cccc5c4[se]c4ccccc45)cc3)cc21 ; c1cc(-c2ccc3ccccc3c2)cc(N(c2ccc3c(c2)oc2ccc4ccc5ccccc5c4c23)c2cccc3c2[se]c2ccccc23)c1]

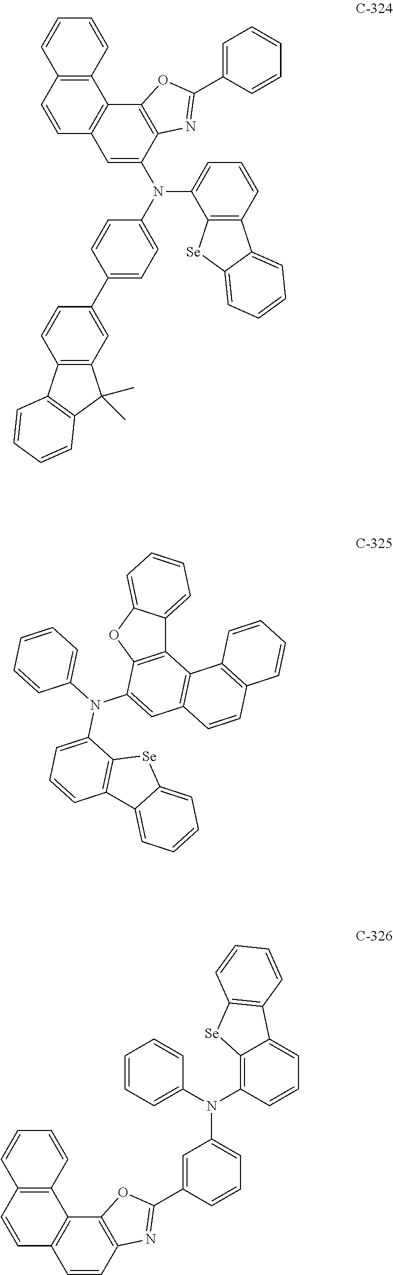 [SMILES: CC1(C)c2ccccc2-c2ccc(-c3ccc(N(c4cc5ccc6ccccc6c5c5oc(-c6ccccc6)nc45)c4cccc5c4[se]c4ccccc45)cc3)cc21 ; c1ccc(N(c2cc3ccc4ccccc4c3c3c2oc2ccccc23)c2cccc3c2[se]c2ccccc23)cc1 ; c1ccc(N(c2cccc(-c3nc4ccc5ccc6ccccc6c5c4o3)c2)c2cccc3c2[se]c2ccccc23)cc1]

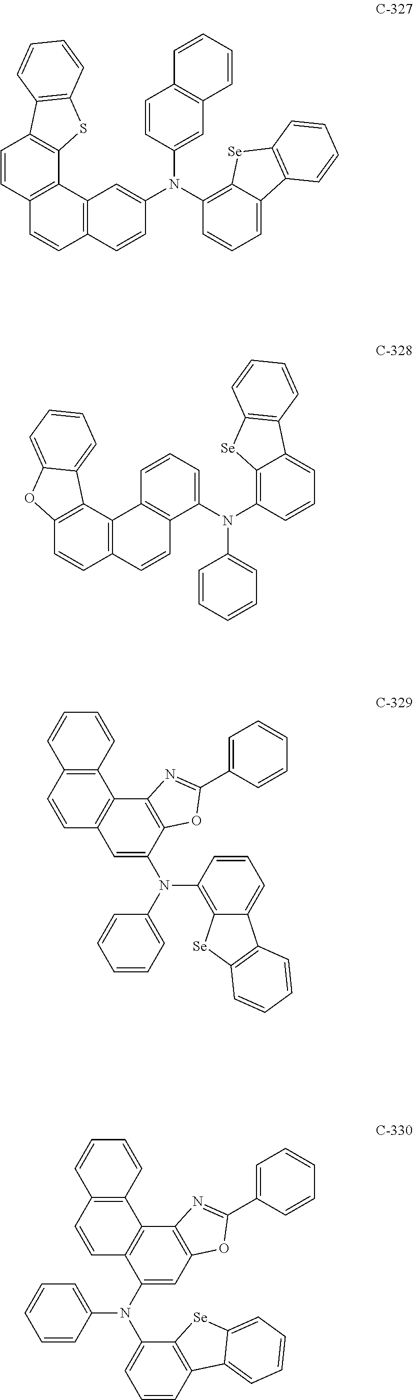 [SMILES: c1ccc2cc(N(c3ccc4ccc5ccc6c7ccccc7sc6c5c4c3)c3cccc4c3[se]c3ccccc34)ccc2c1 ; c1ccc(N(c2cccc3c2ccc2ccc4oc5ccccc5c4c23)c2cccc3c2[se]c2ccccc23)cc1 ; c1ccc(-c2nc3c(o2)c(N(c2ccccc2)c2cccc4c2[se]c2ccccc24)cc2ccc4ccccc4c23)cc1 ; c1ccc(-c2nc3c(cc(N(c4ccccc4)c4cccc5c4[se]c4ccccc45)c4ccc5ccccc5c43)o2)cc1]

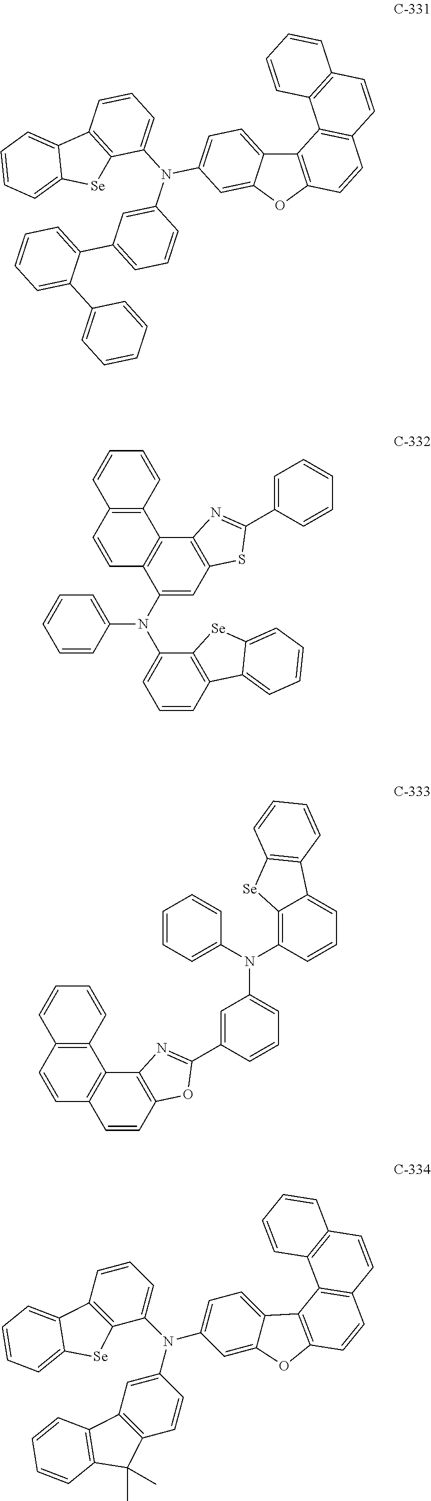 [SMILES: c1ccc(-c2ccccc2-c2cccc(N(c3ccc4c(c3)oc3ccc5ccc6ccccc6c5c34)c3cccc4c3[se]c3ccccc34)c2)cc1 ; c1ccc(-c2nc3c(cc(N(c4ccccc4)c4cccc5c4[se]c4ccccc45)c4ccc5ccccc5c43)s2)cc1 ; c1ccc(N(c2cccc(-c3nc4c(ccc5ccc6ccccc6c54)o3)c2)c2cccc3c2[se]c2ccccc23)cc1 ; CC1(C)c2ccccc2-c2cc(N(c3ccc4c(c3)oc3ccc5ccc6ccccc6c5c34)c3cccc4c3[se]c3ccccc34)ccc21]

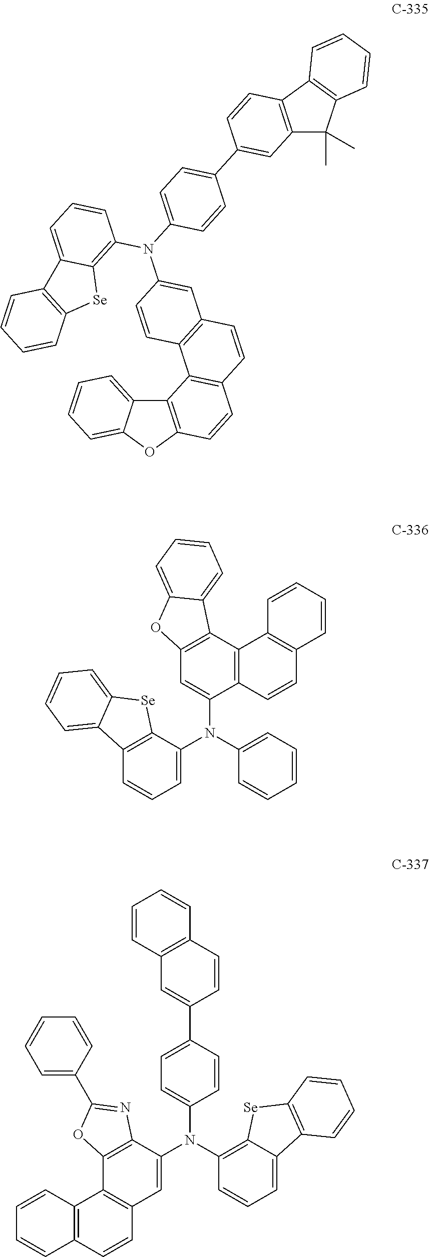 [SMILES: CC1(C)c2ccccc2-c2ccc(-c3ccc(N(c4ccc5c(ccc6ccc7oc8ccccc8c7c65)c4)c4cccc5c4[se]c4ccccc45)cc3)cc21 ; c1ccc(N(c2cc3oc4ccccc4c3c3c2ccc2ccccc23)c2cccc3c2[se]c2ccccc23)cc1 ; c1ccc(-c2nc3c(N(c4ccc(-c5ccc6ccccc6c5)cc4)c4cccc5c4[se]c4ccccc45)cc4ccc5ccccc5c4c3o2)cc1]

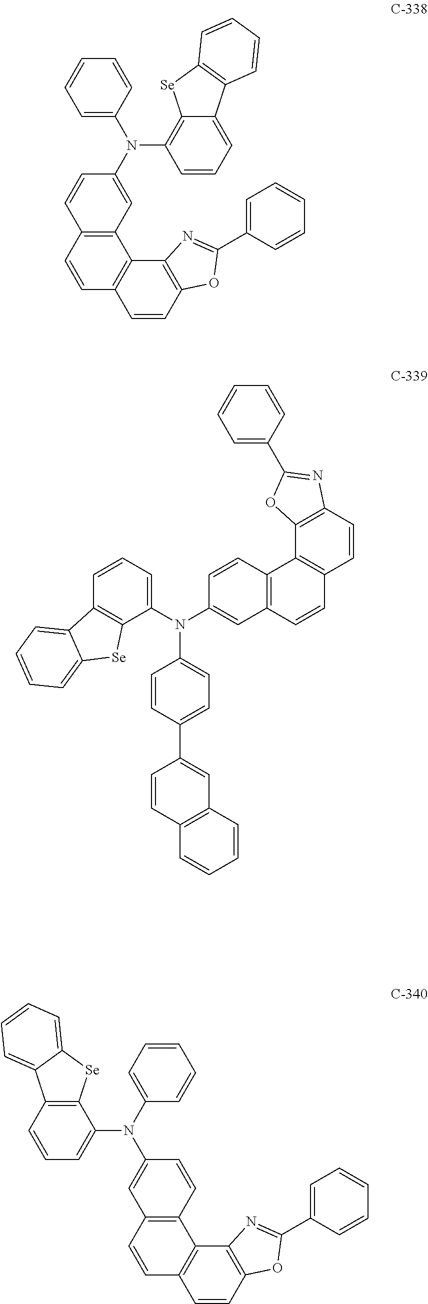 [SMILES: c1ccc(-c2nc3c(ccc4ccc5ccc(N(c6ccccc6)c6cccc7c6[se]c6ccccc67)cc5c43)o2)cc1 ; c1ccc(-c2nc3ccc4ccc5cc(N(c6ccc(-c7ccc8ccccc8c7)cc6)c6cccc7c6[se]c6ccccc67)ccc5c4c3o2)cc1 ; c1ccc(-c2nc3c(ccc4ccc5cc(N(c6ccccc6)c6cccc7c6[se]c6ccccc67)ccc5c43)o2)cc1]

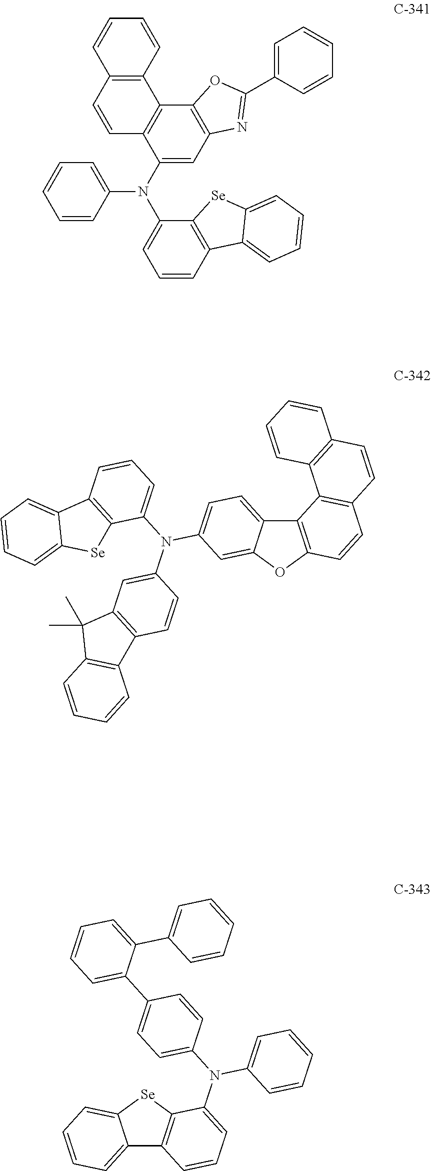 [SMILES: c1ccc(-c2nc3cc(N(c4ccccc4)c4cccc5c4[se]c4ccccc45)c4ccc5ccccc5c4c3o2)cc1 ; CC1(C)c2ccccc2-c2ccc(N(c3ccc4c(c3)oc3ccc5ccc6ccccc6c5c34)c3cccc4c3[se]c3ccccc34)cc21 ; c1ccc(-c2ccccc2-c2ccc(N(c3ccccc3)c3cccc4c3[se]c3ccccc34)cc2)cc1]

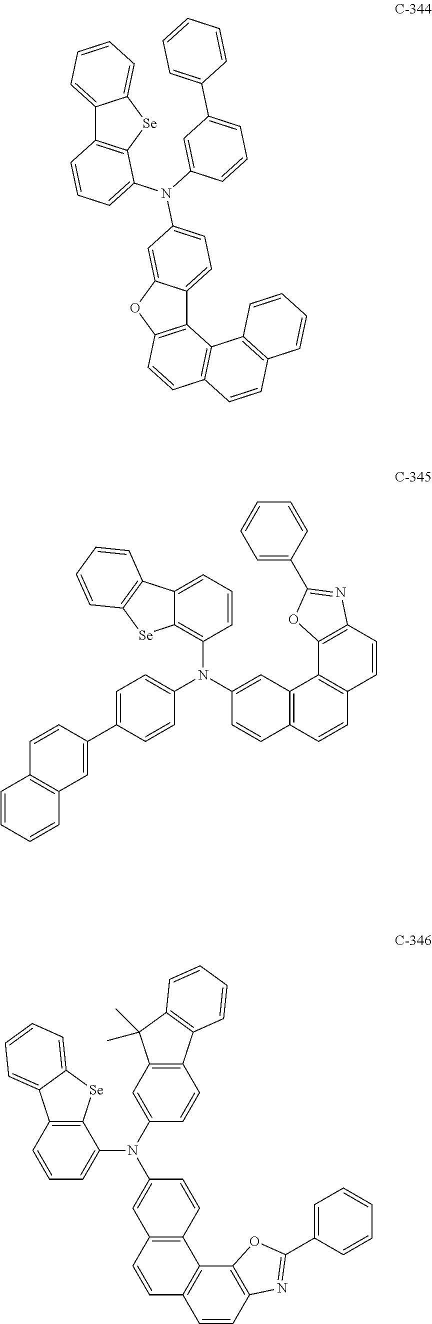 [SMILES: c1ccc(-c2cccc(N(c3ccc4c(c3)oc3ccc5ccc6ccccc6c5c34)c3cccc4c3[se]c3ccccc34)c2)cc1 ; c1ccc(-c2nc3ccc4ccc5ccc(N(c6ccc(-c7ccc8ccccc8c7)cc6)c6cccc7c6[se]c6ccccc67)cc5c4c3o2)cc1 ; CC1(C)c2ccccc2-c2ccc(N(c3ccc4c(ccc5ccc6nc(-c7ccccc7)oc6c54)c3)c3cccc4c3[se]c3ccccc34)cc21]

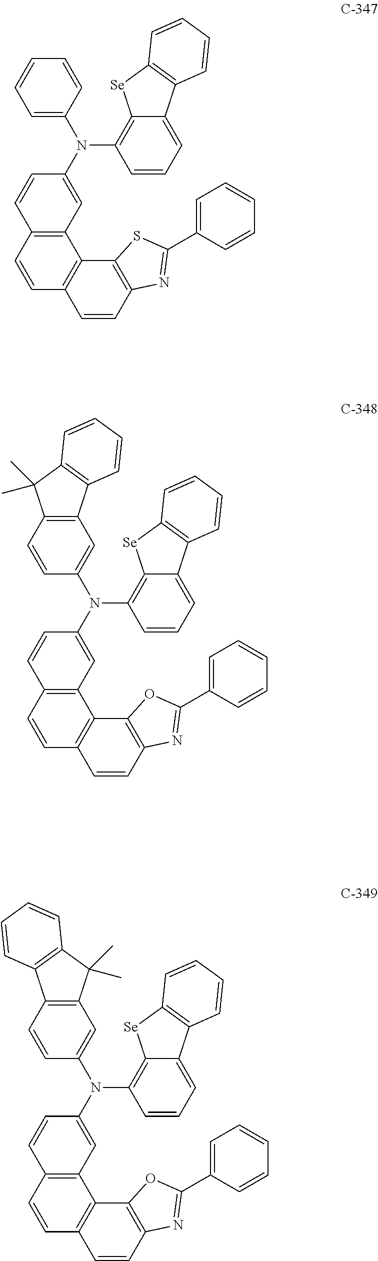 [SMILES: c1ccc(-c2nc3ccc4ccc5ccc(N(c6ccccc6)c6cccc7c6[se]c6ccccc67)cc5c4c3s2)cc1 ; CC1(C)c2ccccc2-c2cc(N(c3ccc4ccc5ccc6nc(-c7ccccc7)oc6c5c4c3)c3cccc4c3[se]c3ccccc34)ccc21 ; CC1(C)c2ccccc2-c2ccc(N(c3ccc4ccc5ccc6nc(-c7ccccc7)oc6c5c4c3)c3cccc4c3[se]c3ccccc34)cc21]

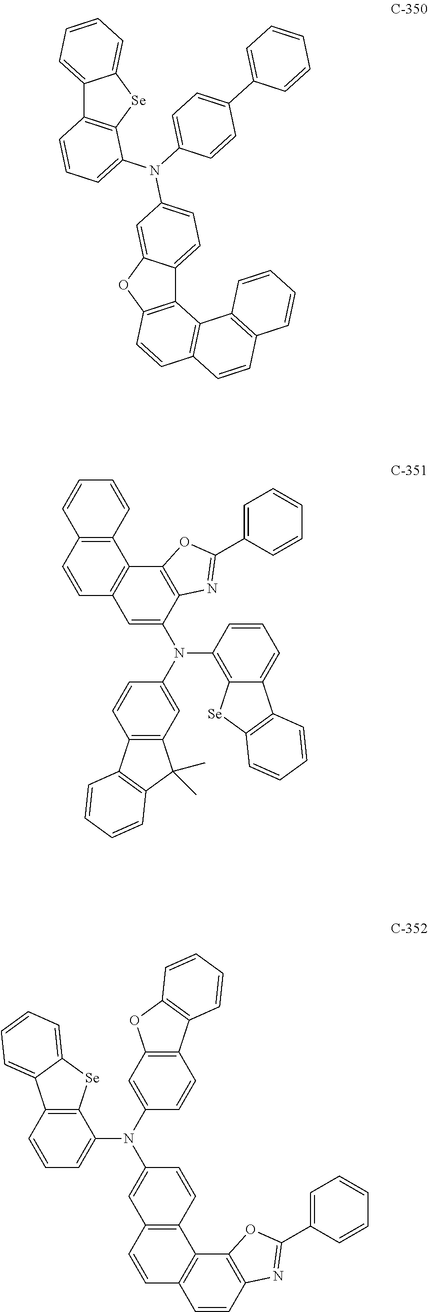 [SMILES: c1ccc(-c2ccc(N(c3ccc4c(c3)oc3ccc5ccc6ccccc6c5c34)c3cccc4c3[se]c3ccccc34)cc2)cc1 ; CC1(C)c2ccccc2-c2ccc(N(c3cc4ccc5ccccc5c4c4oc(-c5ccccc5)nc34)c3cccc4c3[se]c3ccccc34)cc21 ; c1ccc(-c2nc3ccc4ccc5cc(N(c6ccc7c(c6)oc6ccccc67)c6cccc7c6[se]c6ccccc67)ccc5c4c3o2)cc1]

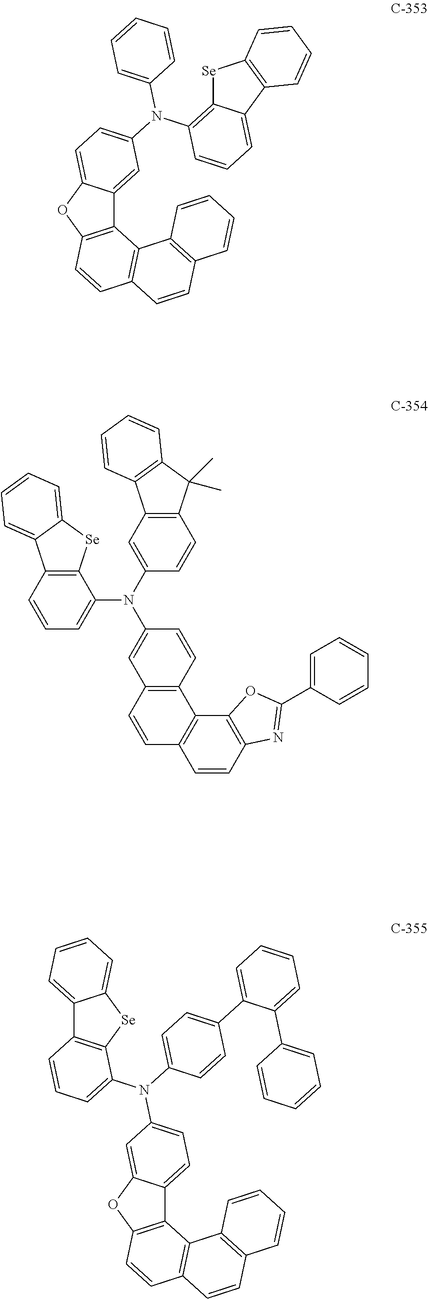 [SMILES: c1ccc(N(c2ccc3oc4ccc5ccc6ccccc6c5c4c3c2)c2cccc3c2[se]c2ccccc23)cc1 ; CC1(C)c2ccccc2-c2cc(N(c3ccc4c(ccc5ccc6nc(-c7ccccc7)oc6c54)c3)c3cccc4c3[se]c3ccccc34)ccc21 ; c1ccc(-c2ccccc2-c2ccc(N(c3ccc4c(c3)oc3ccc5ccc6ccccc6c5c34)c3cccc4c3[se]c3ccccc34)cc2)cc1]

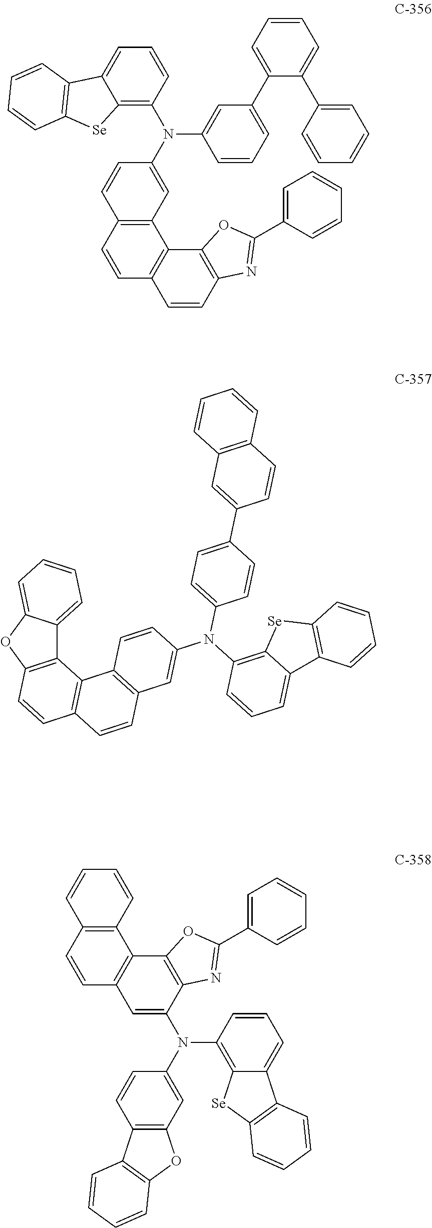 [SMILES: c1ccc(-c2nc3ccc4ccc5ccc(N(c6cccc(-c7ccccc7-c7ccccc7)c6)c6cccc7c6[se]c6ccccc67)cc5c4c3o2)cc1 ; c1ccc2cc(-c3ccc(N(c4ccc5c(ccc6ccc7oc8ccccc8c7c65)c4)c4cccc5c4[se]c4ccccc45)cc3)ccc2c1 ; c1ccc(-c2nc3c(N(c4ccc5c(c4)oc4ccccc45)c4cccc5c4[se]c4ccccc45)cc4ccc5ccccc5c4c3o2)cc1]

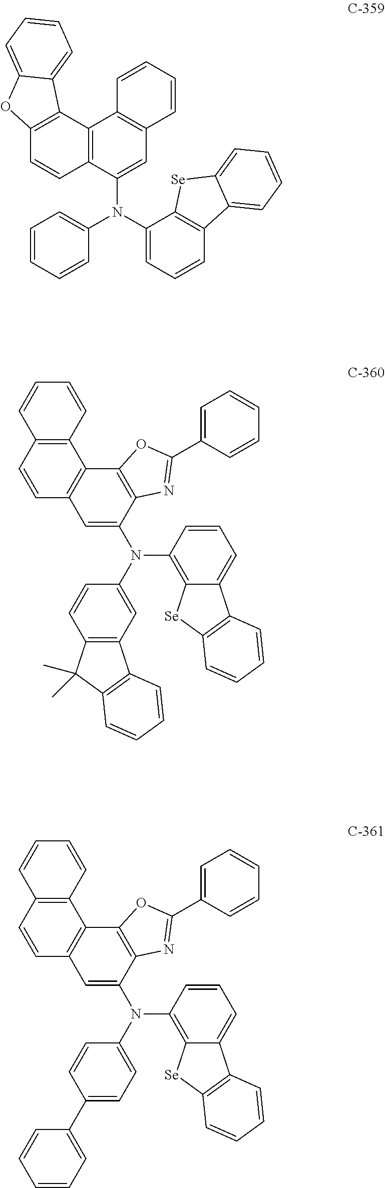 [SMILES: c1ccc(N(c2cc3ccccc3c3c2ccc2oc4ccccc4c23)c2cccc3c2[se]c2ccccc23)cc1 ; CC1(C)c2ccccc2-c2cc(N(c3cc4ccc5ccccc5c4c4oc(-c5ccccc5)nc34)c3cccc4c3[se]c3ccccc34)ccc21 ; c1ccc(-c2ccc(N(c3cc4ccc5ccccc5c4c4oc(-c5ccccc5)nc34)c3cccc4c3[se]c3ccccc34)cc2)cc1]

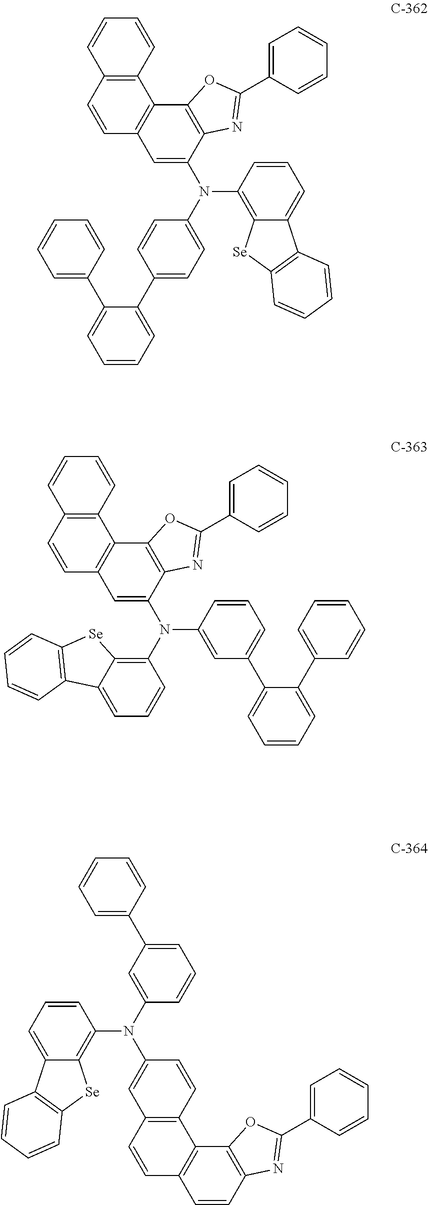 [SMILES: c1ccc(-c2nc3c(N(c4ccc(-c5ccccc5-c5ccccc5)cc4)c4cccc5c4[se]c4ccccc45)cc4ccc5ccccc5c4c3o2)cc1 ; c1ccc(-c2nc3c(N(c4cccc(-c5ccccc5-c5ccccc5)c4)c4cccc5c4[se]c4ccccc45)cc4ccc5ccccc5c4c3o2)cc1 ; c1ccc(-c2cccc(N(c3ccc4c(ccc5ccc6nc(-c7ccccc7)oc6c54)c3)c3cccc4c3[se]c3ccccc34)c2)cc1]

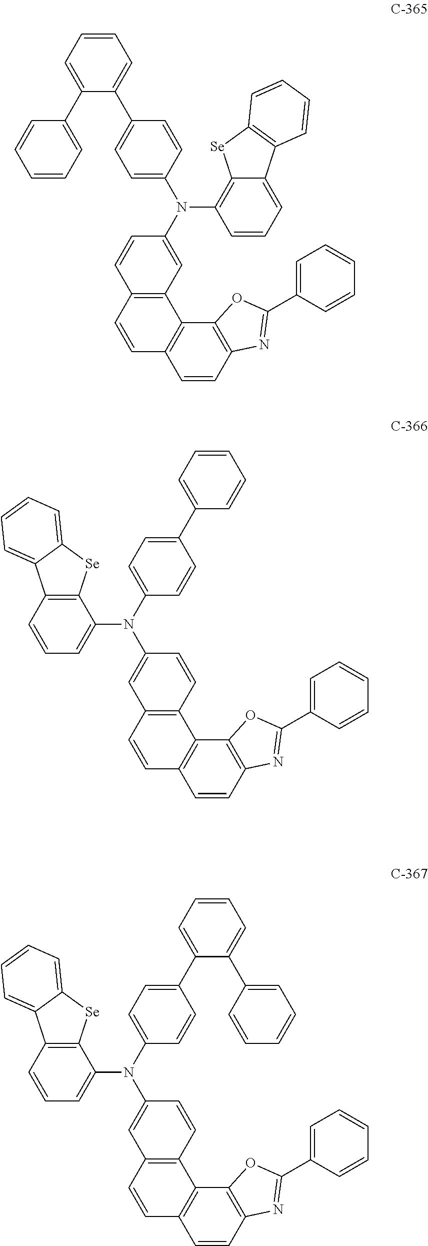 [SMILES: c1ccc(-c2nc3ccc4ccc5ccc(N(c6ccc(-c7ccccc7-c7ccccc7)cc6)c6cccc7c6[se]c6ccccc67)cc5c4c3o2)cc1 ; c1ccc(-c2ccc(N(c3ccc4c(ccc5ccc6nc(-c7ccccc7)oc6c54)c3)c3cccc4c3[se]c3ccccc34)cc2)cc1 ; c1ccc(-c2nc3ccc4ccc5cc(N(c6ccc(-c7ccccc7-c7ccccc7)cc6)c6cccc7c6[se]c6ccccc67)ccc5c4c3o2)cc1]

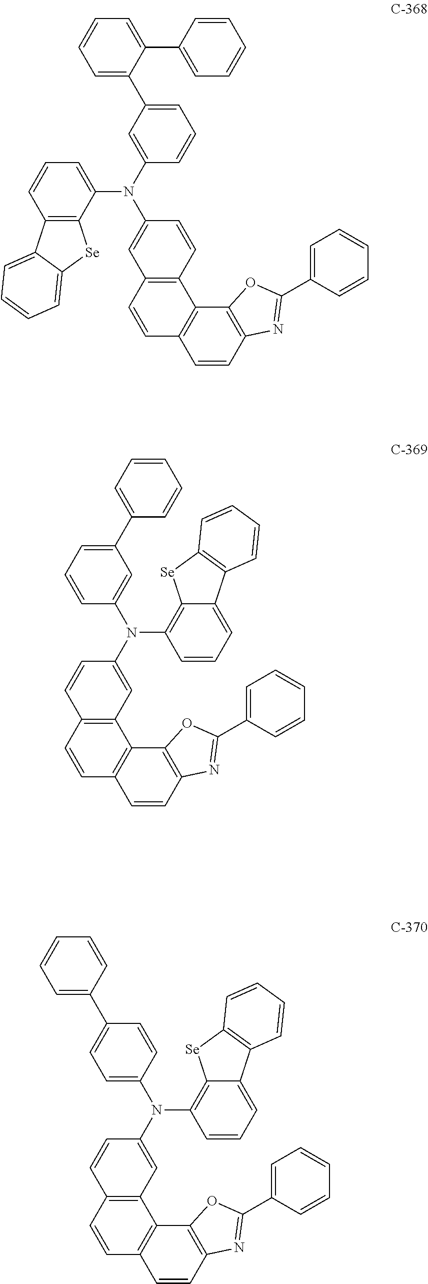 [SMILES: c1ccc(-c2nc3ccc4ccc5cc(N(c6cccc(-c7ccccc7-c7ccccc7)c6)c6cccc7c6[se]c6ccccc67)ccc5c4c3o2)cc1 ; c1ccc(-c2cccc(N(c3ccc4ccc5ccc6nc(-c7ccccc7)oc6c5c4c3)c3cccc4c3[se]c3ccccc34)c2)cc1 ; c1ccc(-c2ccc(N(c3ccc4ccc5ccc6nc(-c7ccccc7)oc6c5c4c3)c3cccc4c3[se]c3ccccc34)cc2)cc1]

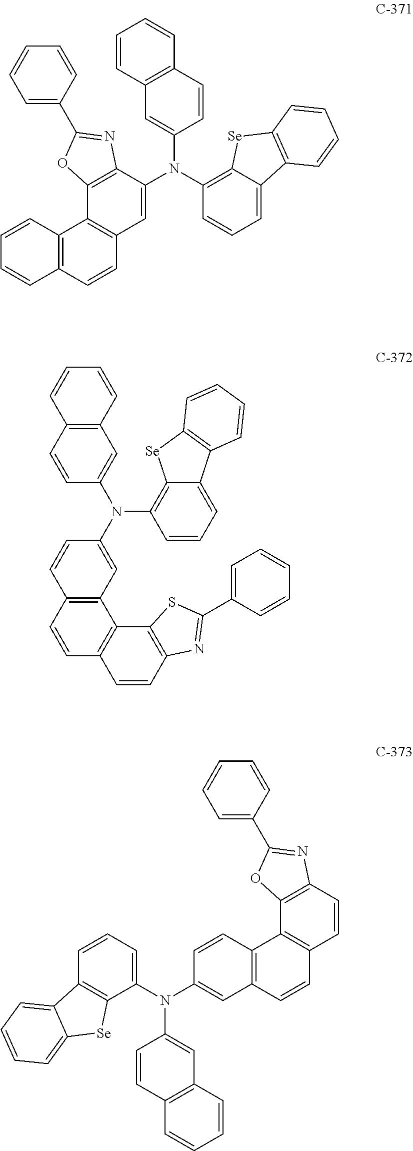 [SMILES: c1ccc(-c2nc3c(N(c4ccc5ccccc5c4)c4cccc5c4[se]c4ccccc45)cc4ccc5ccccc5c4c3o2)cc1 ; c1ccc(-c2nc3ccc4ccc5ccc(N(c6ccc7ccccc7c6)c6cccc7c6[se]c6ccccc67)cc5c4c3s2)cc1 ; c1ccc(-c2nc3ccc4ccc5cc(N(c6ccc7ccccc7c6)c6cccc7c6[se]c6ccccc67)ccc5c4c3o2)cc1]

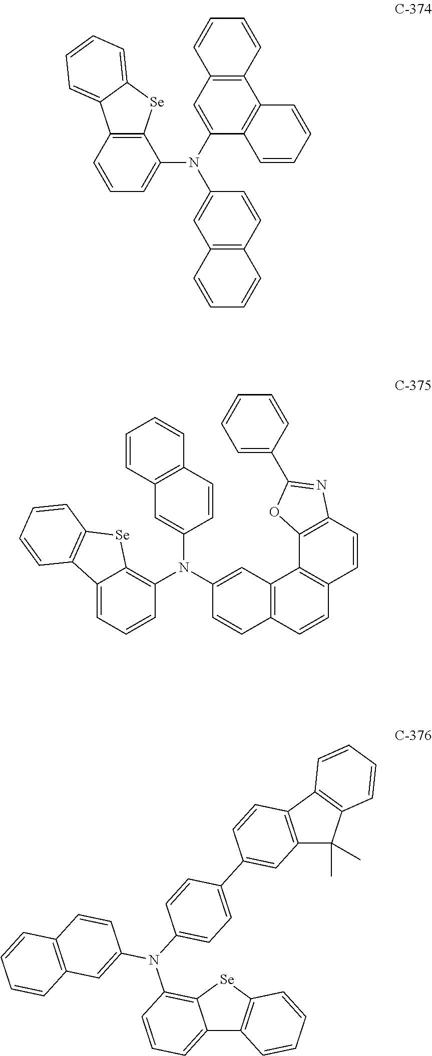 [SMILES: c1ccc2cc(N(c3cc4ccccc4c4ccccc34)c3cccc4c3[se]c3ccccc34)ccc2c1 ; c1ccc(-c2nc3ccc4ccc5ccc(N(c6ccc7ccccc7c6)c6cccc7c6[se]c6ccccc67)cc5c4c3o2)cc1 ; CC1(C)c2ccccc2-c2ccc(-c3ccc(N(c4ccc5ccccc5c4)c4cccc5c4[se]c4ccccc45)cc3)cc21]

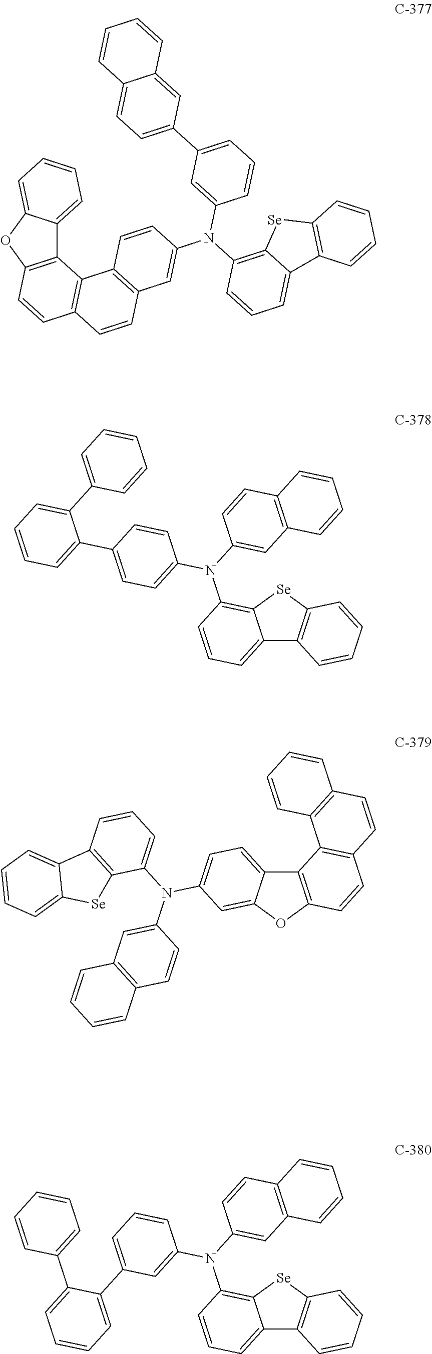 [SMILES: c1cc(-c2ccc3ccccc3c2)cc(N(c2ccc3c(ccc4ccc5oc6ccccc6c5c43)c2)c2cccc3c2[se]c2ccccc23)c1 ; c1ccc(-c2ccccc2-c2ccc(N(c3ccc4ccccc4c3)c3cccc4c3[se]c3ccccc34)cc2)cc1 ; c1ccc2cc(N(c3ccc4c(c3)oc3ccc5ccc6ccccc6c5c34)c3cccc4c3[se]c3ccccc34)ccc2c1 ; c1ccc(-c2ccccc2-c2cccc(N(c3ccc4ccccc4c3)c3cccc4c3[se]c3ccccc34)c2)cc1]

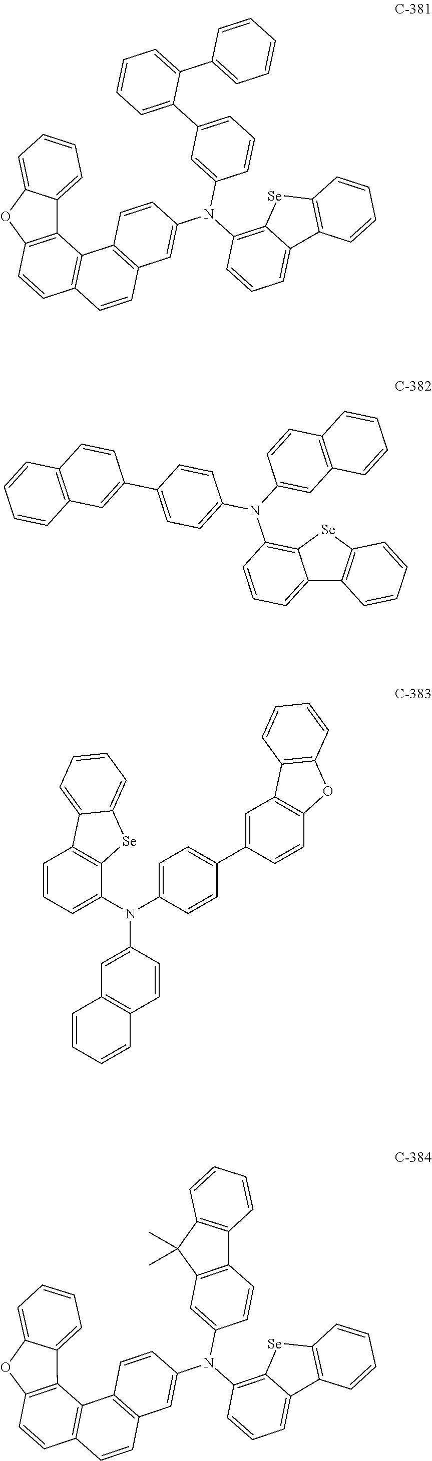 [SMILES: c1ccc(-c2ccccc2-c2cccc(N(c3ccc4c(ccc5ccc6oc7ccccc7c6c54)c3)c3cccc4c3[se]c3ccccc34)c2)cc1 ; c1ccc2cc(-c3ccc(N(c4ccc5ccccc5c4)c4cccc5c4[se]c4ccccc45)cc3)ccc2c1 ; c1ccc2cc(N(c3ccc(-c4ccc5oc6ccccc6c5c4)cc3)c3cccc4c3[se]c3ccccc34)ccc2c1 ; CC1(C)c2ccccc2-c2ccc(N(c3ccc4c(ccc5ccc6oc7ccccc7c6c54)c3)c3cccc4c3[se]c3ccccc34)cc21]

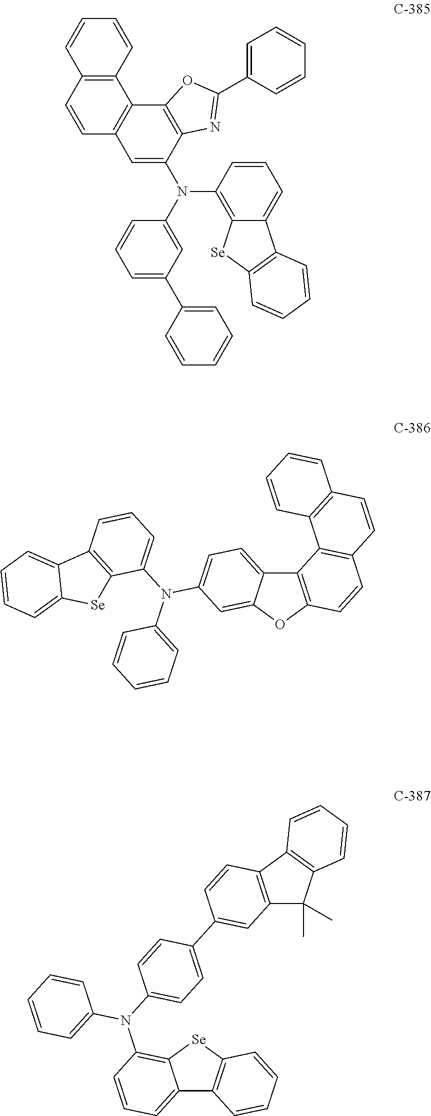 [SMILES: c1ccc(-c2cccc(N(c3cc4ccc5ccccc5c4c4oc(-c5ccccc5)nc34)c3cccc4c3[se]c3ccccc34)c2)cc1 ; c1ccc(N(c2ccc3c(c2)oc2ccc4ccc5ccccc5c4c23)c2cccc3c2[se]c2ccccc23)cc1 ; CC1(C)c2ccccc2-c2ccc(-c3ccc(N(c4ccccc4)c4cccc5c4[se]c4ccccc45)cc3)cc21]

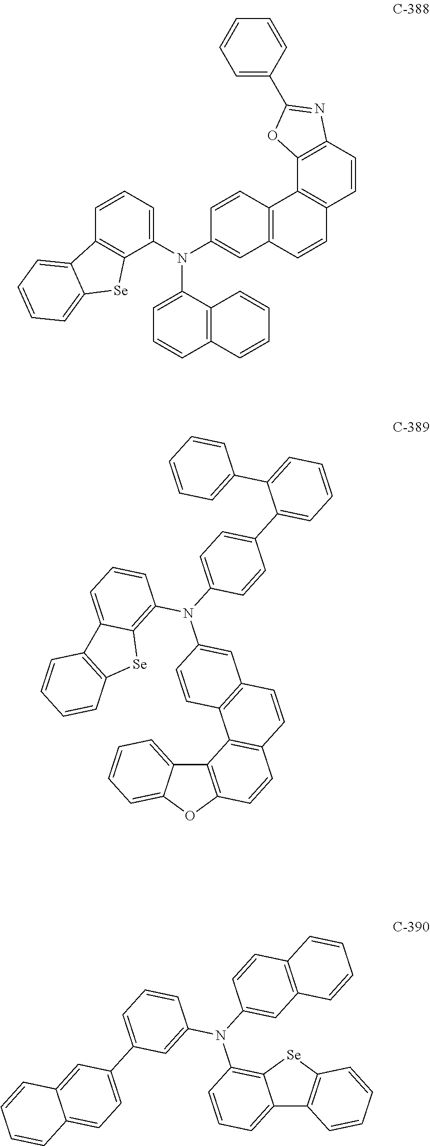 [SMILES: c1ccc(-c2nc3ccc4ccc5cc(N(c6cccc7ccccc67)c6cccc7c6[se]c6ccccc67)ccc5c4c3o2)cc1 ; c1ccc(-c2ccccc2-c2ccc(N(c3ccc4c(ccc5ccc6oc7ccccc7c6c54)c3)c3cccc4c3[se]c3ccccc34)cc2)cc1 ; c1cc(-c2ccc3ccccc3c2)cc(N(c2ccc3ccccc3c2)c2cccc3c2[se]c2ccccc23)c1]

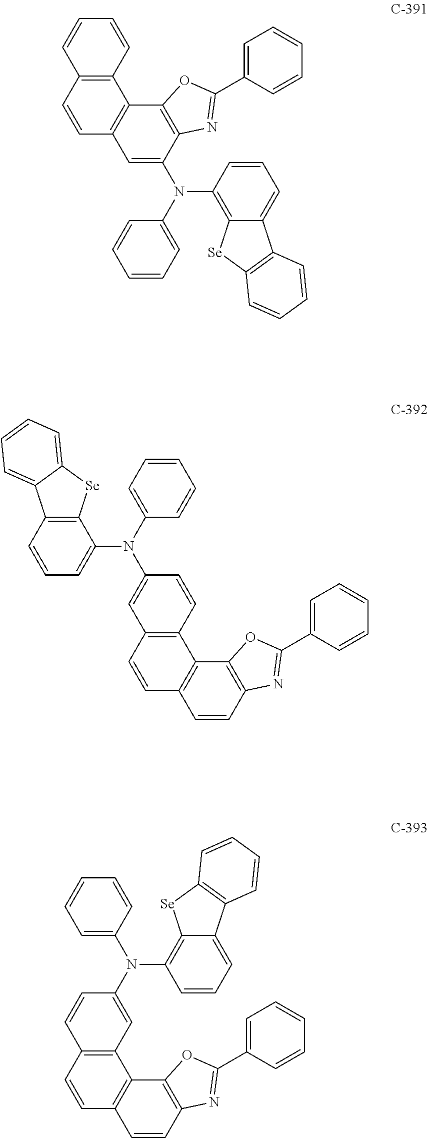 [SMILES: c1ccc(-c2nc3c(N(c4ccccc4)c4cccc5c4[se]c4ccccc45)cc4ccc5ccccc5c4c3o2)cc1 ; c1ccc(-c2nc3ccc4ccc5cc(N(c6ccccc6)c6cccc7c6[se]c6ccccc67)ccc5c4c3o2)cc1 ; c1ccc(-c2nc3ccc4ccc5ccc(N(c6ccccc6)c6cccc7c6[se]c6ccccc67)cc5c4c3o2)cc1]

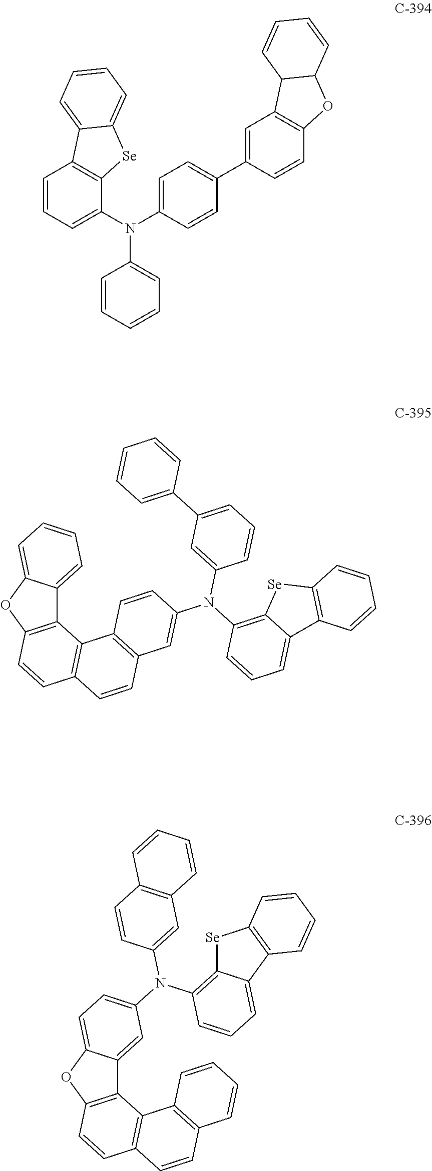 [SMILES: C1=CC2Oc3ccc(-c4ccc(N(c5ccccc5)c5cccc6c5[se]c5ccccc56)cc4)cc3C2C=C1 ; c1ccc(-c2cccc(N(c3ccc4c(ccc5ccc6oc7ccccc7c6c54)c3)c3cccc4c3[se]c3ccccc34)c2)cc1 ; c1ccc2cc(N(c3ccc4oc5ccc6ccc7ccccc7c6c5c4c3)c3cccc4c3[se]c3ccccc34)ccc2c1]

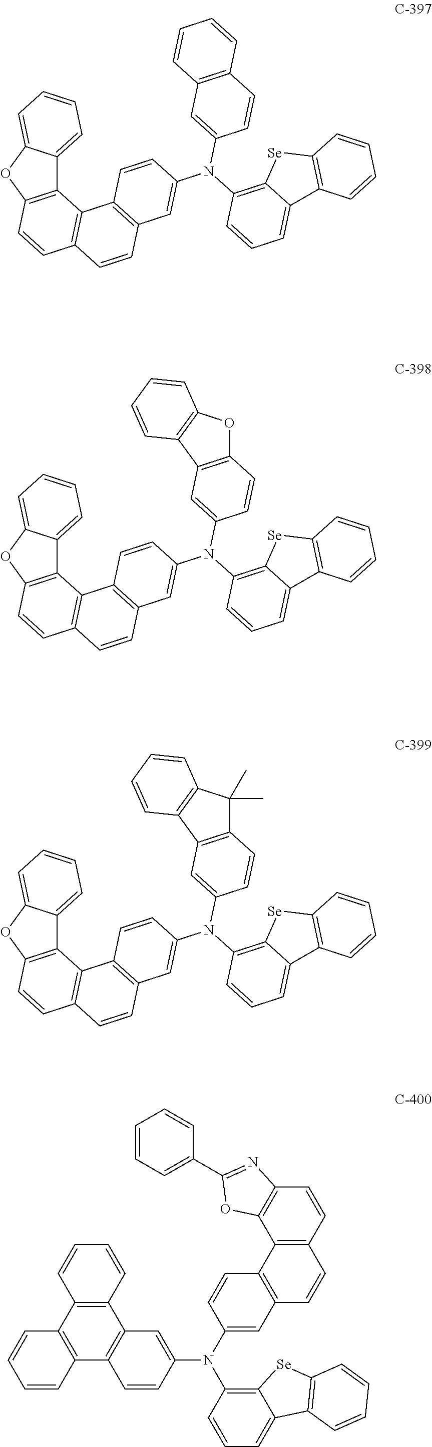 [SMILES: c1ccc2cc(N(c3ccc4c(ccc5ccc6oc7ccccc7c6c54)c3)c3cccc4c3[se]c3ccccc34)ccc2c1 ; c1ccc2c(c1)oc1ccc(N(c3ccc4c(ccc5ccc6oc7ccccc7c6c54)c3)c3cccc4c3[se]c3ccccc34)cc12 ; CC1(C)c2ccccc2-c2cc(N(c3ccc4c(ccc5ccc6oc7ccccc7c6c54)c3)c3cccc4c3[se]c3ccccc34)ccc21 ; c1ccc(-c2nc3ccc4ccc5cc(N(c6ccc7c8ccccc8c8ccccc8c7c6)c6cccc7c6[se]c6ccccc67)ccc5c4c3o2)cc1]

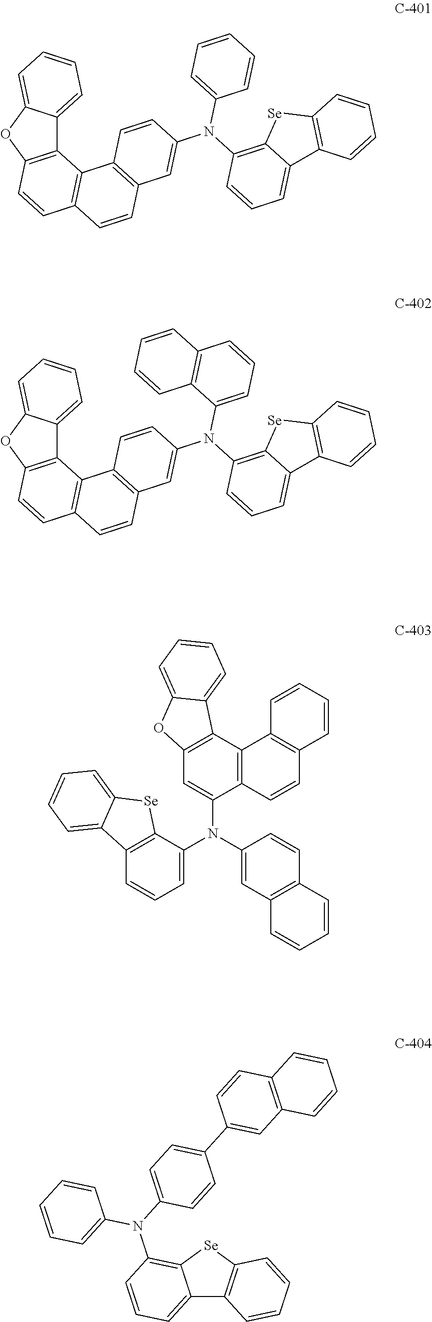 [SMILES: c1ccc(N(c2ccc3c(ccc4ccc5oc6ccccc6c5c43)c2)c2cccc3c2[se]c2ccccc23)cc1 ; c1ccc2c(N(c3ccc4c(ccc5ccc6oc7ccccc7c6c54)c3)c3cccc4c3[se]c3ccccc34)cccc2c1 ; c1ccc2cc(N(c3cc4oc5ccccc5c4c4c3ccc3ccccc34)c3cccc4c3[se]c3ccccc34)ccc2c1 ; c1ccc(N(c2ccc(-c3ccc4ccccc4c3)cc2)c2cccc3c2[se]c2ccccc23)cc1]

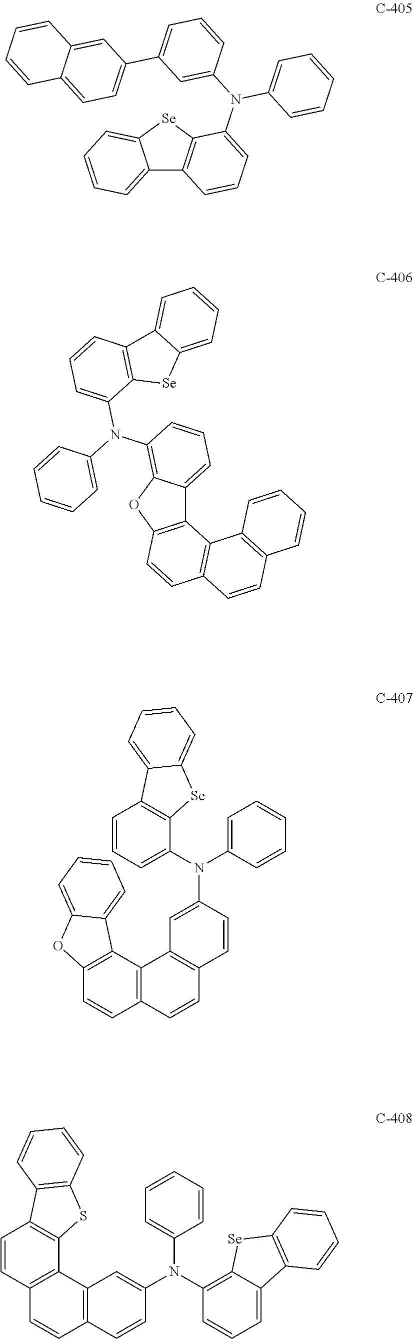 [SMILES: c1ccc(N(c2cccc(-c3ccc4ccccc4c3)c2)c2cccc3c2[se]c2ccccc23)cc1 ; c1ccc(N(c2cccc3c2oc2ccc4ccc5ccccc5c4c23)c2cccc3c2[se]c2ccccc23)cc1 ; c1ccc(N(c2ccc3ccc4ccc5oc6ccccc6c5c4c3c2)c2cccc3c2[se]c2ccccc23)cc1 ; c1ccc(N(c2ccc3ccc4ccc5c6ccccc6sc5c4c3c2)c2cccc3c2[se]c2ccccc23)cc1]

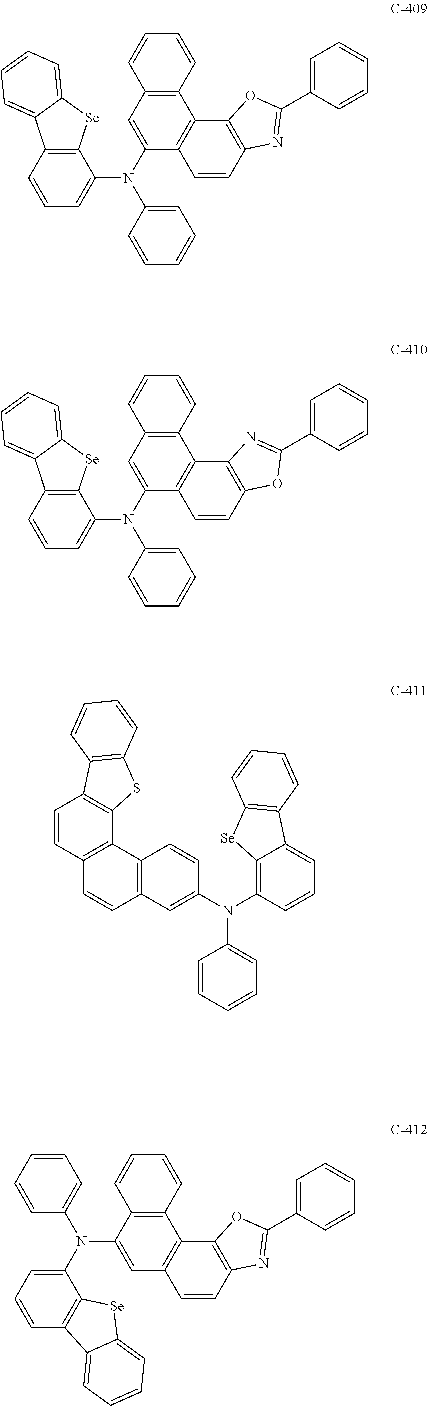 [SMILES: c1ccc(-c2nc3ccc4c(N(c5ccccc5)c5cccc6c5[se]c5ccccc56)cc5ccccc5c4c3o2)cc1 ; c1ccc(-c2nc3c(ccc4c(N(c5ccccc5)c5cccc6c5[se]c5ccccc56)cc5ccccc5c43)o2)cc1 ; c1ccc(N(c2ccc3c(ccc4ccc5c6ccccc6sc5c43)c2)c2cccc3c2[se]c2ccccc23)cc1 ; c1ccc(-c2nc3ccc4cc(N(c5ccccc5)c5cccc6c5[se]c5ccccc56)c5ccccc5c4c3o2)cc1]

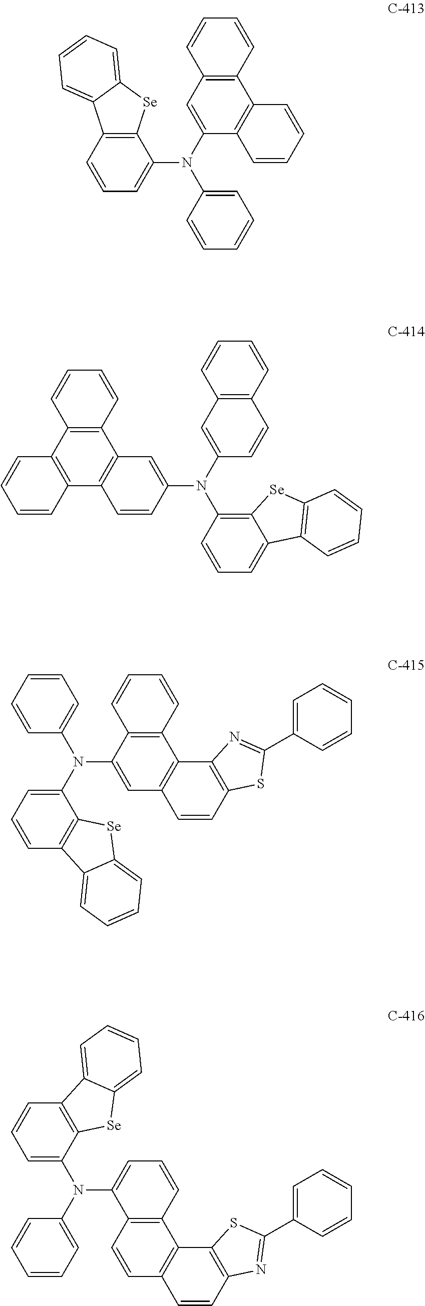 [SMILES: c1ccc(N(c2cc3ccccc3c3ccccc23)c2cccc3c2[se]c2ccccc23)cc1 ; c1ccc2cc(N(c3ccc4c5ccccc5c5ccccc5c4c3)c3cccc4c3[se]c3ccccc34)ccc2c1 ; c1ccc(-c2nc3c(ccc4cc(N(c5ccccc5)c5cccc6c5[se]c5ccccc56)c5ccccc5c43)s2)cc1 ; c1ccc(-c2nc3ccc4ccc5c(N(c6ccccc6)c6cccc7c6[se]c6ccccc67)cccc5c4c3s2)cc1]

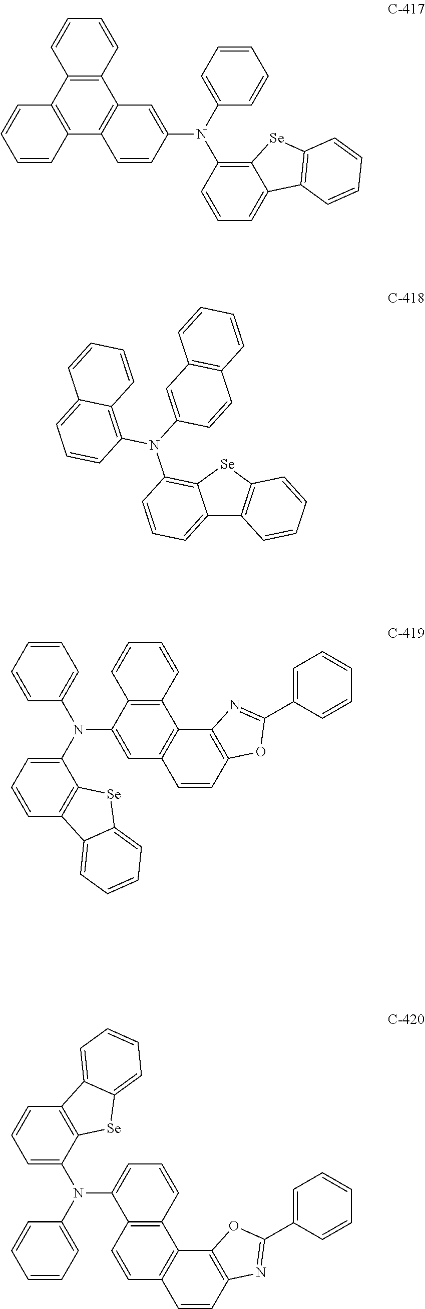 [SMILES: c1ccc(N(c2ccc3c4ccccc4c4ccccc4c3c2)c2cccc3c2[se]c2ccccc23)cc1 ; c1ccc2cc(N(c3cccc4ccccc34)c3cccc4c3[se]c3ccccc34)ccc2c1 ; c1ccc(-c2nc3c(ccc4cc(N(c5ccccc5)c5cccc6c5[se]c5ccccc56)c5ccccc5c43)o2)cc1 ; c1ccc(-c2nc3ccc4ccc5c(N(c6ccccc6)c6cccc7c6[se]c6ccccc67)cccc5c4c3o2)cc1]

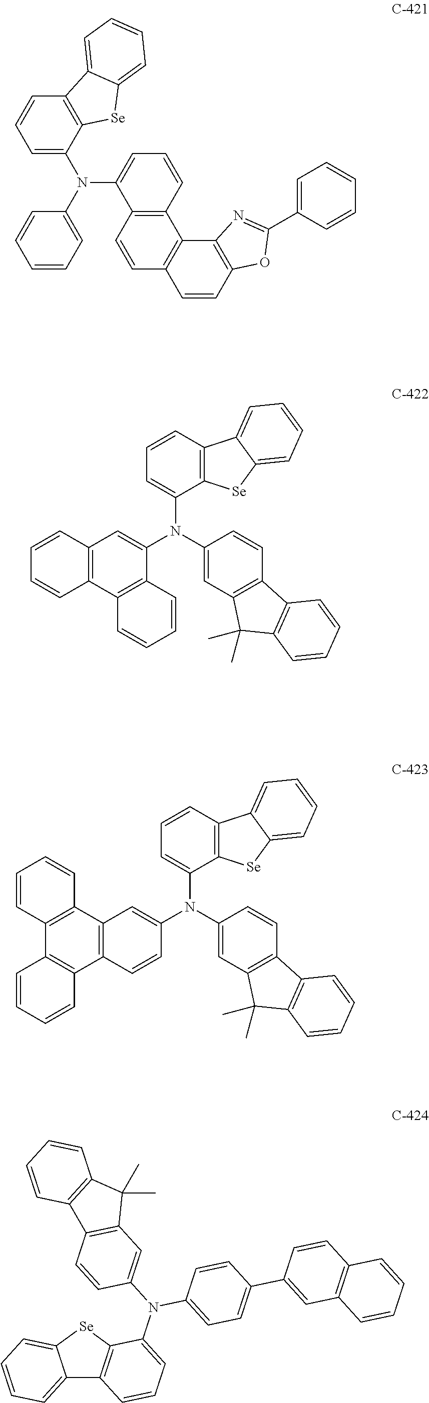 [SMILES: c1ccc(-c2nc3c(ccc4ccc5c(N(c6ccccc6)c6cccc7c6[se]c6ccccc67)cccc5c43)o2)cc1 ; CC1(C)c2ccccc2-c2ccc(N(c3cc4ccccc4c4ccccc34)c3cccc4c3[se]c3ccccc34)cc21 ; CC1(C)c2ccccc2-c2ccc(N(c3ccc4c5ccccc5c5ccccc5c4c3)c3cccc4c3[se]c3ccccc34)cc21 ; CC1(C)c2ccccc2-c2ccc(N(c3ccc(-c4ccc5ccccc5c4)cc3)c3cccc4c3[se]c3ccccc34)cc21]

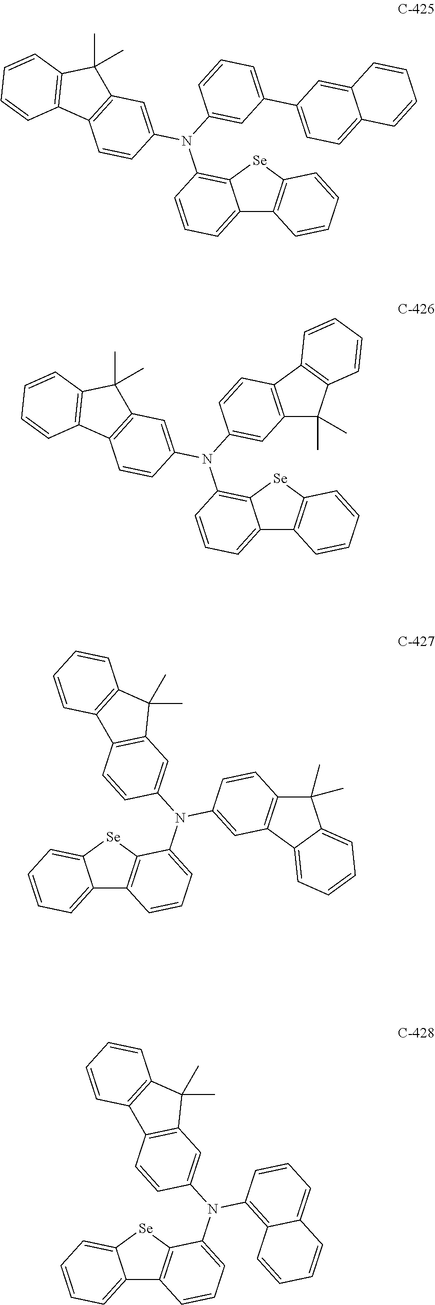 [SMILES: CC1(C)c2ccccc2-c2ccc(N(c3cccc(-c4ccc5ccccc5c4)c3)c3cccc4c3[se]c3ccccc34)cc21 ; CC1(C)c2ccccc2-c2ccc(N(c3ccc4c(c3)C(C)(C)c3ccccc3-4)c3cccc4c3[se]c3ccccc34)cc21 ; CC1(C)c2ccccc2-c2cc(N(c3ccc4c(c3)C(C)(C)c3ccccc3-4)c3cccc4c3[se]c3ccccc34)ccc21 ; CC1(C)c2ccccc2-c2ccc(N(c3cccc4ccccc34)c3cccc4c3[se]c3ccccc34)cc21]

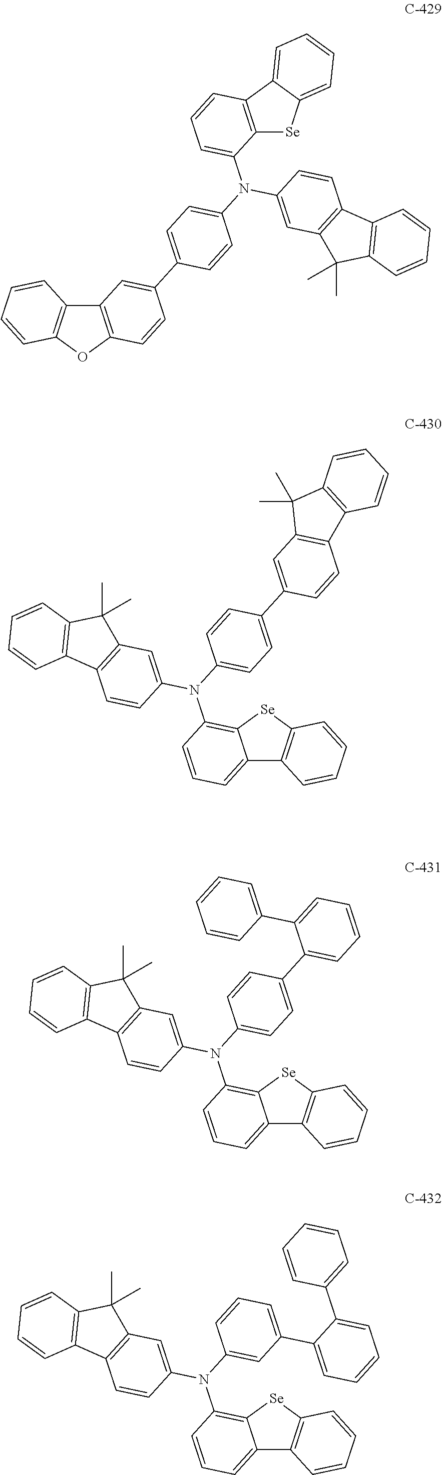 [SMILES: CC1(C)c2ccccc2-c2ccc(N(c3ccc(-c4ccc5oc6ccccc6c5c4)cc3)c3cccc4c3[se]c3ccccc34)cc21 ; CC1(C)c2ccccc2-c2ccc(-c3ccc(N(c4ccc5c(c4)C(C)(C)c4ccccc4-5)c4cccc5c4[se]c4ccccc45)cc3)cc21 ; CC1(C)c2ccccc2-c2ccc(N(c3ccc(-c4ccccc4-c4ccccc4)cc3)c3cccc4c3[se]c3ccccc34)cc21 ; CC1(C)c2ccccc2-c2ccc(N(c3cccc(-c4ccccc4-c4ccccc4)c3)c3cccc4c3[se]c3ccccc34)cc21]

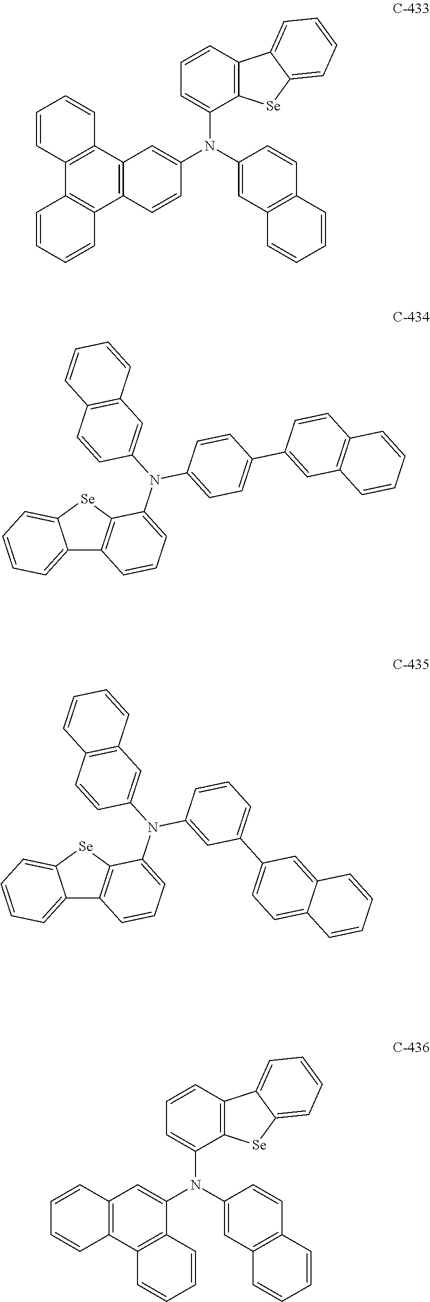 [SMILES: c1ccc2cc(N(c3ccc4c5ccccc5c5ccccc5c4c3)c3cccc4c3[se]c3ccccc34)ccc2c1 ; c1ccc2cc(-c3ccc(N(c4ccc5ccccc5c4)c4cccc5c4[se]c4ccccc45)cc3)ccc2c1 ; c1cc(-c2ccc3ccccc3c2)cc(N(c2ccc3ccccc3c2)c2cccc3c2[se]c2ccccc23)c1 ; c1ccc2cc(N(c3cc4ccccc4c4ccccc34)c3cccc4c3[se]c3ccccc34)ccc2c1]

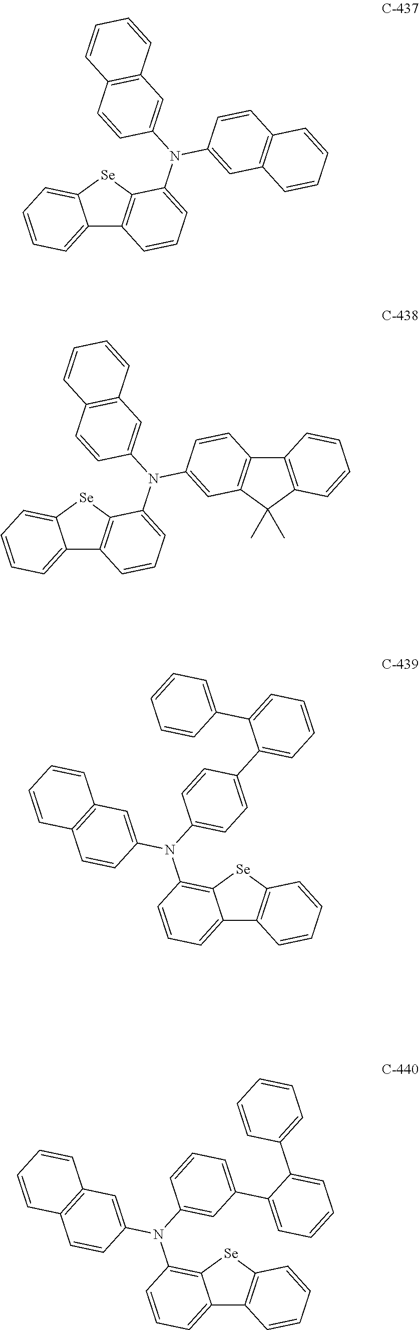 [SMILES: c1ccc2cc(N(c3ccc4ccccc4c3)c3cccc4c3[se]c3ccccc34)ccc2c1 ; CC1(C)c2ccccc2-c2ccc(N(c3ccc4ccccc4c3)c3cccc4c3[se]c3ccccc34)cc21 ; c1ccc(-c2ccccc2-c2ccc(N(c3ccc4ccccc4c3)c3cccc4c3[se]c3ccccc34)cc2)cc1 ; c1ccc(-c2ccccc2-c2cccc(N(c3ccc4ccccc4c3)c3cccc4c3[se]c3ccccc34)c2)cc1]

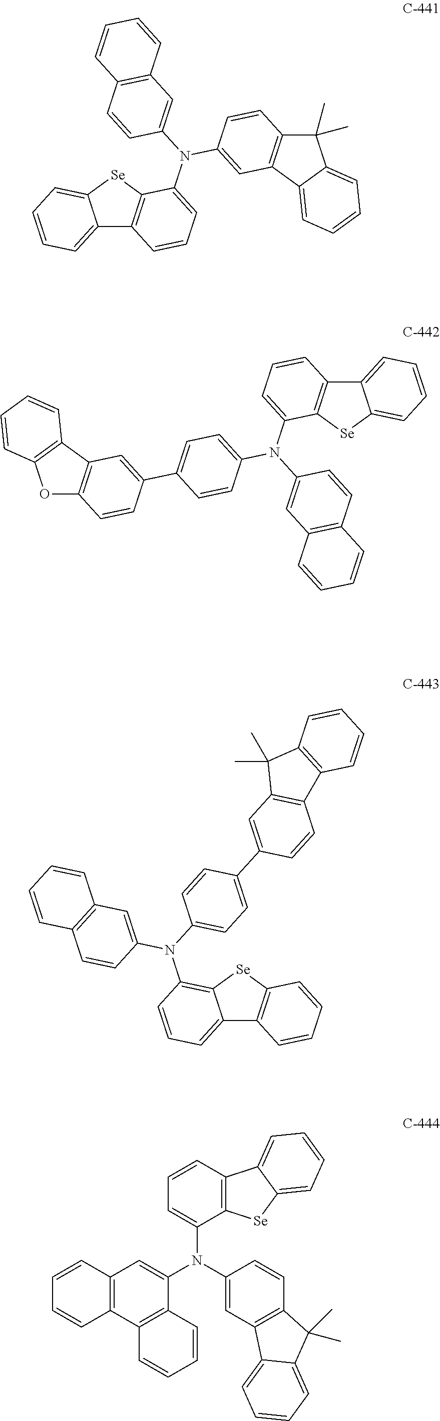 [SMILES: CC1(C)c2ccccc2-c2cc(N(c3ccc4ccccc4c3)c3cccc4c3[se]c3ccccc34)ccc21 ; c1ccc2cc(N(c3ccc(-c4ccc5oc6ccccc6c5c4)cc3)c3cccc4c3[se]c3ccccc34)ccc2c1 ; CC1(C)c2ccccc2-c2ccc(-c3ccc(N(c4ccc5ccccc5c4)c4cccc5c4[se]c4ccccc45)cc3)cc21 ; CC1(C)c2ccccc2-c2cc(N(c3cc4ccccc4c4ccccc34)c3cccc4c3[se]c3ccccc34)ccc21]

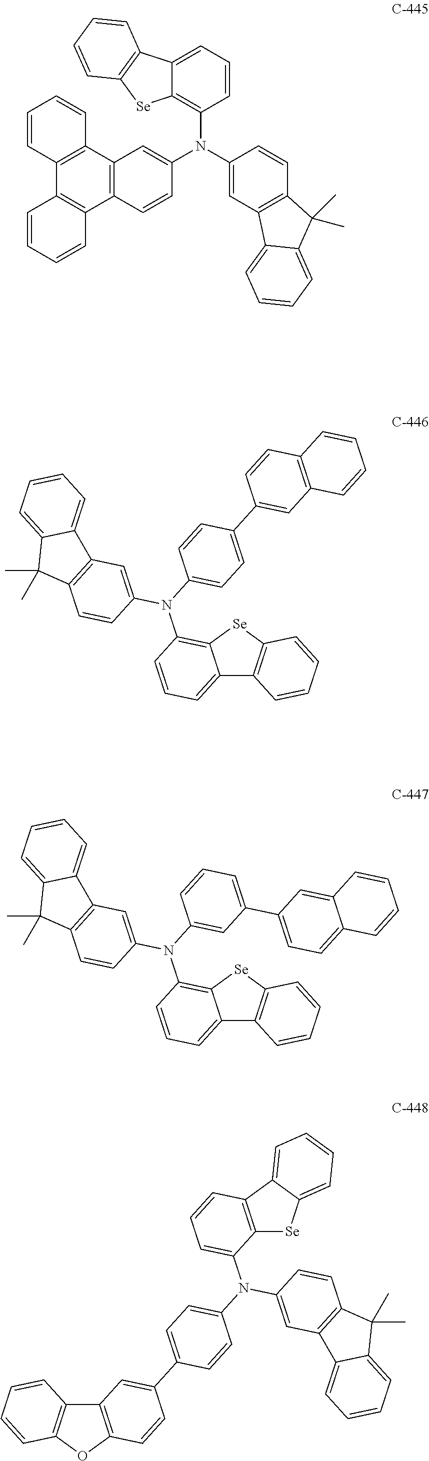

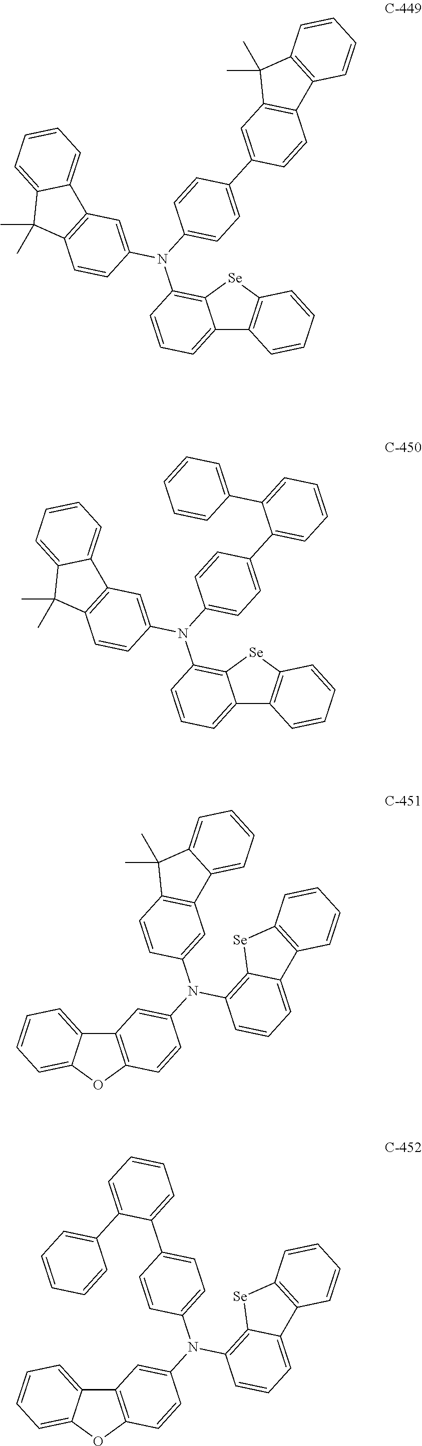 [SMILES: CC1(C)c2ccccc2-c2cc(N(c3ccc(-c4ccc5c(c4)C(C)(C)c4ccccc4-5)cc3)c3cccc4c3[se]c3ccccc34)ccc21 ; CC1(C)c2ccccc2-c2cc(N(c3ccc(-c4ccccc4-c4ccccc4)cc3)c3cccc4c3[se]c3ccccc34)ccc21 ; CC1(C)c2ccccc2-c2cc(N(c3ccc4oc5ccccc5c4c3)c3cccc4c3[se]c3ccccc34)ccc21 ; c1ccc(-c2ccccc2-c2ccc(N(c3ccc4oc5ccccc5c4c3)c3cccc4c3[se]c3ccccc34)cc2)cc1]

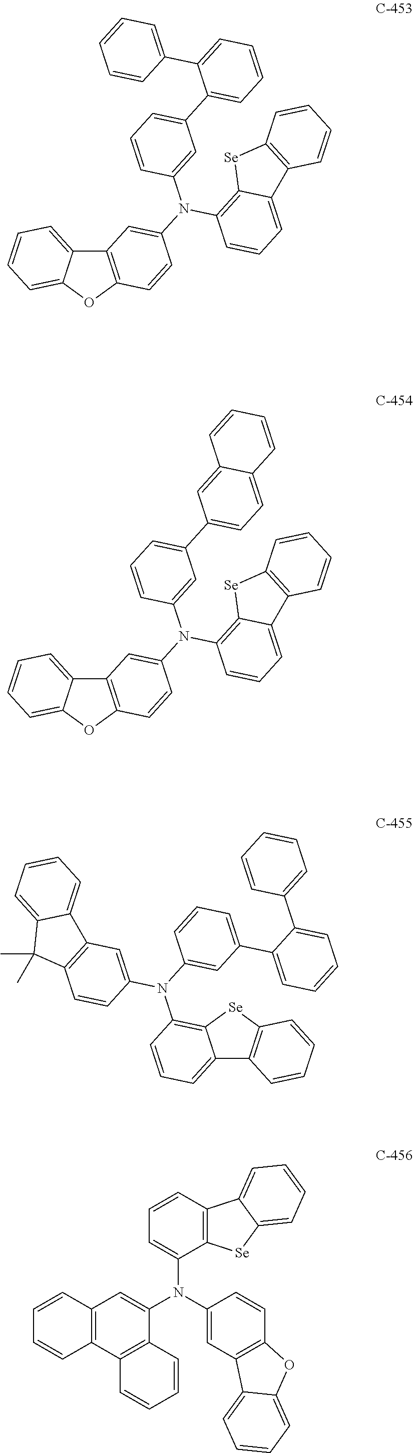 [SMILES: c1ccc(-c2ccccc2-c2cccc(N(c3ccc4oc5ccccc5c4c3)c3cccc4c3[se]c3ccccc34)c2)cc1 ; c1cc(-c2ccc3ccccc3c2)cc(N(c2ccc3oc4ccccc4c3c2)c2cccc3c2[se]c2ccccc23)c1 ; CC1(C)c2ccccc2-c2cc(N(c3cccc(-c4ccccc4-c4ccccc4)c3)c3cccc4c3[se]c3ccccc34)ccc21 ; c1ccc2c(c1)cc(N(c1ccc3oc4ccccc4c3c1)c1cccc3c1[se]c1ccccc13)c1ccccc12]

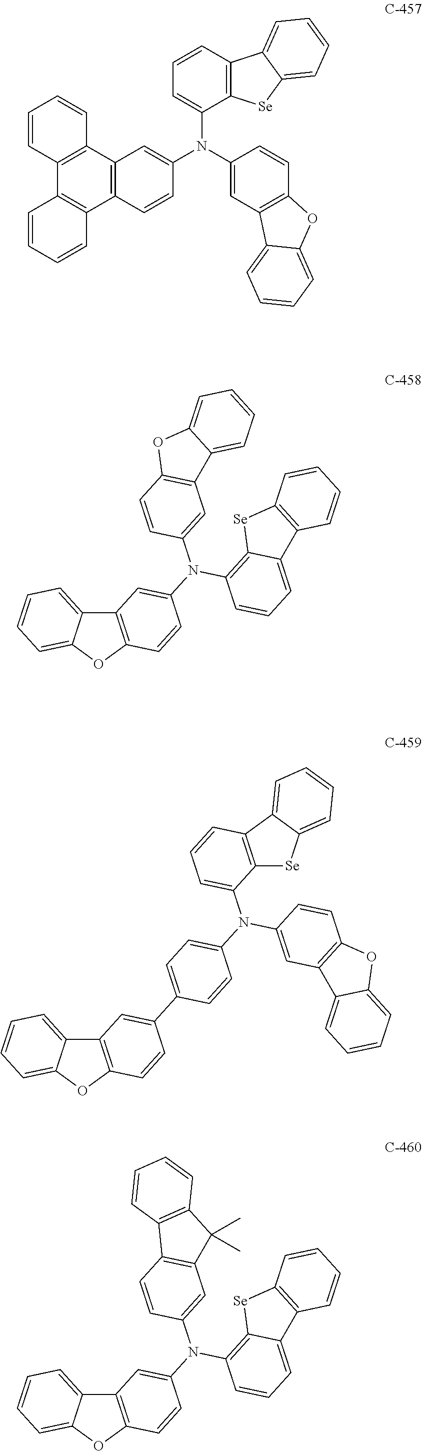 [SMILES: c1ccc2c(c1)oc1ccc(N(c3ccc4c5ccccc5c5ccccc5c4c3)c3cccc4c3[se]c3ccccc34)cc12 ; c1ccc2c(c1)oc1ccc(N(c3ccc4oc5ccccc5c4c3)c3cccc4c3[se]c3ccccc34)cc12 ; c1ccc2c(c1)oc1ccc(-c3ccc(N(c4ccc5oc6ccccc6c5c4)c4cccc5c4[se]c4ccccc45)cc3)cc12 ; CC1(C)c2ccccc2-c2ccc(N(c3ccc4oc5ccccc5c4c3)c3cccc4c3[se]c3ccccc34)cc21]

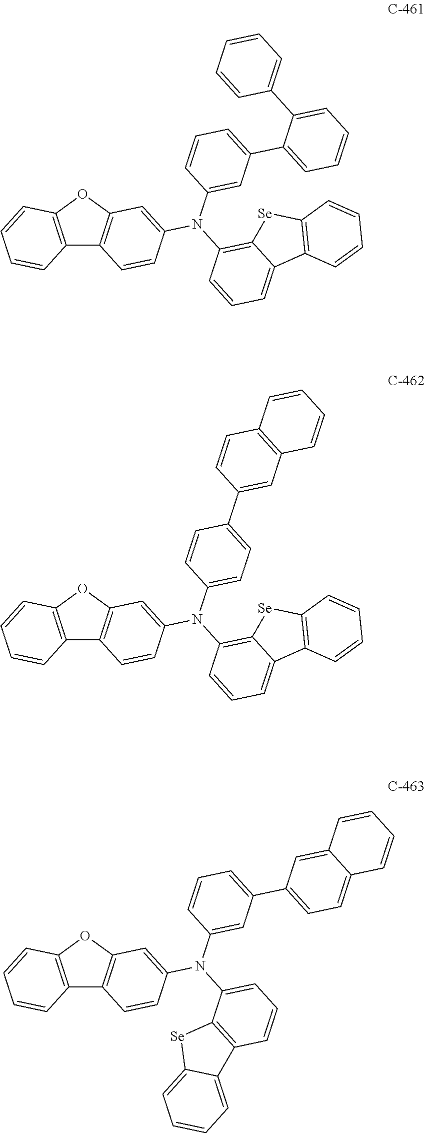 [SMILES: c1ccc(-c2ccccc2-c2cccc(N(c3ccc4c(c3)oc3ccccc34)c3cccc4c3[se]c3ccccc34)c2)cc1 ; c1ccc2cc(-c3ccc(N(c4ccc5c(c4)oc4ccccc45)c4cccc5c4[se]c4ccccc45)cc3)ccc2c1 ; c1cc(-c2ccc3ccccc3c2)cc(N(c2ccc3c(c2)oc2ccccc23)c2cccc3c2[se]c2ccccc23)c1]

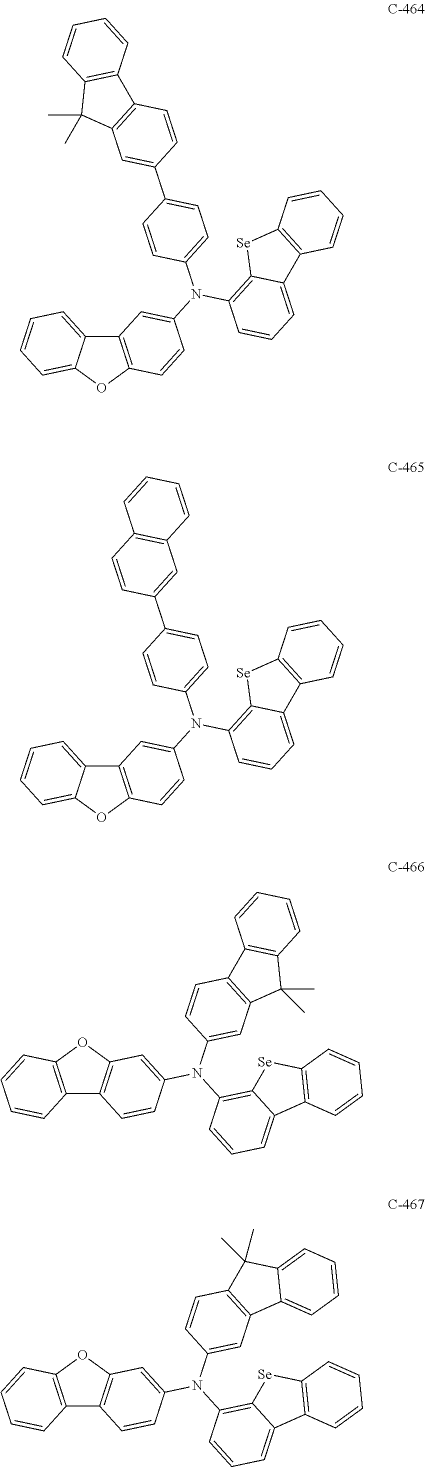 [SMILES: CC1(C)c2ccccc2-c2ccc(-c3ccc(N(c4ccc5oc6ccccc6c5c4)c4cccc5c4[se]c4ccccc45)cc3)cc21 ; c1ccc2cc(-c3ccc(N(c4ccc5oc6ccccc6c5c4)c4cccc5c4[se]c4ccccc45)cc3)ccc2c1 ; CC1(C)c2ccccc2-c2ccc(N(c3ccc4c(c3)oc3ccccc34)c3cccc4c3[se]c3ccccc34)cc21 ; CC1(C)c2ccccc2-c2cc(N(c3ccc4c(c3)oc3ccccc34)c3cccc4c3[se]c3ccccc34)ccc21]

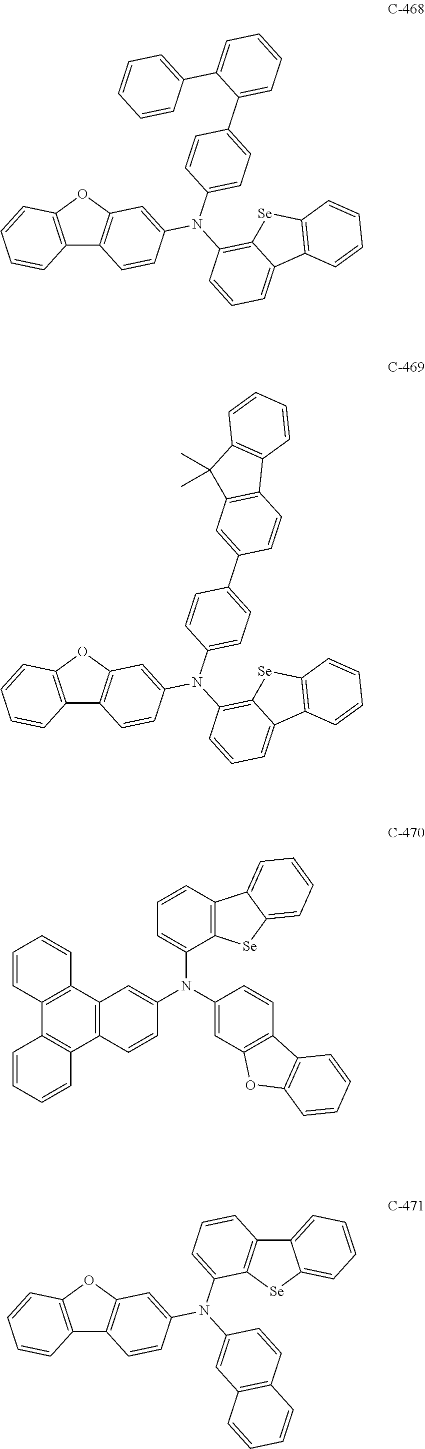 [SMILES: c1ccc(-c2ccccc2-c2ccc(N(c3ccc4c(c3)oc3ccccc34)c3cccc4c3[se]c3ccccc34)cc2)cc1 ; CC1(C)c2ccccc2-c2ccc(-c3ccc(N(c4ccc5c(c4)oc4ccccc45)c4cccc5c4[se]c4ccccc45)cc3)cc21 ; c1ccc2c(c1)oc1cc(N(c3ccc4c5ccccc5c5ccccc5c4c3)c3cccc4c3[se]c3ccccc34)ccc12 ; c1ccc2cc(N(c3ccc4c(c3)oc3ccccc34)c3cccc4c3[se]c3ccccc34)ccc2c1]

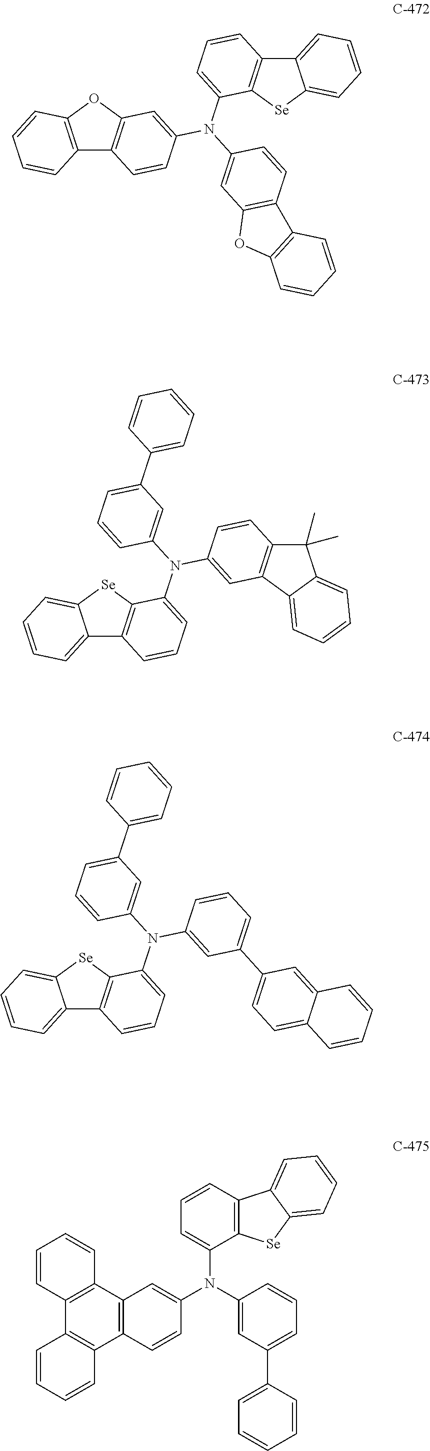 [SMILES: c1ccc2c(c1)oc1cc(N(c3ccc4c(c3)oc3ccccc34)c3cccc4c3[se]c3ccccc34)ccc12 ; CC1(C)c2ccccc2-c2cc(N(c3cccc(-c4ccccc4)c3)c3cccc4c3[se]c3ccccc34)ccc21 ; c1ccc(-c2cccc(N(c3cccc(-c4ccc5ccccc5c4)c3)c3cccc4c3[se]c3ccccc34)c2)cc1 ; c1ccc(-c2cccc(N(c3ccc4c5ccccc5c5ccccc5c4c3)c3cccc4c3[se]c3ccccc34)c2)cc1]

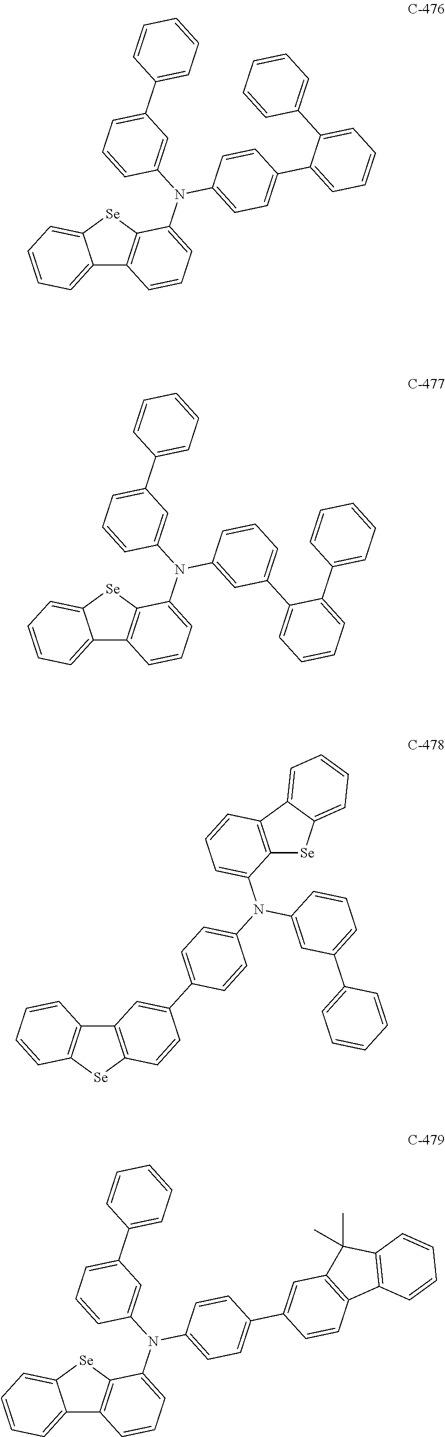 [SMILES: c1ccc(-c2cccc(N(c3ccc(-c4ccccc4-c4ccccc4)cc3)c3cccc4c3[se]c3ccccc34)c2)cc1 ; c1ccc(-c2cccc(N(c3cccc(-c4ccccc4-c4ccccc4)c3)c3cccc4c3[se]c3ccccc34)c2)cc1 ; c1ccc(-c2cccc(N(c3ccc(-c4ccc5[se]c6ccccc6c5c4)cc3)c3cccc4c3[se]c3ccccc34)c2)cc1 ; CC1(C)c2ccccc2-c2ccc(-c3ccc(N(c4cccc(-c5ccccc5)c4)c4cccc5c4[se]c4ccccc45)cc3)cc21]

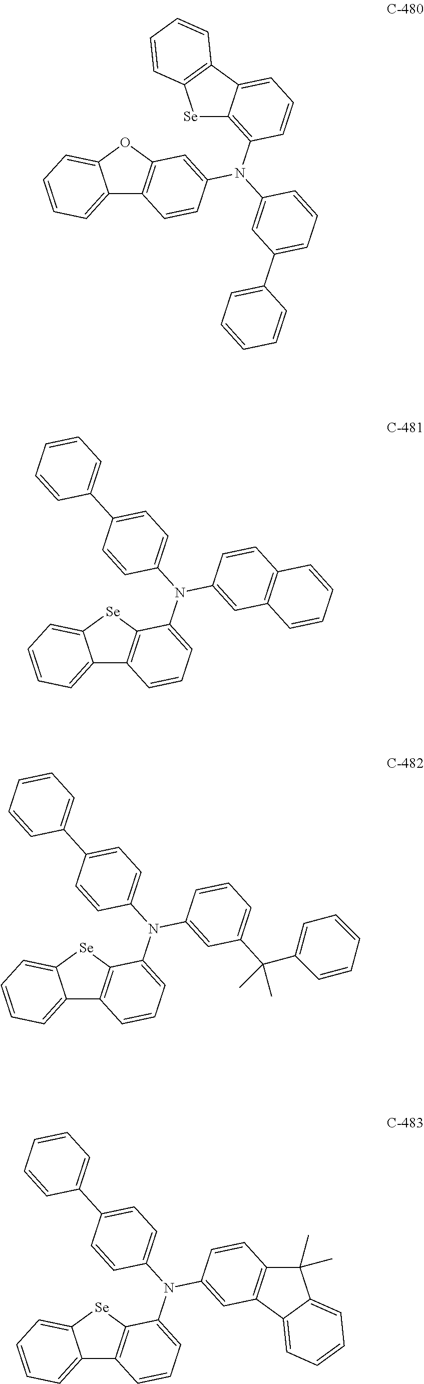 [SMILES: c1ccc(-c2cccc(N(c3ccc4c(c3)oc3ccccc34)c3cccc4c3[se]c3ccccc34)c2)cc1 ; c1ccc(-c2ccc(N(c3ccc4ccccc4c3)c3cccc4c3[se]c3ccccc34)cc2)cc1 ; CC(C)(c1ccccc1)c1cccc(N(c2ccc(-c3ccccc3)cc2)c2cccc3c2[se]c2ccccc23)c1 ; CC1(C)c2ccccc2-c2cc(N(c3ccc(-c4ccccc4)cc3)c3cccc4c3[se]c3ccccc34)ccc21]

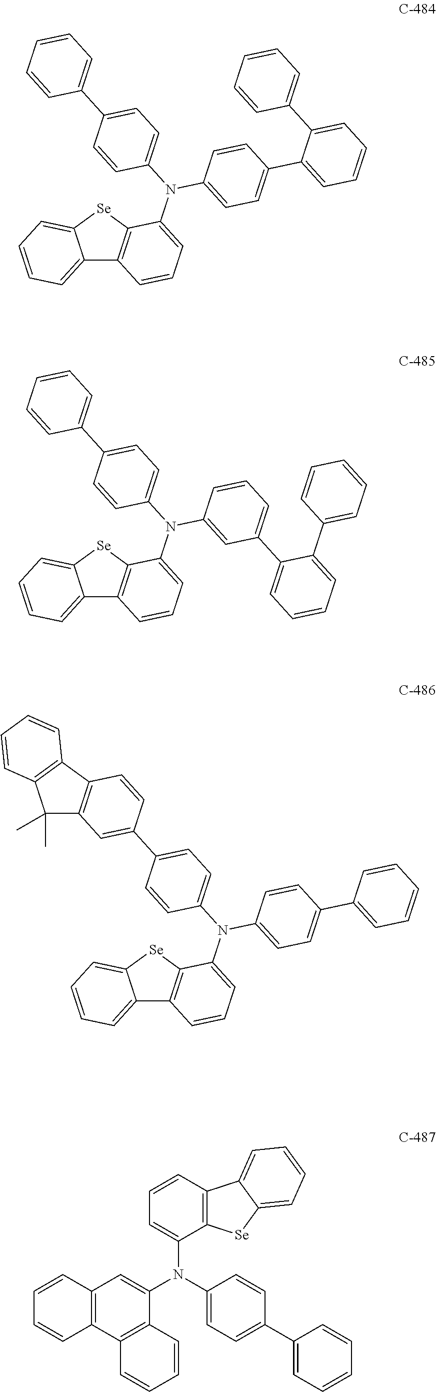 [SMILES: c1ccc(-c2ccc(N(c3ccc(-c4ccccc4-c4ccccc4)cc3)c3cccc4c3[se]c3ccccc34)cc2)cc1 ; c1ccc(-c2ccc(N(c3cccc(-c4ccccc4-c4ccccc4)c3)c3cccc4c3[se]c3ccccc34)cc2)cc1 ; CC1(C)c2ccccc2-c2ccc(-c3ccc(N(c4ccc(-c5ccccc5)cc4)c4cccc5c4[se]c4ccccc45)cc3)cc21 ; c1ccc(-c2ccc(N(c3cc4ccccc4c4ccccc34)c3cccc4c3[se]c3ccccc34)cc2)cc1]

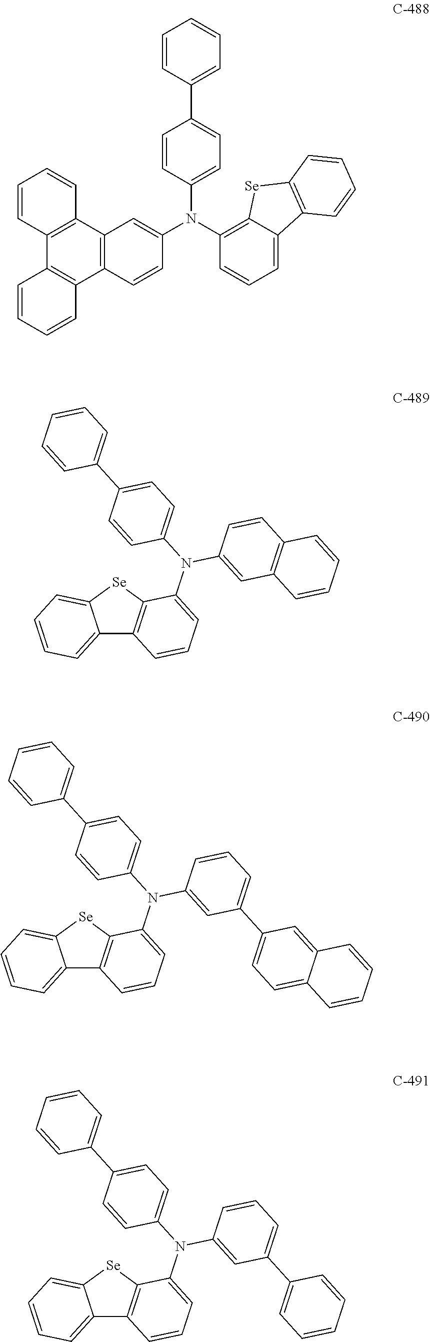 [SMILES: c1ccc(-c2ccc(N(c3ccc4c5ccccc5c5ccccc5c4c3)c3cccc4c3[se]c3ccccc34)cc2)cc1 ; c1ccc(-c2ccc(N(c3ccc4ccccc4c3)c3cccc4c3[se]c3ccccc34)cc2)cc1 ; c1ccc(-c2ccc(N(c3cccc(-c4ccc5ccccc5c4)c3)c3cccc4c3[se]c3ccccc34)cc2)cc1 ; c1ccc(-c2ccc(N(c3cccc(-c4ccccc4)c3)c3cccc4c3[se]c3ccccc34)cc2)cc1]

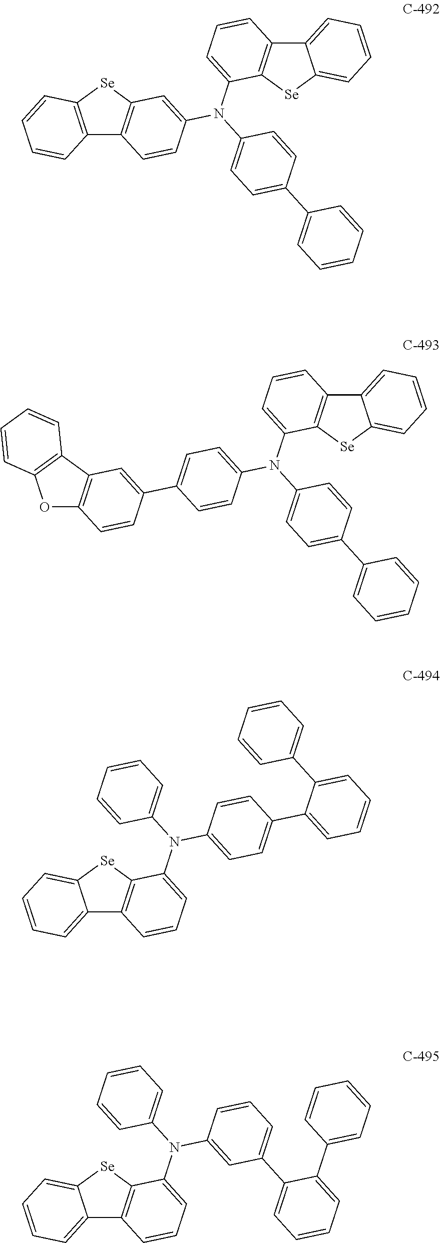 [SMILES: c1ccc(-c2ccc(N(c3ccc4c(c3)[se]c3ccccc34)c3cccc4c3[se]c3ccccc34)cc2)cc1 ; c1ccc(-c2ccc(N(c3ccc(-c4ccc5oc6ccccc6c5c4)cc3)c3cccc4c3[se]c3ccccc34)cc2)cc1 ; c1ccc(-c2ccccc2-c2ccc(N(c3ccccc3)c3cccc4c3[se]c3ccccc34)cc2)cc1 ; c1ccc(-c2ccccc2-c2cccc(N(c3ccccc3)c3cccc4c3[se]c3ccccc34)c2)cc1]

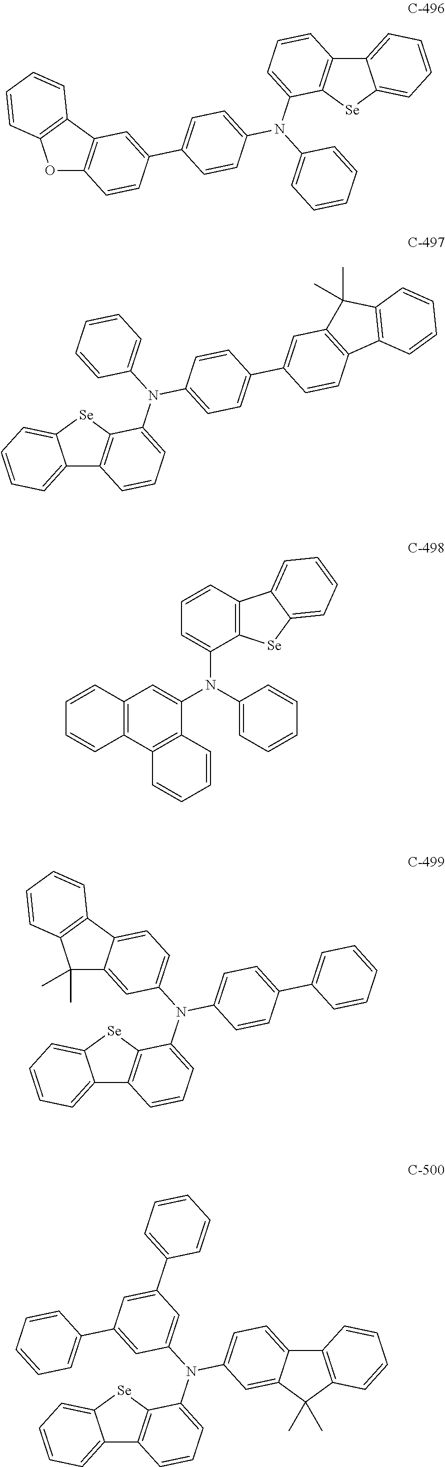 [SMILES: c1ccc(N(c2ccc(-c3ccc4oc5ccccc5c4c3)cc2)c2cccc3c2[se]c2ccccc23)cc1 ; CC1(C)c2ccccc2-c2ccc(-c3ccc(N(c4ccccc4)c4cccc5c4[se]c4ccccc45)cc3)cc21 ; c1ccc(N(c2cc3ccccc3c3ccccc23)c2cccc3c2[se]c2ccccc23)cc1 ; CC1(C)c2ccccc2-c2ccc(N(c3ccc(-c4ccccc4)cc3)c3cccc4c3[se]c3ccccc34)cc21 ; CC1(C)c2ccccc2-c2ccc(N(c3cc(-c4ccccc4)cc(-c4ccccc4)c3)c3cccc4c3[se]c3ccccc34)cc21]

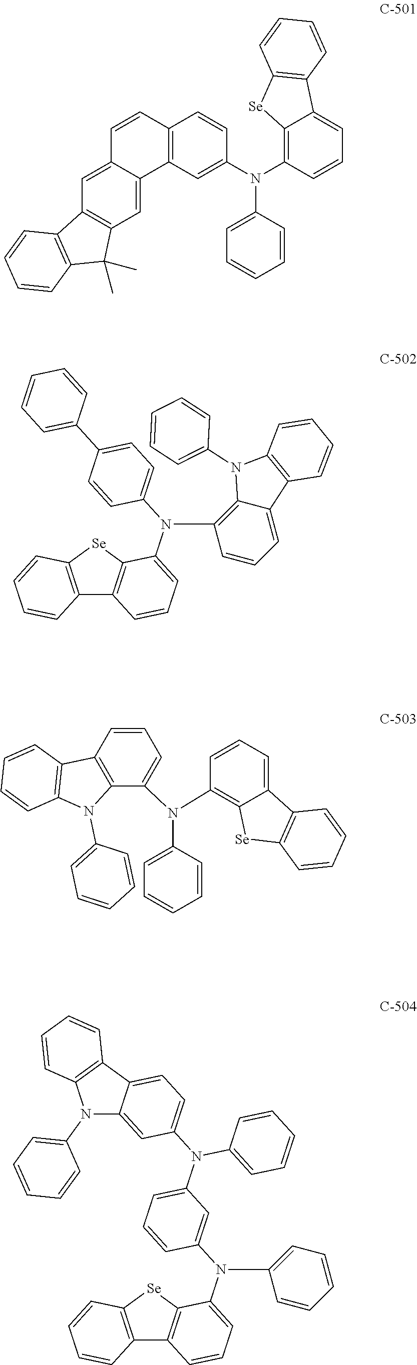 [SMILES: CC1(C)c2ccccc2-c2cc3ccc4ccc(N(c5ccccc5)c5cccc6c5[se]c5ccccc56)cc4c3cc21 ; c1ccc(-c2ccc(N(c3cccc4c3[se]c3ccccc34)c3cccc4c5ccccc5n(-c5ccccc5)c34)cc2)cc1 ; c1ccc(N(c2cccc3c2[se]c2ccccc23)c2cccc3c4ccccc4n(-c4ccccc4)c23)cc1 ; c1ccc(N(c2cccc(N(c3ccccc3)c3cccc4c3[se]c3ccccc34)c2)c2ccc3c4ccccc4n(-c4ccccc4)c3c2)cc1]

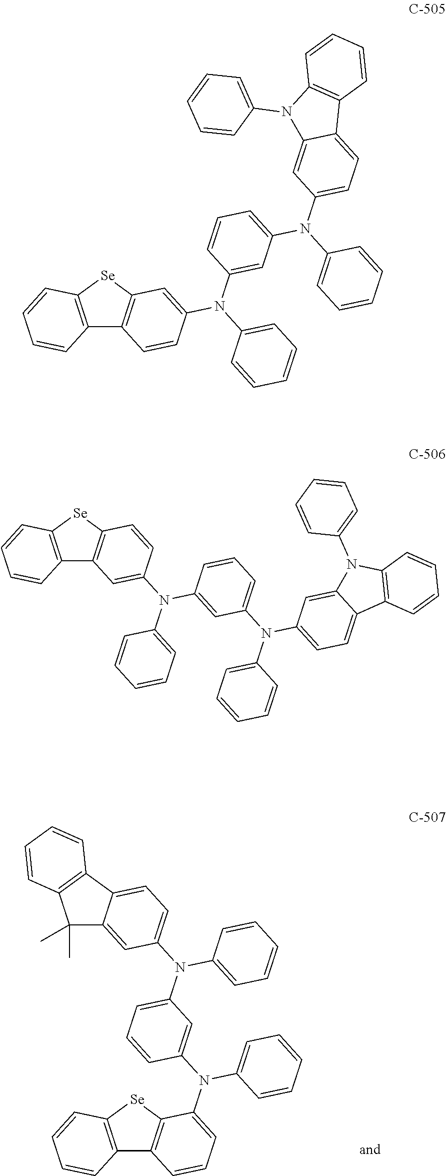 [SMILES: c1ccc(N(c2cccc(N(c3ccccc3)c3ccc4c5ccccc5n(-c5ccccc5)c4c3)c2)c2ccc3c(c2)[se]c2ccccc23)cc1 ; c1ccc(N(c2cccc(N(c3ccccc3)c3ccc4c5ccccc5n(-c5ccccc5)c4c3)c2)c2ccc3[se]c4ccccc4c3c2)cc1 ; CC1(C)c2ccccc2-c2ccc(N(c3ccccc3)c3cccc(N(c4ccccc4)c4cccc5c4[se]c4ccccc45)c3)cc21]

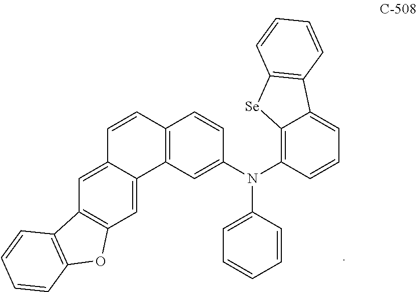 [SMILES: c1ccc(N(c2ccc3ccc4cc5c(cc4c3c2)oc2ccccc25)c2cccc3c2[se]c2ccccc23)cc1]

8. An organic electroluminescent material comprising the organic electroluminescent compound according to claim 1.

9. An organic electroluminescent device comprising the organic electroluminescent compound according to claim 1.

10. The organic electroluminescent device according to claim 9, wherein the organic electroluminescent compound is comprised in at least one layer of a light-emitting layer, a hole transport layer, and a hole auxiliary layer.
